(19) United States
(12) Patent Application Publication
(10) Pub. No.: US 2003/0096952 A1
(43) Pub. Date: May 22, 2003

(54) Novel proteins and nucleic acids encoding same

(76) Inventors: Kumud Majumder, Stamford, CT (US); Steven K. Spaderna, Berlin, CT (US); Raymond J. Taupier JR., Stamford, CT (US); Muralidhara Padigaru, Bronx, NY (US); Catherine Burgess, Wethersfield, CT (US); Richard A. Shimkets, West Haven, CT (US); Kimberly A. Spytek, New Haven, CT (US); Xiaohong Liu, Branford, CT (US); Meera Patturajan, Branford, CT (US); Vladimir Y. Gusev, Madison, CT (US)

Correspondence Address: Ivor R. Elrifi, Mintz, Levin, Cohn, Ferris,, Glovsky and Popeo, P.C., One Financial Center, Boston, MA, 02111, US

(21) Appl. No.: 09/823,187

(22) Filed: Mar. 29, 2001

Related U.S. Application Data
(60) Provisional application No. 60/193,339, filed on Mar. 30, 2000.
(60) Provisional application No. 60/193,205, filed on Mar. 30, 2000.
(60) Provisional application No. 60/195,343, filed on Apr. 5, 2000.
(60) Provisional application No. 60/195,088, filed on Apr. 6, 2000.
(60) Provisional application No. 60/195,005, filed on Apr. 6, 2000.
(60) Provisional application No. 60/195,792, filed on Apr. 10, 2000.
(60) Provisional application No. 60/196,556, filed on Apr. 11, 2000.
(60) Provisional application No. 60/197,081, filed on Apr. 13, 2000.
(60) Provisional application No. 60/197,525, filed on Apr. 14, 2000.
(60) Provisional application No. 60/197,087, filed on Apr. 14, 2000.

(51) Int. Cl. C07K 14/705; C07H 21/04; C12P 21/02; C12N 5/06

(52) U.S. Cl. 530/350; 536/23.5; 435/325; 435/320.1; 435/69.1

(57) ABSTRACT

Disclosed herein are nucleic acid sequences that encode G-coupled protein-receptor related polypeptides. Also disclosed are polypeptides encoded by these nucleic acid sequences, and antibodies, which immunospecifically-bind to the polypeptide, as well as derivatives, variants, mutants, or fragments of the aforementioned polypeptide, polynucleotide, or antibody. The invention further discloses therapeutic, diagnostic and research methods for diagnosis, treatment, and prevention of disorders involving any one of these novel human nucleic acids and proteins.

### RELATED APPLICATIONS

This application claims priority from Provisional Applications U.S. Ser. No. 60/193,339, filed Mar. 30, 2000; U.S. Ser. No. 60/193,205, filed Mar. 30, 2000; U.S. Ser. No. 60/195,343, filed Apr. 5, 2000; U.S. Ser. No. 60/195,088, filed Apr. 6, 2000; U.S. Ser. No. 60/195,005, filed Apr. 6, 2000; U.S. Ser. No. 60/195,792, filed Apr. 10, 2000; U.S. Ser. No. 60/196,556, filed Apr. 11, 2000; U.S. Ser. No. 60/197,081, filed Apr. 13, 2000; U.S. Ser. No. 60/197,525, filed Apr. 14, 2000; and U.S. Ser. No. 60/197,087, filed on Apr. 14, 2000, each of which is incorporated by reference in its entirety.

### FIELD OF THE INVENTION

The invention generally relates to nucleic acids and polypeptides encoded therefrom.

### BACKGROUND OF THE INVENTION

The invention generally relates to nucleic acids and polypeptides encoded therefrom. More specifically, the invention relates to nucleic acids encoding cytoplasmic, nuclear, membrane bound, and secreted polypeptides, as well as vectors, host cells, antibodies, and recombinant methods for producing these nucleic acids and polypeptides.

### SUMMARY OF THE INVENTION

The invention is based in part upon the discovery of nucleic acid sequences encoding novel polypeptides. The novel nucleic acids and polypeptides are referred to herein as NOVX, or NOV1, NOV2, NOV3, NOV4, NOV5, NOV6, NOV7, NOV8, NOV9, and NOV10 nucleic acids and polypeptides. These nucleic acids and polypeptides, as well as derivatives, homologs, analogs and fragments thereof, will hereinafter be collectively designated as “NOVX” nucleic acid or polypeptide sequences.

In one aspect, the invention provides an isolated NOVX nucleic acid molecule encoding a NOVX polypeptide that includes a nucleic acid sequence that has identity to the nucleic acids disclosed in SEQ ID NOS: 1, 3, 5, 7, 9, 11, 13, 15, 17, 19, 21, 23, and 25. In some embodiments, the NOVX nucleic acid molecule will hybridize under stringent conditions to a nucleic acid sequence complementary to a nucleic acid molecule that includes a protein-coding sequence of a NOVX nucleic acid sequence. The invention also includes an isolated nucleic acid that encodes a NOVX polypeptide, or a fragment, homolog, analog or derivative thereof. For example, the nucleic acid can encode a polypeptide at least 80% identical to a polypeptide comprising the amino acid sequences of SEQ ID NOS: 2, 4, 6, 8, 10, 12, 14, 16, 18, 20, 22, 24, and 26. The nucleic acid can be, for example, a genomic DNA fragment or a cDNA molecule that includes the nucleic acid sequence of any of SEQ ID NOS: 1, 3, 5, 7, 9, 11, 13, 15, 17, 19, 21, 23, and 25.

Also included in the invention is an oligonucleotide, e.g., an oligonucleotide which includes at least 6 contiguous nucleotides of a NOVX nucleic acid (e.g., SEQ ID NOS: 1, 3, 5, 7, 9, 11, 13, 15, 17, 19, 21, 23, and 25) or a complement of said oligonucleotide.

Also included in the invention are substantially purified NOVX polypeptides (SEQ ID NOS: 2, 4, 6, 8, 10, 12, 14, 16, 18, 20, 22, 24, and 26). In certain embodiments, the NOVX polypeptides include an amino acid sequence that is substantially identical to the amino acid sequence of a human NOVX polypeptide.

The invention also features antibodies that immunoselectively bind to NOVX polypeptides, or fragments, homologs, analogs or derivatives thereof.

In another aspect, the invention includes pharmaceutical compositions that include therapeutically- or prophylactically-effective amounts of a therapeutic and a pharmaceutically-acceptable carrier. The therapeutic can be, e.g., a NOVX nucleic acid, a NOVX polypeptide, or an antibody specific for a NOVX polypeptide. In a further aspect, the invention includes, in one or more containers, a therapeutically- or prophylactically-effective amount of this pharmaceutical composition.

In a further aspect, the invention includes a method of producing a polypeptide by culturing a cell that includes a NOVX nucleic acid, under conditions allowing for expression of the NOVX polypeptide encoded by the DNA. If desired, the NOVX polypeptide can then be recovered.

In another aspect, the invention includes a method of detecting the presence of a NOVX polypeptide in a sample. In the method, a sample is contacted with a compound that selectively binds to the polypeptide under conditions allowing for formation of a complex between the polypeptide and the compound. The complex is detected, if present, thereby identifying the NOVX polypeptide within the sample.

The invention also includes methods to identify specific cell or tissue types based on their expression of a NOVX.

Also included in the invention is a method of detecting the presence of a NOVX nucleic acid molecule in a sample by contacting the sample with a NOVX nucleic acid probe or primer, and detecting whether the nucleic acid probe or primer bound to a NOVX nucleic acid molecule in the sample.

In a further aspect, the invention provides a method for modulating the activity of a NOVX polypeptide by contacting a cell sample that includes the NOVX polypeptide with a compound that binds to the NOVX polypeptide in an amount sufficient to modulate the activity of said polypeptide. The compound can be, e.g., a small molecule, such as a nucleic acid, peptide, polypeptide, peptidomimetic, carbohydrate, lipid or other organic (carbon containing) or inorganic molecule, as further described herein.

Also within the scope of the invention is the use of a therapeutic in the manufacture of a medicament for treating or preventing disorders or syndromes including, e.g., diabetes, metabolic disturbances associated with obesity, the metabolic syndrome X, anorexia, wasting disorders associated with chronic diseases, metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, or other disorders related to cell signal processing and metabolic pathway modulation. The therapeutic can be, e.g., a NOVX nucleic acid, a NOVX polypeptide, or a NOVX-specific antibody, or biologically-active derivatives or fragments thereof.

For example, the compositions of the present invention will have efficacy for treatment of patients suffering from: developmental diseases, MHCII and III diseases (immune diseases), taste and scent detectability Disorders, Burkitt's lymphoma, corticoneurogenic disease, signal transduction pathway disorders, Retinal diseases including those involving photoreception, Cell growth rate disorders; cell shape disorders, feeding disorders; control of feeding; potential obesity due to over-eating; potential disorders due to starvation (lack of appetite), noninsulin-dependent diabetes mellitus (NIDDMI), bacterial, fungal, protozoal and viral infections (particularly infections caused by HIV-1 or HIV-2), pain, cancer (including but not limited to neoplasm; adenocarcinoma; lymphoma; prostate cancer; uterus cancer), anorexia, bulimia, astluna, Parkinson's disease, acute heart failure, hypotension, hypertension, urinary retention, osteoporosis, Crohn's disease; multiple sclerosis; Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation. Dentatorubro-pallidoluysian atrophy (DRPLA) Hypophosphatemic rickets, autosomal dominant (2) Acrocallosal syndrome and dyskinesias, such as Huntington's disease or Gilles de la Tourette syndrome and/or other pathologies and disorders of the like.

The polypeptides can be used as immunogens to produce antibodies specific for the invention, and as vaccines. They can also be used to screen for potential agonist and antagonist compounds. For example, a cDNA encoding NOVX may be useful in gene therapy, and NOVX may be useful when administered to a subject in need thereof. By way of non-limiting example, the compositions of the present invention will have efficacy for treatment of patients suffering from bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation and dyskinesias, such as Huntington's disease or Gilles de la Tourette syndrome and/or other pathologies and disorders.

The invention further includes a method for screening for a modulator of disorders or syndromes including, e.g., diabetes, metabolic disturbances associated with obesity, the metabolic syndrome X, anorexia, wasting disorders associated with chronic diseases, metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders or other disorders related to cell signal processing and metabolic pathway modulation. The method includes contacting a test compound with a NOVX polypeptide and determining if the test compound binds to said NOVX polypeptide. Binding of the test compound to the NOVX polypeptide indicates the test compound is a modulator of activity, or of latency or predisposition to the aforementioned disorders or syndromes.

Also within the scope of the invention is a method for screening for a modulator of activity, or of latency or predisposition to an disorders or syndromes including, e.g., diabetes, metabolic disturbances associated with obesity, the metabolic syndrome X, anorexia, wasting disorders associated with chronic diseases, metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders or other disorders related to cell signal processing and metabolic pathway modulation by administering a test compound to a test animal at increased risk for the aforementioned disorders or syndromes. The test animal expresses a recombinant polypeptide encoded by a NOVX nucleic acid. Expression or activity of NOVX polypeptide is then measured in the test animal, as is expression or activity of the protein in a control animal which recombinantly-expresses NOVX polypeptide and is not at increased risk for the disorder or syndrome. Next, the expression of NOVX polypeptide in both the test animal and the control animal is compared. A change in the activity of NOVX polypeptide in the test animal relative to the control animal indicates the test compound is a modulator of latency of the disorder or syndrome.

In yet another aspect, the invention includes a method for determining the presence of or predisposition to a disease associated with altered levels of a NOVX polypeptide, a NOVX nucleic acid, or both, in a subject (e.g., a human subject). The method includes measuring the amount of the NOVX polypeptide in a test sample from the subject and comparing the amount of the polypeptide in the test sample to the amount of the NOVX polypeptide present in a control sample. An alteration in the level of the NOVX polypeptide in the test sample as compared to the control sample indicates the presence of or predisposition to a disease in the subject. Preferably, the predisposition includes, e.g., diabetes, metabolic disturbances associated with obesity, the metabolic syndrome X, anorexia, wasting disorders associated with chronic diseases, metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders. Also, the expression levels of the new polypeptides of the invention can be used in a method to screen for various cancers as well as to determine the stage of cancers.

In a farther aspect, the invention includes a method of treating or preventing a pathological condition associated with a disorder in a mammal by administering to the subject a NOVX polypeptide, a NOVX nucleic acid, or a NOVX-specific antibody to a subject (e.g., a human subject), in an amount sufficient to alleviate or prevent the pathological condition. In preferred embodiments, the disorder, includes, e.g., diabetes, metabolic disturbances associated with obesity, the metabolic syndrome X, anorexia, wasting disorders associated with chronic diseases, metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders.

In yet another aspect, the invention can be used in a method to identity the cellular receptors and downstream effectors of the invention by any one of a number of techniques commonly employed in the art. These include but are not limited to the two-hybrid system, affinity purification, co-precipitation with antibodies or other specific-interacting molecules.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The present invention provides novel nucleotides and polypeptides encoded thereby. Included in the invention are the novel nucleic acid sequences and their polypeptides. The sequences are collectively referred to as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein. Table A provides a summary of the NOVX nucleic acids and their encoded polypeptides. Example 1 provides a description of how the novel nucleic acids were identified.

TABLE A
Sequences and Corresponding SEQ ID Numbers

| | | SEQ ID | | |
|---|---|---|---|---|
| | | NO | | |
| NOVX | | (nucleic | SEQ ID NO | |
| Assignment | Internal Identification | acid) | (polypeptide) | Homology |
| 1 | AP001404_A | 1 | 2 | Leupin |
| 2 | Ba3S0p16_A | 3 | 4 | Interferon |
| 3 | 29145493_EXT | 5 | 6 | Tyrosine Kinase Receptor |
| 4 | GM_95074063_A | 7 | 8 | Chloride conductance |
| 5a | GM_83554525_A | 9 | 10 | 5-hydroxytryptamine (serotonin) |
| | (CG54692-01) | | | receptor |
| 5b | (CG54692-O1) | 11 | 12 | Serotonin Receptor |
| 6a | 21639300_EXT | 13 | 14 | Salivary Gland Protein |
| 6b | CG51622-02 | 15 | 16 | (Von Ebner) Salivary Gland Protein |
| 7 | GM_51624520_A | 17 | 18 | CD-81 |
| 8a | 27479850_EXT1 | 19 | 20 | SHD |
| 8b | CG51761-02 | 21 | 22 | SHD |
| 9 | A1284055_EXT | 23 | 24 | Hepatoma-Derived Growth Factor |
| 10 | 95073892_EXT- | 25 | 26 | Salt-Inducible Protein Kinase |
| | REVCOMP | | | |

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

For example, NOV1 is homologous to members of SCCA family of proteins that are important protease inhibitors and cancer antigens. Thus, the NOV1 nucleic acids, polypeptides, antibodies and related compounds according to the invention will be useful in therapeutic and diagnostic applications in disorders characterized by protease inhibition and carcinoma, e.g., squamus cell carcinoma of, for example, cervix, head and neck, lung, and esophagus.

Also, NOV2 is homologous to the interferon family of proteins. Thus NOV2 nucleic acids, polypeptides, antibodies and related compounds according to the invention will be useful in therapeutic and diagnostic applications in disorders characterized by e.g., hyperproliferation, e.g., cancer, neurologic disease, immune disorders, and viral infection.

Further, NOV3 is homologous to a family of tyrosine kinase-like receptor proteins important in cell proliferation and differentiation. Thus, the NOV3 nucleic acids and polypeptides, antibodies and related compounds according to the invention will be useful in therapeutic and diagnostic applications in developmental and proliferative disorders, e.g. angiogenesis, cell signaling disorders, cancer, fertility disorders, reproductive disorders, tissue/cell growth regulation disorders.

Also, NOV4 is homologous to the chloride channel family of proteins important in chloride ion transport. Thus, NOV4 nucleic acids, polypeptides, antibodies and related compounds according to the invention will be useful in therapeutic and diagnostic applications in various disorders, including, for example, cystic fibrosis, congenital myotonia, Dent disease, an X-linked renal tubular disorder, leukoencephalopathy, malignant hyperthermia, and hypertension.

Additionally, NOV5a and NOV5b are homologous to the serotonin receptor family of proteins. Thus NOV5 nucleic acids, polypeptides, antibodies and related compounds according to the invention will be useful in treating a variety of conditions, including, e.g., seizures, Alzheimer's disease, sleep disorders, appetite disorders, thermoregulation, pain perception, hormone secretion and sexual behavior, mental depression, migraine, epilepsy, obsessive-compulsive behavior (schizophrenia), drug addiction, and affective disorders.

Also, NOV6 is homologous to the salivary gland-like, or lipocalin family of proteins. Thus NOV6 nucleic acids, polypeptides, antibodies and related compounds according to the invention will be useful in therapeutic and diagnostic applications in various disorders, including, for example,. olfactory disorders, salivitory disorders, digestive disorders, oral immunologic disorders, poor oral health, inflammatory processes in the airways due to allergy/asthma, emphysema or viral infection, cystic fibrosis, and obesity.

Further, NOV7 is homologous to members of the tetraspannin family of proteins. Thus, the NOV7 nucleic acids, polypeptides, antibodies and related compounds according to the invention will be useful in therapeutic and diagnostic applications in disorders characterized by inflammation, e.g., asthma, arthritis, psoriasis, and inflammatory bowel disease.

Still further, NOV8 is homologous to a family of src homology domain-containing proteins that are important in a variety of functions, including signal transduction. Thus, NOV8 nucleic acids and polypeptides, antibodies and related compounds according to the invention will be useful in therapeutic and diagnostic applications in disorders characterized by altered signal transduction, e.g. cancer, lymphoproliferative syndrome, cerebral palsy, epilepsy, and other and/or other pathologies and disorders.

NOV9 is homologous to the hepatoma-derived growth factor (HDGF) family of proteins. Thus, NOV9 nucleic acids and polypeptides, antibodies and related compounds according to the invention will be useful in therapeutic and diagnostic applications in various disorders including, for example, cell proliferation disorders, development disorders, and nephrogenesis.

Finally, NOV10 is homologous to the salt-inducible kinase family of proteins that are important in adrenocortical functions. Thus, NOV10 nucleic acids and polypeptides, antibodies and related compounds according to the invention will be useful in therapeutic and diagnostic applications in various disorders, e.g. adrenoleukodystrophy, kidney disease, atherosclerosis, and inflammation.

The NOVX nucleic acids and polypeptides can also be used to screen for molecules, which inhibit or enhance NOVX activity or function. Specifically, the nucleic acids and polypeptides according to the invention may be used as targets for the identification of small molecules that modulate or inhibit, e.g., neurogenesis, cell differentiation, cell proliferation, hematopoiesis, wound healing and angiogenesis.

Additional utilities for the NOVX nucleic acids and polypeptides according to the invention are disclosed herein.

NOV1

A NOV1 sequence according to the invention includes a nucleic acid sequence encoding a polypeptide related to the leupin family of proteins. A NOV1 nucleic acid is found on human chromosome 18. A NOV1 nucleic acid and its encoded polypeptide includes the sequence shown in Tables 1A-1B. A disclosed NOV1 nucleic acid of 1200 nucleotides is shown in Table 1A, and is identified as SEQ ID NO: 1. The disclosed NOV1 open reading frame (“ORF”) begins at the ATG initiation codon at nucleotides 7-9, shown in bold in Table 1A. The encoded polypeptide is alternatively referred to herein as NOV1 or as AP001404_A. The disclosed NOV1 ORF terminates at a TAA codon at nucleotides 1192-1194. As shown in Table 1A, putative untranslated regions 5′ to the start codon and 3′ to the stop codon are underlined, and the start and stop codons are in bold letters.

TABLE 1A
NOV1 nucleotide sequence (SEQ ID NO:1).

TTTACAATGGACTCTCTTGTTACAGCAAACACCAAATTTTGCTTTGATCTTTTTCAAGAGATAGGCAAAG
ATGATCGTCATAAAAACATATTTTTCTCTCCCCTGAGCCTCTCAGCTGCCCTTGGTATGGTACGCTTGGG
TGCTAGAAGTGACAGTGCACATCAGATTGATGAGGTACTACACTTCAACGAATTTTCCCAGAATGAAAGC
AAAGAACCTGCTGGGTCCTTAAACAATGAGAGCGGACTGGTCAGCTGCTACTTTGGGCAGCTTCTCTCCA
AATTAGACAGGATCAAGACTGATTACACACTGAGTATTGCCAACAGGCTTTATGGAGAGCAGGAATTCCC
AATCTGTCAGGAATACTTAGATGGTGTGATTCAATTTTACCACACGACGATTGAAAGTGTTGATTTCCAA
AAAAACCCTGAAAAATCCAGACAAGAGATTAACTTCTGGGTTGAATGTCAATCCCAAGGTAAAATCAAGG
ACCTCTTCAGCAAGGACGCTATTAATGCTGAGACTGTGCTGGTACTGGTGAATGCTGTTTACTTCAAGGC
CAAATGGGAAACATACTTTGACCATGAAAACACGGTGGATGCACCTTTCTGTCTAAATCAGAATGAAAAC
AAGAGTGTGAAGATGATGACGCAAAAAGGCCTCTACAGAATTGGCTTCATAGAGGAGGTGAAGGCACAGA
TCCTGGAAATGAGGTACACCAAGGGGAAGCTCAGCATGTTCGTGCTGCTGCCATCTCACTCTAAAGATAA
CCTGAAGGGTCTGGAAGAGCTTGAAAGGAAAATCACCTATGAAAAAATGGTGGCCTGGAGCAGCTCAGAA
AACATGTCAGAAGAATCGGTGGTCCTGTCCTTCCCCCGGTTCACCCTGGAAGACAGCTATGATCTCAATT
CCATTTTACAAGACATGGGCATTACGGATATCTTTGATGAAACGAGGGCTGATCTTACTGGAATCTCTCC
AAGTCCCAATTTGTACTTGTCAAAAATTATCCACAAAACCTTTGTGGAGGTGGATGAAAACGGTACCCAG
GCAGCTGCAGCCACTGGGGCTGTTGTCTCGGAAAGGTCACTACGATCTTGGGTGGAGTTTAATGCCAACC
ACCCTTTTCTCTTTTTCATTAGACACAACAAAACCCAAACCATTCTCTTTTATGGCAGGGTCTGCTCTCC
TTAAAAGGGG

A disclosed encoded NOV1 protein has 395 amino acid residues, referred to as the NOV1 protein. The NOV1 protein was analyzed for signal peptide prediction and cellular localization. SignalP results predict that NOV1 is likely to be localized in the microbody (peroxisome), with a certainty of 0.5007. The disclosed NOV1 polypeptide sequence is presented in Table 1B using the one-letter amino acid code.

TABLE 1B
Encoded NOV1 protein sequence (SEQ ID NO:2).

MDSLVTANTKFCFDLFQEIGKDDHKNIFFSPLSLSAALGMVRLGARSDSAHQIDEVLHFNEFSQNESKE
PAGSLNNESGLVSCYFGQLLSKLDRIKTDYTLSIANRLYGEQEFPICQEYLDGVIQFYHTTIESVDFQKN
PEKSRQEINFWVECQSQGKIKDLFSKDAINAETVLVLVNAVYFKAKWETYFDHENTVDAPFCLNQNENKS
VKMMTQKGLYRIGFIEEVKAQILEMRYTKGKLSMFVLLPSHSKDNLKGLEELERKITYEKMVAWSSSENM
SEESVVLSFPRFTLEDSYDLNSILQDMGITDIFDETRADLTGISPSPNLYLSKIIHKTFVEVDENGTQAA
AATGAVVSERSLRSWVEFNANHPFLFFIRHNKTQTILFYGRVCSP

NOV1a was initially identified on chromosome 18 with a TblastN analysis of a proprietary sequence file for leupin or a homolog, which was run against the Genomic Daily Files made available by GenBank or from files downloaded from the individual sequencing centers. The nucleic acid sequence was predicted from the genomic file GenBank: AP001404 by homology to a known Leupin or homolog. Exons were predicted by homology and the intron/exon boundaries were determined using standard genetic rules. Exons were further selected and refined by means of similarity determination using multiple BLAST (for example, tBlastn, BlastX, Blastn) searches, and, in some instances, GenScan and Grail. Expressed sequences from both public and proprietary databases were also added when available to further define and complete the gene sequence. The DNA sequence was then manually corrected for apparent inconsistencies thereby obtaining the sequences encoding the full-length protein.

A region of the NOV1 nucleic acid sequence has 515 of 789 bases (65%) identical to a 1284 nucleotide sequence coding for Homo sapiens squamus cell carcinoma antigen 2 mRNA (SCCA2), with an E-value of 1.2e⁻⁷⁰ (GENBANK-ID: HSU19557|acc:U19557). Also, in a search of public sequence databases, it was found, for example, that the NOV1 nucleic acid sequence disclosed in this invention has 435 of 447 bases (97%, E=8.6e⁻⁹⁰) identical to an IMAGE clone (Soares_NhHMPu_S1 Homo sapiens cDNA clone IMAGE:668321 5′ similar to SW:SCC2_HUMAN P48594 squamous cell carcinoma antigen 2) (GENBANK-ID: AA242969). The strong (97%) homology of a 435 base pair segment of the current invention with 447 base pair region of this 555 bp RNA GenBank sequence suggests that the current invention represents an expressed gene sequence. Public nucleotide databases include all GenBank databases and the GeneSeq patent database.

In all BLAST alignments herein, the “E-value” or “Expect” value is a numeric indication of the probability that the aligned sequences could have achieved their similarity to the BLAST query sequence by chance alone, within the database that was searched. For example, the probability that the subject (“Sbjct”) retrieved from the NOV1 BLAST analysis, e.g., Homo sapiens squamus cell carcinoma antigen 2 mRNA, matched the Query NOV1 sequence purely by chance is 1.2×10⁻⁷⁰. The Expect value (E) is a parameter that describes the number of hits one can “expect” to see just by chance when searching a database of a particular size. It decreases exponentially with the Score (S) that is assigned to a match between two sequences. Essentially, the E value describes the random background noise that exists for matches between sequences.

The Expect value is used as a convenient way to create a significance threshold for reporting results. The default value used for blasting is typically set to 0.0001. In BLAST 2.0, the Expect value is also used instead of the P value (probability) to report the significance of matches. For example, an E value of one assigned to a hit can be interpreted as meaning that in a database of the current size one might expect to see one match with a similar score simply by chance. An E value of zero means that one would not expect to see any matches with a similar score simply by chance. See, e.g., http://www.ncbi.nlm.nih.gov/Education/BLASTinfo/. Occasionally, a string of X's or N's will result from a BLAST search. This is a result of automatic filtering of the query for low-complexity sequence that is performed to prevent artifactual hits. The filter substitutes any low-complexity sequence that it finds with the letter “N” in nucleotide sequence (e.g., “NNNNNNNNNNNNN”) or the letter “X” in protein sequences (e.g., “XXXXXXXX”). Low-complexity regions can result in high scores that reflect compositional bias rather than significant position-by-position alignment. Wootton and Federhen, Methods Enzymol 266:554-571, 1996.

A BLASTX search was performed against public protein databases. The disclosed NOV1 protein (SEQ ID NO: 2) has good identity with a number of leupin-like proteins. For example, the full amino acid sequence of the protein of the invention was found to have 196 of 395 amino acid residues (49%) identical to, and 270 of 395 residues (68%) positive with, the 390 amino acid squamus cell carcinoma antigen 2 (SCCA-2, leupin) protein from Homo sapiens (ptnr:SWISSPROT-ACC: P48594, E=4.8 e-93). Public amino acid databases include the GenBank databases, SwissProt, PDB and PIR.

Other BLAST results include sequences from the Patp database, which is a proprietary database that contains sequences published in patents and patent publications. Patp results include those listed in Table 1 C.

TABLE 1C
Patp alignments of NOV1

| | | | Smallest |
|---|---|---|---|
| | | | Sum |
| | Reading | High | Prob. |
| Sequences producing High-scoring Segment Pairs: | Frame | Score | P (N) |
| Patp:Y25927 Human SCCA2 protein—Homo sapiens, 390 aa. | +1 | 932 | 8.0e − 93 |
| Patp:W15242 Psoriastatin type II—Homo sapiens, 390 aa. | +1 | 928 | 2.1e − 92 |
| Patp:R25276 SCC antigen—Synthetic. 390 aa. | +1 | 910 | 1.7e − 90 |
| Patp:Y32077 Hepatitis B virus receptor SCCA1 Homo sapiens | +1 | 910 | 1.7e − 90 |

For example, a BLAST against patp: Y25927, a 390 amino acid SCCA2 from Homo sapiens, produced good identity, E=8.0e⁻⁹³).

The disclosed protein is also similar to the leupin-like proteins in Table 1D.

TABLE 1D
BLAST results for NOV1

| Gene Index/ | | Length | Identity | Positives | Expect |
|---|---|---|---|---|---|
| Identifier | Protein/Organism | (ae) | (%) | (%) | |
| Gi \| 1710877 \| sp \| P4859 | SQUAMOUS CELL | 390 | 181/396 | 252/396 | 2e − 85 |
| 4 \| SCC2^{—}HUMAN | CARCINOMA ANTIGEN | | (45%) | (62%) | |
| (X89015), (U19557) | 2 (SCCA-2) | | | | |
| (U19576), | (LEUPIN) | | | | |
| (AB035089) | Homo sapiens | | | | |
| Gi \| 2118384 \| pir \| \| I38 | leupin precursor | 390 | 181/396 | 252/396 | 3e − 85 |
| 202 | Homo sapiens | | (45%) | (62%) | |
| Gi \| 2118383 \| pir \| \| I38 | Squamous cell | 390 | 179/396 | 252/396 | 4e − 83 |
| 201 | carcinoma antigen | | (45%) | (63%) | |
| | 1 | | | | |
| | Homo sapiens | | | | |
| Gi \| 1172087 \| gb \| AAA86 | Squamous cell | 390 | 179/396 | 252/396 | 4e − 83 |
| 317.1 \| (U19568); | carcinoma | | (45%) | (63%) | |
| (U19556) | antigen-1 Homo | | | | |
| | sapiens; serine | | | | |
| | (or cysteine) | | | | |
| | proteinase | | | | |
| | inhibitor, clade | | | | |
| | B (ovalbumin) | | | | |
| | member 3 | | | | |

A ClustalW analysis comparing disclosed proteins of the invention with related leupin protein sequences is given in Table 1E, with NOV1 shown on line 1.

In the ClustalW alignment of the NOV1 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be mutated to a much broader extent without altering protein structure or function.

The NOV1 protein has significant homology to leupin-like proteins.

TABLE 1E
ClustalW Analysis of NOV1

1) Novel NOV1 (SEQ ID NO:2)
2) gi|1710877|sp|P48594|SCC2 SQUAMOUS CELL CARCINOMA ANTIGEN 2 (SCCA-2) (LEUPIN) (SEQ ID NO:27)
3 gi|2118384|pir||I38202 leupin precursor (SEQ ID NO:28)
4) gi|2118383|pir||I38201 squamous cell carcinoma antigen 1 (SEQ ID NO:29)
5) gi|5902072|ref|NP_008850.1|serine (or cysteine) proteinase inhibitor, clade B (ovalbumin), member 3; SCCA-1 (SEQ ID NO:30)

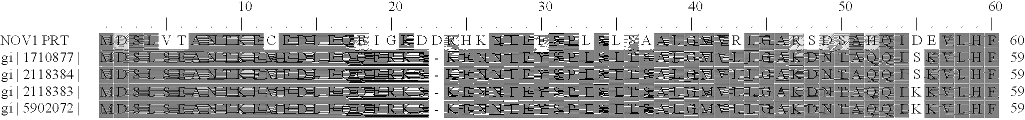

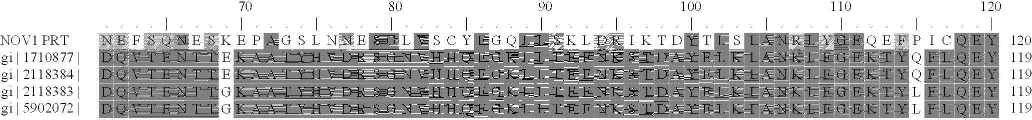

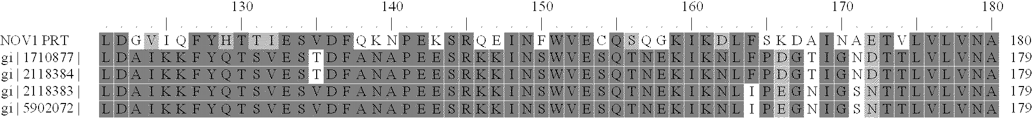

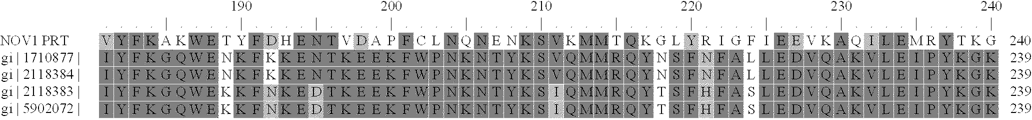

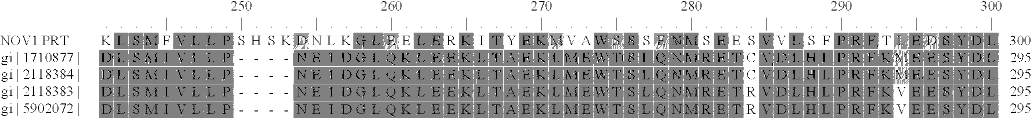

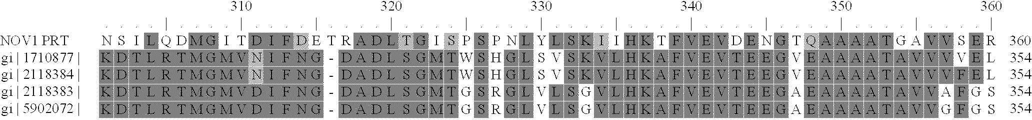

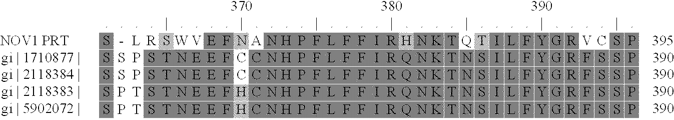

The presence of identifiable domains in NOV1, as well as all other NOVX proteins, was determined by searches using software algorithms such as PROSITE, DOMAIN, Blocks, Pfam, ProDomain, and Prints, and then determining the Interpro number by crossing the domain match (or numbers) using the Interpro website (http:www.ebi.ac.uk/interpro). DOMAIN results, e.g., for NOV1 as disclosed in Table 1F, were collected from the Conserved Domain Database (CDD) with Reverse Position Specific BLAST analyses. This BLAST analysis software samples domains found in the Smart and Pfam collections. For Table 1 E and all successive DOMAIN sequence alignments, fully conserved single residues are indicated by black shading and “strong” semi-conserved residues are indicated by grey shading. The “strong” group of conserved amino acid residues may be any one of the following groups of amino acids: STA, NEQK, NHQK, NDEQ, QHRK, MILV, MILF, HY, FYW.

Table 1F lists the domain description from DOMAIN analysis results against NOV1. The region from amino acid residue 13 through 395 (SEQ ID NO: 2) most probably (E=3e⁻⁹⁵) contains a “SERPIN” (Serine proteinase inhibitor) domain, aligned here with the 360 amino acid SERPIN (Smart database), Pfam 00079 (SEQ ID NO: 31). This indicates that the NOV1 sequence has properties similar to those of other proteins known to contain this domain.

TABLE 1F
Domain Analysis of NOV1

gnl | Smart | SERPIN, SERine Proteinase INhibitors
CD-Length = 360 residues, 100.0% aligned
Score = 341 bits (875), Expect = 3e−95

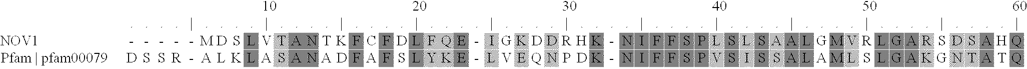

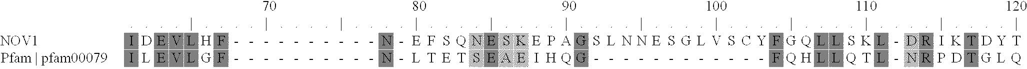

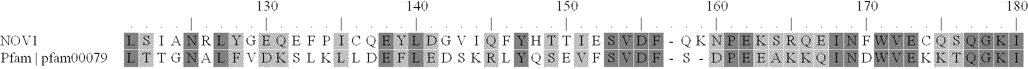

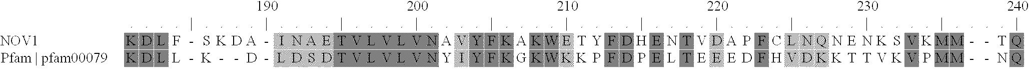

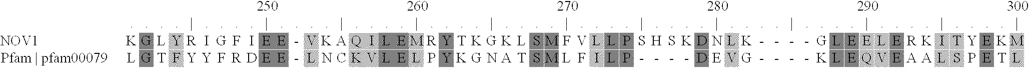

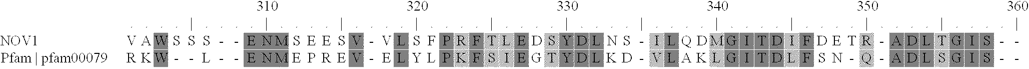

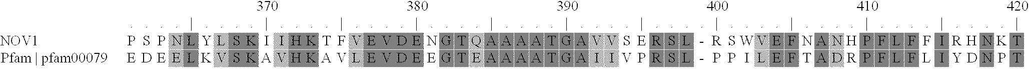

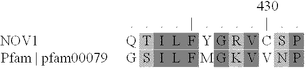

The representative member of the SERPIN family is shown in Table 1F. The family contains 58 sequences, including SCCA and many serine protease inhibitors.

Barnes and Worrall described the cloning of a member of the serpin family of serine protease inhibitors by degenerate PCR and screening of a HeLa cell cDNA library. Barnes and Worrall, FEBS Lett. 373: 61-65, 1999. The isolated cDNA encodes a 390-amino acid protein, designated leupin, that is 91.8% identical to SCCA1. The authors stated that the reactive site of leupin differs from SCCA1 in the active loop region, including the presence of a leucine residue rather than a serine at the P(1) position within the loop region that acts as a pseudo-substrate for the target protease. Barnes and Worrall speculated that leupin may be a cysteine protease inhibitor, and that the isoelectric point is consistent with the acidic form of SCCA associated with squamus cell carcinomas. Barnes and Worrall, supra.

The squamous cell carcinoma antigen (SCCA) is a member of the ovalbumin family of serine proteinase inhibitors (serpins). The protein was isolated from a metastatic cervical squamous cell carcinoma by Kato and Torigoe, Cancer 40:1621-1628, 1977 (See, e.g., Online Mendelian Inheritance in Man (OMIM), available at http://www.ncbi.nlm.nih.gov/., entry 600517 and 600518). SCCA is detected in the superficial and intermediate layers of normal squamous epithelium, whereas the mRNAs is detected in the basal and subbasal levels. The clinical import of SCCA has been as a circulating tumor marker for squamous cell carcinoma, especially those of the cervix, head and neck, lung, and esophagus. The squamous cell carcinoma antigen (SCCA) serves as a serological marker for more advanced squamous cell tumors. Many clinical studies of cervical squamous cell carcinoma show that the percentage of patients with elevated circulating levels of SCCA increases from approximately 12% at stage 0 to more than 90% at stage IV. Levels fall after tumor resection and rise in approximately 90% of the patients with recurrent disease. Similar trends occur in the other types of squamous cell carcinoma, with a maximum sensitivity of approximately 60% for lung, 50% for esophageal, and 55% for head and neck tumors. The neutral form of SCCA is detected in the cytoplasm of normal and some malignant squamous cells, whereas the acidic form is expressed primarily in malignant cells and is the major form found in the plasma of cancer patients. Thus, the appearance of the acidic fraction of SCCA is correlated with more aggressive tumors.

In an analysis of chromosomal aberrations involving human chromosome band 18q21, Silverman et al.(Silverman, et al., Genomics 9:219-228, 1991) identified a DNA fragment, A56R (D18S86), that contained a 56/57-bp match with the published cDNA sequence of SCCA (Suminami et al., Biochem. Biophys. Res. Commun. 181:51-58, 1991). Schneider et al. (Proc. Nat. Acad. Sci. 92:3147-3151, 1995) showed that this fragment contained exon 3 of a new gene, SCCA2 (OMIM- 600518), which was 92% identical to SCCA1. SCCA1 and SCCA2, which map within 18q21.3, are tandemly arrayed and flanked by two members of the ovalbumin family of serine proteinase inhibitors, plasminogen activator inhibitor type 2 (PAI2; OMIM-173390) and maspin (protease inhibitor 5; PI5; OMIM-154790). The predicted pI values and molecular weights of the cDNAs suggested that the neutral and acidic forms of the SCCA were encoded by SCCA1 and SCCA2, respectively. Analysis of the primary amino acid sequences shows that both genes are members of the high molecular weight serpin superfamily of serine proteinase inhibitors.

Although SCCA1 and SCCA2 are nearly identical in primary structure, the reactive site loop of each inhibitor suggests that they may differ in their specificity for target proteinases. SCCA1 has been shown to be effective against papain-like cysteine proteinases. Schick et al. demonstrated that SCCA2 inhibits the chymotrypsin-like proteinases cathepsin G (OMIM-116830) and mast cell chymase (OMIM-118938) in vitro. Schick, et al., J. Biol. Chem. 272:1849-1855, 1997. SCCA2 was ineffective against papain-like cysteine proteinases, which have been shown to be inhibited by SCCA1 (OMIM 600518).

The nucleic acids and proteins of NOV1 are useful in potential therapeutic applications implicated in various leupin- or serpin-related pathologies and/or disorders. For example, a cDNA encoding the leupin-like protein may be useful in gene therapy, and the leupin-like protein may be useful when administered to a subject in need thereof. The novel nucleic acid encoding NOV1 protein, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. The NOVX nucleic acids and proteins are useful in potential diagnostic and therapeutic applications implicated in various diseases and disorders described below and/or other pathologies. The NOV1 nucleic acids and proteins are useful in therapeutic applications implicated in, for example, connective tissue remodeling; Alzheimer's Disease; hypertension; cardiac hypertrophy; coronary heart disease, squamous cell carcinoma, especially those of the cervix, head and neck, lung, and esophagus, and/or other pathologies and disorders.

For example, a cDNA encoding the leupin-like protein may be useful in gene therapy, and the Leupin-like protein may be useful when administered to a subject in need thereof. By way of nonlimiting example, the compositions of the present invention will have efficacy for treatment of patients suffering from connective tissue remodeling; Alzheimer's Disease; hypertension; cardiac hypertrophy; coronary heart disease, squamous cell carcinoma (especially those of the cervix, head and neck, lung, and esophagus). The novel nucleic acid encoding leupin-like protein, and the leupin-like protein of the invention, or fragments thereof, may further be useful in diagnostic applications, wherein the presence or amount of the nucleic acid or the protein are to be assessed.

Further, the protein similarity information, expression pattern, and map location for NOV1 suggests that NOV1 may have important structural and/or physiological functions characteristic of the SCCA family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

These materials are further useful in the generation of antibodies that bind immuno-specifically to the novel NOV1 substances for use in therapeutic or diagnostic methods. These antibodies may be generated according to methods known in the art, using prediction from hydrophobicity charts, as described in the “Anti-NOVX Antibodies” section below. The disclosed NOV1 protein has multiple hydrophilic regions, each of which can be used as an immunogen. In one embodiment, a contemplated NOV1 epitope is from about amino acids 10 to 30. In another embodiment, a NOV1 epitope is from about amino acids 50 to 75. In additional embodiments, NOV1 epitopes are from amino acids 90 to 125, 130- 160, 180-200, and from amino acids 260 to 280. These novel proteins can be used in assay systems for functional analysis of various human disorders, which will help in understanding of pathology of the disease and development of new drug targets for various disorders.

NOV2

A novel nucleic acid was identified on chromosome 9 by TblastN using CuraGen Corporation's sequence file for interferon or homolog as run against the Genomic Daily Files made available by GenBank or from files downloaded from the individual sequencing centers. The nucleic acid sequence was predicted from the genomic file Seq Ctr ACCNO:sggc_draft_ba380p16_{—}20000326 by homology to a known interferon or homolog. Exons were predicted by homology and the intron/exon boundaries were determined using standard genetic rules. Exons were further selected and refined by means of similarity determination using multiple BLAST (tBlastn, BlastX, Blastn) searches, and, in some instances, Genscan and Grail. Expressed sequences from both public and proprietary databases were also added when available to further define and complete the gene sequence. The DNA sequence was then manually corrected for apparent inconsistencies thereby obtaining the sequences encoding the full-length protein. The novel nucleic acid of 695 nucleotides (ba380p16_A, SEQ ID NO: 3) encoding a novel interferon-like protein is shown in Table 2A.

TABLE 2A
NOV2 Nucleotide Sequence (SEQ ID NO:3)

AAAATGGTATTATTAGAACAGGATTTCCAGTTCGGACTCGGTCCCCTCCTGGTGGCCCTGCTGCTTTGCC
ACTGTGGCCCTGTTGGATCTCTGGGCTTTGACCTGCCTCAGAACCATGGCCTACTTAGCAGGAACACCTT
GGCTCTTCTGGGCCAAATGCAGAGAATCTCCCCTTTCTTGTGTCTCAAGGACAGAAGAGACTTCAGGTTC
CCCCTTTTTTTTGTTGATGGCAGCCAGTTGCATAAGGCCCAGGCCCTGTCTGTCCTCCATGAGATGCTTC
AGCAGATCTTCAGCGTCTACCCCACAGAGTGCTCCTCTGCTGCCTGGAACATGACCCTCCTGGACCAGCT
CCACACTGGATTTCATCTGTATCTAGGATGCCTGGAGTCTCGCTTAGGGCAGGCAATAGGAGAGGAAGAA
TCTGTAGGGGTGATTGTGGCCCCTACACTGGCCTTGAGGAGGTACTTCCAGGGAATCCATGGAATCCAGA
GAATCTACCTGAAAGAGAAGAAATACAGTGACTGTGCTTGGGAGGTTCTCAGAGTGGGAATCATGAAATC
CTTCTCTTCATCAACAAACTTGCAAGGACTGAGAAGTAAGGATGAAGACCTGGGGTCTGCTTTAGTCTTT
CTTATTTTCTTCCTCTTCCTTACTATGTGTTTATTCCTTCTTTTTCTAGTTCCTTAACTTGTAAA

In a search of public sequence databases, it was found, for example, that the nucleic acid sequence has 622 of 673 bases (92%) identical to a 3659 bp synthetic omega 4-interferon mRNA (GENBANK-ID: A12146|acc:A12146) (E=2.6 e-122). It was also found, for example, that the nucleic acid sequence of the invention has 233 of 244 bases (95%) identical to Homo sapiens interferon genes LeIF-L, LeIF-J, and pseudogene LeIF-M located on chromosome 9 (9937 bp, GENBANK-ID: HSIFD1|acc:V00531, E=2.2e-42). The strong (95%) homology of a 243 base pair segment of the current invention with 244 base pair region of the above GenBank sequence suggests that the current invention represents an expressed interferon gene and polypeptide. Public nucleotide databases include all GenBank databases and the GeneSeq patent database.

An open reading frame was identified beginning with an ATG initiation codon at nucleotides 4-6 and ending with a TAA codon at nucleotides 685-687. A putative untranslated region upstream from the initiation codon and downstream from the termination codon is underlined in Table 2A, and the start and stop codons are in bold letters. The disclosed NOV2 polypeptide (SEQ ID NO: 4) encoded by SEQ ID NO: 3 is 227 amino acid residues and is presented using the one-letter code in Table 2B. The NOV2 protein was analyzed for signal peptide prediction and cellular localization. SignalPep results predict that NOV2 is cleaved between position 29 and 30 of SEQ ID NO: 4, i.e., at the slash in the amino acid sequence VGS-LG. Psort and Hydropathy profiles also predict that NOV2 contains a signal peptide and is likely to be localized at the plasma membrane (certainty of 0.9190).

TABLE 2B
Encoded NOV2 protein sequence (SEQ ID NO:4).

MVLLEQDFQFGLGPLLVALLLCHCGPVGS/LGFDLPQNHGLLSRNTLALLGQMQRISPFLCLKDRRDFRFP
LFFVDGSQLHKAQALSVLHEMLQQIFSVYPTECSSAAWNMTLLDQLHTGFHLYLGCLESRLGQAIGEEES
VGVIVAPTLALRRYFQGIHGIQRIYLKBKKYSDCAWEVLRVGIMKSFSSSTNLQGLRSKDEDLGSALVFL
IFFLFLTMCLFLLFLVP

The full amino acid sequence of the protein of the invention was found to have 139 of 195 amino acid residues (71%) identical to, and 153 of 195 residues (78%) positive with, the 195 amino acid residue interferon omega-1 precursor (interferon alpha-II-1) protein from Homo sapiens (ptnr: SWISSPROT-ACC:P05000) (E=9.2e-65). Public amino acid databases include the GenBank databases, SwissProt, PDB and PIR.

As shown in Table 2C, Patp analysis shows that NOV2 has significant homology with a number of interferons. Interferons (IFN) produce antiviral and antiproliferative responses in cells. Interferons are classified into five groups, all of them related but gamma-IFN.

TABLE 2C
Patp alignments of NOV2

| | | | Smallest |
|---|---|---|---|
| | | | Sum |
| | Reading | High | Prob. |
| Sequences producing High-scoring Segment Pairs: | Frame | Score | P (N) |
| Patp:P60253 Interferon-omega-1—H. sapiens, 195 aa. | +1 | 665 | 1.6e − 64 |
| Patp:Y22635 Human interferon-omega protein—H. sapiens. | +1 | 665 | 1.6e − 64 |
| Patp:B13433 Human interferon omega—H. sapiens, 195 . . . | +1 | 665 | 1.6e − 64 |
| Patp:P60355 Sequence of human leucocyte interferon . . . | +1 | 657 | 1.1e − 63 |

For example, a BLAST against patp: Y22635, a 195 amino acid interferon omega protein from Homo sapiens, produced 139/195 (71%) identity, and 153/195 (78%) positives (E=1.6e-64). See, PCT application WO 99/26663, describing human interferon-omega and constructs and vectors containing interferon-omega. The compositions containing the constructs are used in human or veterinary medicine for treating a wide variety of cancers, particularly melanoma, glioma, and ovarian carcinoma (also metastases to lung and liver), or pancreatic, gastric, colonic, and mesenteric cancers. The proteins listed in Table 2C show long segments of amino acid identity, as shown by the vertical lines (|) in Table 2D. Conservative substitutions are indicated by a plus sign (+).

TABLE 2D
Alignment of NOV2 Y22635 Human interferon omega (SEQ ID NO:32)

Length = 195 Plus Strand HSPs:
Score = 665 (234.1 bits), Expect = 1.6e-64, P = 1.6e-64
Identities = 139/195 (71%), Positives = 153/195 (78%), Frame = +1

| | |
|---|---|
| NOV2: | 37 LGPLLVALLLCHCGPVGSLGFDLPQNHGLLSRNTLALLGQMQRISPFLCLKDRRDFRFPL 216 |
| \| \|\|\| \|\|++ \|\|\|\|\|\| \|\|\|\|\|\|\|\|\|\|\|\|\|\| \|\| \|\|+\|\|\|\|\|\|\|\|\|\|\|\|\|\|\|\|\| | |
| IFN: | 4 LFPLLAALVMTSYSPVGSLGCDLPQNHGLLSRNTLVLLHQMRRISPFLCLKDRRDFRFPQ 63 |
| NOV2: | 217 FFVDGSQLHKAQALSVLHEMLQQIFSVYPTECSSAAWNMTLLDQLHTGFHLYLGCLESRL 396 |
| \| \|\|\|\| \|\| +\|\|\|\|\|\|\|\|\|\|\|\|++\|\| \|\|\|\|\|\|\|\|\|\|\|\|\|\|\|\| \| \| \|\|+\| | |
| IFN: | 64 EMVKGSQLQKAHVMSVLHEMLQQIFSLFHTERSSAAWNMTLLDQLHTGLHQQLQHLETCL 123 |
| NOV2: | 397 GQAIGEEESVGVIVAPTLALRRYFQGIHGIQRIYLKEKKYSDCAWEVLRVGIMKSFSSST 576 |
| \| +\|\| \|\| \| \| +\| \| \|\|\|\|\|\|\|\| \|+\|\|\|\|\|\|\|\|\|\|\|\|\|\|+\|+\|\|\|\| \|\| | |
| IFN: | 124 LQVVGEGESAGAISSPALTLRRYFQGI----RVYLKEKKYSDCAWEVVRMEIMKSLFLST 179 |
| NOV2: | 577 NLQG-LRSKDEDLGSA 621 |
| \|+\| \|\|\|\|\| \|\|\|\|+ | |
| IFN: | 180 NMQERLRSKDRDLGSS 195 |

Other BLAST results including the sequences used for ClustalW analysis is presented in Table 2E.

TABLE 2E
BLAST results for NOV2

| Gene Index/ | Protein/ | Length | Identity | Positives | |
|---|---|---|---|---|---|
| Identifier | Organism | (aa) | (%) | (%) | Expect |
| gi\|4504605\|ref\|NP_0 | Interferon | 195 | 133/182 | 145/182 | 1e-63 |
| 02168.1\| | (IFN), omega | | (73%) | (79%) | |
| | 1 Homo | | | | |
| | sapiens | | | | |
| | (INTERFERON | | | | |
| | ALPHA-II-l) | | | | |
| gi\|386800\|gb\|AAA527 | IFN-alpha | 195 | 132/182 | 144/182 | 8e-63 |
| 24.1\|(M11003) | Homo sapiens | | (72%) | (78%) | |
| gi\|758083\|emb\|CAA26 | IFN omega | 174 | 129/178 | 141/178 | 7e-61 |
| 501.1\|(X02669) | precursor | | (72%) | (78%) | |
| | Homo sapiens | | | | |
| gi\|847816\|gb\|AAA700 | IFN omega-1 | 174 | 126/175 | 137/175 | 3e-59 |
| 91.1\|(U25670) | Homo sapiens | | (72%) | (78%) | |
| gi\|124502\|sp\|P05002 | IFN-omega-2 | 195 | 117/181 | 136/181 | 2e-53 |
| \|IN02_HORSE | precursor | | (64%) | (74%) | |
| | Equus | | | | |
| | caballus | | | | |

This information is presented graphically in the multiple sequence alignment given in Table 2F (with NOV2 being shown on line 1) as a ClustalW analysis comparing NOV2 with related protein sequences.

TABLE 2F
Information for the ClustalW proteins:

1) NOV2 (SEQ ID NO:4)
2) gi|4504605|ref|NP_002168.1| interferon, omega 1 (SEQ ID NO:33)
3) gi|386800|gb|AAA52724.1| (M11003) interferon-alpha (SEQ ID NO:34)
4) gi|758083|emb|CAA26501.1| (X02669) human interferon omega precursor (SEQ ID NO:35)
5) gi|847816|gb|AAA70091.1|(U25670) interferon omega (SEQ ID NO:36)
6) gi|124502|sp|P05002|INO2_HORSE INTERFERON OMEGA-2 PRECURSOR (INTERFERON ALPHA-II-2) (SEQ ID NO:37)

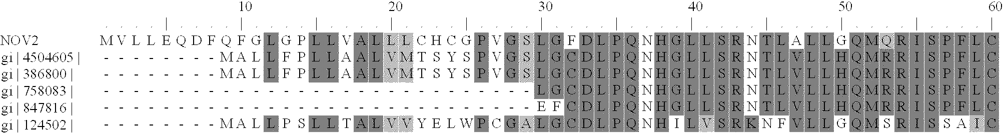

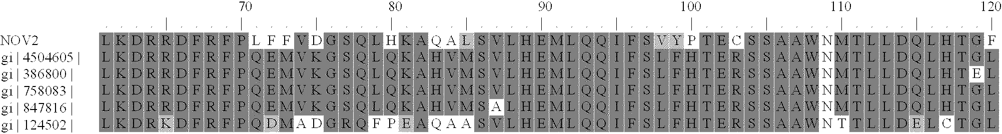

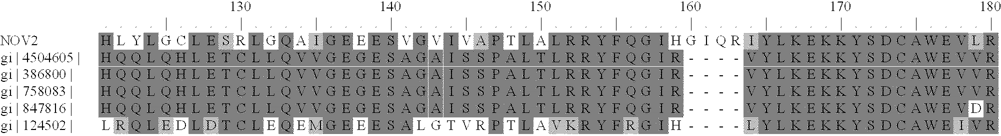

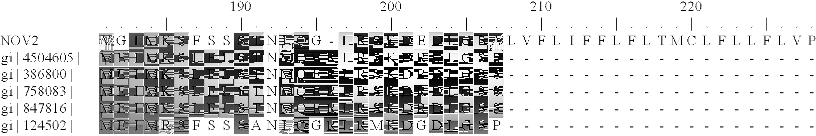

DOMAIN results for NOV2 were collected from the Conserved Domain Database (CDD) with Reverse Position Specific BLAST. This BLAST samples domains found in the Smart and Pfam collections. NOV2 showed significant alignment with Pfam 00143 (interferon, Interferon alpha/beta domain, E=6e-57) and Smart IFabd (Interferon alpha, beta and delta, 117 amino acid residues E=8e-31). The alignment with Pfam00143 is shown in Table 2G. The similarity of NOV2 with the Interferon alpha/beta domain indicates that the NOV2 sequence has properties similar to those of other proteins known to contain this domain as well as to the interferon domain itself.

TABLE 2G
Domain Analysis of NOV2

gnl|Pfam|pfam00143, interferon, Interferon alpha/beta domain
CD-Length = 190 residues, 91.6% aligned
Score = 213 bits (543), Expect = 6e-57

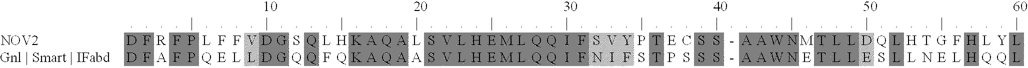

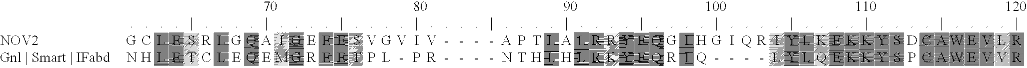

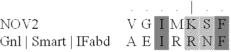

Type I interferons (for example, IFN-alpha, IFN-beta, and IFN-omega) bind to the type I interferon (IFN) receptor and elicit signaling events including activation of the Jak/Stat and IRS pathways (OMIM: 602376). Henco et al. (J Mol Biol. 185:227-260, 1985) compiled partial maps of the interferon gene cluster located on 9p21. These maps showed that members of the two main families of genes in the IFN superfamily, interferon-alpha (OMIM-147660) and IFN-omega, are interspersed. Olopade et al. (Genomics 14:437-443, 1992) studied the deletions of the short arm of chromosome 9 frequently observed in acute lymphoblastic leukemia and in gliomas. These deletions often include the entire interferon gene cluster, which comprises about 26 IFN-alpha, IFN-omega, and IFN-beta 1 (OMIM-147640) genes, as well as the gene for methylthioadenosine phosphorylase (MTAP; OMIM-156540). By comparing microscopic deletions with the genes lost at the molecular level, Olopade et al. determined the order of these genes on 9p to be:

tel—IFN-beta 1—IFN-alpha/IFN-omega cluster—MTAP—cen.

In a few cell lines and in primary leukemia cells, they observed deletions that had breakpoints within the interferon gene cluster and resulted in partial loss of the interferon genes. These partial deletions allowed them to determine the order of some genes or groups of genes in the IFN-alpha/IFN-omega gene cluster. From their deletion analysis, Olopade et al. deduced the following order of the interferon gene on 9p:

pter—IFN-beta 1—(IFN-omega 1, IFN-alpha 21)—IFN-omega P15—IFN-alpha 4—IFN-omega 9—IFN-alpha 7—IFN-alpha 10—IFN-omega P18—IFN-alpha P16—IFN-alpha 17—IFN-alpha 14—IFN-alpha 22, v5, IFN-alpha P20, IFN-alpha 6, IFN-alpha 13, IFN-alpha 2)—(IFN-alpha 8, IFN-omega 2, IFN-omega P19, IFN-alpha 1)—MTAP—cen.

The genes within the large linkage group are arranged in tandem with their 3-prime end pointing toward the telomere of the short arm. Thus, at least two functional interferon-omega genes, IFN-omega 1 and IFN-omega 2, were mapped and several interferon-omega pseudogenes, (e.g., IFN-omega P15) were localized.

Apart from their antiviral activities interferons also possess antiproliferative and immunomodulating activities and influence the metabolism, growth and differentiation of cells in many different ways.

Omega-Interferon (IFN-omega) is a natural component of human leukocyte interferon (LeIFN). This interferon is called alsoIFN-alpha I1. It displays a high degree of homology with various IFN-alpha species including positions of the cysteine residues involved in disulfide bonds. However, sequence divergence allows classification as a unique protein family. IFN-omega binds to the same receptors as IFN-alpha and IFN-beta. To date the exact biological activities and the physiological role of this interferon are unknown. It is thought to influence cell proliferation and differentiation. One related protein is bovine trophoblast protein-1 (TP- 1), which is produced in large quantities during pregnancy, and is a potent antiviral, antiproliferative and immunosuppressive agent. See, generally, http://www.copewithcytokines.de.

Mire-Sluis et al describe bioassays for IFN-alpha, IFN-beta and IFN-omega that exploit the ability of these factors to inhibit proliferation of TF-1 cells (a human premyeloid cell line) induced by GM-CSF. Mire-Sluis, et al., J. of Immunol. Meth. 195:55-61, 1996. The bioassays can be used also with Epo and TF-1 cells, or Epo and Epo-transfected UT-7 cells.

The nucleic acids and proteins of the invention are useful in potential therapeutic applications implicated in various interferon-related pathological disorders, described further below. For example, a cDNA encoding the interferon -like protein may be useful in gene therapy, and the interferon -like protein may be useful when administered to a subject in need thereof. By way of nonlimiting example, the compositions of the present invention will have efficacy for treatment of patients suffering from hyperproliferative disorders, viral or other pathogenic infection, immune disorders, and disorders of the neuroendocrine system.

For example, the nucleic acids and proteins of the invention are useful in potential therapeutic applications implicated in viral infections; neurologic disease, cancer (especially acute lymphoblastic leukemia and in gliomas, malignant melanoma; non-Hodgkin's lymphoma, squamous cell carcinoma); immune disorders; and/or other pathologies and disorders including their immunotherapy. Thus, a cDNA encoding the interferon-like protein may be useful in gene therapy, and the interferon-like protein may be useful when administered to a subject in need thereof. By way of nonlimiting example, the compositions of the present invention will have efficacy for treatment of patients suffering from viral infections; cancer especially acute lymphoblastic leukemia and in gliomas, neurologic disease; and/or immune disorders.

The novel nucleic acid encoding the interferon-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV2 protein has multiple hydrophilic regions, each of which can be used as an immunogen. In one embodiment, a contemplated NOV2 epitope is from about amino acids 40 to 50. In another embodiment, a NOV2 epitope is from about amino acids 55 to 65. In additional embodiments, NOV2 epitopes are from amino acids 75 to 85, and from amino acids 150 to 200. These novel proteins can also be used to develop assay system for functional analysis.

These novel proteins can be used in assay systems for functional analysis of various human disorders, which will help in understanding of pathology of the disease and development of new drug targets for various disorders.

NOV3

NOV3 is a novel Receptor Tyrosine Kinase-like protein and nucleic acid encoding it. This sequence was initially identified by searching CuraGen's Human SeqCalling database for DNA sequences that translate into proteins with similarity to a protein family of interest. SeqCalling assembly 29145493 was identified as having suitable similarity. SeqCalling assembly 29145493 was analyzed further to identify an open reading frame encoding for a novel full length protein and novel splice forms of this gene. This was done by extending the SeqCalling assembly using suitable additional SeqCalling assemblies, publicly available EST sequences and public genomic sequence. Public ESTs and additional CuraGen SeqCalling assemblies were identified by the Curatools program SeqExtend. They were included in the DNA sequence extension for SeqCalling assembly 29145493 only when sufficient identical overlap was found. These inclusions are described below. The genomic clone AC023225 (chromosome 1) was identified as having regions with 100% identity to the SeqCalling assembly 29145493 and were selected for analysis because this identity implied that the clone AC023225 contained the sequence of the genomic locus for SeqCalling assembly 29145493. The genomic clone AC023225 was analyzed by Genscan and Grail to identify exons and putative coding sequences/open reading frames. The clone AC023225 was also analyzed by TblastN, BlastX and other homology programs to identify regions translating to proteins with similarity to the original protein/protein family of interest.

The results of these analyses were integrated and manually corrected for apparent inconsistencies, thereby obtaining the sequence encoding the full-length protein. When necessary, the process to identify and analyze cDNAs/ESTs and genomic clones was reiterated to derive the full-length sequence. NOV3 describes this full-length DNA sequence(s) and the full-length protein sequence(s) which they encode.

The novel nucleic acid of 3003 nucleotides (29145493_EXT, SEQ ID NO: 5) encoding a novel tyrosine kinase-like protein is shown in Table 3A. An open reading frame (ORF) was identified beginning with an ATG initiation codon at nucleotides 1-3 and ending with a TGA codon at nucleotides 3001-3003. In Table 3A, the start and stop codons are in bold letters.

TABLE 3A
NOV3 Nucleotide Sequence (SEQ ID NO:5)

ATGGTATTGACAACTGCTATACCAGCCTGGCTTCTTAGCTGTTCCCTCCCACTCTCATCCTGGGCCCACC
ATGCGACACCGCCCCTCCGTCTAGTAGTTATCCTCCTGGATTCCAAAGCCTCCCAGGCCGAGCTGGGCTG
GACTGCACTGCCAAGTAATGGGTGGGAGGAGATCAGCGGCGTGGATGAACACGACCGTCCCATCCGCACG
TACCAAGTGTGCAATGTGCTGGAGCCCAACCAGGACAACTGGCTGCAGACTGGCTGGATAAGCCGTGGCC
GCGGGCAGCGCATCTTCGTGGAACTGCAGTTCACACTCCGTGACTGCAGCAGCATCCCTGGCGCCGCGGG
TACCTGCAAGGAGACCTTCAACGTCTACTACCTGGAAACTGAGGCCGACCTGGGCCGTGGGCGTCCCCGC
CTAGGCGGCAGCCGGCCCCGCAAAATCGACACGATCGCGGCGGACGAGAGCTTCACGCAGGGCGACCTGG
GTGAGCGCAAGATGAAGCTGAACACAGAGGTGCGCGAGATCGGACCGCTCAGCCGGCGGGGTTTCCACCT
GGCCTTTCAGGACGTGGGCGCATGCGTGGCGCTTGTCTCGGTGCGCGTCTACTACAAGCAGTGCCGCGCC
ACCGTGCGGGGCCTGGCCACGTTCCCAGCCACCGCAGCCGAGAGCGCCTTCTCCACACTGGTGGAAGTGG
CCGGAACGTGCGTGGCGCACTCGGAAGGGGAGCCTGGCAGCCCCCCACGCATGCACTGCGGCGCCGACGG
CGAGTGGCTGGTGCCTGTGGGCCGCTGCAGCTGCAGCGCGGGATTCCAGGAGCGTGGTGACTTCTGCGAA
TGTCCCCCAGGGTTTTACAAGGTGTCCCCGCGGCGGCCCCTCTGCTCACCGTGCCCAGAGCACAGCCGGG
CCCTGGAAAACGCCTCCACCTTCTGCGTGTGCCAGGACAGCTATGCGCGCTCACCCACCGACCCGCCCTC
GGCTTCCTGCACCCGTCCGCCGTCGGCGCCGCGGGACCTGCAGTACAGCCTGAGCCGCTCGCCGCTGGTG
CTGCGACTGCGCTGGCTGCCGCCGGCCGACTCGGGAGGCCGCTCGGACGTCACCTACTCGCTGCTGTGCC
TGCGCTGCGGCCGCGAGGGCCCGGCGGGCGCCTGCGAGCCGTGCGGGCCGCGCGTGGCCTTCCTACCGCG
CCAGGCAGGGCTGCGGGAGCGAGCCGCCACGCTGCTGCACCTGCGGCCCGGCGCGCGCTACACCGTGCGC
GTGGCCGCGCTCAACGGCGTCTCGGGCCCGGCGGCCGCCGCGGGAACCACCTACGCGCAGGTCACCGTCT
CCACCGGGCCCTCAGCGCCCTGGGAGGAGGATGAGATCCGCAGGGACCGAGTGGAACCCCAGAGCGTGTC
CCTGTCGTGGCGGGAGCCCATCCCTGCCGGAGCCCCTGGGGCCAATGACACGGAGTACGAGATCCGATAC
TACGAGAAGCAGAGTGAGCAGACTTACTCCATGGTGAAGACAGGGGCGCCCACAGTCACCGTCACCAACC
TGAAGCCGGCTACCCGCTACGTCTTTCAGATCCGGGCCGCTTCCCCGGGGCCATCCTGGGAGGCCCAGAG
TTTTAACCCCAGCATTGAAGTACAGACCCTGGGGGAGGCTGCCTCAGGGTCCAGGGACCAGAGCCCCGCC
ATTGTCGTCACCGTAGTGACCATCTCGGCCCTCCTCGTCCTGGGCTCCGTGATGAGTGTGCTGGCCATTT
GGAGGAGGAGGCCCTGCAGCTATGGCAAAGGAGGAGGGGATGCCCATGATGAAGAGGAGCTGTATTTCCA
CTGTAAAGTCCCAACACGTCGCACATTCCTGGACCCCCAGAGCTGTGGGGACCTGCTGCAGGCTGTGCAT
CTGTTCGCCAAGGAACTGGATGCGAAAAGCGTCACGCTGGAGAGGAGCCTTGGAGGAGGCAAGTTTGGGG
AGCTGTGCTGTGGCTGCTTGCAGCTCCCCGGTCGCCAGGAGCTGCTCGTAGCCGTGCACATGCTGAGGGA
CAGCGCCTCCGACTCACAGAGGCTCGGCTTCCTGGCCGAGGCCCTCACGCTGGGCCAGTTTGACCATAGC
CACATCGTGCGGCTGGAGGGCGTTGTTACCCGAGGTAGGACCTTGATGATTGTCACCGAGTACATGAGCC
ATGGGGCCCTGGACGGCTTCCTCAGGCACGAGGGGCAGCTGGTGGCTGGGCAACTGATGGGGTTGCTGCC
TGGGCTGGCATCAGCCATGAAGTATCTGTCAGAGATGGGCTACGTTCACCGGGGCCTGGCAGCTCGCCAT
GTGCTGGTCAGCAGCGACCTTGTCTGCAAGATCTCTGGCTTCGGGCGGGGCCCCCGGGACCGATCAGAGG
CTGTCTACACCACTGGCCGGAGCCCAGCGCTATGGGCCGCTCCCGAGACACTTCAGTTTGGCCACTTCAG
CTCTGCCAGTGACGTGTGGAGCTTCGGCATCATCATGTGGGAGGTGATGGCCTTTGGGGAGCGGCCTTAC
TGGGACATGTCTGGCCAAGACGTGAAGGCTGTGGAGGATGGCTTCCGGCTGCCACCCCCCAGGAACTGTC
CTAACCTTCTGCACCGACTAATGCTCGACTGCTGGCAGAAGGACCCAGGTGAGCGGCCCAGGTTCTCCCA
GATCCACAGCATCCTGAGCAAGATGGTGCAGGACCCAGAGCCCCCCAAGTGTGCCCTGACTACCTGTCCC
AGGCCTCCCACTCCACTAGCCGACCGTGCCTTCTCCACCTTCCCCTCCTTTGGCTCTGTGGGCGCCTGGC
TGGAGGCCCTGGACCTGTGCCGCTACAAGGACAGCTTCGCGGCTGCTGGCTATGGGAGCCTGGAGGCCGT
GGCCGAGATGACTGCCCAGGACCTGGTGAGCCTAGGCATCTCTTTGGCTGAACATCGAGAGGCCCTCCTC
AGCGGGATCAGCGCCCTGCAGGCACGAGTGCTCCAGCTGCAGGGCCAGGGGGTGCAGGTGTGA

The disclosed 29145493_EXT nucleic acid sequence has that the nucleic acid sequence has 735 of 1211 nucleotides (60%) identical to Kinase 1 Mus musculus (GENBANK-ID:MMKIN1).

The disclosed NOV3 polypeptide (SEQ ID NO: 6) encoded by SEQ ID NO: 9 is 1000 amino acid residues and is presented using the one-letter code in Table 3B. The first 70 amino acids of the disclosed NOV3 protein were analyzed for signal peptide prediction and cellular localization. SignalP results predict that NOV3 is cleaved between position 22 and 23 of SEQ ID NO: 6, i.e., at the slash in the amino acid sequence SWA-HH. Psort and Hydropathy profiles also predict that NOV3 contains a signal peptide and is likely to be localized at the plasma membrane (certainty of 0.4600).

TABLE 3B
Encoded NOV3 protein sequence (SEQ ID NO:6).

MVLTTAIPAWLLSCSLPLSSWA/HHATPPLRLVVILLDSKASQAELGWTALPSNGWEEISGVDEHDRPIRT
YQVCNVLEPNQDNWLQTGWISRGRGQRIFVELQFTLRDCSSIPGAAGTCKETFNVYYLETEADLGRGRPR
LGGSRPRKIDTIAADESFTQGDLGERKMKLNTEVREIGPLSRRGFHLAFQDVGACVALVSVRVYYKQCRA
TVRGLATFPATAAESAFSTLVEVAGTCVAHSEGEPGSPPRMHCGADGEWLVPVGRCSCSAGFQERGDFCE
CPPGFYKVSPRRPLCSPCPEHSRALENASTFCVCQDSYARSPTDPPSASCTRPPSAPRDLQYSLSRSPLV
LRLRWLPPADSGGRSDVTYSLLCLRCGREGPAGACEPCGPRVAFLPRQAGLRERAATLLHLRPGARYTVR
VAALNGVSGPAAAAGTTYAQVTVSTGPSAPWEEDEIRRDRVEPQSVSLSWREPIPAGAPGANDTEYEIRY
YEKQSEQTYSMVKTGAPTVTVTNLKPATRYVFQIRAASPGPSWEAQSFNPSIEVQTLGEAASGSRDQSPA
IVVTVVTISALLVLGSVMSVLAIWRRRPCSYGKGGGDAHDEEELYFHCKVPTRRTFLDPQSCGDLLQAVH
LFAKELDAKSVTLERSLGGGKFGELCCGCLQLPGRQELLVAVHMLRDSASDSQRLGFLAEALTLGQFDHS
HIVRLEGVVTRGRTLMIVTEYMSHGALDGFLRHEGQLVAGQLMGLLPGLASAMKYLSEMGYVHRGLAARH
VLVSSDLVCKISGFGRGPRDRSEAVYTTGRSPALWAAPETLQFGHFSSASDVWSFGIIMWEVMAFGERPY
WDMSGQDVKAVEDGFRLPPPRNCPNLLHRLMLDCWQKDPGERPRFSQIHSILSKMVQDPEPPKCALTTCP
RPPTPLADRAFSTFPSFGSVGAWLEALDLCRYKDSFAAAGYGSLEAVAEMTAQDLVSLGISLAEHREALL
SGISALQARVLQLQGQGVQV

A BLASTX search was performed against public protein databases. The full amino acid sequence of the protein of the invention was found to have 537 of 1000 amino acid residues (53%) identical to, and 717 of 1000 residues (71%) positive with, the 993 amino acid residue tyrosine kinase receptor protein from Gallus gallus (ptnr:SPTREMBL-ACC:O42422 EPH-LIKE RECEPTOR TYROSINE KINASE PRECURSOR (EC 2.7.1.112) (TYROSINE- PROTEIN KINASE RECEPTOR CEPHA7), SEQ ID NO: 39 (E=1.8 e⁻²⁸⁸). These proteins have large regions of identity, as shown in Table 3C. For example, the region from NOV3 amino acids 148 to 181 has a stretch of 34 identical amino acids.

TABLE 3C
Alignment of NOV3 with O42422 (SEQ ID NO:39).

Score = 2779 (978.3 bits), Expect = 1.8e-288, P = 1.8e-288
Identities = 537/1000 (53%), Positives = 721/ 00 (72%), Frame = +1

| | | | |
|---|---|---|---|
| NOV3: | 1 | MVLTTAIPAWLLSCSLPLSSWAHHATPPLRLVVILLDSKASQAELGWTALPSNGWEEISG | 60 |
| | | \|\|\| + +\| \|++ \|\|+ \| +\|\| \|\|\|\|\|\|\|\| \| \|\| \| + \| \|\|\|\|\|\|\|\| | |
| 042422 | 1 | MVLRSRLPPWIMLCSVWLLRFAHTGEAQAAKEVILLDSKAQQTELEWISSPPNGWEEISG | 60 |
| NOV3: | 61 | VDEHDRPIRTYQVCNVLEPNQDNWLQTGWISRGRGQRIFVELQFTLRDCSSIPGAAGTCK | 120 |
| | | +\|\|+ \|\|\|\|\|\|\|\| \|+\| \|\|+\|\|\|+\| \|\|++111\|\|\|\|\|\|\|+\|\|\|\|\|\|+\|+\|\| \|\|\|\| | |
| 042422: | 61 | LDENYTPIRTYQVCQVMESNQNNWLRTNWIAKSNAQRIFVELKFTLRDCNSLPGVLGTCK | 120 |
| NOV3: | 121 | ETFNVYYLETEADLGRGRPRLGGSRPRKIDTIAADESFTQGDLGERKMKLNTEVREIGPL | 150 |
| | | \|\|\|\|+\|\| \|\| \|\|+\| + ++ \|\|\|\|\|\|\|\|\|\|\|\|\|\|\|\|\|\|\|\|\|\|\|\|\|\|\|\|\|\|\|\|\|\| | |
| 042422: | 121 | ETFNLYYYETDYDTGRN---IRENQYVKIDTIAADESFTQGDLGERKMKLNTEVREIGPL | 177 |
| NOV3: | 181 | SRRGFHLAFQDVGACVALVSVRVYYKQCRATVRGLATFPATAAESAFSTLVEVAGTCVAH | 240 |
| | | \|++\|\|+\|\|\|\|\|\|\|\|\|+\|\|\|\|\|+\|\|\|\|+\| + + \|\| \|\| \| \| \|\|+\|\|\|\| \|\|\|\|+ | |
| 042422: | 178 | SKKGFYLAFQDVGACIALVSVKVYYKKCWSIIENLAIFPDTVTGSEFSSLVEVRGTCVSS | 237 |
| NOV3: | 241 | SEGEPGSPPRMHCGADGEWLVPVGRCSCSAGFQERGDFCE-CPPGFYKVSPRRPLCSPCP | 299 |
| | | +\| \| + \|+\|\|\| \|+\|\|\|\|\|\|+\|+\| \| \|\|+\|++\|\| \|\| \| \|\|\|\| \| + \|\| \|\| | |
| 042422: | 238 | AEEEAENSPKMHCSAEGEWLVPIGKCICKAGYQQKGDTCEPCGRGFYKSSSQDLQCSRCP | 297 |
| N0V3: | 300 | EHSRALENASTFCVCQDSYARSPTDPPSASCTRPPSAPRDLQYSLSRSPLVLRLRWLPPA | 359 |
| | | \|\| + + \|+ \| \|+\|\|\| \|+\|+\|\|\| +\|\|\|\|\|\|\|\|++\| ++++++ + \| \| \|\|\| | |
| 042422: | 298 | THSFSDKEGSSRCDCEDSYYRAPSDPPYVACTRPPSAPQNLIFNINQT--TVSLEWSPPA | 355 |
| N0V3: | 360 | DSGGRSDVTYSLLCLRCGREGPAGACEPCGPRVAFLPRQAGLRERAATLLHLRPGARYTV | 419 |
| | | \|+\|\|\|+\|\|\|\| +\|\| \|\| \| \| \| \| \|\|\| + ++\|+\| \|\| \|++ \| \| \|\| | |
| 042422: | 356 | DNGGRNDVTYRILCKRCSWE--QGECVPCGSNIGYMPQQTGLVDNYVTVMDLLAHANYTF | 413 |
| NOV3: | 420 | RVAALNGVSGPAAAAGTTYAQVTVSTGPSAPWEEDEIRRDRVEPQSVSLSWREPIPAGAP | 479 |
| | | \| \|+\|\|\|\| + + +\| \|+++\|\| +\|\|1+ + ++\|\| +\|\| \|\|\|+\|\| \| | |
| 042422: | 414 | EVEAVNGVSD-LSRSQRLFAAVSITTGQAAPSQVSGVMKERVLQRSVELSWQEP---EHP | 469 |
| NOV3: | 480 | GANDTEYEIRYYEK-QSEQTYSMVKTGAPTVTVTNLKPATRYVFQIRAASPGPSWEAQSF | 538 |
| | | \|\|\|\|\|+\|\|\|\| \| \|+\|\|\| \|\|\| + + ++ \|\|\|\| \| \|\|\|\|\|\|\| + ++ | |
| 042422: | 470 | NGVITEYEIKYYEKDQRERTYSTVKTKSTSASINNLKPGTVYVFQIRAFTAAGYG---NY | 526 |
| NOV3: | 539 | NPSIEVQTLGEAASG--SRDQSPATVVTVVTISALLVLGSVMSVLAIWRRRPCSYGKGGG | 596 |
| | | +\| ++\| \|\| \|\| + \| +\|+\| \|++ \|\| ++ ++\| ++ \| \|\| \| \| \| | |
| 042422: | 527 | SPRLDVATLEEATATAVSSEQNPVIIIAVVAVAGTIILVFMVFGFIIGRRH-CGYSKA-- | 583 |
| NOV3: | 597 | DAHDEEELYFHCKVPTRRTFLDPQSCGDLLQAVHLFAKELDAKSVTLERSLGGGKFGELC | 656 |
| | | \|111+\|\|\|\|\|\| \| \| +\|++\|\|++ \| +\|\|\| \|\|\|\|\|\|\| + +\|\| +\| \|+\|\|\|+\| | |
| 042422: | 584 | DQEGDEELYFHFKFPGTKTYIDPETYEDPNRAVHQFAKELDASCIKIERVIGAGEFGEVC | 643 |
| NOV3: | 657 | CGCLQLPGRQELLVAVHMLRDSASDSQRLGFLAEALTLGQFDHSHIVRLEGVVTRGRTLM | 716 |
| | | \| \|+\|\|\|++++ \|\|+ \|+ ++ \|\| \|\| \|\| +\|\|\|\|\| ++\| \|\|\|\|\|\|\|\|+ +\| | |
| 042422: | 644 | SGRLKLPGKRDVAVAIKTLKVGYTEKQRRDFLCEASIMGQFDHPNVVHLEGVVTRGKPVM | 703 |
| NOV3: | 717 | IVTEYMSHGALDGFLR-HEGQLVAGQLMGLLPGLASAMKYLSEMGYVHRGLAARHVLVSS | 775 |
| | | \|\| \|\|\| +\|\|\|\| \|\|\| \|+\|\| \|\|+\|+\| \|+\|+ \|+\|\|++\|\|\|\|\|\| \|\|\|\|++\|\|+\| | |
| 042422: | 704 | IVIEYMENGALDAFLRKHDGQFTVIQLVGMLRGIAAGMRYLADMGYVHRDLAARNILVNS | 763 |
| NOV3: | 776 | DLVCKISGFG--RGPRDRSEAVYTT--GRSPALWAAPETLQFGHFSSASDVWSFGIIMWE | 831 |
| | | +\|\|\|\|+\| \|\| \| \| \|\|\|\|\|\| \|+ \| \| \|\|\| +\|+ \|+\|\|\|\|\|\|\|+\|\|+\|\|\| | |
| 042422: | 764 | NLVCKVSDFGLSRVIEDDPEAVYTTTGGKIPVRWTAPEAIQYRKFTSASDVWSYGIVMWE | 823 |
| NOV3: | 832 | VMAFGERPYWDMSGQDV-KAVEDGFRLPPPRNCPNLLHRLMLDCWQKDPGERPRFSQIHS | 890 |
| | | \|\|++\|\|\|\|\|\|\|\|\| \|\|\| \|\|+\|+\|+\|\|\| \| +\|\| \|\|+\|\|\|\|\|\|\|\|+ \|\|\|\|+\| \|\| | |
| 042422: | 824 | VMSYGERPYWDMSNQDVIKAIEEGYRLPAPMDCPAGLHQLMLDCWQKERGERPKFEQIVG | 883 |
| NOV3: | 891 | ILSKMVQDPEPPKCALTTCPRPPTPLADRAFSTFPSFGSVGAWLEALDLCRYKDSFAAAG | 950 |
| | | \|\| \|\|+++\| \| \| \|\| \|\| +\|\| \|+ \| +\| \|\|\| \|\|+\|+ + \|\|\|\|+\| \|\|\| | |
| 042422: | 884 | ILDKMIRNPNSLKTPLGTCSRPISPLLDQNTPDFTTFCSVGEWLQAIKMERYKDNFTAAG | 943 |
| NOV3: | 951 | YGSLEAVAEMTAQDLVSLGISLAEHREALLSGISALQARVLQLQGQGVQV | 1000 |
| | | \| \|\|\|+\|\| \|\| +\|++\|\|\|\|+\| \|++ ++\| \| ++\|++\| \| \| \|+\|\| | |
| 042422: | 944 | YNSLESVARMTIEDVMSLGITLVGHQKKIMSSIQTMRAQMLHLHGTGIQV | 993 |

Patp results include those listed in Table 3D.

TABLE 3D
Patp alignments of NOV3

| | Reading | High | Smallest Sum |
|---|---|---|---|
| Sequences producing High-scoring Segment Pairs: | Frame | Score | Prob P(N) |
| patp: R85092 EPH-like receptor protein tyrosine kinase . . . | +1 | 2768 | 2.2e-287 |
| patp: W03421 Mouse developmental kinase 1 - Mus sp, 998aa. | +1 | 2762 | 9.6e-287 |
| patp: R85090 EPH-like receptor protein tyrosine kinase . . . | +1 | 2395 | 7.5e-248 |
| patp: R75711 Eph-related PTK Cek4 - Gallus sp, 983 aa. | +1 | 2320 | 6.6e-240 |
| patp: W83147 Rat receptor tyrosine kinase Ehk-1 - Rattus. | +1 | 2307 | 1.6e-238 |
| patp: R85936 Protein tyrosine-kinase bpTK7 - H. sapiens. . . | +1 | 2269 | 1.7e-234 |

The disclosed NOV3 protein (SEQ ID NO: 6) also has good identity with a number of olfactory receptor proteins, as shown in Table 3E.

This information is presented graphically in the multiple sequence alignment given in Table 3F (with NOV3 being shown on line 1) as a ClustalW analysis comparing NOV3 with related protein sequences.

TABLE 3E
BLAST results for NOV3

| Gene Index/ | Protein/ | Length | Identity | Positives | |
|---|---|---|---|---|---|
| Identifier | Organism | (aa) | (%) | (%) | Expect |
| Gi\|2497572\|sp\|Q15 | EPERIN TYPE-A | 998 | 513/998 | 674/998 | 0.0 |
| 375\|EPA7_HUMAN | RECEPTOR 7 | | (51%) | (67%) | |
| | PRECURSOR | | | | |
| | (TYROSINE- | | | | |
| | PROTEIN KINASE) | | | | |
| | Homo sapiens | | | | |
| Gi\|2462302\|emb\|CA | Eph-like | 993 | 513/993 | 676/993 | 0.0 |
| A74643.l\| | receptor | | (51%) | (67%) | |
| (Y14271) | tyrosine kinase | | | | |
| | Gallus gallus | | | | |
| Gi\|2497573\|sp\|Q6l | EPHRIN TYPE-A | 998 | 512/998 | 673/998 | 0.0 |
| 772\|EPA7_MOUSE | RECEPTOR 7 | | (51%) | (67%) | |
| | PRECURSOR | | | | |
| | (TYROSINE- | | | | |
| | PROTEIN KINASE | | | | |
| | RECEPTOR EHK-3; | | | | |
| | EPH HOMOLOGY | | | | |
| | KINASE-3; | | | | |
| | EMBRYONIC BRAIN | | | | |
| | KINASE; EBK; | | | | |
| | DEVELOPMENTAL | | | | |
| | KINASE 1; MOK | | | | |
| | 1) Mus | | | | |
| | musculus | | | | |
| Gi\|1706631\|sp\|P54 | Ehk-3, full | 998 | 510/998 | 674/998 | 0.0 |
| 759\|EPA7_RAT | length form | | (51%) | (67%) | |
| (U21954) | Rattus | | | | |
| | norvegicus | | | | |
| Gi\|7434436\|pir\|\|I | receptor | 991 | 452/961 | 621/961 | 0.0 |
| 78843 (L36644) | protein- | | (47%) | (64%) | |
| | tyrosine kinase | | | | |
| | Homo sapiens | | | | |

TABLE 3F
Information for the ClustalW proteins:

1) Novel NOV3 (SEQ ID NO:6)
2) gi|4758282|ref|NP_004431.1|EphA; Hek11; ephrin receptor EphA7 (SEQ ID NO:40)
3) gi|8134447|sp|O42422|EPA7 Ephrin Type-A Receptor 7 Precursor (Tyrosine-PK Receptor Cepha7) (CEK11) (SEQ ID NO:41)
4) gi|2497573|Sp|Q61772|Epa7 Mouse Ephrin Type-A Receptor 7 Precursor (Tyrosine-PK Receptor Ehk-3) (Eph Homology
Kinase-3) (Embryonic Brainkinase) (EBK) (Developmental Kinase 1) (MDK-1) (SEQ ID NO:42)
5) (gi|1706631|sp|P54759|EPA7_RAT Ephrin Type-A Receptor 7 Precursor (Tyrosine-Protein Kinase Receptor Ehk-3) (Eph
Homology Kinase-3) (SEQ ID NO:43)
6) gi|7434436|pir||I78843 receptor protein-tyrosine kinase - human (fragment) (SEQ ID NO:44)

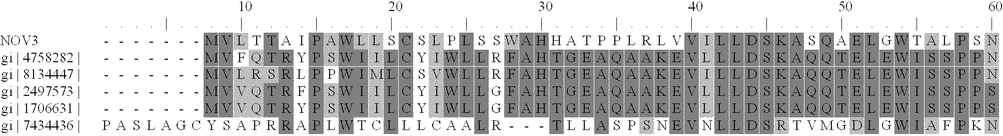

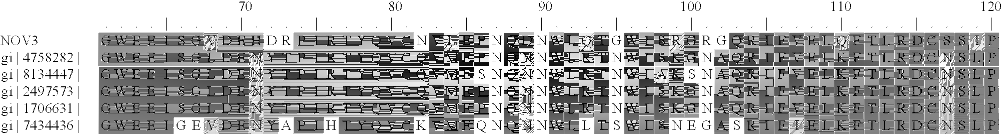

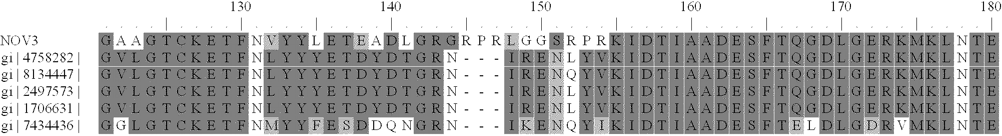

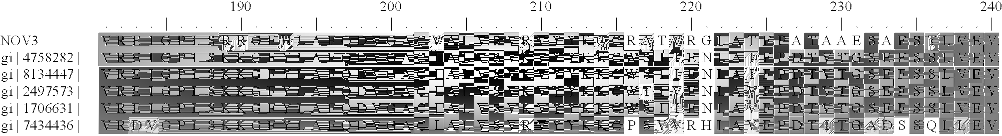

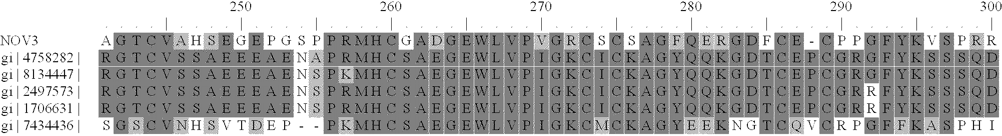

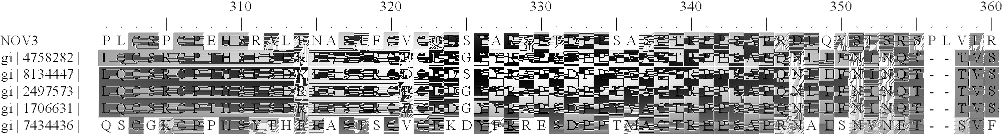

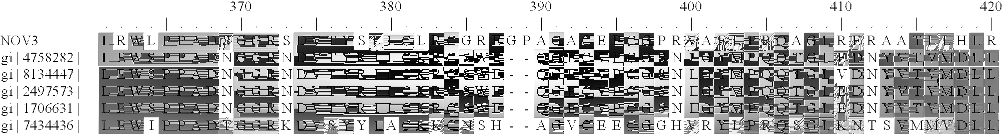

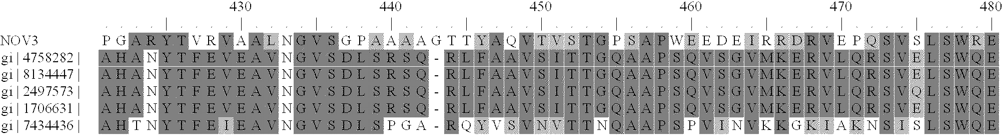

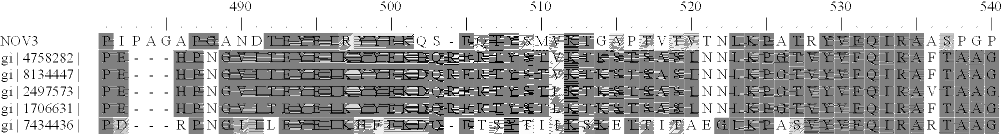

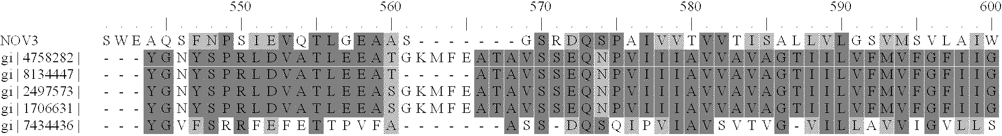

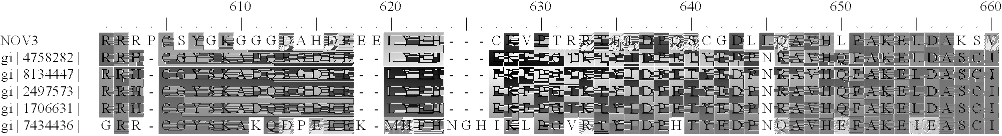

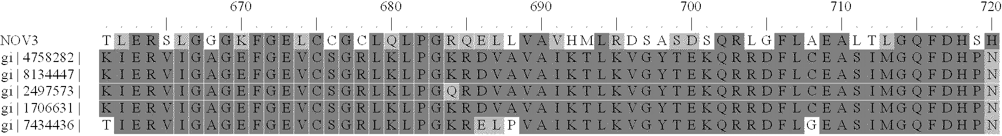

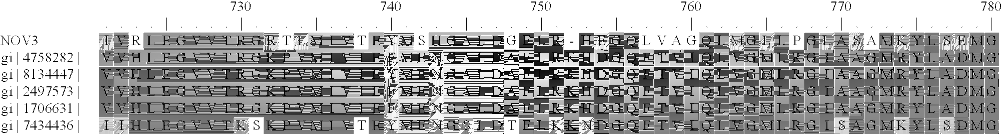

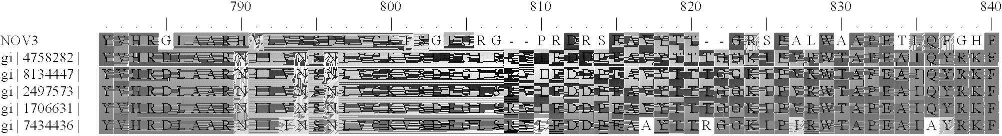

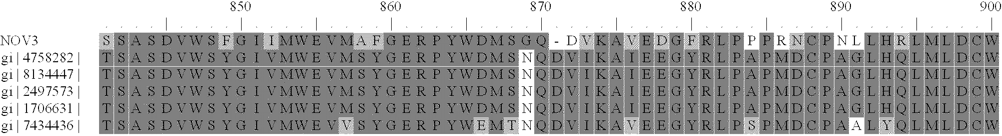

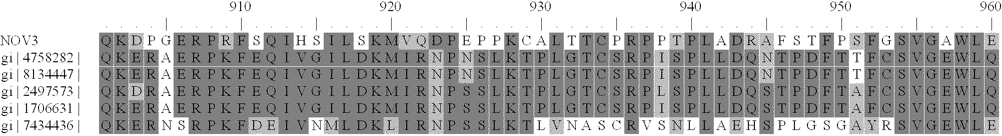

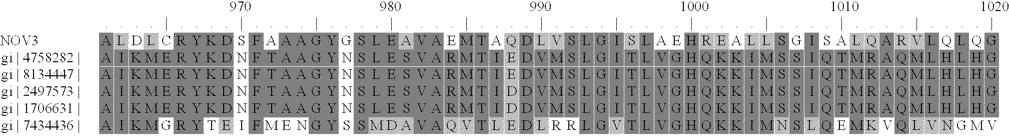

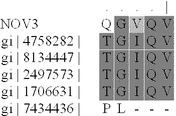

DOMAIN results for NOV3 were collected from the Conserved Domain Database (CDD) with Reverse Position Specific BLAST. This BLAST samples domains found in the Smart and Pfam collections. The NOV3 protein aligned with a number of related domains in both collections.

TABLE 3G
Domain analysis for NOV3

| Gene index identifier | Results |
|---|---|
| Gnl \| Pfam \| pfam01404, EPH_lbd, Ephrin | CD-Length = 174 residues, 99.4% aligned |
| receptor ligand binding domain | Score = 301 bits (772), Expect = 8e − 83 |
| Gnl \| Smart \| TyrKc, Tyrosine kinase, | CD-Length = 257 residues, 100.0% aligned |
| catalytic domain; Phosphotransferases. | Score = 253 bits (645), Expect = 4e − 68 |
| Tyrosine-specific kinase subfamily. | |
| Gnl \| Pfam \| pfam00069, pkinase, Eukaryotic | CD-Length = 256 residues, 97.3% aligned |
| protein kinase domain | Score = 162 bits (411), Expect = 5e − 41 |
| Gnl \| Smart \| S_TKc, Serine/Threonine protein | CD-Length = 256 residues, 97.3% aligned |
| kinases, catalytic domain; | Score = 133 bits (334), Expect = 5e − 32 |
| Phosphotransferases. Serine or threonine- | |
| specific kinase subfamily. | |
| Gnl \| Smart \| SAM, Sterile alpha motif. | CD-Length = 68 residues, 86.8% aligned |
| | Score = 65.1 bits (157), Expect = 2e − 11 |
| Gnl \| Pfam \| pfam00536, SAM, SAM domain | CD-Length = 64 residues, 89.1% aligned |
| (Sterile alpha motif) | Score = 59.7 bits (143), Expect = 7e − 10 |

NOV3 shows similarity with the Ephrin receptor ligand binding domain, which is a type of tyrosine kinase. Also, NOV3 has similarity to the sterile alpha motif.

Amino acids 33 through 208 of NOV3 align with the 174 amino acid ephrin receptor ligand binding domain (SEQ ID NO: 45), as shown in Table 3H. Amino acids 641 through 892 align with amino acids 1 through 257 of the 257 amino acid tyrosine kinase catalytic domain (SEQ ID NO: 46), as shown in Table 31. Additionally, amino acids 925 through 983 of NOV3 align with amino acids 4 through 62 of the 68 amino acid sterile alpha motif (SEQ ID NO: 47), which is a widespread domain in signaling and nuclear proteins. In EPH-related tyrosine kinases, SAM appears to mediate cell-cell initiated signal transduction via the binding of SH2-containing proteins to a conserved tyrosine that is phosphorylated. In many cases, SAM mediates homodimerisation. The alignment of NOV3 with the SAM domain is shown in Table 3J. These similarities indicate that the NOV3 sequence has properties similar to those of other proteins known to contain these domains.

TABLE 3H
Domain Analysis of NOV3
Ephrin receptor ligand binding domain (SEQ ID NO:45)

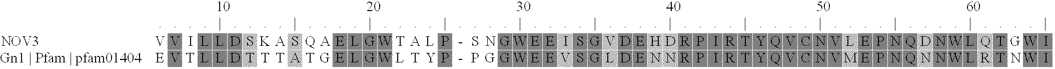

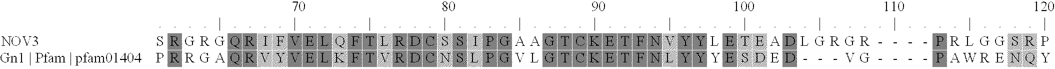

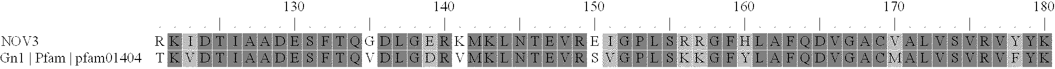

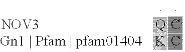

TABLE 3I
Domain Analysis of NOV3
Tyrosine Kinase catalytic domain (SEQ ID NO:46)

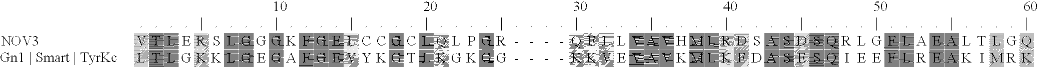

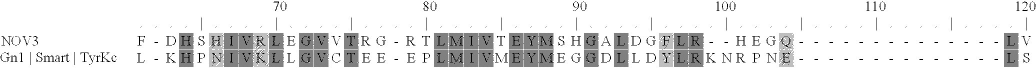

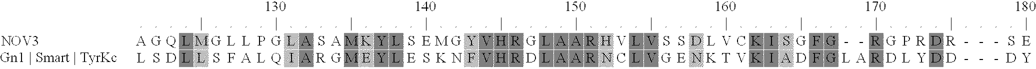

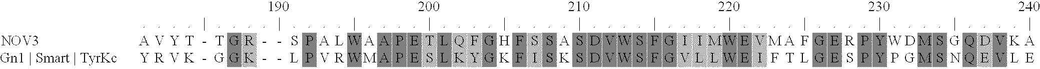

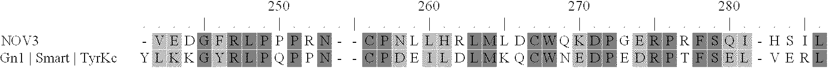

TABLE 3J
Domain Analysis of NOV3
Sterile alpha motif domain (SEQ ID NO:47)

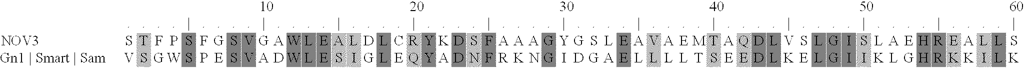

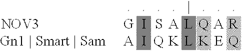

Recent research has been directed to elucidating the developmental functions and biochemistry of Eph receptor tyrosine kinases and their membrane-bound ligands, ephrins. See, generally, Wilkinson, Int. Rev. Cytol. 196:177-244, 2000. The crystal structure of the amino -terminal ligand-binding domain of the receptor tyrosine kinase EphB2 (also known as Nuk) has been determined. Himanen, et al., Nature 396:486-491, 1988. The Eph receptors, which bind a group of cell-membrane-anchored ligands known as ephrins, represent the largest subfamily of receptor tyrosine kinases (RTKs). They are predominantly expressed in the developing and adult nervous system and are important in contact-mediated axon guidance, axon fasciculation and cell migration. Eph receptors are unique among other RTKs in that they fall into two subclasses with distinct ligand specificities, and in that they can themselves function as ligands to activate bidirectional cell-cell signaling. The N-terminal domain folds into a compact jellyroll beta-sandwich composed of 11 antiparallel beta-strands. An extended loop that is important for ligand binding and class specificity has been identified. This loop, which is conserved within but not between Eph RTK subclasses, packs against the concave beta-sandwich surface near positions at which missense mutations cause signaling defects, localizing the ligand-binding region on the surface of the receptor.

EphA receptors bind to GPI-anchored ephrin-A ligands, while EphB receptors bind to ephrin-B proteins that have a transmembrane and cytoplasmic domain. Ephrin-B proteins transduce signals, such that bidirectional signaling can occur upon interaction with Eph receptor. In many tissues, specific Eph receptors and ephrins have complementary domains, whereas other family members may overlap in their expression. An important role of Eph receptors and ephrins is to mediate cell-contact-dependent repulsion. Complementary and overlapping gradients of expression underlie establishment of a topographic map of neuronal projections in the retinotectal system. Eph receptors and ephrins also act at boundaries to channel neuronal growth cones along specific pathways, restrict the migration of neural crest cells, and via bidirectional signaling prevent intermingling between hindbrain segments. Eph receptors and ephrins can also trigger an adhesive response of endothelial cells and are required for the remodeling of blood vessels. Biochemical studies suggest that the extent of multimerization of Eph receptors modulates the cellular response and that the actin cytoskeleton is one major target of the intracellular pathways activated by Eph receptors. Eph receptors and ephrins have thus emerged as key regulators of the repulsion and adhesion of cells that underlie the establishment, maintenance, and remodeling of patterns of cellular organization.

The nucleic acids and proteins of the invention are useful in potential therapeutic applications implicated in various tyrosine kinase-related pathological disorders and/or ephrin-related pathological disorders, described further below. For example, a cDNA encoding the kinase-like protein may be useful in gene therapy, and the kinase -like protein may be useful when administered to a subject in need thereof. SeqCalling expression data and the expression of tyrosine kinase family members suggest that NOV3 is expressed in mammary tissue, breast cancer tissues, endothelial cells, and multiple embryonic and developmental tissues.

By way of nonlimiting example, the compositions of the present invention will have efficacy for treatment of patients suffering from various disorders, including, for example, angiogenesis, cell signaling disorders, cancer, fertility disorders, reproductive disorders, tissue/cell growth regulation disorders, developmental disorders and resulting disorders derived from the above conditions. Other kinase-related diseases and disorders are contemplated.

The novel nucleic acid encoding the tyrosine kinase-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example, the disclosed NOV3 protein has multiple hydrophilic regions, each of which can be used as an immunogen. The novel NOV3 protein can be used in assay systems for functional analysis of various human disorders, which will help in understanding of pathology of the disease and development of new drug targets for various disorders.

NOV4

The novel NOV4 nucleic acid was identified on chromosome 6 by TblastN using CuraGen Corporation's sequence file for chloride conductance regulatory or homolog as run against the Genomic Daily Files made available by GenBank or from files downloaded from the individual sequencing centers. The nucleic acid sequence was predicted from the genomic file Sequencing Center_nh0124i04 by homology to a known chloride conductance regulatory gene or homolog. Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

The disclosed nucleic acid of 742 nucleotides (designated GM_{—}95074063_A, SEQ ID NO: 7) encoding a novel chloride conductance regulatory -like protein is shown in Table 4A. An open reading frame was identified beginning with an ATG initiation codon at nucleotides 28-30 and ending with a TGA codon at nucleotides 724-726. A putative untranslated region upstream from the initiation codon and downstream from the termination codon is underlined in Table 4A, and the start and stop codons are in bold letters. The encoded protein having 223 amino acid residues is presented using the one-letter code in Table 4C (SEQ ID NO: 8).

TABLE 4A
NOV4 Nucleotide Sequence (SEQ ID NO:7).

TTAACATTGTGGTACATTGAAAAATACATGCCGAATAGTTTCCTGCTACCTGAGCCAGCAGAGGGGACC
TGCAGCAGCAGCCAGACACCAAGGCTGTGCTGAACAGGAAGGTCCTCCGCACTGGTACCCTTTATATCGC
TGAGAGCCACCTGTCTTGGTTAGATAGCTCTGGATTAGGATTCTCACTGGAATACCCCACCATTAGTTTA
CTTGCATTATCCAGGGACCAAAGTGACTGTCTAGGAGAACATTTGTATGCTATGGTGAATGACAAATTTG
AAGAATCCAAAGAATCTGTTGCTGATGAAGAAGAGGAAGACAGTGATGATGTTGAACTTATTACTGAATT
TATATTTGTACCTAGTGATAAATCAGCACTGGGGGCAATGTTCACTGCAATGTGTGAATGCCAGGCCTTG
CATCCAGATCCTGAGGATGAGGATGAGGATGACTACGATGGAGAAGAATATGATGTGGAAGCACATGAAC
GAGGAAAAGGGGACATCCTTAAATCTTACACCTATGAAGGATTATCCCATTTAACAGCAGAAGGCCAAGC
CACATTGGAGAGATTAGAAGAAATGCTTTCTCAATCTGTGAGCAGCCAGTATAATATGGCTGGGGTCAGG
ACAGAAGATTCAATAAGGGATTATGAAGATGGGATGGAGGTAGATACCACACCAACAGTTGCTGGACAGT
TTGAGGATACAGATGTTGATCACTGAAAATAATTTATGCAG

The disclosed nucleic acid NOV4 sequence has 620 of 711 bases (87%) identical to a 1579 bp Canisfamiliaris chloride conductance regulatory mRNA (GENBANK-ID: CCCC|acc:X65450 (E=5.4 e-114). In a search of sequence databases, it was also found that the nucleic acid sequence has 460 of 508 bases (90%) identical to a 1368 bp Homo sapiens chloride conductance regulatory mRNA (GENBANK-ID: HS510B21 (E=1.2e-87).

In a search of CuraGen's proprietary human expressed sequence assembly database, assembly s3aq:95074063 (1860 nucleotides) was identified as having >95% homology to this predicted gene sequence (Table 4B). This database is composed of the expressed sequences (as derived from isolated mRNA) from more than 96 different tissues. The mRNA is converted to cDNA and then sequenced. These expressed DNA sequences are then pooled in a database and those exhibiting a defined level of homology are combined into a single assembly with a common consensus sequence. The consensus sequence is representative of all member components. Since the nucleic acid of the described invention has >95% sequence identity with the CuraGen assembly, the nucleic acid of the invention represents an expressed gene sequence. This DNA assembly has 1200 components and was found by CuraGen to be expressed in the following tissues: colon, spleen, lung, small intestine, pancreas, heart, testis, fetal and adult kidney, fetal liver, amygdala, adipose, pituitary gland, lymph node, lung tumor, and bone marrow.

TABLE 4B
NOV4 alignment with S3aq95074063 (SEQ ID NO:48)

S3aq:95074063 Category D: 1200 frag (1 5′sig-CG, 1135 non-5′sig-CG, 57
non-CG EST, 7 non-CG Non-EST), 1860 bp.
Plus Strand HSPS: Score = 832 (124.8 bits), Expect = 5.1e − 32, P = 5.1e − 32
Identities = 172/179 (96%), Positives = 172/179 (96%), Strand = Plus/
Plus

| | | | |
|---|---|---|---|
| NOV4: | 562 | ACATTGGAGAGATTAGAAGAAATGCTTTCTCAATCTGTGAGCAGCCAGTATAATATGGCT | 621 |
| | | \| \|\|\|\|\|\|\|\|\|\|\|\|\|\|\|\|\| \|\|\|\|\|\|\|\|\|\|\|\| \|\|\|\|\|\|\|\|\|\|\|\|\|\|\|\|\|\|\|\|\|\|\|\|\|\|\| | |
| S3aq: | 1108 | ATATTGGAGAGATTAGAAGGAATGCTTTCTCAGTCTGTGAGCAGCCAGTATAATATGGCT | 1167 |
| NOV4: | 622 | GGGGTCAGGACAGAAGATTCAATAAGGGATTATGAAGATGGGATGGAGGTAGATACCACA | 681 |
| | | \|\|\|\|\|\|\|\|\|\|\|\|\|\|\|\|\|\|\|\|\|\|\|\|\|\| \|\|\|\|\|\|\|\|\|\|\|\|\|\|\|\|\|\|\|\|\|\|\| \|\|\|\|\|\|\|\|\| | |
| S3aq: | 1168 | GGGGTCAGGACAGAAGATTCAATAAGAGATTATGAAGATGGGATGGAGGTGGATACCACA | 1227 |
| NOV4: | 682 | CCAACAGTTGCTGGACAGTTTGAGGATACAGATGTTGATCACTGAAAATAATTTATGCA | 740 |
| | | \|\|\|\|\|\|\|\|\|\|\|\|\|\|\|\|\|\|\|\|\|\|\|\|\|\|\| \|\|\|\|\|\|\|\|\|\|\|\|\|\|\|\|\|\|\|\|\| \|\|\|\|\|\|\|\|\| | |
| S3aq: | 1228 | CCAACAGTTGCTGGACAGTTTGAGGATGCAGATGTTGATCACTGAAAATGATTTATGCA | 1286 |

The NOV4 polypeptide (SEQ ID NO: 8) encoded by SEQ ID NO: 7 is presented using the one-letter amino acid code in Table 4C. The Psort profile for NOV4 predicts that this sequence is likely to be localized at the plasma membrane with a certainty of 0.4500.

TABLE 4C
NOV4 protein sequence (SEQ ID NO:8)

MPNSFLLPEPAEGHLQQQPDTKAVLNRKVLRTGTLYIAESHLSWLDSSGLGFSLEYPTISLLALSRDQSDCLGE
HLYAMVNDKFEESKESVADEEEEDSDDVELITEFIFVPSDKSALGAMFTAMCECQALHPDPEDEDEDDYDGEEY
DVEAHERGKGDILKSYTYEGLSHLTAEGQATLERLEEMLSQSVSSQYNMAGVRTEDSIRDYEDGMEVDTTPTVA
GQFEDTDVDH

The full amino acid sequence of the disclosed NOV4 polypeptide has 202 of 232 amino acid residues (87%) identical to, and 207 of 232 residues (89%) positive with, the 237 amino acid residue protein from Homo sapiens chloride channel (chloride conductance regulatory protein, chloride ion current inducer protein), ptnr:SPTREMBL-ACC:P54105, E=2.0e⁻⁹⁹).

BLAST results include sequences from the Patp database, which is a proprietary database that contains sequences published in patents and patent publications. The Patp results include those listed in Table 4D. See, e.g., European Patent 1033401, describing a human secreted protein.

This information is presented graphically in the multiple sequence alignment given in Table 4F (with NOV4 being shown on line 1) as a ClustalW analysis comparing NOV4 with related chloride channel sequences.

TABLE 4F
Information for the ClustalW proteins:

1) NOV4 (SEQ ID NO:8)
2) gi|4502891|ref|NP_001284.1|chloride channel, nucleotide-sensitive, 1A (SEQ ID NO:49)
3) gi|8571386|gb|AAF76859 1|(AF232225) chloride ion current inducer protein I(Cln) (SEQ ID NO:50)
4) gi|8571390|gb|AAF76861.1|(AF232708) chloride ion current inducer protein I(Cln) (SEQ ID NO:51)
5) gi|1095482|prf|2109219A C1 current-related protein (SEQ ID NO:52)
6) gi|1060971|dbj|BAA05069.1| (D26076) chloride channel (SEQ ID NO:53)

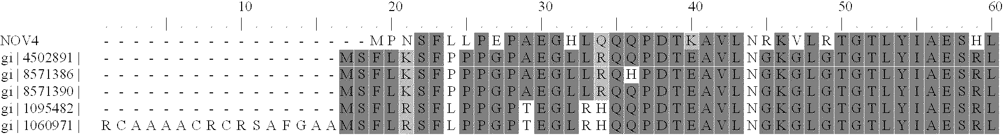

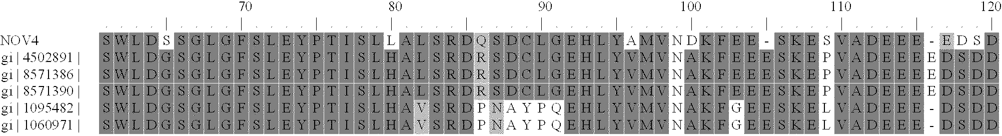

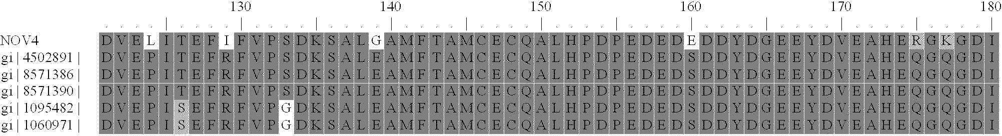

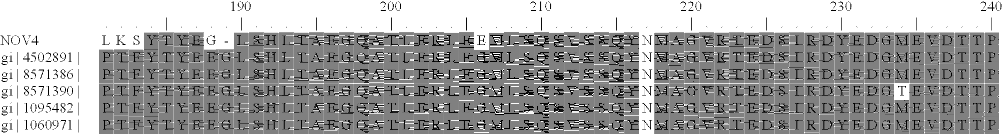

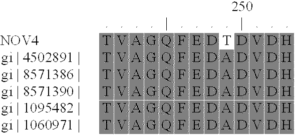

The similarity between the disclosed NOV4 and a number of chloride conductance proteins suggests that NOV4 may function as a member of a chloride conductance regulatory-like protein.

Transporters, channels, and pumps that reside in cell membranes are key to maintaining the right balance of ions in cells, and are vital for transmitting signals from nerves to tissues. The consequences of defects in ion channels and transporters are diverse, depending on where they are located and what their cargo is. In the heart, defects in potassium channels do not allow proper transmission of electrical impulses, resulting in the arrhythmia seen in long QT syndrome. In the lungs, failure of a sodium and chloride transporter found in epithelial cells leads to the congestion of cystic fibrosis, while one of the most common inherited forms of deafness, Pendred syndrome, looks to be associated with a defect in a sulfate transporter. Chloride channels in the ocular ciliary epithelium are believed to play a key role in aqueous humor formation. Anguita et al., Biochem Biophys Res Commun. 208:89-95, 1995.

Chloride channels (CLC) perform important roles in the regulation of cellular excitability, in transepithelial transport, cell volume regulation, and acidification of intracellular organelles. This variety of functions requires a large number of different chloride channels that are encoded by genes belonging to several unrelated gene families. The CLC family of chloride channels has nine known members in mammals that show a differential tissue distribution and function both in plasma membranes and in intracellular organelles. CLC proteins have about 10-12 transmembrane domains. They probably function as dimers and may have two pores. The functional expression of channels altered by site-directed mutagenesis has led to important insights into their structure-function relationship. Their physiological relevance is obvious from three human inherited diseases (myotonia congenita, Dent's disease, and Bartter's syndrome) that result from mutations in some of their members and from a knock-out mouse model. Jentsch et al., Pflugers Arch 437:783-795, 1999.

Recent studies of hereditary renal tubular disorders have facilitated the identification and roles of chloride channels and co-transporters in the regulation of the most abundant anion, Cl-, in the ECF. Thus, mutations that result in a loss of function of the voltage-gated chloride channel, CLC-5, are associated with Dent's disease, which is characterized by low-molecular weight proteinuria, hypercalciuria, nephrolithiasis, and renal failure. Mutations of another voltage-gated chloride channel, CLC-Kb, are associated with a form of Bartter's syndrome, whereas other forms of Bartter's syndrome are caused by mutations in the bumetanide-sensitive sodium-potassium-chloride cotransporter (NKCC2) and the potassium channel, ROMK. Finally, mutations of the thiazide-sensitive sodium-chloride cotransporter (NCCT) are associated with Gitelman's syndrome. Thakker, Adv Nephrol Necker Hosp 29:289-298, 1999. These studies have helped to elucidate some of the renal tubular mechanisms regulating mineral homeostasis and the role of chloride channels.

A more prominent case of chloride channel dysfunction is cystic fibrosis. Cystic fibrosis (CF) is a genetic disease with multi-system involvement in which defective chloride transport across membranes causes dehydrated secretions. Cystic fibrosis (CF) affects approximately 1 in 2000 people making it one of the commonest fatal, inherited diseases in the Caucasian population. Dysfunction of the cystic fibrosis transmembrane conductance regulator (CFTR) Cl- channel is also associated with a wide spectrum of disease. Hwang & Sheppard, Trends Pharmacol Sci 20:448-453, 1999. The protein encoded by the CF gene—the cystic fibrosis transmembrane conductance regulator (CFTR)—functions as a cyclic adenosine monophosphate-regulated chloride channel. The ability to detect CFTR mutations has led to the recognition of its association with a variety of conditions, including chronic bronchitis, sinusitis with nasal polyps, pancreatitis, and, in men, infertility. Choudari et al., Gastroenterol Clin North Am, 28:543-549, vii-viii, 1999. In the search for modulators of CFTR, pharmacological agents that interact directly with the CFTR Cl-channel have been identified. Some agents stimulate CFTR by interacting with the nucleotide-binding domains that control channel gating, whereas others inhibit CFTR by binding within the channel pore and preventing Cl- permeation. Knowledge of the molecular pharmacology of CFTR might lead to new treatments for diseases caused by the dysfunction of CFTR. Chloride channels may participate in cellular volume control by activation of a swelling-induced chloride conductance pathway.

The nucleic acids and proteins of NOV4 are useful in potential therapeutic applications implicated in various chloride channel-related pathological disorders. For example, a cDNA encoding the chloride channel -like protein may be useful in gene therapy, and the chloride channel -like protein may be useful when administered to a subject in need thereof. The protein similarity information, expression pattern, and map location for the chloride channel -like protein and nucleic acid disclosed herein suggest that this chloride channel may have important structural and/or physiological functions characteristic of the chloride channel family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

The nucleic acids and proteins of the invention are useful in potential diagnostic and therapeutic applications implicated in various diseases and disorders described below. For example, the nucleic acids and proteins of the invention are useful in potential therapeutic applications implicated in cystic fibrosis, congenital myotonia, Dent disease, an X-linked renal tubular disorder, leukoencephalopathy, malignant hyperthermia, and hypertension. For example, a cDNA encoding the chloride conductance regulatory -like protein may be useful in gene therapy, and the chloride conductance regulatory -like protein may be useful when administered to a subject in need thereof.

The NOV4 compositions of the present invention will have efficacy for treatment of patients suffering from, for example, cystic fibrosis, congenital myotonia, Dent disease, an X-linked renal tubular disorder, leukoencephalopathy, malignant hyperthermia, hypertension. Other pathologies and disorders are contemplated.

The novel nucleic acid encoding a chloride conductance regulatory -like protein, and the chloride conductance regulatory -like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods and other diseases, disorders and conditions of the like. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below.

For example, the disclosed NOV4 protein has multiple hydrophilic regions, each of which can be used as an immunogen. In one embodiment, a contemplated NOV4 epitope is from about amino acids 5 to 25. In another embodiment, a NOV4 epitope is from about amino acids 65 to 105. In additional embodiments, NOV4 epitopes are from amino acids 125 to 230. These novel proteins can also be used to develop assay system for functional analysis.

NOV5

NOV5 includes a family of two similar nucleic acids and two similar proteins disclosed below. The disclosed nucleic acids encode serotonin receptor-like proteins. The Serotonin Receptor-like gene disclosed in this invention maps to chromosome 2. This assignment was made using mapping information associated with genomic clones, public genes and ESTs sharing sequence identity with the disclosed sequence and CuraGen Corporation's Electronic Northern bioinformatic tool.

NOV5a

The disclosed NOV5a nucleic acid was identified by TblastN using CuraGen Corporation's sequence file for the 5-hydroxytryptamine receptor-like protein or homolog as run against the Genomic Daily Files made available by GenBank or from files downloaded from the individual sequencing centers. The nucleic acid sequence was predicted from the genomic file Seq Ctr ACCNO: nh0028h22 by homology to a known 5-hydroxytryptamine receptor or homolog. Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GenScan and Grail. Expressed sequences from both public and proprietary databases were also added when available to further define and complete the gene sequence. The DNA sequence was then manually corrected for apparent inconsistencies thereby obtaining the sequences encoding the full-length protein.

The disclosed NOV5a nucleic acid of 1150 nucleotides (also referred to as GM_{—}83554525_A, or CG54692-01) is shown in Table 5A. An ORF begins with an ATG initiation codon at nucleotides 24-26 and ends with a TGA codon at nucleotides 1134-1136. A putative untranslated region upstream from the initiation codon and downstream from the termination codon is underlined in Table 5A, and the start and stop codons are in bold letters.

TABLE 5A
NOV5a Nucleotide Sequence

| | |
|---|---|
| CTGGAGCTGCGATCCCAAGCGCCATGGAGGCCGCTAGCCTTTCAGTGGCCACCGCCGGCGTTGCCCTTG | (SEQ ID NO:9) |
| CCCTGGGACCCGAGACCAGCAGCGGGACCCCAAGCCCGAGAGGGATACTCGGTTCGACCCCGAGCGGCG | |
| CCGTCCTGCCGGGCCGAGGGCCGCCCTTCTCTGTCTTCACGGTCCTGGTGGTGACGCTGCTAGTGCTGC | |
| TGATCGCTGCCACTTTCCTGTGGAACCTGCTGGTTCCGGTCACCATCCCGCGGGTCCGTGCCTTCCACC | |
| GCGTGCCGCATAACTTGGTGGCCTCGACGGCCGTCTCGGACGAACTAGTGGCAGCGCTGGCGATGCCAC | |
| CGAGCCTGGCGAGTGAGCTGTCGACCGGGCGACGTCGGCTGCTGGGCCGGAGCCTGTGCCACGTGTGGA | |
| TCTCCTTCGACGCCCTGTGCTGCCCCGCCGGCCTCGGGAACGTGGCGGCCATCGCCCTGGGCCGCGACG | |
| GGGCCATCACACGGCACCTGCAGCACACGCTGCGCACCCGCAGCCGCGCCTCGTTGCTCATGATCGCGC | |
| TCGCCCGGGTGCCGTCGGCGCTCATCGCCCTCGCGCCGCTGCTCTTTGGCCGGGGCGAGGTGTGCGACG | |
| CTCGGCTCCAGCGCTGCCAGGTGAGCCGGGAACCCTCCTATGCCGCCTTCTCCACCCGCGGCGCCTTCC | |
| ACCTGCCGCTTGGCGTGGTGCCGTTTGTCTACCGGAAGATCTACGAGGCGGCCAAGTTTCGTTTCGGCC | |
| GCCGCCGGAGAGCTGTGCTGCCGTTGCCGGCCACCTCCAAGGTAAAGGAAGCACCTGATGAGGCTGAAG | |
| TGGTGTTCACGGCACATTGCAAAGCAACGGTGTCCTTCCAGGTGAGCGGGGACTCCTGGCGGGAGCAGA | |
| AGGAGAGGCGAGCAGCCATGATGGTGGGAATTCTGATTGGCGTGTTTGTGCTGTGCTGGATCCCCTTCT | |
| TCCTGACGGAACTCATCAGCCCACTCTGTGCCTGCAGCCTGCCCCCCATCTGGAAAAGCATATTTCTGT | |
| GGCTTGGCTACTCCAATTCTTTCTTCAACCCCCTGATTTACACAGCTTTTAACAAGAACTACAACAATG | |
| CCTTCAAGAGCCTCTTTACTAAGCAGAGATGAACACAGGGGTTAGA | |

The NOV5a protein encoded by SEQ ID NO: 9 has 370 amino acid residues and is presented using the one-letter code in Table 5B. The Psort profile for NOV5a predicts that this sequence has a signal peptide and is likely to be localized at the endoplasmic reticulum membrane with a certainty of 0.6850, it may also localize to the plasma membrane (certainty of 0.6400). The most likely cleavage site for a peptide is between amino acids 24 and 25, i.e., at the slash in the amino acid sequence SSG-TP (shown as a slash in Table5B) based on the SignalP result.

TABLE 5B
Encoded NOV5a protein sequence

| | |
|---|---|
| MEAASLSVATAGVALALGPETSSG/TPSPRGILGSTPSGAVLPGRGPPFSVFTVLVVTLLVLLIAATFLWNL | (SEQ ID NO:10) |
| LVPVTIPRVRAFHRVPHNLVASTAVSDELVAALAMPPSLASELSTGRRRLLGRSLCHVWISFDALCCPAGLG | |
| NVAAIALGRDGAITRHLQHTLRTRSRASLLMIALARVPSALIALAPLLFGRGEVCDARLQRCQVSREPSYAA | |
| FSTRGAFHLPLGVVPFVYRKIYEAAKFRFGRRRRAVLPLPATSKVKEAPDEAEVVFTAHCKATVSFQVSGDS | |
| WREQKERRAAMMVGILIGVFVLCWIPFFLTELISPLCACSLPPIWKSIFLWLGYSNSFFNPLIYTAFNKNYN | |
| NAFKSLFTKQR | |

The disclosed nucleic acid sequence for NOV5a has 990 of 1230 bases (80%) identical to a Mus musculus, 5-hydroxytryptamine receptor mRNA (GENBANK-ID: X69867) (E=1.1e-167). Additionally, high homology with a portion of the protein of the invention is found with two nucleic acid sequences coding for 335 of 336 bases (99%) identical to a part of a 2061 bp Homo sapiens 5-hydroxytryptamine receptor gene (GENBANK-ID:A39680 Sequence 3 from Patent WO9418319, E=6.4e-69) and also 117 of 117 bases (100%) identical to a 371 bp Homo sapiens expressed sequence tag (EST) (GENBANK-ID:A39680: Soares_testis_NHT Homo sapiens cDNA clone, IMAGE:1641069, E=2.8e-20). This 95-100% homology of the gene of current invention with a public EST sequence strongly suggests that the current invention represents an expressed gene.

The full NOV5a amino acid sequence of the protein of the invention was found to have 295 of 370 amino acid residues (79%) identical to, and 317 of 370 residues (85%) positive with, the 370 amino acid residue 5-hydroxytryptamine receptor protein from Rattus norvegicus (ptnr:SPTREMBL-ACC: P35365) (E=1.9e⁻¹⁵¹), and also, 225 of 348 amino acid residues (64%) identical to, and 261 of 348 residues (75%) positive with, the 357 amino acid residue 5-hydroxytryptamine receptor protein from Homo sapiens (ptnr:SWISSPROT-ACC:P47898) (E=4.5e⁻¹⁰⁹),

NOV5b

NOV5a (GM₈₃₅₅₄₅₂₅_A) was subjected to an exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such suitable sequences were then employed as the forward and reverse primers in a PCR amplification based on a wide range of cDNA libraries.

The cDNA coding for the NOV5b sequence was cloned by the polymerase chain reaction (PCR) using the primers: 5′ CATGGAGGCCGCTAGCCTTT 3′ (SEQ ID NO: 54) and 5′ CCCTGTGTTCATCTCTGCTTAGTAAAGAG 3′ (SEQ ID NO: 55). Primers were designed based on in silico predictions of the full length or some portion (one or more exons) of the cDNA/protein sequence of the invention. These primers were used to amplify a cDNA from a pool containing expressed human sequences derived from the following tissues: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus.

Multiple clones were sequenced and these fragments were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

Variant sequences are also included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, when a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern. Examples include alteration in temporal expression, physiological response regulation, cell type expression regulation, intensity of expression, and stability of transcribed message.

SeqCalling assemblies produced by the exon linking process were selected and extended using the following criteria. Genomic clones having regions with 98% identity to all or part of the initial or extended sequence were identified by BLASTN searches using the relevant sequence to query human genomic databases. The genomic clones that resulted were selected for further analysis because this identity indicates that these clones contain the genomic locus for these SeqCalling assemblies. These sequences were analyzed for putative coding regions as well as for similarity to the known DNA and protein sequences. Programs used for these analyses include Grail, Genscan, BLAST, HMMER, FASTA, Hybrid and other relevant programs.

Some additional genomic regions may have also been identified because selected SeqCalling assemblies map to those regions. Such SeqCalling sequences may have overlapped with regions defined by homology or exon prediction. They may also be included because the location of the fragment was in the vicinity of genomic regions identified by similarity or exon prediction that had been included in the original predicted sequence. The sequence so identified was manually assembled and then may have been extended using one or more additional sequences taken from CuraGen Corporation's human SeqCalling database. SeqCalling fragments suitable for inclusion were identified by the CuraTools™ program SeqExtend or by identifying SeqCalling fragments mapping to the appropriate regions of the genomic clones analyzed. Such sequences were included in the derivation of NOV5b (Acc. No. CG54692-02) only when the extent of identity in the overlap region with one or more SeqCalling assemblies 145286067 was high. The extent of identity may be, for example, about 90% or higher, preferably about 95% or higher, and even more preferably close to or equal to 100%. When necessary, the process to identify and analyze SeqCalling fragments and genomic clones was reiterated to derive the fuill length sequence.

The regions defined by the procedures described above were then manually integrated and corrected for apparent inconsistencies that may have arisen, for example, from miscalled bases in the original fragments or from discrepancies between predicted exon junctions, EST locations and regions of sequence similarity, to derive the final sequence disclosed herein. When necessary, the process to identify and analyze SeqCalling assemblies and genomic clones was reiterated to derive the fuill length sequence. The following public components were thus included in the invention: gb:GENBANK-ID:AC009404|acc:AC009404.5 Homo sapiens BAC clone RP11-28H22 from 2,complete sequence—Homo sapiens, 112883 bp. In addition, the following CuraGen Corporation SeqCalling Assembly ID's were also included in the invention: 145286067.

The resulting amplicon was gel purified, cloned and sequenced to high redundancy to provide NOV5b (SEQ ID NO: 11), which is also referred to as CuraGen Acc. No. CG54692-02.

The nucleotide sequence for NOV5b (1150 bp, SEQ ID NO: 11) is presented in Table 5C. An open reading frame was identified beginning at nucleotides 24-26 and ending at nucleotides 1134-1136. The start and stop codons of the open reading frame are highlighted in bold type, and putative untranslated regions are underlined. The nucleotide sequence of NOV5b differs from NOV5a by six nucleotide changes: T709>C; T795>A; C796>T; C797>G; A798>C; G800>A.

TABLE 5C
NOV5b Nucleotide Sequence

| | | |
|---|---|---|
| CTGGAGCTGCGATCCCAAGCGCCATGGAGGCCGCTAGCCTTTCAGTGGCCACCGCCGGCG | 60 | (SEQ ID NO:11) |
| TTGCCCTTGCCCTGGGACCCGAGACCAGCAGCGGGACCCCAAGCCCGAGAGGGATACTCG | 120 | |
| GTTCGACCCCGAGCGGCGCCGTCCTGCCGGGCCGAGGGCCGCCCTTCTCTGTCTTCACGG | 180 | |
| TCCTGGTGGTGACGCTGCTAGTGCTGCTGATCGCTGCCACTTTCCTGTGGAACCTGCTGG | 240 | |
| TTCCGGTCACCATCCCGCGGGTCCGTGCCTTCCACCGCGTGCCGCATAACTTGGTGGCCT | 300 | |
| CGACGGCCGTCTCGGACGAACTAGTGGCAGCGCTGGCGATGCCACCGAGCCTGGCGAGTG | 360 | |
| AGCTGTCGACCGGGCGACGTCGGCTGCTGGGCCGGAGCCTGTGCCACGTGTGGATCTCCT | 420 | |
| TCGACGCCCTGTGCTGCCCCGCCGGCCTCGGGAACGTGGCGGCCATCGCCCTGGGCCGCG | 480 | |
| ACGGGGCCATCACACGGCACCTGCAGCACACGCTGCGCACCCGCAGCCGCGCCTCGTTGC | 540 | |
| TCATGATCGCGCTCGCCCGGGTGCCGTCGGCGCTCATCGCCCTCGCGCCGCTGCTCTTTG | 600 | |
| GCCGGGGCGAGGTGTGCGACGCTCGGCTCCAGCGCTGCCAGGTGAGCCGGGAACCCTCCT | 660 | |
| ATGCCGCCTTCTCCACCCGCGGCGCCTTCCACCTGCCGCTTGGCGTGGCGCCGTTTGTCT | 720 | |
| ACCGGAAGATCTACGAGGCGGCCAAGTTTCGTTTCGGCCGCCGCCGGAGAGCTGTGCTGC | 780 | |
| CGTTGCCGGCCACCATGCAAGTAAAGGAAGCACCTGATGAGGCTGAAGTGGTGTTCACGG | 840 | |
| CACATTGCAAAGCAACGGTGTCCTTCCAGGTGAGCGGGGACTCCTGGCGGGAGCAGAAGG | 900 | |
| AGAGGCGAGCAGCCATGATGGTGGGAATTCTGATTGGCGTGTTTGTGCTGTGCTGGATCC | 960 | |
| CCTTCTTCCTGACGGAACTCATCAGCCCACTCTGTGCCTGCAGCCTGCCCCCCATCTGGA | 1020 | |
| AAAGCATATTTCTGTGGCTTGGCTACTCCAATTCTTTCTTCAACCCCCTGATTTACACAG | 1080 | |
| CTTTTAACAAGAACTACAACAATGCCTTCAAGAGCCTCTTTACTAAGCAGAGATGAACAC | 1140 | |
| AGGGGTTAGA | 1150 | |

In a search of sequence databases, it was found, for example, that the NOV5b nucleic acid sequence has 920 of 1123 bases (81%) identical to a serotonin receptor mRNA from Mus musculus (gb: GENBANK-ID:MM5HT5BSR|acc:X69867.1, M.musculus mRNA encoding 5-HT5B serotonin receptor, E=1.9e-163).

The encoded NOV5b protein is presented in Table 5D. The disclosed protein is 370 amino acids long and is denoted by SEQ ID NO: 12. NOV5b differs from NOV5a by 3 amino acid residues: V229>A; S258>M; K259>Q.

Like NOV5a, the Psort profile for NOV5b predicts that this sequence has a signal peptide and is likely to be localized at the endoplasmic reticulum membrane with a certainty of 0.6850, or at the plasma membrane, with a certainty of 0.6400. The most likely cleavage site for a peptide is between amino acids 24 and 25, i.e., at the slash in the amino acid sequence SSG-TP (shown as a slash in Table5D) based on the SignalP result.

TABLE 5D
Encoded NOV5b protein sequence

| | | |
|---|---|---|
| MEAASLSVATAGVALALGPETSSG/TPSPRGILGSTPSGAVLPGRGPPFSVFTVLVVTLLV | 60 | (SEQ ID NO:12) |
| LLIAATFLWNLLVPVTIPRVRAFHRVPHNLVASTAVSDELVAALAMPPSLASELSTGRRR | 120 | |
| LLGRSLCHVWISFDALCCPAGLGNVAAIALGRDGAITRHLQHTLRTRSRASLLMIALARV | 180 | |
| PSALIALAPLLFGRGEVCDARLQRCQVSREPSYAAFSTRGAFHLPLGVAPFVYRKIYEAA | 240 | |
| KFRFGRRRRAVLPLPATMQVKEAPDEAEVVFTAHCKATVSFQVSGDSWREQKERRAAMMV | 300 | |
| GILIGVFVLCWIPFFLTELISPLCACSLPPIWKSIFLWLGYSNSFFNPLIYTAFNKNYNN | 360 | |
| AFKSLFTKQR | 370 | |

The full amino acid sequence of the NOV5b protein was found to have 295 of 370 amino acid residues (79%) identical to, and 315 of 370 amino acid residues (85%) similar to, the 370 amino acid residue serotonin receptor protein from Rattus norvegicus (ptnr:SWISSPROT-ACC:P35365, 5-HYDROXYTRYPTAMINE 5B RECEPTOR (5-HT-5B), SEROTONIN RECEPTOR (MR22), E=6.8e-152).

Patp results include those listed in Table 5E.

TABLE 5E
Patp alignments of NOV5a

| | | | Smallest |
|---|---|---|---|
| | | | Sum |
| | Reading | High | Prob |
| Sequences producing High-scoring Segment Pairs: | Frame | Score | P(N) |
| Patp:R58686 Rat MR22 serotonin receptor protein - | ... +3 | 1486 | 1.6e − 151 |
| Patp:R57066 Murine serotoninergic receptor 5H5b - | ... +3 | 1485 | 2.0e − 151 |
| Patp:R45848 Human 5HT5a serotonin receptor - | ... +3 | 1046 | 6.7e − 105 |
| Patp:R45847 Murine 5HT5a serotonin receptor - | ... +3 | 1041 | 2.3e − 104 |
| Patp:R58685 Rat REC17 serotonin receptor protein - | ... +3 | 1038 | 4.7e − 104 |
| Patp:R57067 Human serotoninergic receptor 5HT5b - | ... +3 | 596 | 3.2e − 57 |

For example, a BLAST against R58686, a 370 amino acid serotonin receptor from Rattus rattus, produced 295/370 (79%) identity, and 317/370 (85%) positives (E=1.6e-151), with long segments of amino acid identity, as shown in Table 5F. WO 94/21670. A blast against R57066, a 370 amino acid murine serotoninergic receptor (5HT5b) from Mus musculus produced 297/370 (80%) identity, and 318/370 (85%) positives (E=2.0e-151). WO 94/18319. Additionally, amino acids 260 -320 from NOV5 were found to be identical with a 111 amino acid human serotonergic receptor (E=3.2e-57). WO 94/18319.

Unless specifically addressed as NOV5a or NOV5b any reference to NOV5 is assumed to encompass all variants. Residue differences between any NOVX variant sequences herein are written to show the residue in the “a” variant and the residue position with respect to the “a” variant. NOV residues in all following sequence alignments that differ between the individual NOV variants are highlighted with a box and marked with the (o) symbol above the variant residue in all alignments herein. For example, the protein shown in line 1 of Table 5F depicts the sequence for NOV5a, and the positions where NOV5b differs are marked with a (o) symbol and are highlighted with a box. Both NOV5 proteins have significant homology to serotonin receptor (SR) proteins:

TABLE 5F
NOV5 alignment with R58686 (SEQ ID NO:56)

Score = 1486 (523.1 bits), Expect = 1.6e − 151, P = 1.6e − 151
Identities = 295/370 (79%), Positives = 317/370 (85%), Frame = +3

| | | | |
|---|---|---|---|
| NOV5: | 1 | MEAASLSVATAGVALALGPETSSGTPSPRGILGSTPSGAVLPGRGPPFSVFTVLVVTLLV | 60 |
| | | \|\| ++\|\| \|\| \|+\| \|\|\|+ \| \|\| +\|\|\|\| \| +\| \|\| \|\|\|\| \|\|\|\|\|\|\|\|\|\| | |
| R58686: | 1 | MEVSNLSGATPGIAFPPGPESCSDSPSSGRSMGSTPGGLILSGREPPFSAFTVLVVTLLV | 60 |
| NOV5: | 61 | LLIAATFLWNLLVPVTIPRVRAFHRVPHNLVASTAVSDELVAALAMPPSLASELSTGRRR | 120 |
| | | \|\|\|\|\|\|\|\|\|\|\|\|\| \|\|\| \|\|\|\|\|\|\|\|\|\|\|\|\|\|\|\|\|\|\|\| \|\|\|\|\| \|\| \|\| \|\|\|\| \|\|\| | |
| R58686: | 61 | LLIAATFLWNLLVLVTILRVPAFHRVPHNLVASTAVSDVLVAALVMPLSLVSELSAGRRW | 120 |
| NOV5: | 121 | LLGRSLCHVWISFDALCCPAGLGNVAAIALGRDGAITRHLQHTLRTRSRASLLMIALARV | 180 |
| | | \|\|\|\|\|\|\|\|\|\|\|\|\| \|\|\| \| + \|\|\|\|\|\|\| \| \|\|\|\|\|\|+\|\|\|\|\| \|\|\| \|\|\|\|+ | |
| R58686: | 121 | QLGRSLCHVWISFDVLCCTASIWNVAAIALDRYWTITRHLQYTLRTRRRASALMIAITWA | 180 |
| | | ∘ | |
| NOV5: | 181 | PSALIALAPLLFGRGEVCDARLQRCQVSREPSYAAFSTRGAFHLPLGV\overline{\|V\|}PFVYRKIYEAA | 240 |
| | | \|\|\|\|\|\|\|\|\|\|\|\|\| \|\| \|\|\|\|\|\|\|\|\|\|+\|\|\|\|\| \|\|\| \|\|\|++\|\| \|\| \|\|\| \|\|\|+\|\| | |
| R58686: | 181 | LSALIALAPLLFGWGEAYDARLQRCQVSQEPSYAVFSTCGAFYVPLAVVLFVYWKIYKAA | 240 |
| | | Oo | |
| NOV5: | 241 | KFRFGRRRRAVLPLPAT\overline{\|SK\|}VKEAPDEAEVVFTAHCKATVSFQVSGDSWREQKERRAAMMV | 300 |
| | | \|\|\|\|\|\|\|\|\|\|\|+\|\|\|\|\|++ \|\|\|\| \|+\| \|\|\|\| \|+\|\|\|+\|\| \|\|\|\|\|\|\|\|\|\|+\|\|\|\|\|\| | |
| R58686: | 241 | KFRFGRRRRAVPLPATTQAKEAPQESETVFTARCRATVAFQTSGDSWREQKEKRAAMMV | 300 |
| NOV5: | 301 | GILIGVFVLCWIPFFLTELISPLCACSLPPIWKSIFLWLGYSNSFFNPLIYTAFNKNYNN | 360 |
| | | \|\|\|\|\|\|\|\|\|\|\|\|\|\|\|\|\|\|\|+\|\|\|\|\|\|\|\|\|\|\|\|\|\|\|\|\|\|\|\|\|\|\|\|\|\|\|\|\|\|\|\|\|\|\|\|\|\|\|\| | |
| R58686: | 301 | GILIGVFVLCWIPFFLTELVSPLCACSLPPIWKSIFLWLGYSNSFFNPLIYTAFNKNYNN | 360 |
| NOV5: | 361 | AFKSLFTKQR | 370 |
| | | \|\|\|\|\|\|\|\|\|\| | |
| R58686: | 361 | AFKSLFTKQR 370 | |

The disclosed NOV5 protein has good identity with a number of serotonin receptor proteins. The identity information used for ClustalW analysis is presented in Table 5G.

TABLE 5G
BLAST results for NOV5

| Gene Index/ | | Length | Identity | Positives | |
|---|---|---|---|---|---|
| Identifier | Protein/Organism | (aa) | (%) | (%) | Expect |
| gi\|543730\|sp\|P35365 | 5- | 370 | 265/370 | 287/370 | e − 134 |
| \|5H5B_RAT | HYDROXYTRYPTANINE | | (71%) | (76%) | |
| | 5B RECEPTOR (5- | | | | |
| | HT-5B) (SEROTONIN | | | | |
| | RECEPTOR) (MR22) | | | | |
| | Rattus norvegicus | | | | |
| gi\|6754260\|ref\|NP_0 | 5- | 370 | 267/370 | 288/370 | e − 134 |
| 34613.1/ | hydroxytryptamine | | (72%) | (77%) | |
| | (serotonin) | | | | |
| | receptor 5B | | | | |
| | Mus musculus | | | | |
| gi\|543453\|pir\|\|S387 | serotonin | 369 | 265/370 | 287/370 | e − 133 |
| 44 | receptor 5B | | (71%) | (76%) | |
| | Rattus norvegicus | | | | |
| gi\|13236497\|ref\|NP_ | 5- | 357 | 204/349 | 237/349 | 2e − 97 |
| 076917.1\| | hydroxytryptamine | | (58%) | (67%) | |
| | (serotonin) | | | | |
| | receptor 5A | | | | |
| | Homo sapiens | | | | |

This information is presented graphically in the multiple sequence alignment given in Table 5H (with NOV5a being shown on line 1) as a ClustalW analysis comparing NOV5 with related protein sequences.

[image]

[image]

DOMAIN results for NOV5a were collected from the Conserved Domain Database (CDD) with Reverse Position Specific BLAST. This BLAST samples domains found in the Smart and Pfam collections. The results for NOV5a are listed in Table 5I with the statistics and domain description.

The region from amino acid residue 70 through 351 (SEQ ID NO: 10) most probably (E=6e⁻²⁸) contains a “seven transmembrane receptor (rhodopsin family) fragment” domain, aligned here with residues 2-254 of the 7tm_{—}1 entry (SEQ ID NO: 61, see Table 5J, below) of the Pfam database. This indicates that the GPCR5 sequence has properties similar to those of 5 other proteins known to contain this domain as well as to the 377 amino acid 7tm domain itself. GPCR5b also has identity to the TM7 domain. The regions from amino acid residue 70 through 235 and from 284-351 (of SEQ ID NO: 12) align with amino acid residues of TM7 (E=2.8e⁻⁴⁷).

[image]

The representative member of the 7 transmembrane receptor family is the D2 dopamine receptor from Bos taurus (SWISSPROT: locus D2DR_BOVIN, accession P20288; gene index 118205). The D2 receptor is an integral membrane protein and belongs to Family 1 of G-protein coupled receptors. The activity of the D2 receptor is mediated by G proteins which inhibit adenylyl cyclase. Chio et al., Nature 343:255-269 (1990). Residues 51-427 of this 444 amino acid protein are considered to be the representative TM7 domain, shown in Table 5J.

TABLE 5J
Amino Acid sequence for TM7 (SEQ ID NO:61)

GNVLVCMAVSREKALQTTTNYLIVSLAVADLLVATLVMPWVVYLEVVGEWKFSRIHCDIF
VTLDVMMCTASILNLCAISIDRYTAVAMPMLYNTRYSSKRRVTVMIAIVWVLSFTISCPM
LFGLNNTDQNECIIANPAFVVYSSIVSFYVPFIVTLLVYIKIYIVLRRRRKRVNTKRSSR
AFRANLKAPLKGNCTHPEDMKLCTVIMKSNGSFPVNRRRVEAARRAQELEMEMLSSTSPP
ERTRYSPIPPSHHQLTLPDPSHHGLHSTPDSPAKPEKNGHAKTVNPKIAKIFEIQSMPNG
KTRTSLKTMSRRKLSQQKEKKATQMLAIVLGVFIICWLPFFITHILNIHCDCNIPPVLYS
AFTWLGYVNSAVNPIIY

The 7 transmembrane receptor family includes a number of different proteins, including, for example, hormone, neurotransmitter and light receptors, all of which transduce extracellular signals through interaction with guanine nucleotide-binding proteins. Although the activating ligands for this class of proteins vary widely in structure and character, the amino acid sequences for the receptors are very similar and are believed to adopt a common structural framework comprising seven transmembrane helices. Included in this family are serotonin receptors, dopamine receptors, histamine receptors, andrenergic receptors, cannabinoid receptors, angiotensin II receptors, chemokine receptors, opioid receptors, G-protein coupled receptor (GPCR) proteins, olfactory receptors (OR), and the like. Some proteins and the Protein Data Base Ids/gene indexes include, for example: rhodopsin (129209); 5-hydroxytryptaminereceptors; (112821,8488960, 112805, 231454, 1168221, 398971, 112806); G protein-coupled receptors (119130, 543823, 1730143, 132206, 137159, 6136153, 416926, 1169881, 136882, 134079); gustatory receptors (544463, 462208); c-x-c chemokine receptors (416718, 128999, 416802, 548703, 1352335); opsins (129193, 129197, 129203); and olfactory receptor-like proteins (129091, 1171893, 400672, 548417).

Based on sequence homology with other serotonin receptors, as well as domain information, the disclosed NOV5 proteins likely function as serotonin (5-hydroxytryptamine) receptors. The neurotransmitter serotonin (5-hydroxytryptamine; 5-HT) exerts a wide variety of physiologic functions through a multiplicity of receptors and may be involved in human neuropsychiatric disorders such as anxiety, depression, or migraine. These receptors consist of 4 main groups, 5-HT-1, 5-HT-2, 5-HT-3, and 5 -HT4, subdivided into several distinct subtypes on the basis of their pharmacologic characteristics, coupling to intracellular second messengers, and distribution within the nervous system. Zifa and Fillion, Pharm. Rev. 44:401-458, 1992. The serotonergic receptors belong to the multi5-Hydroxytryptamine Receptor family of receptors coupled to guanine nucleotide-binding proteins. See, generally, OMIM ID: 182131 and Demchyshyn, et al., Proc. Natl. Acad. Sci. 89:5522-5526, 1992.

Potential transmembrane regions of NOV5 include amino acids 48-64 (likelihood—12.10), 135-151 (likelihood—0.48), 172-188 (likelihood—4.94), and 300-316 (likelihood—9.66).

The nucleic acids and proteins of NOV5 are useful in potential therapeutic applications implicated in various pathological disorders, described further below. For example, a cDNA encoding the serotonin receptor-like protein may be useful in gene therapy, and the serotonin receptor -like protein may be useful when administered to a subject in need thereof.

The nucleic acids and proteins of the invention have applications in the diagnosis and/or treatment of various diseases and disorders. For example, the compositions of the present invention will have efficacy for the treatment of patients suffering from: seizures, Alzheimer's disease, sleep disorders, appetite disorders, thermoregulation, pain perception, hormone secretion and sexual behavior, mental depression, migraine, epilepsy, obsessive-compulsive behavior (schizophrenia), and affective disorders as well as other diseases, disorders and conditions.

The polypeptides can be used as immunogens to produce antibodies specific for the invention, and as vaccines. They can also be used to screen for potential agonist and antagonist compounds. For example, a cDNA encoding the serotonin receptor-like protein may be useful in gene therapy, and the receptor-like protein may be useful when administered to a subject in need thereof. The novel nucleic acid encoding serotonin receptor-like protein, and the serotonin receptor-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV5 protein has multiple hydrophilic regions, each of which can be used as an immunogen. In one embodiment, a contemplated NOV5 epitope is from about amino acids 10 to 40. In another embodiment, a NOV5 epitope is from about amino acids 110 to 130. In additional embodiments, NOV5 epitopes are from amino acids 150 to 175, 190 to 200, 240-270 and from amino acids 280 to 320. This novel protein also has value in development of powerful assay system for functional analysis of various human disorders, which will help in understanding of pathology of the disease and development of new drug targets for various disorders.

NOV6

NOV6a

NOV6a was initially identified by searching CuraGen's Human SeqCalling database for DNA sequences that translate into proteins with similarity to a protein family of interest. SeqCalling assembly 21639300 was identified as having suitable similarity. SeqCalling assembly 21639300 was analyzed further to identify an open reading frame encoding for a novel full length protein and novel splice forms of this gene. This was done by extending the SeqCalling assembly using suitable additional SeqCalling assemblies, publicly available EST sequences and public genomic sequence. Public ESTs and additional CuraGen SeqCalling assemblies were identified by the Curatools program SeqExtend. They were included in the DNA sequence extension for SeqCalling assembly 21639300 only when sufficient identical overlap was found. These inclusions are described below.

The genomic clone ALI 21901 was identified as having regions with 100% identity to the SeqCalling assembly 21639300 and were selected for analysis because this identity implied that the clone AL121901 contained the sequence of the genomic locus for SeqCalling assembly 21639300.

The genomic clone AL 121901 was analyzed by Genscan and Grail to identify exons and putative coding sequences/open reading frames. The clone AL121901 was also analyzed by publicly available ThlastN, BlastX, and other homology programs to identify regions translating to proteins with similarity to the original protein/protein family of interest.

The results of these analyses were integrated and manually corrected for apparent inconsistencies, thereby obtaining the sequence encoding the full-length protein. When necessary, the process to identify and analyze cDNAs/ESTs and genomic clones was reiterated to derive the full-length sequence. This invention describes this full-length DNA sequence(s) and the full-length protein sequence(s) which they encode. This gene belongs to genomic clone AL121901 on Chromosome 20.

The disclosed novel NOV6a nucleic acid of 963 nucleotides (Accession Number 21639300_EXT, SEQ ID NO: 13) is shown in Table 6A. An open reading begins with an ATG initiation codon at nucleotides 1-3 and ends with a TAA codon at nucleotides 961-963. A putative untranslated region upstream from the initiation codon and downstream from the termination codon are underlined in Table 6A, and the start and stop codons are in bold letters.

TABLE 6A
NOV6a Nucleotide Sequence (SEQ ID NO:13)

ATGGCCGGCCCGTGGACCTTCACCCTTCTCTGTGGTTTGCTGGCAGCCACCTTGATCCAAGCCACCCTCA
GTCCCACTGCAGTTCTCATCCTCGGCCCAAAAGTCATCAAAGAAAGTCTGACACAGGAGCTGAAGGACCA
CAACGCCACCAGCATCCTGCAGCAGCTGCCGCTGCTCAGTGCCATGCGGGAAAAGCCAGCCGGAGGCATC
CCTGTGCTGGGCAGCCTGGTGAACACCGTCCTGAAGCACATCACCCCATCCAGGCTGAAGGTCATCACAG
CTAACATCCTCCAGCTGCAGGTGAAGCCCTCGGCCAATGACCAGGAGCTGCTAGTCAAGATCCCCCTGGA
CATGGTGGCTGGATTCAACACGCCCCTGGTCAAGACCATCGTGGAGTTCCACATGACGACTGAGGCCCAA
GCCACCATCCGCATGGACACCAGTGCAAGTGGCCCCACCCGCCTGGTCCTCAGTGACTGTGCCACCAGCC
ATGGGAGCCTGCGCATCCAACTGCTGCATAAGCTCTCCTTCCTGGTGAACGCCTTAGCTAAGCAGGTCAT
GAACCTCCTAGTGCCATCCCTGCCCAATCTAGTGAAAAACCAGCTGTGTCCCGTGATCGAGGCTTCCTTC
AATGGCATGTATGCAGACCTCCTGCAGCTGGTGAAGGGTAGGTGCTCTGCTCTCTCTCCCACTTTTTCCT
TTACTACGGAGCTGGCCTCCAGACCCGGAAAGGTGACCAAGTGGTTCAATAACTCTGCAGCTTCCCTGAC
AATGCCCACCCTGGACAACATCCCGTTCAGCCTCATCGTGAGTCAGGACGTGGTGAAAGCTGCAGTGGCT
GCTGTGCTCTCTCCAGAAGAATTCATGGTCCTGTTGGACTCTGTGGTAAACCTCAGCACAAGGCAGAGAA
TAGGGCCGCCCAGGCCACATCATAGGAATTTCCTGAACACAGGGTGCCCCTAA

The disclosed nucleic acid sequence has 506 of 660 nucleotides (76%) identical to a 1683 bp Mus musculus von Ebner minor salivary gland protein (GENBANK-ID:MMU46068|acc:U46068) (E value=4.0e-⁷⁶).

The NOV6a protein encoded by SEQ ID NO: 13 has 320 amino acid residues, and is presented using the one-letter code in Table 6B (SEQ ID NO: 14). The SignalP, Psort and/or Hydropathy profile for NOV6a predict that NOV6a is likely to be localized at the lysozyme lumen with a certainty of 0.8279, or the lysozyme outside, with a certainty of 0.6138. A cleavage site is indicated at the slash in the sequence TLS-PT, between amino acids 24 and 25 in Table 6B. The hydropathy profile of the NOV6a salivary gland protein-like protein indicates that this sequence has a strong signal peptide toward the 5′ terminal supporting extracellular localization. It is very likely that the membrane-bound peptide as predicted here is similar to the salivary gland protein gene family, some members of which are localized at the plasma membrane. Therefore it is likely that this novel gene is available at the appropriate sub-cellular localization and hence accessible for the therapeutic uses described in this application.

TABLE 6B
Encoded NOV6 protein sequence (SEQ ID NO:14).

MAGPWTFTLLCGLLAATLIQATLS/PTAVLILGPKVIKESLTQELKDHNATSILQQLPLLSAMREKPAGGI
PVLGSLVNTVLKHITPSRLKVITANILQLQVKPSANDQELLVKIPLDMVAGFNTPLVKTIVEFHMTTEAQ
ATIRMDTSASGPTRLVLSDCATSHGSLRIQLLHKLSFLVNALAKQVMNLLVPSLPNLVKNQLCPVIEASF
NGMYADLLQLVKGRCSALSPTFSFTTELASRPGKVTKWFNNSAASLTMPTLDNIPFSLIVSQDVVKAAVA
AVLSPEEFMVLLDSVVNLSTRQRIGPPRPHHRNFLNTGCP

The full amino acid sequence of the disclosed NOV6a protein was found to have 164 of 302 amino acid residues (54%) identical to, and 208 of 302 amino acid residues (68%) positive with, the 310 amino acid residue protein von Ebner minor salivary gland protein from Mus musculus (SPTREMBL-ACC:Q61114) (E value=3.4e-72).

NOV6b

The NOV6a target sequence identified previously was subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached.

The cDNA coding for the NOV6b (CG5 1622-02) sequence was cloned by the polymerase chain reaction (PCR) using the primers: 5′ CCAGCCCCGAATCTTGTGTTGACT 3′ (SEQ ID NO: 62) and 5′ AGAGCGTTGGGTCACGTGAGGACT 3′ (SEQ ID NO: 63). Primers were designed based on in silico predictions of the full length or some portion (one or more exons) of the cDNA/protein sequence of the invention. These primers were used to amplify a cDNA from a pool containing expressed human sequences derived from the following tissues: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raj i, mammary gland, pancreas, pituitary gland, placenta, prostate, salivary gland, skeletal muscle, small intestine, spinal cord, spleen, stomach, testis, thyroid, trachea and uterus.

Multiple clones were sequenced and these fragments were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

SeqCalling assemblies produced by the exon linking process were selected and extended using the following criteria. Genomic clones having regions with 98% identity to all or part of the initial or extended sequence were identified by BLASTN searches using the relevant sequence to query human genomic databases. The genomic clones that resulted were selected for further analysis because this identity indicates that these clones contain the genomic locus for these SeqCalling assemblies. These sequences were analyzed for putative coding regions as well as for similarity to the known DNA and protein sequences. Programs used for these analyses include Grail, Genscan, BLAST, HMMER, FASTA, Hybrid and other relevant programs.

Some additional genomic regions may have also been identified because selected SeqCalling assemblies map to those regions. Such SeqCalling sequences may have overlapped with regions defined by homology or exon prediction. They may also be included because the location of the fragment was in the vicinity of genomic regions identified by similarity or exon prediction that had been included in the original predicted sequence. The sequence so identified was manually assembled and then may have been extended using one or more additional sequences taken from CuraGen Corporation's human SeqCalling database. SeqCalling fragments suitable for inclusion were identified by the CuraTools™ program SeqExtend or by identifying SeqCalling fragments mapping to the appropriate regions of the genomic clones analyzed. Such sequences were included in the derivation of NOV6b only when the extent of identity in the overlap region with one or more SeqCalling assemblies was high. The extent of identity may be, for example, about 90% or higher, preferably about 95% or higher, and even more preferably close to or equal to 100%. When necessary, the process to identify and analyze SeqCalling fragments and genomic clones was reiterated to derive the full length sequence.

The regions defined by the procedures described above were then manually integrated and corrected for apparent inconsistencies that may have arisen, for example, from miscalled bases in the original fragments or from discrepancies between predicted exon junctions, EST locations and regions of sequence similarity, to derive the final sequence disclosed herein. When necessary, the process to identify and analyze SeqCalling assemblies and genomic clones was reiterated to derive the full length sequence. The following public components were thus included in the invention: GenBank: gb_AL121901.20 PRI/HTG Homo sapiens|Human DNA sequence from clone RP11-49G10 on chromosome 20, complete sequence, 161593 bp.

The DNA and protein sequences for the novel Von Ebner Minor Salivary Gland Protein-like gene are reported here as CuraGen Acc. No. CG51622-02, or NOV6b. The disclosed novel NOV6b nucleic acid of 1035 nucleotides (SEQ ID NO: 15) is shown in Table 6C. An open reading begins with an ATG initiation codon at nucleotides 79-81 and ends with a TAA codon at nucleotides 1033-1035. A putative untranslated region upstream from the initiation codon and downstream from the termination codon are underlined in Table 6C, and the start and stop codons are in bold letters. NOV6b differs from NOV6a in the following ways: NOV6b has 78 nucleotides at the 5′ UTR, and ten base changes or deletions, numbered with respect to NOV6b: G194>A; T195>G; C332>T; C334>T; C335>Δ;A336>Δ; T337>Δ; C338>Δ; C339>Δ; A340>Δ; (where Δ designates a base deletion).

TABLE 6C

| | |
|---|---|
| NOV6b Nucleotide Sequence | (SEQ ID NO:15) |

| | |
|---|---|
| AGAGCCGTTGGGTCACGTGAGGACTCCAGCGTGCCCAGGTCTGGCATCCTGCACTTACTGC | 60 |
| CCTCTTGACACCTGGGAAGATGGCCGGCCCGTGGACCTTCACCCTTCTCTGTGGTTTGCTG | 120 |
| GCAGCCACCTTGATCCAAGCCACCCTCAGTCCCACTGCAGTTCTCATCCTCGGCCCAAAA | 180 |
| GTCATCAAAGAAAAGCTGACACAGGAGCTGAAGGACCACAACGCCACCAGCATCCTGCAG | 240 |
| CAGCTGCCGCTGCTCAGTGCCATGCGGGAAAAGCCAGCCGGAGGCATCCCTGTGCTGGGC | 300 |
| AGCCTGGTGAACACCGGCCTGAAGCACATCATCTGGCTGAAGGTCATCACAGCTAACATC | 360 |
| CTCCAGCTGCAGGTGAAGCCCTCGGCCAATGACCAFFAFCTGCTAGTCAAGATCCCCCTG | 420 |
| GACATGGTGGCTGGATTCAACACGCCCCTGGTCAAGACCATCGTGGAGTTCCACATGACG | 480 |
| ACTGAGGCCCAAGCCACCATCCGCATGGACACCAGTGCAAGTGGCCCCACCCGCCTGGTC | 540 |
| CTCAGTGACTGTGCCACCAGCCATGGGAGCCTGCGCATCCAACTGCTGCATAAGCTCTCC | 600 |
| TTCCTGGTGAACGCCTTAGCTAAGCAGGTCATGAACCTCCTAGTGCCATCCCTGCCCAAT | 660 |
| CTAGTGAAAAACCAGCTGTGTCCCGTGATCGAGGCTTCCTTCAATGGCATGTATGCAGAC | 720 |
| CTCCTGCAGCTGGTGAAGGGTAGGTGCTCTGCTCTCTCTCCCACTTTTTCCTTTACTACG | 780 |
| GAGCTGGCCTCCAGACCCGGAAAGGTGACCAAGTGGTTCAATAACTCTGCAGCTTCCCTG | 840 |
| ACAATGCCCACCCTGGACAACATCCCGTTCAGCCTCATCGTGAGTCAGGACGTGGTGAAA | 900 |
| GCTGCAGTGGCTGCTGTGCTCTCTCCAGAAGAATTCATGGTCCTGTTGGACTCTGTGGTA | 960 |
| AACCTCAGCACAAGGCAGAGAATAGGGCCGCCCAGGCCACATCATAGGAATTTCCTGAAC | 1020 |
| ACAGGGTGCCCCTAA | 1035 |

The disclosed nucleic acid sequence has 538 of 698 nucleotides (77%) identical to a 1683 bp Mus musculus von Ebner minor salivary gland protein (GENBANK-ID:MMU46068|acc:U46068) (E value=4.0e-⁸⁴).

The NOV6a protein encoded by SEQ ID NO: 13 has 318 amino acid residues, and is presented using the one-letter code in Table 6D (SEQ ID NO: 16). The SignalP, Psort and/or Hydropathy profile for NOV6b predict that NOV6a is likely to be localized extracellularly, with a certainty of 0.6138. A cleavage site is indicated at the slash in the sequence TLS-PT, between amino acids 24 and 25 in Table 6D. NOV6b differs from NOV6a at five positions: S39>K; T85>I; P86>Δ; S87>Δ; R88>W.

TABLE 6B
Encoded NOV6b protein sequence (SEQ ID NO:14).

| | |
|---|---|
| MAGPWTFTLLCGLLAATLIQATLS/PTAVLILGPKVIKEKLTQELKDHNATSILQLPLLS | 60 |
| AMREKPAGGIPVLGSLVNTVLKHIIWLKVITANILQLQVKPSANDOELLVKIPLDMVAGF | 120 |
| NTPLVKTIVEFHMTTEAQATIRMDTSASGPTRLVLSDCATSHGSLRIQLLHKLSFLVNAL | 180 |
| AKQVMNLLVPSLPNLVKNQLCPVIEASFNGMYADLLQLVKGRCSALSPTFSFTTELASRP | 240 |
| GKVTKWFNNSAASLTMPTLDNIPFSLIVSQDVVKAAVAAVLSPEEFMVLLDSVVNLSTRQ | 300 |
| RIGPPRPHHRNFLNTGCP 318 | |

The full amino acid sequence of the disclosed NOV6b protein was found to have 165 of 302 amino acid residues (54%) identical to, and 209 of 302 amino acid residues (69%) positive with, the 310 amino acid residue protein von Ebner minor salivary gland protein from Mus musculus (SPTREMBL-ACC:Q61114) (E value=1.1 e-73).

Patp results include those listed in Table 6E.

TABLE 6E
Patp alignments of NOV6

| | | | Smallest |
|---|---|---|---|
| | | | Sum |
| | Reading | High | Prob |
| Sequences producing High-scoring Segment Pairs: | Frame | Score | P(N) |
| Patp:Y77126 Human neurotransmission-associated protein | . . .+1 | 1282 | 6.5e-130 |
| patp:Y99375 Human PRO1357 (UNQ706) amino acid sequence | . . .+1 | 1276 | 2.8e-129 |
| patp:Y86219 Human secreted protein HBHMA23, | . . .+1 | 920 | 1.5e-91 |
| patp:B58378 Lung cancer associated polypeptide sequence | . . .+1 | 920 | 1.5e-91 |
| patp:B40750 Human ORFX ORF5l4 polypeptide sequence | . . .+1 | 679 | 5.2e-66 |
| patp:Y86310 Human secreted protein HBHMA23, | . . .+1 | 334 | 3.5e-33 |

For example, a BLAST against Y77126, a 484 amino acid neurotransmission-associated protein from Homo sapiens, produced 275/310 (88%) identity, and 277/310 (89%) positives (E=6.5e-130), with long segments of amino acid identity. WO 00/01821. Y77126 is described as a putative odorant-binding protein whose cDNA was isolated from nasal polyp tissue. NOV6 also has significant homology with a number of secreted proteins. WO 00/12708; WO 99/66041; WO 00/55180; and WO 00/54873.

The disclosed NOV6 protein (SEQ ID NO: 25) has good identity with salivary gland proteins. The identity information used for ClustalW analysis is presented in Table 6F.

TABLE 6F
BLAST results for NOV6

| Gene Index/ | | Length | Identity | Positives | |
|---|---|---|---|---|---|
| Identifier | Protein/Organism | (aa) | (%) | (%) | Expect |
| Gi\|9938033\|ref\|NP_0 | Von Ebner minor | 474 | 175/319 | 222/319 | 7e-80 |
| 61205.2\| | salivary gland | | (54%) | (68%) | NOV6a |
| | protein Mus | | NOV6a | NOV6a | |
| | musculus | | | | |
| | | 474 | 177/317 | 224/317 | 9e-84 |
| | | | (55%) | (69%) | NOV6b |
| | | | NOV6b | NOV6b | |
| Gi\|13274680\|emb\|CAC | novel protein | 285 | 79/111 | 81/111 | 1e-23 |
| 34050.1\| | similar to mouse | | (71%) | (72%) | NOV6a |
| | von Ebner | | NOV6a | NOV6a | |
| | salivary gland | | | | |
| | protein, isoform | | | | |
| | 1.) Homo sapiens | | | | |
| | | 285 | 79/111 | 81/111 | 1e-23 |
| | | | (71%) | (72%) | NOV6b |
| | | | NOV6b | NOV6b | |

This information is presented graphically in the multiple sequence alignment given in Table 6G (with NOV6a being shown on line 1, and NOV6b shown on line 2) as a ClustalW analysis comparing NOV6 with related protein sequences.

[image]

The presence of identifiable domains in NOV6 was determined by searches using algorithms such as PROSITE, Blocks, Pfam, ProDomain, Prints and then determining the Interpro number by crossing the domain match (or numbers) using the Interpro website (http:www.ebi.ac.uk/interpro/).

DOMAIN results for NOV6 were collected from the Conserved Domain Database (CDD) with Reverse Position Specific BLAST. This BLAST samples domains found in the Smart and Pfam collections. The results are listed in Table 6H with the statistics and domain description. The results indicate that this protein contains the BPI/LBP/CTEP N-terminal domain, bactericidal permeability increasing protein/lipopolysaccharide-binding protein/cholesteryl ester transferase domain. The von Ebner minor salivary gland protein also contains this domain. Amino acids 35-243 NOV6a aligns with amino acids 2-206 of this 225 residue protein, as indicated in Table 6H as SEQ ID NO: 66. The E value for NOV6a is 8e-15, and 6e-15 for NOV6b. This indicates that the sequence of NOV6 has properties similar to those of other proteins known to contain this domain.

[image]

Von Ebner glands (VEG) are small lingual salivary glands. Their ducts open into trenches of circumvallate and foliate papillae, and their secretions influence the milieu where the interaction between taste receptor cells and sapid (taste-processing) molecules takes place. The major secretion of human VEG is a protein with a molecular mass of 18 kD. This VEG protein is identical to lipocalin-1. Blaker et al. isolated a cDNA clone from a human VEG library and showed that it contained an insert of 735 bp, including an open reading frame that encodes the human VEG protein of 176 amino acids. Blaker et al., Biochim. Biophys. Acta 1172:131-137, 1993. The VEG proteins are members of the lipocalin protein superfamily; together with odorant-binding protein, they constitute a new subfamily. Sequence similarity to proteins such as retinol binding protein and odorant binding protein suggests a possible function for the human VEG protein in taste perception.

Lipocalins are a group of extracellular proteins, first described by Pervaiz and Brew that are able to bind lipophiles by enclosure within their structures, minimizing solvent contact. Pervaiz and Brew, FASEB J. 1:209-214, 1987. The lipocalins make up a heterogeneous superfamily of proteins. Although showing almost no sequence homology, they share very similar secondary and tertiary structures. Their ability to bind hydrophobic ligands is well established, but the physiological function of most lipocalins remains unclear. The lipocalin from the human Von Ebner's Gland of the tongue (VEGh) contains three sequence motifs corresponding with the papain-binding domains of cystatins, a family of naturally occurring cysteine proteinase inhibitors. VEGh was shown to inhibit papain activity to a similar extent as salivary cystatin S. Furthermore, synthetic peptides derived from VEGh and cystatin C, comprising these three motifs, inhibited papain, too. VEGh is a physiological inhibitor of cysteine proteinases and therefore can play a role in the control of inflammatory processes in oral and ocular tissues. Van't Hoff, et al. J. Biol. Chem. 272:1837-1841, 1997.

Furthermore, Redl et al. found enhanced LCN1 secretion in the airways of patients with cystic fibrosis. Redl, et al., Lab. Invest. 78:1121-1129, 1998. Northern blot analysis of RNA from normal trachea and RNA isolated from tracheal biopsies of patients with CF indicated that the enhanced secretion was due to an upregulated expression of the LCN1 gene. Thus, these investigators presented the first clear evidence that LCN1 is induced in infection or inflammation and supported the idea that this lipocalin functions as a physiologic protection factor of epithelia in vivo.

NOV6 has been analyzed for tissue expression profiles. The quantitative expression of various clones was assessed using microtiter plates containing RNA samples from a variety of normal and pathology-derived cells, cell lines and tissues using real time quantitative PCR (RTQ PCR; TAQMAN®). RTQ PCR was performed on a Perkin-Elmer Biosystems ABI PRISM® 7700 Sequence Detection System. Various collections of samples are assembled on the plates, and referred to as Panel 1 (containing cells and cell lines from normal and cancer sources), Panel 2 (containing samples derived from tissues, in particular from surgical samples, from normal and cancer sources), Panel 3 (containing samples derived from a wide variety of cancer sources) and Panel 4 (containing cells and cell lines from normal cells and cells related to inflammatory conditions). See Taqman Example.

As shown in Table 6J, below, this 96 well plate (4 control wells, 92 test samples) for panel 1.2, and its variants are composed of RNA/cDNA isolated from various human cell lines that have been established from normal and malignant human tissues. These cell lines have been extensively characterized by investigators in both academia and the commercial sector regarding their tumorgenicity, metastatic potential, drug resistance, invasive potential and other cancer-related properties. They serve as suitable tools for pre-clinical evaluation of anti-cancer agents and promising therapeutic strategies.

As shown in Table 26 below, panel 4 includes samples on a 96 well plate (2 control wells, 94 test samples) composed of RNA (Panel 4r) or cDNA (Panel 4d) isolated from various human cell lines or tissues related to inflammatory conditions.

TaqMan oligo set Ag719 for the NOV6 gene include the forward probe and reverse oligomers. Sequences for the oligos are shown in Table 6I.

TABLE 6I
Taqman primers

| Position | Primers | Sequences | | Length |
|---|---|---|---|---|
| 260 | Forward | 5′-CCAGGCTGAAGGTCATCAC-3′ | SEQ ID NO:67 | 19 |
| 281 | Probe | FAM-5′-CTAACATCCTCCAGCTGCAGGTGAAG | SEQ ID NO:68 | 26 |
| | | -3′-TAMRA | | |
| 316 | Reverse | 5′-GACTAGCAGCTCCTGGTCATT-3′ | SEQ ID NO:69 | 21 |

TABLE 6J
TaqMan Results

| PANEL 1.2 | | | | Panel 4D | | | |
|---|---|---|---|---|---|---|---|
| | % Rel. | % Rel. | % Rel. | | % Rel. | % Rel. | % Rel. |
| | Expn. | Expn. | Expn. | | Expn. | Expn. | Expn. |
| Tissue Name | Run 1 | Run 2 | Run 3 | Tissue Name | Run 1 | Run 2 | Run 3 |
| | | | | 93768_Secondary Th1_anti- | 0.0 | 0.0 | 0.0 |
| Endothelial cells | 0.0 | 0.0 | 0.0 | CD28/anti-CD3 | | | |
| | | | | 93769_Secondary TH2_anti- | 0.0 | 0.0 | 0.3 |
| Endothelial cells (treated) | 0.0 | 0.0 | 0.0 | CD28/anti-CD3 | | | |
| | | | | 93770_Secondary Tr1_anti- | 0.0 | 0.0 | 0.0 |
| Pancreas | 0.1 | 0.0 | 0.0 | CD28/anti-CD3 | | | |
| | | | | 93573_Secondary Th1_resting | 0.0 | 0.0 | 0.0 |
| Pancreatic ca.CAPAN 2 | 0.0 | 0.0 | 0.0 | day 4-6 in IL-2 | | | |
| | | | | 93572_Secondary Th2_resting | 0.0 | 0.0 | 0.0 |
| Adrenal Gland (new lot*) | 0.0 | 0.0 | 0.0 | day 4-6 in IL-2 | | | |
| | | | | 93571_Secondary Tr1_resting day | 0.0 | 0.0 | 0.0 |
| Thyroid | 0.1 | 0.0 | 0.0 | 4-6 in IL-2 | | | |
| | | | | 93568_primary Th1_anti- | 0.5 | 0.0 | 0.0 |
| Salavary gland | 1.6 | 0.8 | 1.8 | CD28/anti-CD3 | | | |
| | | | | 93569_primary Th2_anti- | 0.5 | 0.0 | 0.0 |
| Pituitary gland | 0.3 | 0.0 | 0.0 | CD28/anti-CD3 | | | |
| Brain (fetal) | 0.0 | 0.0 | 0.0 | 93570_primary Tr1_anti- | 0.0 | 0.0 | 0.0 |
| | | | | CD28/anti-CD3 | | | |
| | | | | 93565_primary Th1_resting dy 4- | 0.3 | 0.0 | 0.0 |
| Brain (whole) | 0.0 | 0.0 | 0.0 | 6 in IL-2 | | | |
| | | | | 93566_primary Th2_resting dy 4- | 0.0 | 0.0 | 0.0 |
| Brain (amygdala) | 0.0 | 0.0 | 0.0 | 6 in IL-2 | | | |
| | | | | 93567_primary Tr1_resting dy 4-6 | 0.0 | 0.0 | 0.0 |
| Brain (cerebellum) | 0.0 | 0.0 | 0.0 | in IL-2 | | | |
| | | | | 93351_CD45RA CD4 | 0.0 | 0.0 | 0.0 |
| Brain (hippocampus) | 0.0 | 0.0 | 0.0 | lymphocyte_anti-CD28/anti-CD3 | | | |
| | | | | 93352_CD45RO CD4 | 0.0 | 0.0 | 0.0 |
| Brain (thalamus) | 0.0 | 0.0 | 0.0 | lymphocyte_anti-CD28/anti-CD3 | | | |
| | | | | 93251_CD8 Lymphocytes_anti- | 0.0 | 0.0 | 0.0 |
| Cerebral Cortex | 0.0 | 0.0 | 0.0 | CD28/anti-CD3 | | | |
| | | | | 93353_chronic CDS Lymphocytes | 0.0 | 0.0 | 0.0 |
| Spinal cord | 0.1 | 0.0 | 0.1 | 2ry_resting dy 4-6 in IL-2 | | | |
| | | | | 93574_chronic CD8 Lymphocytes | 0.2 | 0.0 | 0.0 |
| CNS ca. (glio/astro) U87-MG | 0.0 | 0.0 | 0.0 | 2ry_activated CD3/CD28 | | | |
| CNS ca. (glio/astro)U-118- | | | | 93354_CD4_none | 0.0 | 0.0 | 0.0 |
| MG | 0.0 | 0.0 | 0.0 | | | | |
| | | | | 93252_Secondary | 0.0 | 0.4 | 0.0 |
| CNS ca.(astro) SW1783 | 0.0 | 0.0 | 0.0 | Th1/Th2/Tr1_anti-CD95 CH11 | | | |
| CNS ca.*(neuro; met) SK-N- | | | | 93103_LAK cells_resting | 0.0 | 0.0 | 0.0 |
| AS | 0.0 | 0.0 | 0.0 | | | | |
| CNS ca. (astro)SF-539 | 0.0 | 0.0 | 0.0 | 93788_LAK cells_IL-2 | 0.2 | 0.0 | 0.0 |
| CNS ca. (astro) SNB-75 | 0.0 | 0.0 | 0.0 | 93787_LAK cells_IL-2+IL-12 | 0.0 | 0.0 | 0.0 |
| | | | | 93789_LAK cells_IL-2+IFN | 0.3 | 0.0 | 0.2 |
| CNS ca. (glio) SNB-19 | 0.0 | 0.0 | 0.0 | gamma | | | |
| CNS ca. (glio) U251 | 0.0 | 0.0 | 0.0 | 93790_LAK cells_IL-2+IL-18 | 0.0 | 0.0 | 0.0 |
| | | | | 93104_ | 0.0 | 0.0 | 0.0 |
| CNS ca. (glio) SF-295 | 0.0 | 0.0 | 0.0 | cells_PMA/ionomycin and IL-18 | | | |
| Heart | 0.0 | 0.0 | 0.0 | 93578_NK Cells IL-2_resting | 0.0 | 0.0 | 0.0 |
| | | | | 93109_Mixed Lymphocyte | 0.0 | 0.0 | 0.0 |
| Skeletal Muscle (new lot*) | 0.0 | 0.0 | 0.0 | Reaction_Two Way MLR | | | |
| | | | | 93110_Mixed Lymphocyte | 0.0 | 0.0 | 0.0 |
| Bone marrow | 0.0 | 0.0 | 0.0 | Reaction_Two Way MLR | | | |
| | | | | 93111_MixedLymphocyte | 0.0 | 0.0 | 0.0 |
| Thymus | 0.0 | 0.0 | 0.0 | Reaction_Two Way MLR | | | |
| | | | | 93112_Mononuclear Cells | 0.0 | 0.0 | 0.0 |
| Spleen | 0.0 | 0.0 | 0.0 | (PBMCs)_resting | | | |
| | | | | 93113_Mononuclear Cells | 0.0 | 0.0 | 0.0 |
| Lymph node | 0.3 | 0.1 | 0.2 | (PBMCs)_PWM | | | |
| | | | | 93114_Mononuclear Cells | 0.0 | 0.0 | 0.0 |
| Colorectal | 0.0 | 0.0 | 0.0 | (PBMCs)_PHA-L | | | |
| Stomach | 2.3 | 3.4 | 7.3 | 93249_Ramos (B cell)_none | 0.0 | 0.0 | 0.0 |
| Small intestine | 0.0 | 0.0 | 0.0 | 93250_Ramos (B cell)_ionomycin | 0.0 | 0.0 | 0.0 |
| Colon ca. SW480 | 0.0 | 0.0 | 0.0 | 93349_B lymphocytes_PWM | 0.0 | 0.0 | 0.0 |
| Colon ca.* (SW480 | | | | 93350_B lymphoytes_CD40L and | 0.0 | 0.0 | 0.0 |
| met)SW620 | 0.0 | 0.0 | 0.0 | IL-4 | | | |
| | | | | 92665_EOL-1 | 0.0 | 0.0 | 0.0 |
| | | | | (Eosinophil)_dbcAMP | | | |
| Colon ca. HT29 | 0.0 | 0.0 | 0.0 | differentiated | | | |
| | | | | 93248_EOL-1 | 0.0 | 0.0 | 0.0 |
| | | | | (Eosinophil)_dbcAMP/PMAiono | | | |
| Colon ca. HCT-116 | 0.0 | 0.0 | 0.0 | mycin | | | |
| Colon ca. CaCo-2 | 0.0 | 0.0 | 0.0 | 93356_Dendritic Cells_none | 0.0 | 0.0 | 0.0 |
| 83219 CC Well to Mod Diff | | | | 93355_Dendritic Cells_LPS 100 | 0.0 | 0.2 | 0.0 |
| (ODO3866) | 0.0 | 0.0 | 0.0 | ng/ml | | | |
| Colon ca. HCC-2998 | 0.0 | 0.0 | 0.0 | 93775_Dendritic Cells_anti-CD40 | 0.0 | 0.0 | 0.0 |
| Gastric ca.* (liver met) NCI- | | | | 93774_Monocytes_resting | 0.0 | 0.0 | 0.0 |
| N87 | 0.1 | 0.0 | 0.1 | | | | |
| Bladder | 0.0 | 0.0 | 0.0 | 93776_Monocytes_LPS 50 ng/ml | 0.3 | 0.0 | 0.0 |
| Trachea | 100.0 | 100.0 | 100.0 | 93581_Macrophages_resting | 0.0 | 0.0 | 0.0 |
| | | | | 93582_Macrophages_LPS 100 | 0.0 | 0.0 | 0.0 |
| Kidney | 0.0 | 0.0 | 0.0 | ng/ml | | | |
| | | | | 93098_HUVEC | 0.0 | 0.0 | 0.0 |
| Kidney (fetal) | 0.0 | 0.0 | 0.0 | (Endothelial)_none | | | |
| | | | | 93099_HUVEC | 0.0 | 0.4 | 0.0 |
| Renal ca. 786-0 | 0.0 | 0.0 | 0.0 | (Endothelial)_starved | | | |
| | | | | 93100_HUVEC (Endothelial)_IL- | 0.0 | 0.0 | 0.0 |
| Renal ca. A498 | 0.0 | 0.0 | 0.0 | 1b | | | |
| | | | | 93779_HUVEC | 0.0 | 0.0 | 0.5 |
| Renal ca. RXF 393 | 0.0 | 0.0 | 0.0 | (Endothelial)_IFN gamma | | | |
| | | | | 93102_HUVEC | 0.5 | 0.0 | 0.0 |
| | | | | (Endothelial)_TNF alpha+IFN | | | |
| Renal ca. ACHN | 0.0 | 0.0 | 0.0 | gamma | | | |
| | | | | 93101_HUVEC | 0.0 | 0.0 | 0.0 |
| Renal ca. UO-31 | 0.0 | 0.0 | 0.0 | (Endothelial)_TNF alpha+IL4 | | | |
| | | | | 93781_HUVEC (Endothelial)_IL- | 0.0 | 0.0 | 0.0 |
| Renal ca. TK-10 | 0.0 | 0.0 | 0.0 | 11 | | | |
| | | | | 93583_Lung Microvascular | 0.0 | 0.3 | 0.0 |
| Liver | 0.0 | 0.0 | 0.0 | Endothelial Cells none | | | |
| | | | | 93584_Lung Microvascular | 0.0 | 0.0 | 0.0 |
| | | | | Endothelial Cells_TNFa (4 ng/ml) | | | |
| Liver (fetal) | 0.0 | 0.0 | 0.0 | and IL1b (1 ng/ml) | | | |
| | | | | 92662_Microvascular Dermal | 0.0 | 0.0 | 0.0 |
| Liver ca. (hepatoblast) HepG2 | 0.0 | 0.0 | 0.0 | endothelium_none | | | |
| | | | | 92663_Microsvasular Dermal | 0.3 | 0.0 | 0.0 |
| | | | | endothelium_TNFa (4 ng/ml) and | | | |
| Lung | 5.2 | 2.3 | 4.9 | IL 1b (1 ng/ml) | | | |
| | | | | 93773_Bronchial | 0.0 | 0.0 | 0.0 |
| | | | | epithelium_TNFa (4 ng/ml) and | | | |
| Lung (fetal) | 1.0 | 0.3 | 0.6 | IL 1b (1 ng/ml)** | | | |
| | | | | 93347_Small Airway | 0.0 | 0.2 | 0.0 |
| Lung ca. (small cell)LX-1 | 0.0 | 0.0 | 0.0 | Epithelium_none | | | |
| | | | | 93348_Small Airway | 0.0 | 0.2 | 0.0 |
| | | | | Epithelium_TNFa (4 ng/ml) and | | | |
| Lung ca. (small cell) NCI-H69 | 0.0 | 0.0 | 0.0 | IL1b (1 ng/ml) | | | |
| | | | | 92668_Coronery Artery | 0.0 | 0.0 | 0.0 |
| Lung ca. (s.cell var.) SHP-77 | 0.0 | 0.0 | 0.0 | SMC_resting | | | |
| | | | | 92669_Coronery Artery | 0.0 | 0.0 | 0.0 |
| | | | | SMC_TNFa (4 ng/ml) and IL1b (1 | | | |
| Lung ca. (large cell)NCI-H460 | 0.0 | 0.0 | 0.0 | ng/ml) | | | |
| Lung ca. (non-sm. cell) A549 | 0.1 | 0.0 | 0.1 | 93107_astrocytes_resting | 0.0 | 0.2 | 0.0 |
| | | | | 93108_astrocytes_TNFa (4 ng/ml) | 0.5 | 0.0 | 0.0 |
| Lung ca. (non-s.cell) NCI-H23 | 0.0 | 0.0 | 0.0 | and IL1b (1 ng/ml) | | | |
| | | | | 92666_KU-812 | 0.0 | 0.0 | 0.0 |
| Lung ca (non-s.cell) HOP-62 | 0.0 | 0.0 | 0.0 | (Basophil)_resting | | | |
| | | | | 92667_KU-812 | 0.0 | 0.0 | 0.0 |
| Lung ca. (non-s.cl) NCI-H522 | 0.0 | 0.0 | 0.0 | (Basophil)_PMA/ionoycin | | | |
| | | | | 93579_CCD11O6 | 0.0 | 0.0 | 0.0 |
| Lung ca. (squam.) SW 900 | 0.3 | 0.1 | 0.4 | (Keratinocytes)_none | | | |
| | | | | 93580_CCD1106 | 0.3 | 0.0 | 0.0 |
| | | | | (Keratinocytes)_TNFa and TFNg | | | |
| Lung ca. (squam.) NCI-H596 | 0.0 | 0.0 | 0.0 | ** | | | |
| Mammary gland | 0.1 | 0.0 | 0.0 | 93791_Liver Cirrhosis | 1.6 | 2.3 | 1.9 |
| Breast ca.*(pl. effusion) | | | | 93792_Lupus Kidney | 0.3 | 0.0 | 0.0 |
| MCF-7 | 0.0 | 0.0 | 0.0 | | | | |
| Breast ca.*(pl.ef) MDA-MB- | | | | 93577_NCI-H292 | 88.9 | 78.5 | 86.5 |
| 231 | 0.0 | 0.0 | 0.0 | | | | |
| Breast ca.* (pl. effusion) | | | | 93358_NCI-H292_IL-4 | 64.2 | 100.0 | 56.6 |
| T47D | 0.0 | 0.0 | 0.0 | | | | |
| Breast ca. BT-549 | 0.0 | 0.0 | 0.0 | 93360_NCI-H292_IL-9 | 68.8 | 54.0 | 55.1 |
| Breast ca. MDA-N | 0.0 | 0.0 | 0.0 | 93359_NCI-H292_Il-13 | 47.0 | 13.1 | 53.2 |
| Ovary | 0.0 | 0.0 | 0.0 | 93357_NCI-H292_IFN gamma | 30.6 | 10.4 | 19.2 |
| Ovarian ca.OVCAR-3 | 0.0 | 0.0 | 0.0 | 93777_HPAEC_- | 0.0 | 0.0 | 0.0 |
| | | | | 93778_HPAEC_IL-1 beta/TNA | 0.0 | 0.0 | 0.0 |
| Ovarian ca.OVCAR-4 | 0.0 | 0.0 | 0.0 | alpha | | | |
| | | | | 93254_Normal Human Lung | 0.0 | 0.0 | 0.0 |
| Ovarian ca.OVCAR-5 | 0.9 | 0.3 | 0.4 | Fibroblast_none | | | |
| | | | | 93253_Normal Human Lung | 0.2 | 0.0 | 0.0 |
| | | | | Fibroblast_TNFa (4 ng/ml) and | | | |
| Ovarian ca. OVCAR-8 | 0.0 | 0.0 | 0.0 | IL-1b (1 ng/ml) | | | |
| | | | | 93257 Normal Human Lung | 0.0 | 0.0 | 0.0 |
| Ovarian ca.IGROV-1 | 0.0 | 0.0 | 0.0 | Fibroblast_IL-4 | | | |
| Ovarian ca.* (ascites) SK-OV- | | | | 93256_Normal Human Lung | 0.1 | 0.0 | 0.0 |
| 3 | 0.0 | 0.0 | 0.0 | Fibroblast_IL-9 | | | |
| | | | | 93255_Normal Human Lung | 0.0 | 0.0 | 0.0 |
| Uterus | 0.0 | 0.0 | 0.0 | Fibroblast_IL-13 | | | |
| | | | | 93258_Normal Human Lung | 0.0 | 0.0 | 0.0 |
| Plancenta | 0.0 | 0.0 | 0.0 | Fibroblast_IFN gamma | | | |
| | | | | 93106_Dermal Fibroblasts | 0.0 | 0.0 | 0.0 |
| Prostate | 0.0 | 0.0 | 0.0 | CCD1070_resting | | | |
| | | | | 93361_Dermal Fibroblasts | 0.0 | 0.0 | 0.0 |
| Prostate ca.* (bone met)PC-3 | 0.0 | 0.0 | 0.0 | CCD1070_TNF alpha 4 ng/ml | | | |
| | | | | 93105_Dermal Fibroblasts | 0.0 | 0.0 | 0.0 |
| Testis | 0.2 | 0.0 | 0.1 | CCD1070_IL-1 beta 1 ng/ml | | | |
| | | | | 93772_dermal fibroblast_IFN | 0.0 | 0.4 | 0.0 |
| Melanoma Hs688(A).T | 0.0 | 0.0 | 0.0 | gamma | | | |
| Melanoma* (met) Hs688(B).T | 0.0 | 0.0 | 0.0 | 93771_dermal fibroblast_IL-4 | 0.0 | 0.0 | 0.0 |
| Melanoma UACC-62 | 0.1 | 0.0 | 0.1 | 93259_IBD Colitis 1** | 1.6 | 1.2 | 1.4 |
| Melanoma M14 | 0.0 | 0.0 | 0.0 | 93260_IBD Colitis 2 | 0.0 | 0.0 | 0.0 |
| Melanoma LOX IMVI | 0.0 | 0.0 | 0.0 | 93261_IBD Crohns | 1.3 | 3.3 | 0.4 |
| Melanoma* (met) SK-MEL-5 | 0.0 | 0.0 | 0.0 | 735010_Colon_normal | 0.3 | 0.0 | 1.4 |
| Adipose | 0.0 | 0.0 | 0.2 | 735019_Lung_none | 100.0 | 72.2 | 100.0 |
| | | | | 64028-1_Thymus_none | 1.0 | 0.4 | 0.0 |
| | | | | 64030-1_Kidney_none | 0.8 | 0.4 | 0.8 |

In Table 6J the following abbreviations are used: ca. carcinoma, * = established from metastasis, met = metastasis, s cell var = small cell variant, non-s = non-sm = non-small, squam = squamous, pl. eff = pl effusion = pleural effusion, glio = glioma, astro = astrocytoma, and neuro = neuroblastoma.

The results from Panel 1.2 indicate that NOV6 is expressed in normal trachea, salivary gland and lung, but NOV6 is not expressed on any tumor tissues. The results from panel 4D indicate that NOV6 is expressed highly in lung and in the lung airway epithelial cell line NCI-H292, and that with treatment with gamma interferon reduces NOV6 expression 3-10 fold in these cells. NOV6 is expressed in normal airway tissue such as the lung and trachea and expression is down regulated in gamma interferon treated tissues. The reduction in NOV6 may contribute to the inflammatory processes in the airways due to allergy/asthma, emphysema or viral infection. Protein therapeutics derived from NOV6 might reduce or eliminate inflammation in the lung due to asthma/allergy, emphysema, or viral infection. Since it is known that gamma interferon treatment stimulates the expression of the cell adhesion molecule ICAM-1 on NCI-H292 cells, it is possible that treatment with NOV6 would prevent the expression of cell adhesion molecules and reduce or prevent leukocyte infiltration into the lung. See, e.g., Togas, et al., Euro J Pharmacol 345:199-206, 1998.

The similarity information for the NOV6 protein and nucleic acid disclosed herein suggest that NOV6 may have important structural and/or physiological functions characteristic of the salivary gland protein family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon. The novel nucleic acid encoding NOV6, and the NOV6 protein of the invention, or fragments thereof, may further be useful in diagnostic applications, wherein the presence or amount of the nucleic acid or the protein are to be assessed.

The nucleic acids and proteins of the invention are useful in potential diagnostic and therapeutic applications implicated in various diseases and disorders described below and/or other pathologies. For example, the compositions of the present invention will have efficacy for treatment of patients suffering from olfactory disorders, salivitory disorders, digestive disorders, oral immunologic disorders, poor oral health, inflammatory processes in the airways due to allergy/asthma, emphysema or viral infection, cystic fibrosis, obesity and/or other pathologies and disorders of the like.

The polypeptides can be used as immunogens to produce antibodies specific for the invention, and as vaccines. They can also be used to screen for potential agonist and antagonist compounds. For example, a cDNA encoding the salivary gland-like protein may be useful in gene therapy, and the salivary gland-like protein may be useful when administered to a subject in need thereof. By way of nonlimiting example, the compositions of the present invention will have efficacy for treatment of patients suffering from bacterial, fungal, protozoal and viral infections, olfactory disorders, salivitory disorders, digestive disorders, oral immunologic disorders, poor oral health, inflammatory processes in the airways due to allergy/asthma, emphysema or viral infection, cystic fibrosis, obesity and/or other pathologies and disorders of the like.

The novel nucleic acid encoding salivary gland-like protein, and the salivary gland-like protein of the invention, or fragments thereof, may further be useful in diagnostic applications, wherein the presence or amount of the nucleic acid or the protein are to be assessed.

These materials are further useful in the generation of antibodies that bind immuno-specifically to the novel NOV6 substances for use in therapeutic or diagnostic methods. These antibodies may be generated according to methods known in the art, using prediction from hydrophobicity charts, as described in the “Anti-NOVX Antibodies” section below. In one embodiment, a contemplated NOV6 epitope is from about aa 25 to 65. In another embodiment, a NOV6 epitope is from about aa 95 to 105. In additional embodiments, NOV6 epitopes are from aa 135 to 160, 225-260, and from 290 to 310.

NOV7

A novel nucleic acid was identified on chromosome 11 by TblastN using CuraGen Corporation's sequence file for CD-81 or homolog as run against the Genomic Daily Files made available by GenBank or from files downloaded from the individual sequencing centers. The nucleic acid sequence was predicted from the genomic file GenBank Accession Number: AC016702, by homology to a known CD-81 or homolog. Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein

The disclosed NOV7 nucleic acid of 754 nucleotides (also referred to as GM_{—}51624520_A, or CG54665-01) is shown in Table 7A. An open reading begins with an ATG initiation codon at nucleotides 5-7 and ends with a ATG codon at nucleotides 746-748. A putative untranslated region upstream from the initiation codon and downstream from the termination codon are underlined in Table 7A, and the start and stop codons are in bold letters.

TABLE 7A
NOV7 Nucleotide Sequence (SEQ ID NO:17)

CACCATGGAAGGCGACTGTCTGAGCTGCATGAAGTATCTGATGTTTGTATTCAATTTCTTCATATTTCTG
GGCGGGGCCTGCCTGCTGGCCATCGGCATCTGGGTCATGGTGGACCCCACCGGCTTCCGGGAGATCGTGG
CTGCCAATCCTCTGCTCCTCACGGGCGCCTACATCCTCCTGGCCATGGGGGGCCTGCTCTTTCTGCTCGG
CTTCCTGGGCTGCTGCGGGGCCGTCCGTGAGAACAAGTGTCTGCTGCTATTTTTCTTCCTGTTCATCCTG
ATCATCTTCCTGGCAGAGCTCTCAGCAGCCATCCTGGCCTTCATCTTCAGGGAAAATCTCACCCGAGAAT
TCTTCACCAAGGGGCTCACCAAGCACTACCAGGGCAATAACGACACAGACGTCTTCTCTGCCACCTGGAA
CTCGGTCATGATCACATTTGGTTGCTGCGGGGTCAACGGGCCTGAAGACTTTAAGTTTGCACCCTGGATA
GTGAAGAGGTGCCGGCGCCTGCTGCCGGAGGAACCCCAAAGTCGGGACGGGGTCCTGCTGAGCCGGGAGG
AGTGCCTCCTGGGAAGGAGCCTATTCCTAAACAAGCAGCAGGGCTGTTACACGGTGATCCTCAACACCTT
CGAGACCTACGTCTACTTGGCCGGAGCCCTTGCCATCGGGGTACTGGCCATCGAGGTATTTCGCCATGAT
CTTTGCCATGTGCCTCTTCCGGGGCATCCAGTAGAGGGTATGGCCTGAAGCCTG

The disclosed nucleic acid sequence has 512 of 711 bases (72%) identical to a 935 bp Gallus gallus CD-81 mRNA (gb:GENBANK-ID:AF206661|acc:AF206661 Gallus gallus neuronal tetraspanin mRNA, complete cds) (E value=2.4e-⁶⁴).

The NOV7 protein encoded by SEQ ID NO: 17 has 247 amino acid residues, and is presented using the one-letter code in Table 7B (SEQ ID NO: 18). The SignalP, Psort and/or Hydropathy profile for NOV7 predict that NOV7 has a signal peptide and is likely to be localized at the plasma membrane with a certainty of 0.6400. The SignalP shows a signal sequence is coded for in the first 28 amino acids, i.e., with a cleavage site at the slash in the sequence ACL-LA, between amino acids 27 and 28 in Table 7B.

TABLE 7B
Encoded NOV7 protein sequence (SEQ ID NO:18).

MEGDCLSCMKYLMFVFNFFIFLGGACL/LAIGIWVMVDPTGFREIVAANPLLLTGAYILLAMGGLLFLLGF
LGCCGAVRENKCLLLFFFLFILIIFLAELSAAILAFIFRENLTREFFTKGLTKHYQGNNDTDVFSATWNS
VMITFGCCGVNGPEDFKFAPWIVKRCRRLLPEEPQSRDGVLLSREECLLGRSLFLNKQQGCYTVILNTFE
TYVYLAGALAIGVLAIEVFRHDLCHVPLPGHPVEGMA

The full amino acid sequence of the protein of the invention was found to have 180 of 234 amino acid residues (76%) identical to, and 199 of 234 residues (85%) positive with, the 247 amino acid residue neuronal tetraspanin protein from Gallus gallus (ptnr: TREMBLNEW-ACC:AAF19031) (E value=2.0e-⁹²).

Patp results include those listed in Table 7C.

TABLE 7C

Patp alignments of NOV7

| | | | |
|---|---|---|---|
| | | | Smallest |
| | | | Sum |
| | Reading | High | Prob |
| Sequences producing High-scoring Segment Pairs: | Frame | Score | P(N) |

| | | | |
|---|---|---|---|
| Patp:B49503 Clone HCE1K90 #1-Homo sapiens, 248 aa. | +2 | 1080 | 1.7e−108 |
| Patp:B49509 Clone HCE1K90 #2-Homo sapiens, 164 aa. | +2 | 835 | 1.5e−82 |
| Patp:W61618 Clone HPWAE25 of TM4SF superfamily H. sapiens | +2 | 328 | 8.1e−29 |

For example, NOV7 shows good homology with two receptor proteins from the 4 transmembrane superfamily (B49503 and B49509). PCT application WO 00/70076. The alignments of with these proteins are shown in Table 7D and 7E.

TABLE 7D
Alignment of NOV7 with B49503 (SEQ ID NO:70).

Length = 248 Plus Strand HSPs:
Score = 1080 (380.2 bits), Expect = 1.7e−108, P = 1.7e−108
Identities = 218/235 (92%), Positives = 220/235 (93%), Frame = +2

| | | | |
|---|---|---|---|
| NOV7: | 1 | MEGDCLSCMKYLMFVFNFFIFLGGACLLAIGIWVMVDPTGFREIVAANPLLLTGAYILLA | 60 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| B49503: | 1 | MEGDCLSCMKYLMFVFNFFIFLGGACLLAIGIWVMVDPTGFREIVAANPLLLTGAYILLA | 60 |
| NOV7: | 61 | MGGLLFLLGFLGCCGAVRENKCLLLFFFLFILIIFLAELSAAILAFIFRENLTREFFTKG | 120 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| B49503: | 61 | MGGLLFLLGFLGCCGAVRENKCLLLFFFLFILIIFLAELSAAILAFIFRENLTREFFTKE | 120 |
| NOV7: | 121 | LTKHYQGNNDTDVFSATWNSVMITFGCCGVNGPEDFKFAPWIVKRCRRL----LPE---- | 172 |
| | | \|\|\|\|\|\|\|\|\|\|\|\|\|\|\|\|\|\|\|\|\|\|\|\|\|\|\|\|\|\|\|\|\|\|\|\|\|\|\| \| \| \| +\|\| | |
| B49503: | 121 | LTKHYQGNNDTDVFSATWNSVMITFGCCGVNGPEDFKFAS--VFRLLTLDSEEVPEACCR | 178 |
| NOV7: | 173 | -EPQSRDGVLLSREECLLGRSLFLNKQQGCYTVILNTFETYVYLAGALAIGVLAIEVF | 228 |
| | | \|\|\|\|\|\|\|\|\|\|\|\|\|\|\|\|\|\|\|\|\|\|\|\|\|\| \|\|\|\|\|\|\|\|\|\|\|\|\|\|\|\|\|\|\|\|\|\|\|\|\|\|\|\|+\| | |
| B49503: | 179 | REPQSRDGVLLSREECLLGRSLFLNKQ-GCYTVILNTFETYVYLAGALAIGVLAIELF | 235 |

TABE 7E
Alignment of NOV7 with B49509 (SEQ ID NO:71).

Length = 164 Plus Strand HSPs:
Score = 835 (293.9 bits), Expect = 1.5e−82. P = 1.5e−82
Identities = 158/159 (99%), Positives = 158/159 (99%), Frame = +2

| | | | |
|---|---|---|---|
| NOV7: | 1 | MEGDCLSCMKYLMFVFNFFIFLGGACLLAIGIWVMVDPTGFREIVAANPLLLTGAYILLA | 60 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| B49509: | 1 | MEGDCLSCMKYLMFVFNFFIFLGGACLLAIGIWVMVDPTGFREIVAANPLLLTGAYILLA | 60 |
| NOV7: | 61 | MGGLLFLLGFLGCCGAVRENKCLLLFFFLFILIIFLAELSAAILAFIFRENLTREFFTKG | 120 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| B49509: | 61 | MGGLLFLLGFLGCCGAVRENKCLLLFFFLFILIIFLAELSAAILAFIFRENLTREFFTKE | 120 |
| NOV7: | 121 | LTKHYQGNNDTDVFSATWNSVMITFGCCGVNGPEDFKFA | 481 |
| | | \|\|\|\|\|\|\|\|\|\|\|\|\|\|\|\|\|\|\|\|\|\|\|\|\|\|\|\|\|\|\|\|\|\|\|\|\|\|\| | |
| B49509: | 121 | LTKHYQGNNDTDVFSATWNSVMITFGCCGVNGPEDFKFA | 159 |

Further BLAST analysis produced the significant results listed in Table 7F. The disclosed NOV7 protein (SEQ ID NO: 18) has good identity with proteins.

TABLE 7F

BLAST results for NOV7

| | | | | | |
|---|---|---|---|---|---|
| Gene Index/ | Protein/ | Length | Identity | Positives | |
| Identifier | Organism | (aa) | (%) | (%) | Expect |

| | | | | | |
|---|---|---|---|---|---|
| Gi\|6601561\|gb\|AAF190 | neuronal | 247 | 128/235 | 143/235 | 4e−59 |
| 31.1\|AP206661_1 | tetraspanin | | (54%) | (60%) | |
| (AF206661) | Gallus gallus | | | | |
| Gi\|6685175\|gb\|AAF238 | tetraspanin | 267 | 42/185 | 71/185 | 6e−07 |
| 28.1\|AP220044_1 | Drosophila | | (22%) | (37%) | |
| (AF220044) | melanogaster | | | | |
| Gi\|13097420\|gb\|AAH03 | Similar to | 240 | 56/211 | 77/211 | 6e−06 |
| 448.1\|AAH03448 | tetraspan 1 | | (26%) | (35%) | |
| (BCoo3448) | Mus musculus | | | | |
| Gi\|10834972\|ref\|NP_0 | LEUKOCYTE | 219 | 165/304 | 206/304 | 6e−05 |
| 00551.1\| | SURFACE ANTIGEN | | (54%) | (67%) | |
| | CD53 | | | | |
| | Homo sapiens | | | | |

This information is presented graphically in the multiple sequence alignment given in Table 7G (with NOV7 being shown on line 1) as a ClustalW analysis comparing NOV7 with related protein sequences.

TABLE 7G
Information for the ClustalW proteins:

1) NOV7 (SEQ ID NO:18)
2) gi|6601561|gb|AAF19031.1|AF206661_1 (AF206661) neuronal tetraspanin [Gallus gallus] (SEQ ID NO:72)
3) gi|6685175|gb|AAF23828.1|AF220044_1 (AF220044) tetraspanin [Drosophila melanogaster] (SEQ ID NO:73)
4) gi|13097420|gb|AAH03448.1|AAH03448 (BC003448) Similar to tetraspan 1 [Mus musculus] (SEQ ID NO:74)
5) gi|10834972|ref|NP_000551.1| CD53 antigen [Homo sapiens] (SEQ ID NO:75)

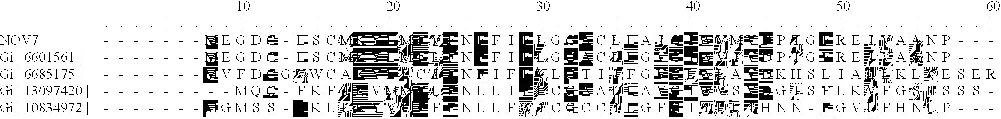

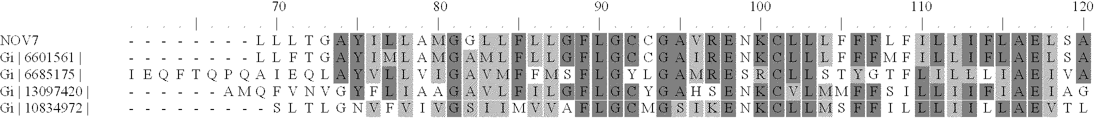

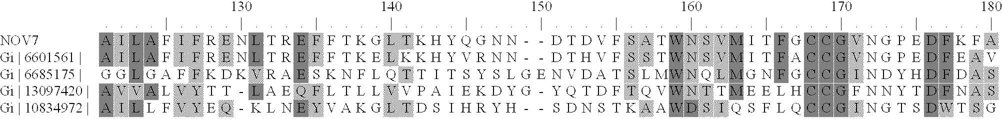

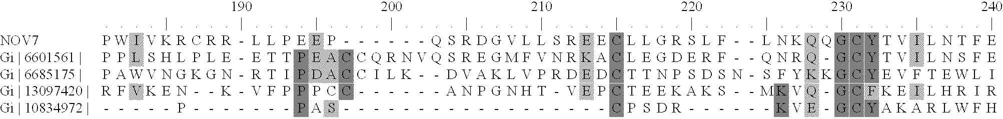

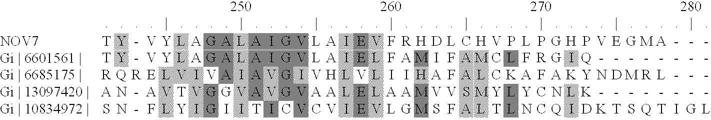

The presence of identifiable domains in NOV7 was determined by searches using algorithms such as PROSITE, Blocks, Pfam, ProDomain, Prints and then determining the Interpro number by crossing the domain match (or numbers) using the Interpro website (http:www.ebi.ac.uk/interpro/).

DOMAIN results for NOV7 were collected from the Conserved Domain Database (CDD) with Reverse Position Specific BLAST. This BLAST samples domains found in the Smart and Pfam collections. The results are listed in Table 7H with the statistics and domain description. The results indicate that this protein contains the transmembrane -4 domain at the positions indicated in Table7H. Residues 10-180 of NOV7 are aligned with residues 1- 153 of the Transmembrane family (SEQ ID NO: 76) (E=1e-13). This indicates that the sequence of NOV7 has properties similar to those of other proteins known to contain this domain and similar to the properties of this domain.

TABLE 7F
DOMAIN results for NOV7

gnl|Pfam|pfam00335, transmembrane4, Transmembrane 4 family
CD-Length = 226 residues, only 67.7% aligned
Score = 70.1 bits (170), Expect = 1e−13

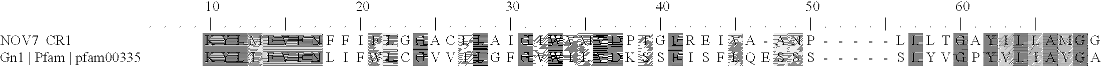

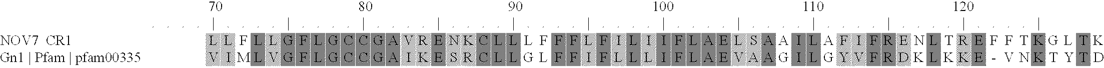

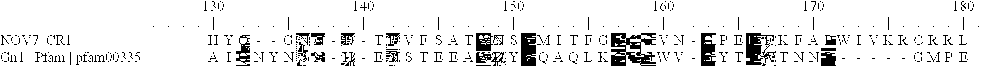

The tetraspanin superfamily includes membrane proteins, such as Leukocyte surface antigen CD37 (OMIM 151523) CD9 (OMIM 143030), CD53 (OMIM 151525), CD81 (OMIM 186845), and the R2 antigen (KAI1; OMIM 600623), among others. See also, OMIM 300096 and 300191, describing members of the transmembrane 4 superfamily, which includes tetraspanin. Many of these molecules are expressed on leukocytes and have been implicated in signal transduction, cell-cell interactions, and cellular activation and development.

CD81 antigen (or TAPA1) is a 26-kD integral membrane protein expressed on many human cell types. Antibodies against TAPA1 induce homotypic aggregation of cells and can inhibit their growth. Oren et al. isolated a cDNA coding for TAPA1. The highly hydrophobic TAPA1 protein contains four putative transmembrane domains and a potential N-myristoylation site. Oren, et al., Molec. Cell. Biol. 10:4007-4015, 1990. TAPA1 showed strong homology with the CD37 leukocyte antigen (OMIM-151523) and with the ME491 melanoma-associated antigen (OMIM- 155740), both of which have been implicated in the regulation of cell growth. Andria et al. cloned the murine homolog of TAPA1 from both cDNA and genomic DNA libraries and demonstrated a very high level of homology between human and mouse genes. Andria et al., J. Immun. 147: 1030-1036, 1991. See, for example, OMIM: 186845.

CD81 is a member of the transmembrane pore integral membrane protein family. It has broad tissue distribution, but its function had not been identified. Boismenu et al. obtained a complete gene from mouse CD81 by RT-PCR. Boismenu et al. Science 271: 198-200, 1996.

A monoclonal antibody specific for mouse CD81 blocked the appearance of alpha-beta T cells but not gamma-delta T cells in fetal organ cultures initiated with day 14.5 thymus lobes. In re-aggregation cultures with CD81 -transfected fibroblasts, CD4-/CD8-thymocytes differentiated into CD4+/CD8+T cells. The authors therefore concluded that interaction between immature thymocytes and stromal cells expressing CD81 are required and may be sufficient to induce early events associated with T-cell development.

Chronic hepatitis C virus (HCV) infection occurs in about 3% of the world's population and is a major cause of liver disease. HCV infection is also associated with cryo-globulinemia, a B lymphocyte proliferative disorder. Virus tropism and the mechanisms of cell entry are not completely understood. Pileri et al. demonstrated that the HCV envelope protein E2 binds human CD81, a tetraspanin expressed on various cell types including hepatocytes and B lymphocytes. Pileri, et al., Science 282: 938-941, 1998. Binding of E2 was mapped to the major extracellular loop of CD81. Recombinant molecules containing this loop bound HCV and antibodies that neutralize HCV infection in vivo inhibited virus binding to CD81 in vitro.

Through eukaryotic expression cloning with an antimetastatic monoclonal antibody Testa et al. have recently identified a tetraspanin member, PETA-3/CD151, as an effector of human tumor cell migration and metastasis. Testa, et al., Cancer Res 59:3812-3820, 1999.

NOV7 has been analyzed for tissue expression profiles. See Examples.

As shown in Table 7H, below, this 96 well plate for panel 1.1, and its variants are composed of RNA/cDNA isolated from various human cell lines that have been established from normal and malignant human tissues. Panel 4 contains cells and cell lines from normal cells and cells related to inflammatory conditions.

The TaqMan oligo set Ag6 10 for the NOV7 gene includes the forward probe and reverse oligomers. Sequences for the oligos are shown in Table 7G.

TABLE 6G

Taqman primers

| | | | |
|---|---|---|---|
| Position | Primers | Sequences | Length |

| | | | | |
|---|---|---|---|---|
| 373 | Forward | 5′ - GCACTACCAGGGCAATAACGA - 3′ | SEQ ID NO:77 | 21 |
| 399 | Probe | FAM-5′ - ACGTCTTCTCTGCCACCTGGAACTCG - 3′-TAMRA | SEQ ID NO:78 | 26 |
| 427 | Reverse | 5′ - GCAGCAACCAAATGTGATCATG - 3′ | SEQ ID NO:79 | 22 |

Taqman results are shown below in TABLE 7H.

| | | | |
|---|---|---|---|
| | % Rel. | % Rel. | |
| Panel 1.1 Tissue Name | Expn. | Panel 4D Tissue Name | Expn. |

| | | | | |
|---|---|---|---|---|
| Adipose | 1.8 | 93768_Secondary Th1_anti-CD28/anti-CD3 | 0.5 | |
| Adrenal gland | 30.6 | 93769_Secondary Th2_anti-CD28/anti-CD3 | 0.5 | |
| Bladder | 5.5 | 93770_Secondary Tr1_anti-CD28/anti-CD3 | 0.4 | |
| Brain (amygdala) | 1.7 | 93573_Secondary Th1_resting day 4-6 in IL-2 | 8.3 | |
| Brain (cerebellum) | 85.3 | 93572_Secondary Th2_resting day 4-6 in IL-2 | 6.8 | |
| Brain (hippocampus) | 8.3 | 93571_Secondary Tr1_resting day 4-6 in IL-2 | 9.1 | |
| Brain (substantia nigra) | 7.5 | 93568_primary Th1_anti-CD28/anti-CD3 | 0.3 | |
| Brain (thalamus) | 5.7 | 93569_primary Th2_anti-CD28/anti-CD3 | 0.6 | |
| Cerebral Cortex | 2.6 | 93570_primary Tr1_anti-CD28/anti-CD3 | 0.5 | |
| Brain (fetal) | 23.8 | 93565_primary Th1_resting dy 4-6 in IL-2 | 52.7 | |
| Brain (whole) | 6.9 | 93566_primary Th2_resting dy 4-6 in IL-2 | 15.7 | |
| CNS ca. (glio/astro) U-118-MG | 0.0 | 93567_primary Tr1_resting dy 4-6 in IL-2 | 15.6 | |
| CNS ca. (astro) SF-539 | 0.8 | 93351_CD45RA CD4 lymphocyte_anti-CD28/anti-CD3 | 0.6 | |
| CNS ca. (astro) SNB-75 | 1.2 | 93352_CD45RO CD4 lymphocyte_anti-CD28/anti-CD3 | 1.6 | |
| CNS ca. (astro) SW1783 | 2.3 | 93251_CD8 Lymphocytes_anti-CD28/anti-CD3 | 0.2 | |
| CNS ca. (glio) U251 | 0.0 | 93353_chronic CD8 Lymphocytes 2ry_resting dy 4-6 in IL-2 | 2.0 | |
| CNS ca. (glio) SF-295 | 9.0 | 93574_chronic CD8 Lymphocytes 2ry_activated CD3/CD28 | 0.4 | |
| CNS ca. (glio) SNB-19 | 0.1 | 93354_CD4_none | 9.4 | |
| CNS ca. (glia/astro) U87-MG | 0.0 | 93252_Secondary Th1/Th2/Tr1_anti-CD95 CH11 | 13.7 | |
| CNS ca.* (neuro; met) SK-N-AS | 49.7 | 93103_LAK cells_resting | 2.0 | |
| Mammary gland | 9.7 | 93788_LAK cells_IL-2 | 0.7 | |
| Breast ca. BT-549 | 0.0 | 93787_LAK cells_IL-2+IL-12 | 1.4 | |
| Breast ca. MDA-N | 0.0 | 93789_LAK cells_IL-2+IFN gamma | 2.5 | |
| Breast ca.* (pl. effusion) T47D | 0.0 | 93790_LAK cells_IL-2+IL-18 | 1.4 | |
| Breast ca.* (pl. effusion) MCF-7 | 0.0 | 93104_LAK cells_PMA | ionomycin and IL-18 | 0.5 |
| Breast ca.* (pl.ef) MDA-MB-231 | 0.0 | 93578_NK Cells IL-2_resting | 0.6 | |
| Small intestine | 17.6 | 93109_Mixed Lymphocyte Reaction_Two Way MLR | 1.2 | |
| Colorectal | 4.0 | 93110_Mixed Lymphocyte Reaction_Two Way MLR | 0.8 | |
| Colon Ca. HT29 | 0.0 | 93111_Mixed Lymphocyte Reaction_Two Way MLR | 0.2 | |
| Colon ca.CaCo-2 | 5.4 | 93112_Mononuclear Cells (PBMCs)_resting | 6.0 | |
| Colon ca.HCT-15 | 0.0 | 93113_Mononuclear Cells (PBMCs)_PWM | 2.3 | |
| Colon ca.HCT-116 | 4.7 | 93114_Mononuclear Cells (PBMCs)_PHA-L | 3.6 | |
| Colon ca. HCC-2998 | 0.0 | 93249_Ramos (B cell)_none | 0.0 | |
| Colon ca. SW480 | 0.0 | 93250_Ramos (B cell)_ionomycin | 0.0 | |
| Colon ca.* (SW480 met)SW620 | 0.0 | 93349_B lymphocytes_PWM | 2.1 | |
| Stomach | 9.9 | 93350_B lymphoytes_CD40L and IL-4 | 0.7 | |
| Gastric ca.* (liver met) NCI-N87 | 0.0 | 92665_EOL-1 (Eosinophil)_dbcAMP differentiated | 0.0 | |
| Heart | 100.0 | 93248_EOL-1 (Eosinophil)_dbcAMP/PMAionomycin | 0.0 | |
| Fetal Skeletal | 27.4 | 93356_Dendritic Cells_none | 0.0 | |
| Skeletal muscle | 16.6 | 93355_Dendritic Cells_LPS 100 ng/ml | 0.1 | |
| Endothelial cells | 84.7 | 93775_Dendritic Cells_anti-CD40 | 0.0 | |
| Endothelial cells (treated) | 55.1 | 93774_Monocytes_resting | 0.3 | |
| Kidney | 43.8 | 93776_Monocytes_LPS 50 ng/ml | 0.0 | |
| Kidney (fetal) | 12.3 | 93581_Macrophages_resting | 0.4 | |
| Renal ca. 786-0 | 0.0 | 93582_Macrophages_LPS 100 ng/ml | 0.0 | |
| Renal ca. A498 | 0.1 | 93098_HUVEC (Endothelial)_none | 25.0 | |
| Renal ca. ACHN | 2.2 | 93099_HUVEC (Endothelial)_starved | 70.2 | |
| Renal ca.TK-10 | 12.0 | 93100_HUVEC (Endothelial)_IL-1b | 24.4 | |
| Renal ca.UO-31 | 8.0 | 93779_HUVEC (Endothelial)_IFN gamma | 36.6 | |
| Renal ca. RXF 393 | 5.2 | 93102_HUVEC (Endothelial)_TNF alpha+IFN gamma | 6.6 | |
| Liver | 8.5 | 93101_HUVEC (Endothelial)_TNF alpha+IL4 | 4.8 | |
| Liver (fetal) | 3.7 | 93781_HUVEC (Endothelial)_IL-11 | 32.6 | |
| Liver ca. (hepatoblast) HepG2 | 0.0 | 93583_Lung Microvascular Endothelial Cells_none | 89.1 | |
| Lung | 9.2 | 93584_Lung Microvascular Endothelial Cells_TNFa (4 ng/ml) | 38.0 | |
| | | and IL1b (1 ng/ml) | | |
| Lung (fetal) | 13.0 | 92662_Microvascular Dermal endothelium_none | 100.0 | |
| Lung ca (non-s.cell) HOP-62 | 15.3 | 92663_Microsvasular Dermal endothelium_TNFa (4 ng/ml) and | 45.0 | |
| | | IL1b (1ng/ml) | | |
| Lung ca. (large cell) NCI-H460 | 0.1 | 93773_Bronchial epithelium_TNFa (4 ng/ml) and IL1b (1 ng/ | 0.0 | |
| | | ml)** | | |
| Lung ca. (non-s.cell) NCI-H23 | 1.3 | 93347_Small Airway Epithelium_none | 0.1 | |
| Lung ca. (non-s.cl) NCI-H522 | 4.6 | 93348_Small Airway Epithelium_TNFa (4 ng/ml) and IL1b (1 | 0.1 | |
| | | ng/ml) | | |
| Lung ca. (non-sm. cell) A549 | 0.3 | 92668_Coronery Artery SMC_resting | 0.2 | |
| Lung ca. (s.cell var.) SHP-77 | 0.0 | 92669_Coronery Artery SMC_TNFa (4 ng/ml) and IL1b (1 ng/ | 0.0 | |
| | | ml) | | |
| Lung ca. (small cell)LX-1 | 0.0 | 93107_astrocytes_resting 11.7 | | |
| Lung ca. (small cell) NCI-H69 | 0.4 | 93108_astrocytes_TNFa (4 ng/ml) and IL1b (1 ng/ml) | 9.0 | |
| Lung ca. (squam.)SW 900 | 0.2 | 92666_KU-812 (Basophil)_resting | 1.9 | |
| Lung ca. (squam.) NCI-H596 | 0.6 | 92667_KU-812 (Basophil)_PMA/ionoycin | 4.6 | |
| Lymph node | 4.6 | 93579_CCD1106 (Keratinocytes)_none | 0.0 | |
| Spleen | 3.3 | 93580_CCD1106 (Keratinocytes)_TNFa and IFNg** | 0.0 | |
| Thymus | 1.0 | 93791_Liver Cirrhosis | 1.4 | |
| Ovary | 12.1 | 93792_Lupus Kidney | 3.0 | |
| Ovarian ca. IGROV-1 | 1.6 | 93577_NCI-H292 | 1.1 | |
| Ovarian ca. OVCAR-3 | 4.9 | 93358_NCI-H292_IL-4 | 1.9 | |
| Ovarian ca. OVCAR-4 | 0.5 | 93360_NCI-H292_IL-9 | 1.7 | |
| Ovarian ca. OVCAR-5 | 2.5 | 93359_NCI-H292_IL-13 | 1.1 | |
| Ovarian ca.OVCAR-8 | 0.0 | 93357_NCI-H292_IFN gamma | 1.4 | |
| Ovarian ca.* (ascites) SK-OV-3 | 8.8 | 93777_HPAEC_- | 21.4 | |
| Pancreas | 8.3 | 93778_HPAEC_IL-1 beta/TNA alpha | 8.9 | |
| Pancreatic ca. CAPAN 2 | 9.7 | 93254_Normal Human Lung Fibroblast_none | 0.0 | |
| Pituitary gland | 6.5 | 93253_Normal Human Lung Fibroblast_TNFa (4 ng/ml) and IL-b (1 ng/ml) | 0.0 | |
| Plancenta | 15.8 | 93257_Normal Human Lung Fibroblast_IL-4 | 0.1 | |
| Prostate | 4.8 | 93256_Normal Human Lung Fibroblast_IL-9 | 0.1 | |
| Prostate ca.* (bone met)PC-3 | 0.0 | 93255_Normal Human Lung Fibroblast_IL-13 | 0.2 | |
| Salavary gland | 4.1 | 93258_Normal Human Lung Fibroblast_IFN gamma | 0.0 | |
| Trachea | 2.9 | 93106_Dermal Fibroblasts CCD1070_resting | 2.1 | |
| Spinal cord | 7.2 | 93361_Dermal Fibroblasts CCD1070_TNF alpha 4 ng/ml | 10.5 | |
| Testis | 4.1 | 93105_Dermal Fibroblasts CCD1070_IL-1 beta 1 ng/ml | 0.7 | |
| Thyroid | 10.1 | 93772_dermal fibroblast_IFN gamma | 0.1 | |
| Uterus | 11.1 | 93771_dermal fibroblast_IL-4 | 0.2 | |
| Melanoma M14 | 0.0 | 93259_IBD Colitis 1** | 8.1 | |
| Melanoma LOX IMVI | 0.0 | 93260_IBD Colitis 2 | 1.5 | |
| Melanoma UACC-62 | 0.0 | 93261_IBD Crohns | 2.5 | |
| Melanoma SK-MEL-28 | 0.0 | 735010_Colon_normal | 26.9 | |
| Melanoma* (met) SK-MEL-y5 | 2.0 | 735019_Lung_none | 62.1 | |
| Melanoma Hs688(A).T | 10.1 | 64028-1_Thymus_none | 41.9 | |
| Melanoma* (met) | | | | |
| Hs688(B).T | 3.7 | 64030-1_Kidney_none | 3.4 | |

In Table 6J the following abbreviations are used: Ca.= carcinoma, * = established from metastasis, met = metastasis, s cell var = small cell variant, non-s = non-sm = non-small, squam = squamous, pl. eff = pl effusion = pleural effusion, glio = glioma, astro = astrocytoma, and neuro = neuroblasloma.

The data from panel 1.1 indicate that expression of Ag610 is primarily in normal tissues including the kidney, endothelial cells, heart, brain, skeletal muscle, and the adrenal gland. The only tumor which highly expresses Ag610 is mel SK_N_AS.

The data from panel 4D indicate that the Ag610 transcript is highly expressed in resting primary and secondary T cells, but expression is almost absent in activated cells. This is particularly striking in primary Thl cells where there is a greater than 50 fold difference in transcript levels between primary activated Th1 cells and primary resting Th1 cells. The only activated T cell populations that expresses this antigen are Th1/Tr1/Th2 cells activated in the presence of anti-CD95, an antibody which blocks FasL-mediated apoptosis. Normal colon also highly expresses this transcript, but expression of this transcript is reduced 3-10 fold in colon tissue from patients with IBD or Crohn's disease. Untreated HUVEC, and lung microvascular endothelial cells also highly express this transcript that is down regulated after activation in these tissues. The expression of this molecule suggests that it is down regulated in response to inflammation.

In some embodiments, a protein therapeutic derived from NOV7 prevents the activation of Th1, Th2, and Tr1 cells, thereby reducing or inhibiting inflammation in chronic autoimmune diseases mediated by activated T cells such as asthma, arthritis, psoriasis, and inflammatory bowel disease. The applicability of this molecule in inflammatory bowel disease (IBD) is further suggested by the absence of this transcript in tissue from patients with Crohn's disease and colitis. VanCompernolle et al., Eur J Immunol 31:823-31, 2001; Kitadokoro et al., EMBO J 20:12-8, 2001.

The similarity information for the NOV7 protein and nucleic acid disclosed herein suggest that NOV7 may have important structural and/or physiological functions characteristic of the 4 transmembrane family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon. The novel nucleic acid encoding NOV7, and the disclosed NOV7 protein, or fragments thereof, may further be useful in diagnostic applications, wherein the presence or amount of the nucleic acid or the protein are to be assessed.

The nucleic acids and proteins of the invention are useful in potential therapeutic applications implicated in HCV infection, Burkitt Lymphoma, and metastatic tumors, immunological disorders particularly those involving T-cells, and/or other pathologies and disorders. For example, a cDNA encoding the tetraspanin-like protein may be useful in gene therapy, and the tetraspanin-like protein may be useful when administered to a subject in need thereof. By way of nonlimiting example, the NOV7 compositions will have efficacy for treatment of patients suffering from HCV infection, Burkitt Lymphoma metastatic tumors and immunological disorders particularly those involving T-cells. The novel nucleic acid encoding tetraspanin-like protein, and the tetraspanin-like protein of the invention, or fragments thereof, may further be useful in diagnostic applications, wherein the presence or amount of the nucleic acid or the protein are to be assessed.

The disclosed NOV7 polypeptides can be used as immunogens to produce vaccines. The novel nucleic acid encoding NOV-like protein, and the NOV-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. For example the disclosed NOV7 protein has multiple hydrophilic regions, each of which can be used as an immunogen. In one embodiment, a contemplated NOV7 epitope is from about amino acids 110 to 140. In another embodiment, a NOV7 epitope is from about amino acids 155 to 180. In additional embodiments, NOV7 epitopes are from amino acids 190 to 200. These novel proteins can also be used to develop assay system for functional analysis. These antibodies may be generated according to methods known in the art, using prediction from hydrophobicity charts, as described in the “Anti-NOVX Antibodies” section below.

NOV8

NOV8a

NOV8a was initially identified by searching CuraGen's Human SeqCalling database for DNA sequences which translate into proteins with similarity to a protein family of interest. SeqCalling assembly 27479850_EXT1 was identified as having suitable similarity. SeqCalling assembly 27479850_EXT1 has one component. In a search of CuraGen's human expressed sequence assembly database, assembly s3aq: 27479850 (507 nucleotides) was identified as having identical homology to this predicted gene sequence. This sequence is derived from a publicly available Homo sapiens expressed sequence tag (EST) incorporated into the CuraGen database. This database is composed of the expressed sequences (as derived from isolated mRNA) from more than 96 different tissues. The MRNA is converted to cDNA and then sequenced. These expressed DNA sequences are then pooled in a database and those exhibiting a defined level of homology are combined into a single assembly with a common consensus sequence. The consensus sequence is representative of all member components. Since the nucleic acid of the described invention has identical sequence identity with the CuraGen assembly, the nucleic acid of the invention represents an expressed gene sequence.

SeqCalling assembly 27479850_EXT1 was analyzed further to identify open reading frame(s) encoding for a novel full length protein(s) and novel splice forms of these SHDs. This was done by extending the SeqCalling assembly using suitable additional SeqCalling assemblies, publicly available EST sequences as well public genomic sequence. Public ESTs and additional CuraGen SeqCalling assemblies were identified by the CuraTools program SeqExtend™. They were included in the DNA sequence extension for SeqCalling assembly 27479850_EXT1 only when sufficient identical overlap was found. These inclusions are described below:

Genomic clone AC008616 was identified as having regions with 100% identity to the SeqCalling assembly 27479850_EXT1 and was selected for analysis because this identity implied that this clone contained the sequence of the genomic locus for SeqCalling assembly 27479850_EXT1.

The genomic clone was analyzed by Genscan and Grail to identify exons and putative coding sequences/open reading frames. This clone was also analyzed by TblastN, BlastX, and other homology programs to identify regions translating to proteins with similarity to the original protein/protein family of interest

The results of these analyses were integrated and manually corrected for apparent inconsistencies, thereby obtaining the sequences encoding the full-length protein. When necessary, the process to identify and analyze cDNAs/ESTs and genomic clones was reiterated to derive the full length sequence. This invention describes this full-length DNA sequence and the full-length protein sequence which it encodes. These nucleic acids and protein sequences for each splice form are referred to here as 27479850_EXT1, or NOV8a. Specifically, CuraGen's SeqCalling Assembly 27479850_EXT1 is made up of one 507 bp fragment. SeqCalling Assembly 27479850_EXT1 lists lung, testis and B-cell as tissue sources. Literature sources mentioned above cite brain and the central nervous system as tissue sources for SHD and SHD-like proteins. SeqCalling assembly 24111358_EXT1 showed initial homology, by searching with BLASTX, to a M. musculus (Mouse) protein: SHD PROTEIN (SPTREMBL-ACC:088834; 343 aa). Using BlastN, this SeqCalling Assembly was identical at the nucleotide level to a GenBank genomic sequence: Homo sapiens chromosome 19 clone CIT978SKB_{—}144D21, 49 unordered pieces—112626 base pairs (bp)(GENBANKNEW-ID: AC008616|acc:AC008616). AC008616 was processed with GenScan™ and the predicted coding regions were analyzed using BlastX, BlastN and TBlastN to find exons with homologies to M. musculus SHD PROTEIN. The genomic clone matched identically to the SeqCalling Assembly 27479850_EXT1. AC008616 was used to extend 27479850_EXT1. This was accomplished by using the protein sequence of O88834 and Curatoo's TblastN against the GBNEW database. Intron/exon junctions were determined by manual inspection and corrected for apparent inconsistencies. BlastX of this sequence showed the correct full-length protein, 27479850_EXT1. The base pair (bp) regions used from the genomic clone were: 67447-67770, 70280-70357, 70436-70624, 72160-72288, 75627-75746, 77831-78016. The disclosed NOV-8 is expressed in at least the following tissues: brain and central nervous system derived from literature sources and lung, testis and B-cell derived from 27479850_EXT1.

The novel nucleic acid was identified on chromosome 19. The disclosed NOV8a nucleic acid of 1026 nucleotides (also referred to as 27479850_EXT1) is shown in Table 8A. An open reading begins with an ATG initiation codon at nucleotides 1-3 and ends with a TGA codon at nucleotides 1024-1026.

TABLE 8A
NOV8a Nucleotide Sequence (SEQ ID NO:19)

ATGGCCAAGTGGCTACGGGACTACCTGAGCTTTGGGGGTCGGAGGCCCCCTCCGCAGCCGCCCACCCCGG
ACTACACCGAGAGCGACATCCTGAGGGCCTACCGCGCGCAGAAGAACCTGGACTTCGAGGACCCCTATGA
GGACGCGGAGAGCCGCTTGGAGCCGGACCCCGCGGGCCCTGGGGACTCCAAGAACCCCGGAGATGCCAAG
TATGGTTCTCCCAAGCACCGGCTCATCAAGGTGGAGGCTGCGGATATGGCCAGAGCCAAGGCCCTTCTGG
GCGGCCCCGGGGAGGAGGTGCGTGGCTGGGTGGCCTGGGGAGACCCCTTTGATGCTCAGCCTCATCCTGC
ACCCCCGGATGATGGGTACATGGAGCCCTACGATGCCCAATGGGTCATGAGTGAACTTCCCGGCAGAGGG
GTGCAGCTCTATGACACCCCTTATGAGGAACAGGACCCAGAGACAGCAGATGGACCCCCTTCTGGGCAGA
AGCCTCGGCAGAGCCGGATGCCCCAGGAAGATGAACGGCCAGCAGATGAGTATGATCAGCCCTGGGAGTG
GAAGAAAGACCACATCTCCAGGGCGTTTGCACCAGTGCAGTTTGACAGTCAGAGTGGGAGAGACTCTCCA
GGCTCAGCCAAGGAGCTCCGGAGACCTCCGCCCAGAAGCCCCCAGCCTGCGGAGCGTGTGGACCCAGCCC
TGCCCCTGGAGAAACAGCCGTGGTTTCATGGCCCCCTGAACAGGGCGGATGCAGAGAGCCTCCTGTCCCT
CTGCAAGGAAGGCAGCTACCTAGTGCGGCTCAGTGAGACCAACCCCCAGGACTGCTCCTTGTCTCTCAGG
AGCAGCCAGGGCTTCCTGCATCTGAAGTTCGCGCGGACCCGTGAGAACCAGGTGGTGCTGGGCCAACACA
GCGGGCCCTTCCCCAGCGTGCCCGAGCTCGTCCTCCACTACAGTTCACGCCCACTGCCGGTGCAGGGTGC
CGAGCATCTGGCTCTGCTGTACCCCGTGGTCACGCAGACCCCCTGA

The disclosed nucleic acid sequence has 299 of 360 bases (83%) identical to a 1529 bp Mus musculus src homology domain (SHD) mRNA. (GENBANK-ID:AB018423) (E value=7.1e-¹⁰⁵).

The NOV8a protein encoded by SEQ ID NO: 19 has 341 amino acid residues, and is presented using the one-letter code in Table 8B (SEQ ID NO: 20). The SignalP, Psort and/or Hydropathy profile for NOV8 predict that NOV8 has a signal peptide and is likely to be localized in the cytoplasm with a certainty of 0.5050.

TABLE 8B
Encoded NOV8a protein sequence (SEQ ID NO:20).

MAKWLRDYLSFGGRRPPPQPPTPDYTESDILRAYRAQKNLDFEDPYEDAESRLEPDPAGPGDSKNPGDA
KYGSPKHRLIKVEAADMARAKALLGGPGEEVRGWVAWGDPFDAQPHPAPPDDGYMEPYDAQWVMSELPG
RGVQLYDTPYEEQDPETADGPPSGQKPRQSRMPQEDERPADEYDQPWEWKKDHISRAFAPVQFDSPEWE
RTPGSAKELRRPPPRSPQPAERVDPALPLEKQPWFHGPLNRADAESLLSLCKEGSYLVRLSETNPQDCS
LSLRSSQGFLHLKFARTRENQVVLGQHSGPFPSVPELVLHYSSRPLPVQGAEHLALLYPVVTQTP

The full amino acid sequence of the protein of the invention was found to have 257 of 338 amino acid residues (76%) identical to, and 275 of 338 residues (81%) positive with, the 343 amino acid residue SHD protein from Mus musculus (ptnr:SPTREMBL-ACC:O88834) (E value=5.5e-¹³⁴).

NOV8b

NOV8b

The sequence of Acc. No. CG5 1761-02 (NOV8b) was derived by laboratory cloning of cDNA fragments, by in silico prediction of the sequence, and refining the information obtained for NOV8a. cDNA fragments covering either the full length of the DNA sequence, or part of the sequence, or both, were cloned. In silico prediction was based on sequences available in CuraGen's proprietary sequence databases or in the public human sequence databases, and provided either the full length DNA sequence, or some portion thereof. The laboratory cloning was performed using one or more of the methods summarized below:

SeqCalling™ Technology: cDNA was derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then sequenced using CuraGen's proprietary SeqCalling technology. Sequence traces were evaluated manually and edited for corrections if appropriate. cDNA sequences from all samples were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

RACE: Techniques based on the polymerase chain reaction such as rapid amplification of cDNA ends (RACE), were used to isolate or complete the predicted sequence of the cDNA of the invention. Usually multiple clones were sequenced from one or more human samples to derive the sequences for fragments. The following human samples from different donors were used adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus for the RACE reaction. The sequences derived from these procedures were included in the SeqCalling Assembly process described in the preceding paragraph.

Multiple clones were sequenced and these fragments were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

The DNA sequence and protein sequence for a novel SHD protein-like gene were obtained by exon linking and extended by RACE and are reported here as CuraGen Acc. No. CG51761-02, or NOV8b.

The disclosed NOV8 gene is expressed in, for example, the following tissues: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus. This expression information was derived from the tissue sources of the sequences that were included in the derivation of the sequence of NOV8.

The 1223 bp nucleic acid for NOV8b (SEQ ID NO: 21) is shown in Table 8C. An open reading frame was identified beginning at nucleotides 101-103 and ending at nucleotides 1124-1126. The start (ATG) and stop (TAG) codons of the open reading frame are highlighted in bold type. Putative untranslated regions are underlined. NOV8b differs from NOV8a by having a 100 bp 5′ UTR and a 97 bp 3′ UTR. Additionally, there are 20 nucleotide differences, all located between nucleotides 247 and 420 (numbered with respect to NOV8b).

TABLE 8C
NOV8b Nucleotide Sequence (SEQ ID NO:21)

| | |
|---|---|
| CTTCCTCTCCACCTCCTCCTCCTCCTTGGGGAAAGGGGCCCGGAGAAGGGCATGTGGGGG | 60 |
| CCCCTCTGACAGTGGCCCGATTGGGGTGACAGGCGCCCAAATGGCCAAGTGGCTACGGGA | 120 |
| CTACCTGAGCTTTGGGGGTCGGAGGCCCCCTCCGCAGCCGCCCACCCCGGACTACACCGA | 180 |
| GAGCGACATCCTGAGGGCCTACCGCGCGCAGAAGAACCTGGACTTCGAGGACCCCTATGA | 240 |
| GGACGCCGAGAGCCGCTTGGAGCCGGACCCCGCGGGCCCTGGGGACTCCAAGAACCCCGG | 300 |
| AGATGCCAAGTATGGTTCTCCCAAACACCGGCTCATCAAGGTGGAGGCTGCGGATATGGC | 360 |
| CAGAGCCAAGACCCTTCTGGGCGGCCCCGGGGAGGAGCTGGAAGCCGACACTGAGTATTT | 420 |
| AGACCCCTTTGATGCTCAGCCTCATCCTGCACCCCCGGATGATGGGTACATGGAGCCCTA | 480 |
| CGATGCCCAATGGGTCATGAGTGAACTTCCCGGCAGAGGGGTGCAGCTCTATGACACCCC | 540 |
| TTATGAGGAACAGGACCCAGAGACAGCAGATGGACCCCCTTCTGGGCAGAAGCCTCGGCA | 600 |
| GAGCCGGATGCCCCAGGAAGATGAACGGCCAGCAGATGAGTATGATCAGCCCTGGGAGTG | 660 |
| GAAGAAAGACCACATCTCCAGGGCGTTTGCACCAGTGCAGTTTGACAGTCCAGAGTGGGA | 720 |
| GAGGACTCCAGGCTCAGCCAAGGAGCTCCGGAGACCTCCGCCCAGAAGCCCCCAGCCTGC | 780 |
| GGAGCGTGTGGACCCAGCCCTGCCCCTGGAGAAACAGCCGTGGTTTCATGGCCCCCTGAA | 840 |
| CAGGGCGGATGCAGAGAGCCTCCTGTCCCTCTGCAAGGAAGGCAGCTACCTAGTGCGGCT | 900 |
| CAGTGAGACCAACCCCCAGGACTGCTCCTTGTCTCTCAGGAGCAGCCAGGGCTTCCTGCA | 960 |
| TCTGAAGTTCGCGCGGACCCGTGAGAACCAGGTGGTGCTGGGCCAACACAGCGGGCCCTT | 1020 |
| CCCCAGCGTGCCCGAGCTCGTCCTCCACTACAGTTCACGCCCACTGCCGGTGCAGGGTGC | 1080 |
| CGAGCATCTGGCTCTGCTGTACCCCGTGGTCACGCAGACCCCCTGACAGTGACCCTCGGC | 1140 |
| CCCCTTTTGAGTCCTCGGGCCCAGAATCGTATCCCAAAGCCCTCCCATGGCCTAGAAAAT | 1200 |
| AAATAAGTTATTGTTTGTCTTAG | 1223 |

The disclosed nucleic acid sequence has 309 of 377 bases (81%) identical to a 1529 bp Mus musculus src homology domain (SHD) mRNA. (GENBANK-ID:AB018423) (E value=3.0e-¹¹⁰).

The NOV8b protein encoded by SEQ ID NO: 21 has 341 amino acid residues, and is presented using the one-letter code in Table 8D (SEQ ID NO: 22). The SignalP, Psort and/or Hydropathy profile for NOV8 predict that NOV8 has a signal peptide and is likely to be localized in the cytoplasm with a certainty of 0.5050, NOV8b differs from NOV8a at 9 positions: T91>A; L100>V; E101>R; A102>G; D103>W; T104>V; E105>A; Y106>W and L107>G.

TABLE 8D
Encoded NOV8a protein sequence (SEQ ID NO:22).

| | |
|---|---|
| MAKWLRDYLSFGGRRPPPQPPTPDYTESDILRAYRAQKNLDFEDPYEDAESRLEPDPAGP | 60 |
| GDSKNPGDAKYGSPKHRLIKVEAADMARAKTLLGGPGEELEADTEYLDPFDAQPHPAPPD | 120 |
| DGYMEPYDAQWVMSELPGRGVQLYDTPYEEQDPETADGPPSGQKPRQSRMPQEDERPADE | 180 |
| YDQPWEWKKDHISRAFAPVQFDSPEWERTPGSAKELRRPPPRSPQPAERVDPALPLEKQP | 240 |
| WFHGPLNRADAESLLSLCKEGSYLVRLSETNPQDCSLSLRSSQGFLHLKFARTRENQVVL | 300 |
| GQHSGPFPSVPELVLHYSSRPLPVQGAEHLALLYPVVTQTP | 341 |

The full amino acid sequence of the protein of the invention was found to have 261 of 338 amino acid residues (77%) identical to, and 279 of 338 residues (82%) positive with, the 343 amino acid residue SHD protein from Mus musculus (ptnr:SPTREMBL-ACC:O88834) (E value=4.3e-¹³⁷).

Patp results include those listed in Table 8C.

TABLE 8E
Patp alignments of NOV8

| | | | Smallest |
|---|---|---|---|
| | | | Sum |
| | Reading | High | Prob |
| Sequences producing High-scoring Segment Pairs: | Frame | Score | P(N) |
| patp:Y07040 Breast cancer associated antigen precursor... | +1 | 521 | 4.4e-64 |
| patp:B54255 Human pancreatic cancer antigen protein se... | +1 | 347 | 1.7e-33 |
| patp:R3774E Collagen-like polymer DCP5 encoded by clon... | −3 | 166 | 1.6e-08 |
| patp:R93257 Collagen-like polymer sequence D gene 5 po... | −3 | 166 | 1.6e-08 |

Further BLAST analysis produced the significant results listed in Table 8F. The disclosed NOV8 protein has good identity with a number of src domain-containing proteins.

TABLE 8F
BLAST results for NOV8

| Gene Index/ | | Length | Identity | Positives | |
|---|---|---|---|---|---|
| Identifier | Protein/Organism | (aa) | (%) | (%) | Expect |
| gi\|6677939\|ref\|NP_03 | src homology 2 | 343 | 248/338 | 266/338 | e-114 |
| 3194.1\| (AB018423) | domain-containing | | (73%) | (78%) | |
| | transforming | | | | |
| | protein D Mus | | | | |
| | musculus | | | | |
| gi\|9368520\|emb\|CAB98 | similar to | 247 | 238/255 | 241/255 | e-106 |
| 202.1\| (AL390078) | (NP_033194.1) src | | (93%) | (94%) | |
| | homology 2 | | | | |
| | Homo sapiens | | | | |
| gi\|545100\|gb\|AAB2978 | Shb=Src homology | 309 | 126/279 | 176/279 | 3e-50 |
| 0.1\| | 2 protein [mice, | | (45%) | (62%) | |
| | Peptide Partial] | | | | |
| gi\|4506935\|ref\|NP_00 | SHB adaptor | 596 | 142/339 | 189/339 | 4e-48 |
| 3019.1\| (X75342) | protein (a Src | | (41%) | (54%) | |
| | homology 2 | | | | |
| | protein) Homo | | | | |
| | sapiens | | | | |

This information is presented graphically in the multiple sequence alignment given in Table 8G (with NOV8a being shown on line 1) as a ClustalW analysis comparing NOV8 with related protein sequences.

TABLE 8F
Information for the ClustalW proteins:

1) NOV8a (SEQ ID NO:20)
2) NOV8b (SEQ ID NO:22)
3) gi|6677939|ref|NP_033194.1 src homology 2 domain-containing transforming protein D [Mus musculus] (SEQ ID NO:80)
4) gi|9368520|emb|CAB98202.1| (AL390078) similar to (NP_033194.1) src homology 2 domain-containing transforming protein D [Mus musculus] (SEQ ID NO:81)
5) gi|545100|gb|AAB29780.1| Shb = Src homology 2 protein [mice, Peptide Partial, 309 aa] (SEQ ID NO:82)
6) gi|4506935|ref|NP_003019.1| SHB adaptor protein (a Src homology 2 protein) [Homo sapiens] (SEQ ID NO:83)

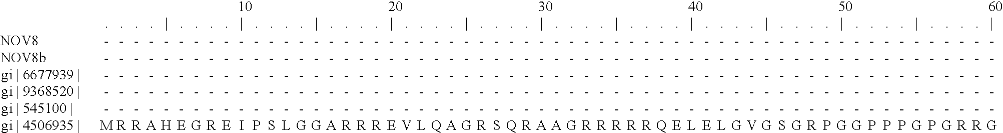

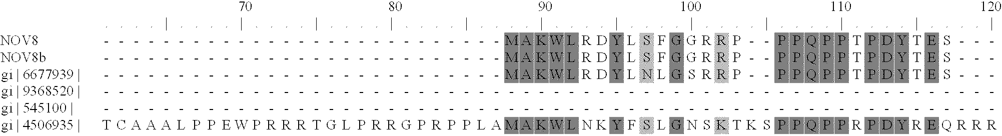

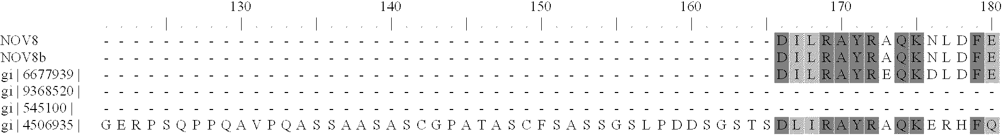

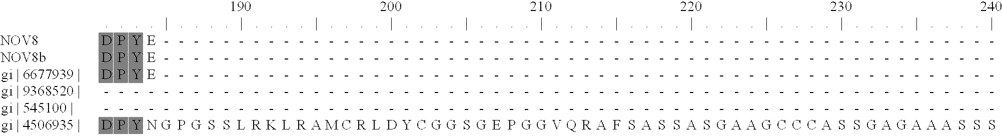

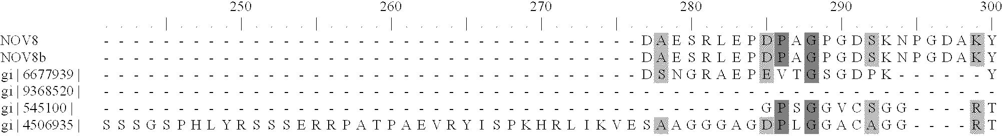

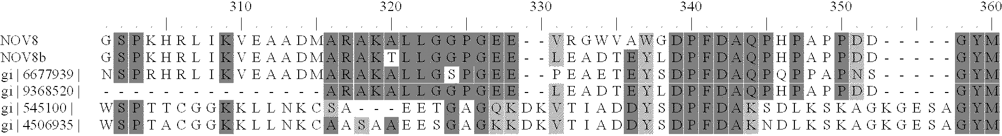

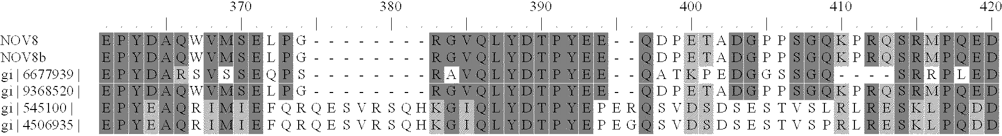

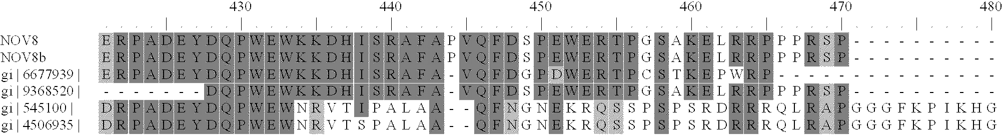

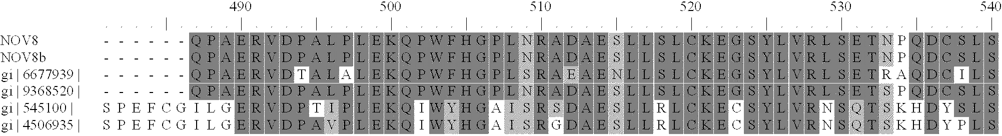

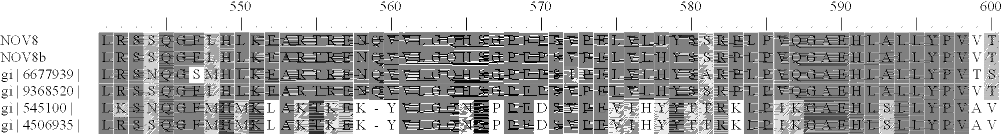

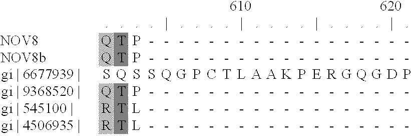

The presence of identifiable domains in NOV8 was determined by searches using algorithms such as PROSITE, Blocks, Pfam, ProDomain, Prints and then determining the Interpro number by crossing the domain match (or numbers) using the Interpro website (http:www.ebi.ac.uk/interpro/).

DOMAIN results for NOV8 were collected from the Conserved Domain Database (CDD) with Reverse Position Specific BLAST. This BLAST samples domains found in the Smart and Pfam collections. The results are listed in Table 8H with the statistics and domain description. The results indicate that NOV8 contains Src homology 2 domain (gn1|Smart|SH2, Src homology 2 domain) at amino acid positions 239-323, which align with residues 1-85 of this domain (SEQ ID NO: 84). This indicates that the sequence of NOV8 has properties similar to those of other proteins known to contain this domain. NOV8b also shows homology to this domain, with an E value of 3.1e-22. Src homology 2 domains bind phosphotyrosine-containing polypeptides via 2 surface pockets. Specificity is provided via interaction with residues that are distinct from the phosphotyrosine.

TABLE 8H
DOMAIN results for NOV8

CD-Length = 85 residues, 100.0% aligned
Score = 86.3 bits (212), Expect = 2e−18

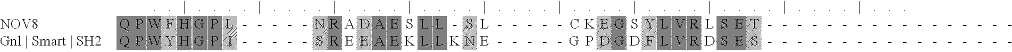

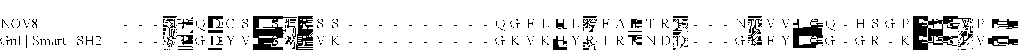

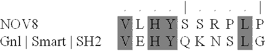

The Src homology 2 (SH2) is a protein domain of about 85 amino-acid residues first identified as a conserved sequence region between the oncoproteins Src and Fps. Pawson et al., Mol. Cell. Biol. 6:4396-4408, 1986. Similar sequences were later found in many other intracellular signal-transducing proteins. Barton et al., FEBS Lett. 304: 15-20, 1992. SH2 domains function as regulatory modules of intracellular signaling cascades by interacting with high affinity to phosphotyrosine-containing target peptides in a sequence-specific and strictly phosphorylation-dependent manner. Pawson and Schlessinger, Curr. Biol. 3:434-442, 1993; Baltimore and Mayer, Trends Cell Biol. 3: 8-13, 1993; Pawson, Nature 373: 573-580, 1995. They are found in a wide variety of protein contexts e.g., in association with catalytic domains of phospholipase Cy (PLCy) and the nonreceptor protein tyrosine kinases; within structural proteins such as fodrin and tensin; and in a group of small adaptor molecules, i.e. Crk and Nck. In many cases, when an SH2 domain is present so too is an SH3 domain, suggesting that their functions are inter-related.

Adaptor proteins link catalytic signaling proteins to cell surface receptors or downstream effector proteins. Using a subtractive hybridization strategy to identify genes that are specifically expressed in activated CD8+ T cells, Spurkland et al. (J. Biol. Chem. 273: 4539-4546, 1998) isolated cDNAs encoding SH2D2A, which they named TSAD. The predicted 389-amino acid SH2D2A protein contains an Src homology-2 (SH2) domain, putative SH3 domain-binding motifs, and putative phosphotyrosine-binding domain (PTB)-binding motifs, but no known catalytic domains. The authors also isolated cDNAs representing alternatively spliced SH2D2A transcripts that encode deduced 361- and 399-amino acid proteins. Northern blot analysis detected an approximately 1.7-kb SH2D2A transcript in peripheral blood leukocytes, thymus, and spleen. SH2D2A was expressed in activated T cells, but not in resting T cells or in B cells. Its expression was rapidly induced after activation of T cells. Antiserum raised against SH2D2A reacted with a 52-kD protein on Western blots of T-cell lysates. Recombinant SH2D2A expressed in mammalian cells localized to the cytoplasm. Spurkland et al. (J. Biol. Chem. 273: 4539-4546, 1998) showed that SH2D2A is tyrosine-phosphorylated in vivo. They suggested that SH2D2A is an adaptor protein involved in T cell signaling.

By searching an EST database for sequences with signal transduction motifs, Lu et al. (J. Biol. Chem. 274: 10047-10052, 1999) identified a cDNA encoding a deduced 698-amino acid protein, which they named NSP3 (novel SH2-containing protein-3). Sequence analysis revealed that NSP3 also contains a potential SH3 interaction domain. Northern blot analysis detected significant levels of a 3.2- and a 3.8-kb NSP3 transcript in a wide variety of tissues.

Further, Lu et al. (supra) also identified a cDNA encoding a deduced 576-amino acid protein, which they named NSP1 (novel SH2-containing protein-1). Sequence analysis revealed that NSP1 also contains a potential SH3 interaction domain. Northern blot analysis detected significant levels of a 2.7-kb NSP1 transcript only in placenta, pancreas, kidney, lung, fetal kidney, and fetal lung. Treatment with insulin or epidermal growth factor (EGF) resulted in rapid tyrosine phosphorylation of NSP 1 and increased association of the 64-kD NSP1 with p130-Cas. In contrast, contact with fibronectin resulted in little phosphorylation of NSP1 but increased phosphorylation of the p130-Cas associated with NSP1. The authors determined that expression of NSP1 leads to activation of the stress-activated protein kinase JNK1 (MAPK8) but not ERK2 (MAPK1).

Many proteins involved in the regulation of cellular proliferation contain sequence motifs are named SH2 and SH3. Pawson and Gish, Cell 71: 359-362, 1992. These domains mediate interaction with other proteins; the SH2 domain interacts with tyrosine phosphorylation sites, while SH3 domains interact with proline-rich sequences. Many signal transduction pathways involve the induction of the formation of complexes of proteins such as growth factor receptors, adaptor proteins, and target enzymes through SH2 and SH3 interactions. Adaptor proteins are molecules with multiple protein interaction motifs that do not appear to have catalytic activity of their own but mediate the interaction of other proteins. The SHB gene encodes two such adaptor proteins (from two different start methionines) of 67 and 56 kD. Welsh et al., Oncogene 9: 19-27, 1994. By PCR analysis of a somatic cell hybrid mapping panel, Yulug et al. (Genomics 24: 615-617,1994) mapped the SHB gene to chromosome 9. By fluorescence in situ hybridization, they regionalized the gene to 9p12-p 11.

Oda et al. (Oncogene 11:1255-62, 1997) used a yeast two hybrid screen to identify proteins binding to the Ab1 tyrosine kinase in order to understand the molecular mechanism of Bcr-Ab1 mediated transformation. Two partial cDNAs encoding novel SH2 domain-containing proteins were cloned and designated SHD and SHE. Both have homology to SHB, a previously reported SH2 domain-containing protein. Northern blot analysis showed that SHE is expressed in heart, lung, brain, and skeletal muscle, while expression of SHD is restricted to the brain. The deduced amino acid sequence of the full length mouse SHD cDNA contains an amino-terminal proline-rich region, and a carboxy-terminal SH2 domain. A bacterially expressed SHD domain bound multiple tyrosine-phosphorylated proteins with relative molecular weights of 200, 170, 130, 100, 90, 78, 72 and 32 kDa from K562 cell lysates. SHD contains five YXXP motifs, a substrate sequence preferred by Ab1 tyrosine kinases. The domains are frequently found as repeats in a single protein sequence. The structure of the SH2 domain belongs to the alpha+beta class, its overall shape forming a compact flattened hemisphere. The core structural elements comprise a central hydrophobic anti-parallel beta-sheet, flanked by 2 short alpha-helices. In the v-src oncogene product SH2 domain, the loop between strands 2 and 3 provides many of the binding interactions with the phosphate group of its phosphopeptide ligand, and is hence designated the phosphate binding loop. SHD was tyrosine phosphorylated in COS-7 cells co-transfected with SHD and c-Ab1 or Bcr-Ab1. These results suggest that SHD may be a physiological substrate of c-Ab1 and may function as an adapter protein in the central nervous system.

The similarity information for the NOV8 protein and nucleic acid disclosed herein suggest that NOV8 may have important structural and/or physiological functions characteristic of the src homology domain (SHD) family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon. The novel nucleic acid encoding NOV8, and the NOV8 protein of the invention, or fragments thereof, may further be useful in diagnostic applications, wherein the presence or amount of the nucleic acid or the protein are to be assessed.

The disclosed NOV8 nucleic acids and proteins of the invention are useful in potential therapeutic applications implicated in cancer and lymphoproliferative syndrome, as well as, Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection, myasthenia gravis, and other and/or other pathologies and disorders.

For example, a cDNA encoding the SHD-like protein may be useful in gene therapy, and the SHD-like protein may be useful when administered to a subject in need thereof. By way of nonlimiting example, the compositions of the present invention will have efficacy for treatment of patients suffering from cancer, lymphoproliferative syndrome, Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection, myasthenia gravis, and other and/or other pathologies and disorders. The novel nucleic acid encoding SHD-like protein, and the SHD-like protein of the invention, or fragments thereof, may further be useful in diagnostic applications, wherein the presence or amount of the nucleic acid or the protein are to be assessed. These materials are further useful in the generation of antibodies that bind immuno-specifically to the novel NOV8 substances for use in therapeutic or diagnostic methods. These antibodies may be generated according to methods known in the art, using prediction from hydrophobicity charts, as described in the “Anti-NOVX Antibodies” section below. For example the disclosed NOV8 protein has multiple hydrophilic regions, each of which can be used as an immunogen. These novel proteins can also be used to develop assay system for functional analysis.

NOV9

A disclosed novel NOV9 nucleic acid is 2031 nucleotides long (also referred to as AI284055_EXT) is shown in Table 9A (SEQ ID NO: 23). An ORF begins with an ATG initiation codon at nucleotides 1-3 and ends with a TGA codon at nucleotides 2029-2031. The start and stop codons are in bold letters in Table 9A.

TABLE 9A
NOV9 Nucleotide Sequence (SEQ ID NO:23)

ATGCCACACGCCTTCAAGCCCGGGGACTTGGTGTTCGCTAAGATGAAGGGCTACCCTCACTGGCCTGCCA
GGATCGACGACATCGCGGATGGCGCCGTGAAGCCCCCACCCAACAAGTACCCCATCTTTTTCTTTGGCAC
ACACGAAACGGCCTTCCTGGGACCCAAGGACCTGTTCCCCTACGACAAATGTAAAGACAAGTACGGGAAG
CCCAACAAGAGGAAAGGCTTCAATGAAGGGCTGTGGGAGATCCAGAACAACCCCCACGCCAGCTACAGCG
CCCCTCCGCCAGTGAGCTCCTCCGACAGCGAGGCCCCCGAGGCCAACCCCGCCGACGGCAGTGACGCTGA
CGAGGACGATGAGGACCGGGGGGTCATGGCCGTCACAGCGGTAACCGCCACAGCTGCCAGCGACAGGATG
GAGAGCGACTCAGACTCAGACAAGAGTAGCGACAACAGTGGCCTGAAGAGGAAGACGCCTGCGCTAAAGG
TATCGGTCTCGAAACGAGCCCGAAAGGCCTCCAGCGACCTGGATCAGGCCAGCGTGTCCCCATCCGAAGA
GGAGAACTCGGAAAGCTCATCTGAGTCGGAGAAGACCAGCGACCAGGACTTCACACCTGAGAAGAAAGCA
GCGGTCCGGGCGCCACGGAGGGGCCCTCTGGGGGGACGGAAAAAAAAGAAGGCGCCATCAGCCTCCGACT
CCGACTCCAAGGCCGATTCGGACGGGGCCAAGCCTGAGCCGGTGGCCATGGCGCGGTCGGCGTCCTCCTC
CTCCTCTTCCTCCTCCTCCTCCGACTCCGATGTGTCTGTGAAGAAGCCTCCGAGGGGCAGGAAGCCAGCG
GAGAAGCCTCTCCCGAAGCCGCGAGGGCGGAAACCGAAGCCTGAACGGCCTCCGTCCAGCTCCAGCAGTG
ACAGTGACAGCGACGAGGTGGACCGCATCAGTGAGTGGAAGCGGCGGGACGAGGCGCGGAGGCGCGAGCT
GGAGGCCCGGCGGCGGCGAGAGCAGGAGGAGGAGCTGCGGCGCCTGCGGGAGCAGGAGAAGGAGGAGAAG
GAGCGGAGGCGCGAGCGGGCCGACCGCGGGGAGGCTGAGCGGGGCAGCGGCGGCAGCAGCGGGGACGAGC
TCAGGGAGGACGATGAGCCCGTCAAGAAGCGGGGACGCAAGGGCCGGGGCCGGGGTCCCCCGTCCTCCTC
TGACTCCGAGCCCGAGGCCGAGCTGGAGAGAGAGGCCAAGAAATCAGCGAAGAAGCCGCAGTCCTCAAGC
ACAGAGCCCGCCAGGAAACCTGGCCAGAAGGAGAAGAGAGTGCGGCCCGAGGAGAAGCAACAAGCCAGGC
CCGTGAAGGTGGAGCGGACCCGGAAGCGGTCCGAGGGCTTCTCGATGGACAGGAAGGTAGAGAAGAAGAA
AGAGCCCTCCGTGGAGGAGAAGCTGCAGAAGCTGCACAGTGAGATCAAGTTTGCCCTAAAGGTCGACAGC
CCGGACGTGAAGAGGTGCCTGAATGCCCTAGAGGAGCTGGGAACCCTGCAGGTGACCTCTCAGATCCTCC
AGAAGAACACAGACGTGGTGGCCACCTTGAAGAAGATTCGCCGTTACAAAGCGAACAAGGACGTAATGGA
GAAGGCAGCAGAAGTCTATACCCGGCTCAAGTCGCGGGTCCTCGGCCCAAAGATCGAGGCGGTGCAGAAA
GTGAACAAGGCTGGGATGGAGAAGGAGAAGGCCGAGGAGAAGCTGGCCGGGGAGGAGCTGGCCGGGGAGG
AGCTGGCCGGGGAGGAGGCCCCCCAGGAGAAGGCGGAGGACAAGCCCAGCACCGATCTCTCAGCCCCAGT
GAATGGCGAGGCCACATCACAGAAGGGGGAGAGCGCAGAGGACAAGGAGCACGAGGAGGGTCGGGACTCG
GAGGAGGGGCCAAGGTGTGGCTCCTCTGAAGACCTGCACGAGAGCGTACGGGAGGGTCCCGACCTGGACA
GGCCTGGGAGCGACCGGCAGGAGCGCGAGAGGGCACGGGGGGACTCGGAGGCCCTGGACGAGGAGAGCTGA

A disclosed NOV9 protein encoded by SEQ ID NO: 24 has 676 amino acid residues, and is presented using the one-letter code in Table 9B (SEQ ID NO: 24). The SignalP, Psort and/or Hydropathy profile for NOV9 predict that NOV9 has no signal peptide and is likely to be localized at the nucleus with a certainty of 0.9866; the mitrochondrial matrix space with a certainty of 0.1000; the lysosome (lumen) with a certainty of 0.1000; and the endoplasmic reticulum (membrane) with a certainty of 0.0000.

The disclosed NOV9 protein is similar to the Mus musculus hepatoma-derived growth factor, related protein 2 (SPTREMBL-ACC:035540).

TABLE 9B
Encoded NOV9 protein sequence (SEQ ID NO:24).

MPHAFKPGDLVFAKMKGYPHWPARIDDIADGAVKPPPNKYPIFFFGTHETAFLGPKDLFPYDKCKDKYGK
PNKRKGFNEGLWEIQNNPHASYSAPPPVSSSDSEAPEANPADGSDADEDDEDRGVMAVTAVTATAASDRM
ESDSDSDKSSDNSGLKRKTPALKVSVSKRARKASSDLDQASVSPSEEENSESSSESEKTSDQDFTPEKKA
AVRAPRRGPLGGRKKKKAPSASDSDSKADSDGAKPEPVAMARSASSSSSSSSSSDSDVSVKKPPRGRKPA
EKPLPKPRGRKPKPERPPSSSSSDSDSDEVDRISEWKRRDEARRRELEARRRREQEEELRRLREQEKEEK
ERRRERADRGEAERGSGGSSGDELREDDEPVKKRGRKGRGRGPPSSSDSEPEAELEREAKKSAKKPQSSS
TEPARKPGQKEKRVRPEEKQQARPVKVERTRKRSEGFSMDRKVEKKKEPSVEEKLQKLHSEIKFALKVDS
PDVKRCLNALEELGTLQVTSQILQKNTDVVATLKKIRRYKANKDVMEKAAEVYTRLKSRVLGPKIEAVQK
VNKAGMEKEKAEEKLAGEELAGEELAGEEAPQEKAEDKPSTDLSAPVNGEATSQKGESAEDKEHEEGRDS
EEGPRCGSSEDLHESVREGPDLDRPGSDRQERERARGDSEALDEES

Hepatoma-derived growth factor (HDGF) and HDGF-related proteins (HRP) belong to a gene family with a well-conserved amino acid sequence at the N-terminus (the hath region). A new member of the HDGF family in humans and mice was identified and cloned; we call it HRP-3. The deduced amino acid sequence from HRP-3 cDNA contained 203 amino acids without a signal peptide for secretion. HRP-3 has its 97-amino-acid sequence at the N-terminus, which is highly conserved with the hath region of the HDGF family proteins. It also has a putative bipartite nuclear localizing signal (NLS) sequence in a similar location in its self-specific region of HDGF and HRP-1. Northern blot analysis shows that HRP-3 is expressed predominantly in the testis and brain, to an intermediate extent in the heart, and to a slight extent in the ovaries, kidneys, spleen, and liver in humans. Transfection of green fluorescent protein (GFP)-tagged HRP-3 cDNA showed that HRP-3 translocated to the nucleus of 293 cells. GFP-HRP-3 transfectants significantly increased their DNA synthesis more than cells transfected with vector only. The HRP-3 gene was mapped to chromosome 15, region q25 by FISH analysis. These findings suggest that a new member of the HDGF gene family, HRP-3, may function mainly in the nucleus of the brain, testis, and heart, probably for cell proliferation. See Ikegame et al., Biochem Biophys Res Commun 266(1):81-87 (1999).

Hepatoma-derived growth factor (HDGF)-related protein (HRP)-1, a member of the HDGF gene family, showed testis-specific expression in mice. HRP-1 expression in spermatogenesis was analyzed in the testis of normal and azoospermic mice by Northern blot and immunohistochemistry. HRP-1 gene message was not expressed in the ovary and its product was detected only in the nuclei of germ cells, not in somatic cells. The HRP-1 gene is expressed through pachytene spermatocyte to round spermatid. HRP-1 gene expression was not detected in the testis of cryptorchid mice or in some strains of mutant mice. These findings suggest that the testis-specific HRP-1 gene may play an important role in the phase around meiotic cell division. See Kuroda et al., Biochem. Biophys Res Commun 262(2):433-37 (1999).

Hepatoma-derived growth factor (HDGF) is an acidic polypeptide with mitogenic activity for fibroblasts performed outside the cells despite the presence of a putative nuclear localization signal (NLS). Three related mouse cDNAs have been cloned: one for a mouse homologue of human HDGF and two for additional HDGF-related proteins provisionally designated HDGF-related proteins 1 and 2 (HRP-1 and -2). Their deduced sequences have revealed that HDGF belongs to a new gene family with a highly conserved 98-amino-acid sequence at the amino terminus (hath region, for homologous to the amino terminus of HDGF). HRP-1 and HRP-2 proteins are 46 and 432 amino acids longer than mouse HDGF, respectively, and have no conserved amino acid sequence other than the hath region. HRP-1 is a highly acidic protein (26% acidic) and also has a putative NLS. HRP-2 protein carries a mixed charge cluster, a sharp switch of positive-to negative-charge residues, which is often found in some nuclear proteins. Northern blotting shows that mouse HDGF and HRP-2 are expressed predominantly in testis and skeletal muscle, to intermediate extents in heart, brain, lung, liver, and kidney, and to a minimal extent in spleen. HRP-1 is expressed specifically in testis. These findings suggest that the HDGF gene family might play a new role in the nucleus especially in testis. See Izumoto et al., Biochem Biophys Res Commun 238(1):26-32 (1997).

Hepatoma-derived growth factor (HDGF) is the first member identified of a new family of secreted heparin-binding growth factors highly expressed in the fetal aorta. The biologic role of HDGF in vascular growth is unknown. Here, HDGF mRNA is expressed in smooth muscle cells (SMCs), most prominently in proliferating SMCs, 8-24 hours after serum stimulation. Exogenous HDGF and endogenous overexpression of HDGF stimulated a significant increase in SMC number and DNA synthesis. Rat aortic SMCs transfected with a hemagglutinin-epitope-tagged rat HDGF cDNA contain HA-HDGF in their nuclei during S-phase. Native HDGF was detected in nuclei of cultured SMCs, of SMCs and endothelial cells from 19-day fetal (but not in the adult) rat aorta, of SMCs proximal to abdominal aortic constriction in adult rats, and of SMCs in the neointima formed after endothelial denudation of the rat common carotid artery. Moreover, HDGF colocalizes with the proliferating cell nuclear antigen (PCNA) in SMCs in human atherosclerotic carotid arteries, suggesting that HDGF helps regulate SMC growth during development and in response to vascular injury. See Everett et al., J. Clin Invest 105(5):567-75 (2000).

In the kidney, there is a close and intricate association between epithelial and endothelial cells, suggesting that a complex reciprocal interaction may exist between these two cell types during renal ontogeny. Thus, it was examined whether metanephrogenic mesenchymal cells secrete endothelial mitogens. With an endothelial mitogenic assay and sequential chromatography of the proteins in the media conditioned by a cell line of rat metanephrogenic mesenchymal cells (7.1.1 cells), a protein whose amino acid analysis identified it as hepatoma-derived growth factor (HDGF) was isolated. Media conditioned with Cos-7 cell transfected with HDGF cDNA stimulated endothelial DNA synthesis. With immunoaffinity purified antipeptide antibodies, HDGF was found to be widely distributed in the renal anlage at early stages of development but soon concentrated at sites of active morphogenesis and, except for some renal tubules, disappeared from the adult kidney. From a 7.1.1 cells cDNA library, a clone of most of the translatable region of HDGF was obtained and used to synthesize digoxigenin-labeled riboprobes. In situ hybridization showed that during kidney development mRNA for HDGF was most abundant at sites of nephron morphogenesis and in ureteric bud cells while in the adult kidney transcripts disappeared except for a small population of distal tubules. Thus, HDGF is an endothelial mitogen that is present in embryonic kidney, and its expression is synchronous with nephrogenesis. See Oliver et al., J. Clin Invest 102(6):1208-19 (1998).

A human hepatoma cell line synthesizes, as evidenced by metabolic labeling, an endothelial cell mitogen that is found to be mostly cell associated. The hepatoma-derived growth factor (HDGF) has been purified to homogeneity by a combination of Bio-Rex 70, heparin-Sepharose, and reverse-phase chromatography; it is a cationic polypeptide with a molecular weight of about 18,500-19,000. HDGF is structurally related to basic fibroblast growth factor (FGF). Immunological analysis demonstrates that antiserum prepared against a synthetic peptide corresponding to the amino-terminal sequence of basic FGF cross-reacts with HDGF when analyzed by electrophoretic blotting and by immunoprecipitation. Sequence analysis of tryptic fragments demonstrates that HDGF contains sequences that are homologous to both amino-terminal and carboxyl-terminal sequences of basic FGF. See Klagsbrun et al., Proc Natl Acad Sci USA 83(8):2448-52 (1986).

According to the OMIM database entry 300043 for hepatoma-derived growth factor, Nakumura et al. purified a novel hepatoma-derived growth factor from the conditioned medium of human hepatoma-derived cell line HuH-7. See Nakamura et al., J Biol Chem 269:25143-49 (1994). Molecular cloning of a cDNA from the cDNA library of the same cell line was done on the basis of the N-terminal amino acid sequence. The cDNA was 2.4 kb long and the deduced amino acid sequence contained 240 amino acids without a signal peptide-like N-terminal hydrophobic sequence. The primary sequence shared homology with the high mobility group-l protein (See OMIM database entry 163905); they showed 23.4% amino acid identity and 35.6% amino acid similarity. Immunofluorescence study showed that HDGF is localized in the cytoplasm of hepatoma cells and northern blots showed that it is expressed ubiquitously in normal tissues and tumor cell lines. Nakamura et al. (1994) suggested that it is a novel heparin-binding protein with mitogenic activity for fibroblasts.

HDGF is ubiquitously expressed in normal tissues and tumor cell lines. By PCR screening of a commercial monochromosomal hybrid panel, Wanschura et al. (1996) mapped HDGF to the X chromosome. See Wanschura et al., Genomics 32:298-300 (1996). By fluorescence in situ hybridization, they determined the subchromosomal localization to be Xq25. Whereas a major group of the HMG protein family has been mapped to chromosomal segments frequently involved in the tumorigenesis of benign solid tumors, no tumor association for the Xq25 region was known.

NOV9 is very likely a nuclear localized peptide as the NOV9 polypeptide is similar to the hepatoma-derived growth factor related protein gene family, some members of which are localized in the nucleus. Hepatoma-derived growth factor related protein genes are temporarily available extracellularly for growth factor signaling. Therefore, it is likely that this novel gene is available at the appropriate subcellular localization and hence accessible for the therapeutic uses described in this application.

This invention describes the following novel hepatoma-derived growth factor related protein-like protein and nucleic acid encoding same (designated CuraGen Accession Number AI284055_EXT). This sequence was initially identified by searching public genomic databases for DNA sequences that translate into proteins with similarity to a protein family of interest. SeqCalling assembly AI284055 (derived from an Image clone) was identified as having suitable similarity. SeqCalling assembly AI284055 was analyzed further to identify an open reading frame encoding for a novel full length protein and novel splice forms of this gene.

The genomic clone AC011498 was analyzed by GenScan and Grail to identify exons and putative coding sequences/open reading frames. The clone ACO011498 was also analyzed by TblastN, BlastX and other homology programs to identify regions translating to proteins with similarity to the original protein/protein family of interest.

The results of these analyses were integrated and manually corrected for apparent inconsistencies, thereby obtaining the sequence encoding the full-length protein. When necessary, the process to identify and analyze cDNAs/ESTs and genomic clones was reiterated to derive the full-length sequence. This invention describes this full-length DNA sequence(s) and the full-length protein sequence(s) which they encode.

The gene encoding NOV9 belongs to genomic clone AC011498 on Chromosome 19.

Based on information available from the expression of ESTs with 100% homologous sequence to AI284055_EXT, it is highly probable that NOV9 is expressed in, for example, but not limited to, blood, brain, colon, esophagus, foreskin, genn cell, lung, nose, ovary, pancreas, prostate, spleen, tonsil, uterus, and lung.

Patp results for NOV9 include those listed in Table 9C.

TABLE 9C
Patp alignments of NOV9

| | | | |
|---|---|---|---|
| | | | Smallest |
| | | | Sum |
| | Reading | High | Probab. |
| Sequences producing High-scoring Segment Pairs: | Frame | Score | P(N) |

| | | | |
|---|---|---|---|
| patp:Y99426 Human PRO1604 (UNQ785) amino acid sequence . . . | +1 | 3406 | 0.0 |
| patp:W37483 Mouse liver cancer-originated culture cell. . . | +1 | 2769 | 1.7e−287 |
| patp:B53322 Human colon cancer antigen protein sequence . . | +1 | 2261 | 1.9e−257 |
| patp:B41868 Human ORFX ORF1632 polypeptide sequence . . . | +1 | 1496 | 1.4e−152 |
| patp:B42974 Human ORFX ORF2738 polypeptide sequence . . . | +1 | 1068 | 3.1e−107 |
| patp:B13522 Human hepatoma-derived growth factor homolog . . . | +1 | 543 | 1.3e−51 |

For example, a BLAST against Y99426, a 671 amino acid hepatoma-derived growth factor from Homo sapiens, produced 668/676 (98%) identity, and 671/676 (99%) positives (E=0.0), with long segments of amino acid identity, as shown in Table 9D. WO 00/12708-A2.

TABLE 9D
Blast Results of NOV9 and Y99426 (SEQ ID NO:85)

Score = 34O6 (1199.0 bits), Expect = 0.0, p = 0.0
Identities = 668/676 (98%), Positives = 671/676 (99%), Frame = +1

| | | | |
|---|---|---|---|
| NOV9: | 1 | MPHAFKPGDLVFAKMKGYPHWPARIDDIADGAVKPPPNKYPIFFFGTHETAFLGPKDLFP | 60 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Y99426: | 1 | MPHAFKPGDLVFAKMKGYPHWPARIDDIADGAVKPPPNKYPIFFFGTHETAFLGPKDLFP | 60 |
| NOV9: | 61 | YDKCKDKYGKPNKRKGENEGLWEIQNNPHASYSAPPPVSSSDSEAPEANPADGSDADEDD | 120 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Y99426: | 61 | YDKCKDKYGKPNKRKGFNEGLWEIQNNPHASYSAPPPVSSSDSEAPEANPADGSDADEDD | 120 |
| NOV9: | 121 | EDRGVMAVTAVTATAASDRMESDSDSDKSSDNSGLKRKTPALKVSVSKRARKASSDLDQA | 180 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|+\|\|\|\|\|\|\|\|\|\|\|\|\|\|\|\| | |
| Y99426: | 121 | EDRGVMAVTAVTATAASDRMESDSDSDKSSDNSGLKRKTPALKMSVSKRARKASSDLDQA | 180 |
| NOV9: | 181 | SVSPSEEENSESSSESEKTSDQDFTPEKKAAVRAPRRGPLGGRKKKKAPSASDSDSKADS | 240 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Y99426: | 181 | SVSPSEEENSESSSESEKTSDQDFTPEKKAAVRAPRRGPLGGRKKKKAPSASDSDSKADS | 240 |
| NOV9: | 241 | DGAKPEPVAMARSASSSSSSSSSSDSDVSVKKPPRGRKPAEKPLPKPRGRKPKPERPPSS | 300 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Y99426: | 241 | DGAKPEPVAMARSASSSSSSSSSSDSDVSVKKPPRGRKPAEKPLPKPRGRKPKPERPPSS | 300 |
| NOV9: | 301 | SSSDSDSDEVDRISEWKRRDEARRRELEARRRREQEEELRRLREQEKEEKERRRERADRG | 360 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Y99426: | 301 | SSSDSDSDEVDRISEWKRRDEARRRELEARRRREQEEELRRLREQEKEEKERRRERADRG | 360 |
| NOV9: | 361 | EAERGSGGSSGDELREDDEPVKKRGRKGRGRGPPSSSDSEPEAELEREAKKSAKKPQSSS | 420 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Y99426: | 361 | EAERGSGGSSGDELREDDEPVKKRGRKGRGRGPPSSSDSEPEAELEREAKKSAKKPQSSS | 420 |
| NOV9: | 421 | TEPARKPGQKEKRVRPEEKQQARPVKVERTRKRSEGFSMDRKVEKKKEPSVEEKLQKLHS | 480 |
| | | \|\|\|\|\|\|\|\|\|\|\|\|\|\|\|\|\|\|\|\|\|\|+\|\|\|\|\|\|\|\|\|\|\|\|\|\|\|\|\|\|\|\|\|\|\|\|\|\|\|\|\|\|\|\|\|\|\|\|\| | |
| Y99426: | 421 | TEPARKPGQKEKRVRPEEKQQAKPVKVERTRKRSEGFSMDRKVEKKKEPSVEEKLQKLHS | 480 |
| NQV9: | 481 | EIKFALKVDSPDVKRCLNALEELGTLQVTSQILQKNTDVVATLKKIRRYKANKDVMEKAA | 540 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Y99426: | 481 | EIKFALKVDSPDVKRCLNALEELGTLQVTSQILQKNTDVVATLKKIRRYKANKDVMEKAA | 540 |
| NOV9: | 541 | EVYTRLKSRVLGPKIEAVQKVNKAGMEKEKAEEKLAGEELAGEELAGEEAPQEKAEDKPS | 600 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\| \|\|\|\|\|\|\|\|\|\|\| | |
| Y99426: | 541 | EVYTRLKSRVLGPKIEAVQKVNKAGMEKEKAEEKLAGEELAGEE-----APQEKAEDKPS | 595 |
| NOV9: | 601 | TDLSAPVNGEATSQKGESAEDKEHEEGRDSEEGPRCGSSEDLHESVREGPDLDRPGSDRQ | 660 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|+\|\|\|\|\|\|\|\|\|\|\|\|\|\|\|\| | |
| Y99426: | 596 | TDLSAPVNGEATSQKGESAEDKEHEEGRDSEEGPRCGSSEDLHDSVREGPDLDRPGSDRQ | 655 |
| NOV9: | 661 | ERERARGDSEALDEES | 676 |
| | | \|\|\|\|\|\|\|\|\|\|\|\|\|\|\|\|\| | |
| Y99426: | 656 | ERERARGDSEALDEES | 671 |

Additionally, NOV9 also showed a large degree of homology with W37483, a 669 amino acid mouse liver cancer-originated culture cell growth factor. Specifically, a BLAST produced 553/676 (81%) identity, and 603/676 (89%) positives (E=1.7e-287), with long segments of amino acid identity. See JP09313185-A.

A BLAST against B53322, a 518 amino acid human colon cancer antigen protein sequence from Homo sapiens, produced 458/465 (98%) identity and 460/465 (98%) positives (E=1.9e-257), with long segments of amino acid identity from nucleic acid residues 388 to 1782. Additionally, this BLAST produced 53/80 (66%) identity and 58/80 (72%) positives (E=2.3e-25) from nucleic acid residues 1677 to 1916; 64/260 (24%) identity and 111/260 (42%) positives (E=2.3e-25) from nucleic acid residues 310 to 1089; 68/296 (22%) identity and 124/296 (41%) positives (E=4.7e-25) from nucleic acid residues 292 to 1161; 59/245 (24%) identity and 101/245 (41%) positives (E=3.2e-24) from nucleic acid residues 709 to 1443; 19/51 (37%) identity and 27/51 (52%) positives (E=1.8e-239) from nucleic acid residues 1638 to 1790; 21/77 (27%) identity and 37/77 (48%) positives (E=2.8e-18) from nucleic acid residues 110 to 340; 18/58 (31%) identity and 28/58 (48%) positives (E=5.0e-17) from nucleic acid residues 195 to 368; and 17/61 (27%) identity and 24/61 (39%) positives (E=1.0e-16) from nucleic acid residues 204 to 383. See WO 00/55351-A1.

A BLAST against B41868, a 308 amino acid human ORFX polypeptide sequence, produced 458/465 (98%) identity and 460/465 (98%) positives (E=1.9e-257), with long segments of amino acid identity from nucleic acid residues 1105 to 2028. See WO 00/58473-A2.

A BLAST against B42974, a 209 amino acid human ORFX polypeptide sequence, produced 208/209 (99%) identity and 209/209 (100%) positives (E=3.1e-107), with long segments of amino acid identity. See WO 00/58473-A2.

The disclosed NOV9 protein (SEQ ID NO: 24) has good identity with hepatoma-derived growth factors. The identity information used for ClustalW analysis is presented in Table 9E. Where indicated, there were two significant regions of homology.

TABLE 9E
BLAST results for NOV9

| Gene Index/ | | Length | Identity | Positives | | |
|---|---|---|---|---|---|---|
| Identifier | Protein/Organism | (aa) | (%) | (%) | Expect | Gaps |
| Gi\|12653923 | Similar to | 670 | 220/272 | 222/272 | 5e−83 | 6/272 |
| \|gb\| | hepatoma-derived | (from | (80%) | (80%) | | (2%) |
| AAH00755.1\| | growth factor, | aa | | | | |
| AH00755 | related protein 2 | 405- | | | | |
| (BC000755) | Homo sapiens | 670) | | | | |
| Gi\|12653923 | Similar to | 670 | 148/280 | 149/280 | 2e−53 | — |
| \|gb\| | hepatoma-derived | (1- | (52%) | (52%) | | |
| AAH00755.1\| | growth factor, | 280) | | | | |
| AH00755 | related protein 2 | | | | | |
| (BC000755) | Homo sapiens | | | | | |
| Gi\|l3277669 | Similar to | 678 | 167/256 | 197/256 | 3e−64 | 6/256 |
| \|gb\| | hepatoma-derived | (426- | (65%) | (76%) | | (2%) |
| AAH03741.1\| | growth factor, | 675) | | | | |
| AAH03741 | related protein 2 | | | | | |
| (BC003741) | Mus musculus | | | | | |
| Gi\|13277669 | Similar to | 678 | 124/209 | 126/209 | 7e−46 | 1/209 |
| \|gb\| | hepatoma-derived | (1- | (59%) | (59%) | | (0%) |
| AAH03741.1\| | growth factor, | 208) | | | | |
| AAH03741 | related protein 2 | | | | | |
| (BC003741) | Mus musculus | | | | | |
| Gi\|6680201 | Hepatoma-derived | 669 | 167/256 | 197/256 | 7e−64 | 6/256 |
| ref\| | growth factor, | | (65%) | (76%) | | (2%) |
| NP_032259.1 | related protein 2 | | | | | |
| | Mus musculus | | | | | |

This information is presented graphically in the multiple sequence alignment given in Table 9F (with NOV9 being shown on line 1) as a ClustalW analysis comparing NOV9 with related protein sequences.

TABLE 9F

Information for the ClustalW proteins:
1) NOV9 (SEQ ID NO:24)
2) gi|12653923|gb|AAH00755.1|AAH00755 (BC000755) Similar to hepatoma-derived growth factor, related protein 2
(Homo sapiens) (SEQ ID NO:86)
3) gi|13277669|gb|AAH03741.1|AAH03741 (BC003741) Similar to hepatoma-derived growth factor, related protein 2
(Mus musculus) (SEQ ID NO:87)
4) gi|6680201|ref|NP_032259.1| Hepatoma-derived growth factor, related protein 2 (Mus musculus) (SEQ ID NO:88)

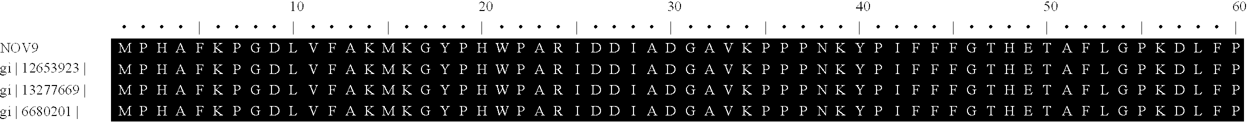

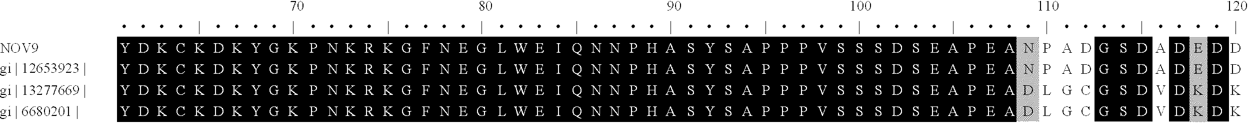

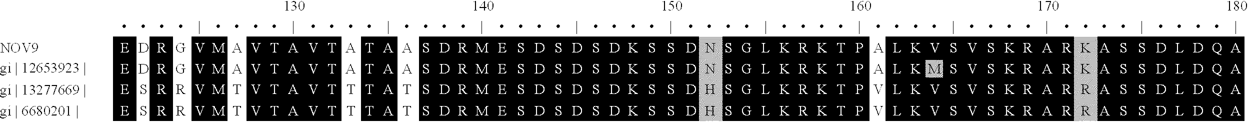

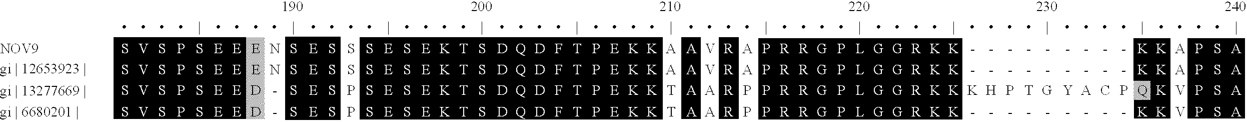

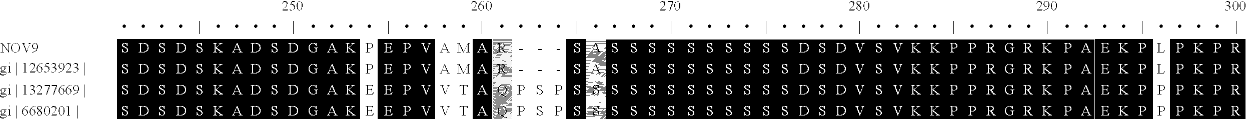

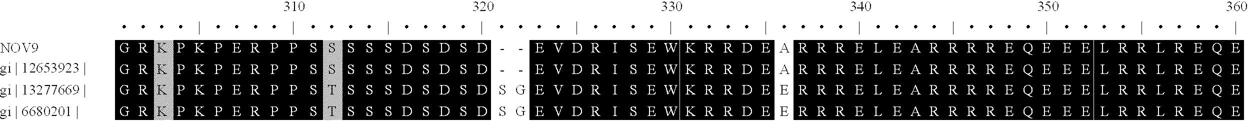

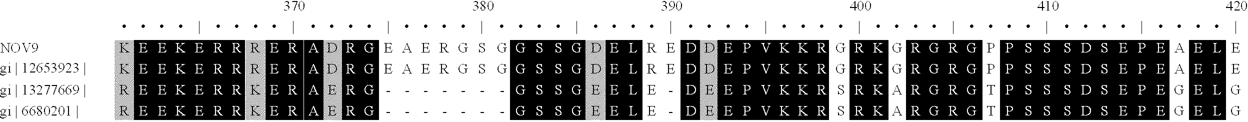

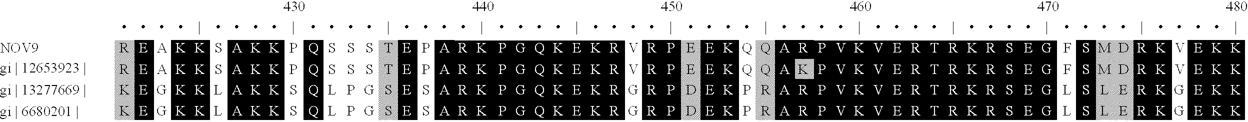

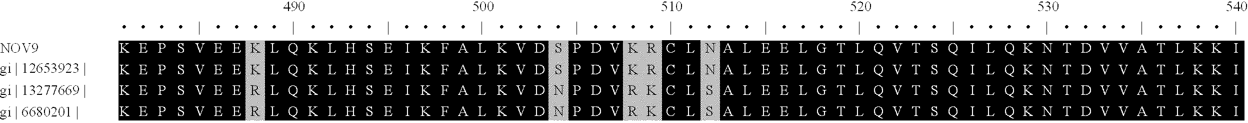

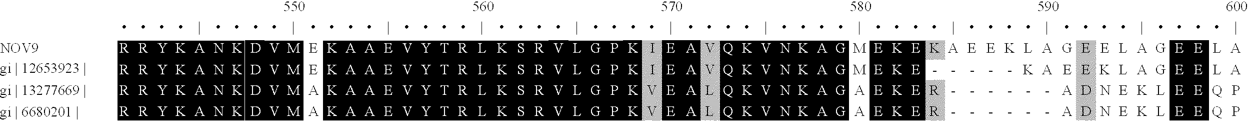

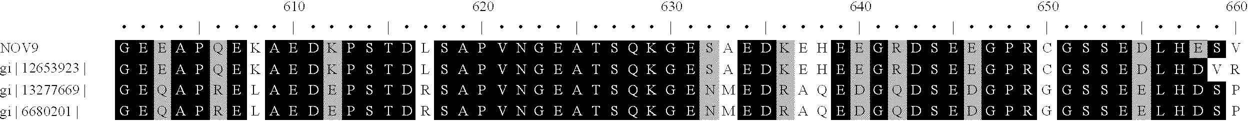

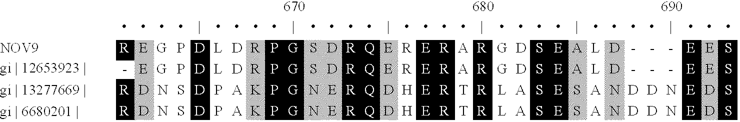

The presence of identifiable domains in NOV9 was determined by searches using algorithms such as PROSITE, Blocks, Pfam, ProDomain, Prints and then determining the Interpro number by crossing the domain match (or numbers) using the Interpro website (http:www.ebi.ac.uk/interpro/).

DOMAIN results for NOV9 were collected from the Conserved Domain Database (CDD) with Reverse Position Specific BLAST. This BLAST samples domains found in the Smart and Pfam collections. The results are listed in Table 9G with the statistics and domain description. The results indicate that this protein contains the following protein domains (as defined by Interpro) at the indicated positions: PWWP domain. This indicates that the sequence of NOV9 has properties similar to those of other proteins known to contain this domain and similar to the properties of this domain.

TABLE 9G
DOMAIN results for NOV9

| Domain | Name | Score (bits) | E Value |
|---|---|---|---|
| Gn\|pfam\|pfam00855 | PWWP, PWWP domain | 97.1 | 2e−21 |
| Gn\|Smart\|PWWP | Domain with conserved | 73.2 | 4e−14 |
| | PWWP motif, conservation | | |
| | of Pro-Trp-Trp-Pro | | |
| | residues | | |

For example, the results of a BLAST of amino residues 5-76 of NOV9 against the 74 amino acid long domain gnl lPfamipfam00855 (SEQ ID NO: 89) are shown in Table 9H.

TABLE 9H
BLAST of NOV9 against gn1|Pfam|pfam00855
CD-Length = 74 residues, 98.6% aligned
Score = 97.1 bits (240), Expect = 2e−21

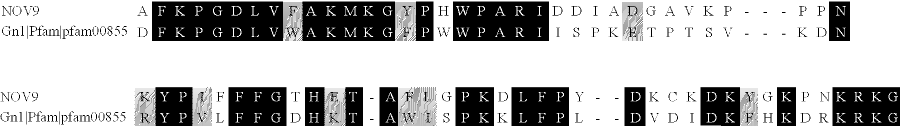

The pattern of expression of this gene and its family members, and its similarity to the hepatoma-derived growth factor related protein-like protein family of genes suggests that it may function as a hepatoma-derived growth factor related protein-like protein in the tissues of expression. Therefore it is implicated in disorders involving these tissues. Some of the diseases include, but are not limited to, Endometriosis, Fertility Anemia, Ataxia-telangiectasia, Autoimmune disease, Immunodeficiencies Systemic lupus erythematosus, Asthma, Emphysema, Scleroderma Von Hippel-Lindau (VHL) syndrome, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Leukodystrophies, Behavioral disorders, Addiction, Anxiety, Pain, Neuroprotection Hemophilia, Hypercoagulation, Idiopathic thrombocytopenic purpura, Graft versus host Hirschsprung's disease, Crohn's Disease, Appendicitis, Cancer, and other diseases and disorders. Family members are known to stimulate endothelial cell mitogenesis, and be involved in nephrogenesis, therefore this novel gene may also be involved in these activities and therapeutic applications derived from these activities.

The expression pattern, map location and protein similarity information for the invention suggests that this gene may function as “Hepatoma-Derived Growth Factor Related Protein”. Therefore, the nucleic acids and proteins of the invention is useful in potential therapeutic applications implicated in Endometriosis, Fertility Anemia, Ataxia-telangiectasia, Autoimmune disease, Immunodeficiencies Systemic lupus erythematosus, Asthma, Emphysema, Scleroderma Von Hippel-Lindau (VHL) syndrome, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Leukodystrophies, Behavioral disorders, Addiction, Anxiety, Pain, Neuroprotection Hemophilia, Hypercoagulation, Idiopathic thrombocytopenic purpura, Graft vesus host Hirschsprung's disease, Crohn's Disease, Appendicitis, Cancer, endothelial cell mitogenesis, nephrogenesis, and other diseases and disorders.

Potential therapeutic uses for the invention(s): Protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vitro and in vivo (regeneration for all these tissues and cell types composing these tissues and cell types derived from these tissues).

The nucleic acids and proteins of the invention are useful in potential therapeutic applications implicated in, for example, but not limited to, Endometriosis, Fertility Anemia, Ataxia-telangiectasia, Autoimmune disease, Immunodeficiencies Systemic lupus erythematosus, Asthma, Emphysema, Scleroderma Von Hippel-Lindau (VHL) syndrome, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Leukodystrophies, Behavioral disorders, Addiction, Anxiety, Pain, Neuroprotection Hemophilia, Hypercoagulation, Idiopathic thrombocytopenic purpura, Graft vesus host Hirschsprung's disease, Crohn's Disease, Appendicitis, Cancer, endothelial cell mitogenesis, nephrogenesis, and other diseases and disorders. For example, a cDNA encoding the hepatoma-derived growth factor related proteinlike protein may be useful in gene therapy, and the hepatoma-derived growth factor related proteinl ike protein may be useful when administered to a subject in need thereof. By way of non-limiting example, the compositions of the present invention will have efficacy for treatment of patients suffering from the pathologies described above. The novel nucleic acid encoding the hepatoma-derived growth factor related protein-like protein, and the hepatoma-derived growth factor related protein-like protein of the invention, or fragments thereof, may further be useful in diagnostic applications, wherein the presence or amount of the nucleic acid or the protein are to be assessed.

These materials are further useful in the generation of antibodies that bind immuno-specifically to the novel NOV9 substances for use in therapeutic or diagnostic methods. These antibodies may be generated according to methods known in the art, using prediction from hydrophobicity charts, as described in the “Anti-NOVX Antibodies” section below. For example the disclosed NOV9 protein has multiple hydrophilic regions, each of which can be used as an immunogen. In one embodiment, a contemplated NOV9 epitope is from about amino acids 5 to about amino acid 60. In another embodiment, a NOV9 epitope is from about amino acids 65 to 110. In additional embodiments, NOV9 epitopes are from about amino acids 115 to 500 and from about amino acids 520 to 680. These novel proteins can also be used to develop assay systems for functional analysis.

NOV10

A disclosed novel NOV 10 nucleic acid of 2349 nucleotides long (also referred to as 95073892_EXT_REVCOMP) is shown in Table 10A (SEQ ID NO: 25). An ORF begins with an ATG initiation codon at nucleotides 1-3 and ends with a TGA codon at nucleotides 2347-2349. The start and stop codons are in bold letters in Table 10A.

TABLE 10A
NOV10 Nucleotide Sequence (SEQ ID NO:25)

ATGGTTATCATGTCGGAGTTCAGCGCGGACCCCGCGGGCCAGGGTCAGGGCCAGCAGAACCCCCTCCGGG
TGGGTTTTTACGACATCGAGCGGACCCTGGGCAAAGGCAACTTCGCGGTGGTGAAGCTGGCGCGGCATCG
AGTCACCAAAACGCAGGTTGCAATAAAAATAATTGATAAAACACGATTAGATTCAAGCAATTTGGAGAAA
ATCTATCGTGAGGTTCAGCTGATGAAGCTTCTGAACCATCCACACATCATAAAGCTTTACCAGGTTATGG
AAACAAAGGACATGCTTTACATCGTCACTGAATTTGCTAAAAATGGAGAAATGTATTATTTGACTTCCAA
CGGGCACCTGAGTGAGAACGAGGCGCGGAAGAAGTTCTGGCAAATCCTGTCGGCCGTGGAGTACTGTCAC
GACCATCACATCGTCCACCGGGACCTCAAGACCGAGAACCTCCTGCTGGATGGCAACATGGACATCAAGC
TGGCAGATTTTGGATTTGGGCAATTTCTACAAGTCAGGAGAGCCTGTCCACGTGGTGTGGGAGCCCCCC
GTATGCCGCCCCGGAAGTCTTTGAGGGGAAGGAGTATGAAGGCCCCCAGCTGGACATCTGGGTAGGCCTG
GGCGTGGTGCTGTACGTCCTGGTCTGCGGTTCTCTCCCCTTCGATGGGCCTAACCTGCCGACGCTGAGAC
AGCGGGTGCTGGAGGGCCGCTTCCGCATCCCCTTCTTCATGTCTCAAGACTGTGAGAGCCTGATCCGCCG
CATGCTGGTGGTGGACCCCGCCAGGCGCATCACCATCGCCCAGATCCGGCAGCACCGGTGGATGCGGGCT
GAGCCCTGCTTGCCGGGACCCGCCTGCCCCGCCTTCTCCGCACACAGCTACACCTCCAACCTGGGCGACT
ACGATGAGCAGGCGCTGGGTATCATGCAGACCCTGGGCGTGGACCGGCAGAGGACGGTGGAGTCACTGCA
AAACAGCAGCTATAACCACTTTGCTGCCATTTATTACCTCCTCCTTGAGCGGCTCAAGGAGTATCGGAAT
GCCCAGTGCGCCCGCCCCGGGCCTGCCAGGCAGCCGCGGCCTCGGAGCTCGGACCTCAGTGGTTTGGAGG
TGCCTCAGGAAGGTCTTTCCACCGACCCTTTCCGACCTGCCTTGCTGTGCCCGCAGCCGCAGACCTTGGT
GCAGTCCGTCCTCCAGGCCGAGATGGACTGTGAGCTCCAGAGCTCGCTGCAGCCCTTGTTCTTCCCGGTG
GATGCCAGCTGCAGCGGAGTGTTCCGGCCCCGGCCCGTGTCCCCAAGCAGCCTGCTGGACACAGCCATCA
GTGAGGAGGCCAGGCAGGGGCCGGGCCTAGAGGAGGAGCAGGACACGCAGGAGTCCCTGCCCAGCAGCAC
GGGCCGGAGGCACACCCTGGCCGAGGTCTCCACCCGCCTCTCCCCACTCACCGCGCCATGTATAGTCGTC
TCCCCCTCCACCACGGCAAGTCCTGCAGAGGGAACCAGCTCTGACAGTTGTCTGACCTTCTCTGCGAGCA
AAAGCCCCGCGGGGCTCAGTGGCACCCCGGCCACTCAGGGGCTGCTGGGCGCCTGCTCCCCGGTCAGGCT
GGCCTCGCCCTTCCTGGGGTCGCAGTCCGCCACCCCAGTGCTGCAGGCTCAGGGGGGCTTGGGAGGAGCT
GTTCTGCTCCCTGTCAGCTTCCAGGAGGGACGGCGGGCGTCGGACACCTCACTGACTCAAGGGCTGAAGG
CCTTTCGGCAGCAGCTGAGGAAGACCACGCGGACCAAAGGGTTTCTGGGACTGAACAAAATCAAGGGGCT
GGCTCGCCAGGTGTGCCAGGCCCCCGCCAGCCGGGCCAGCAGGGGCGGCCTGAGCCCCTTCCACGCCCCT
GCACAGAGCCCAGGCCTGCACGGCGGCGCAGCCGGCAGCCGGGAGGGCTGGAGCCTGCTGGAGGAGGTGC
TAGAGCAGCAGAGGCTGCTCCAGTTACAGCACCACCCGGCCGCTGCACCCGGCTGCTCCCAGGCCCCCCA
GCCGGCCCCTGCCCCGTTTGTGATCGCCCCCTGTGATGGCCCTGGGGCTGCCCCGCTCCCCAGCACCCTC
CTCACGTCGGGGCTCCCGCTGCTGCCGCCCCCACTCCTGCAGACCGGCGCGTCCCCGGTGCCCTCAGCGG
CGCAGCTCCTGGACACACACCTGCACATTGGCACCGGCCCCACCGCCCTCCCCGCTGTGCCCCCACCACG
CCTGGCCAGGCTGGCCCCAGGTTGTGAGCCCCTGGGGCTGCTGCAGGGGGACTGTGAGATGGAGGACCTG
ATGCCCTGCTCCCTAGGCACGTTTGTCCTGGTGCAGTGA

A disclosed NOV10 protein encoded by SEQ ID NO: 25 has 782 amino acid residues, and is presented using the one-letter code in Table 10B (SEQ ID NO: 26). The SignalP, Psort and/or Hydropathy profile for NOV10 predict that NOV10 has no signal peptide and is likely to be localized at the endoplasmic reticulum (membrane) with a certainty of 0.6000; the microbody (peroxisome) with a certainty of 0.3000; the mitochondrial inner membrane with a certainty of 0.1000; and the plasma membrane with a certainty of 0.1000. The disclosed NOV10 protein is similar to the SNF1/AMPK family, some members of which show nuclear localization. Therefore, it is likely that this novel human salt-inducible protein kinase-like protein is available at the appropriate sub-cellular localization and hence is accessible for the therapeutic uses described herein.

The disclosed NOV10 sequence was initially identified by searching CuraGen's Human SeqCalling database for DNA sequences which translate into proteins with similarity to the protein kinase protein family. SeqCalling assembly 95073892 was identified as having suitable similarity. SeqCalling assembly 95073892 has seven components. This assembly was analyzed further to identify open reading frame(s) encoding for a novel full-length protein by extending the SeqCalling assembly using (i) suitable additional SeqCalling assemblies, (ii) publicly available EST sequences, as well as (iii) public genomic sequences.

Two genomic clones, GenBank Accession Numbers AP001046 and AC012140 were identified as having regions with 100% identity to the SeqCalling assembly 95073892 and were selected for analysis because this identity implied that these clones contained the sequence of the genomic locus for this SeqCalling assembly.

The genomic clones were analyzed by Genscan and Grail to identify exons and putative coding sequences/open reading frames. These clones were also analyzed by TblastN, BlastX, and other homology programs to identify regions translating to proteins with similarity to the original protein/protein family of interest. This was found to reside in the following genomic clone regions: in AC001046 from nucleotide 149360-149735, 150161-150392, 150878-151159, 151639-151855, 151974-152096, 152477-152623, 152852-153075, 153628-153750, 153857-153985, 154256-154417, 154595-154655, and in AC012140 from nucleotide 50609-50725, 51225-51380.

The results of these analyses were integrated with SeqCalling assembly information and manually corrected for apparent inconsistencies, thereby obtaining the sequences encoding the full-length cDNA and protein. When necessary, the process to identify and analyze cDNAs/ESTs and genomic clones was reiterated to derive the full-length sequence. This invention describes this full-length DNA sequence(s) and their splice forms and the full-length protein sequence(s) that they encode. These nucleic acids and protein sequences for each splice form are referred to here NOV10.

TABLE 10B
Encoded NOV10 protein sequence (SEQ ID NO:26).

MVIMSEFSADPAGQGQGQQKPLRVGFYDIERTLGKGNFAVVKLARHRVTKTQVAIKIIDKTRLDSSNL
EKIYREVQLMKLLNHPHIIKLYQVMETKDMLYIVTEFAKNGEMYYLTSNGHLSENEARKKFWQILSAV
EYCHDHHIVHRDLKTENLLLDGNMDIKLADFGFGNFYKSGEPLSTWCGSPPYAAPEVFEGKEYEGPQL
DIWVGLGVVLYVLVCGSLPFDGPNLPTLRQRVLEGRFRIPFFMSQDCESLIRRMLVVDPARRITIAQI
RQHRWMRAEPCLPGPACPAFSAHSYTSNLGDYDEQALGIMQTLGVDRQRTVESLQNSSYNHFAAIYYL
LLERLKEYRNAQCARPGPARQPRPRSSDLSGLEVPQEGLSTDPFRPALLCPQPQTLVQSVLQAEMDCE
LQSSLQPLFFPVDASCSGVFRPRPVSPSSLLDTAISEEARQGPGLEEEQDTQESLPSSTGRRHTLAEV
STRLSPLTAPCIVVSPSTTASPAEGTSSDSCLTFSASKSPAGLSGTPATQGLLGACSPVRLASPFLGS
QSATPVLQAQGGLGGAVLLPVSFQEGRRASDTSLTQGLKAFRQQLRKTTRTKGFLGLNKIKGLARQVC
QAPASRASRGGLSPFHAPAQSPGLHGGAAGSREGWSLLEEVLEQQRLLQLQHHPAAAPGCSQAPQPAP
APFVIAPCDGPGAAPLPSTLLTSGLPLLPPPLLQTGASPVASAAQLLDTHLHIGTGPTALPAVPPPRL
ARLAPGCEPLGLLQGDCEMEDLMPCSLGTFVLVQ

PCR-coupled cDNA subtraction hybridization was adapted to identify the genes expressed in the adrenocortical tissues from high salt diet-treated rat. A novel cDNA clone, termed salt-inducible kinase (SIK), encoding a polypeptide (776 amino acids) with significant similarity to protein serine/threonine kinases in the SNF1/AMPK family was isolated. An in vitro kinase assay demonstrated that SIK protein had autophosphorylation activity. Northern blot revealed that SIK mRNA levels were markedly augmented by ACTH treatment both in rat adrenal glands and in Y1 cells. Thus, SIK may play an important role in the regulation of adrenocortical functions in response to high plasma salt and ACTH stimulation. See Wang et al., FEBS Lett 453:135-39 (1999).

The gene encoding the novel human salt-inducible protein kinase-like protein of this invention maps to chromosome 21 between markers MX1-D21S171.

The human salt-inducible protein kinase-like protein disclosed in this invention was found to be expressed in the endocrine system (for example, adrenal gland/supradrenal gland), and in the urinary system (for example, kidney). In addition, the rat and mouse homologs of this gene are expressed in the nervous system (for example, brain) and in the cardiovascular system (for example, heart). Therefore, it is likely that the gene encoding the novel human salt-inducible protein kinase-like protein of this invention (i.e., the gene encoding the NOV10 polypeptide) is also expressed in these tissues in humans.

Patp results for NOV10 include those listed in Table 10C.

TABLE 10C
Patp alignments of NOV10

| | | | Smallest |
|---|---|---|---|
| | | | Sum |
| | Reading | High | Prob. |
| Sequences producing High-scoring Segment Pairs: | Frame | Score | P(N) |
| Patp:W90878 Human keratinocyte derived pKe#122 protein #1 . . | +1 | 776 | 0.0 |
| Patp:W90879 Human keratinocyte derived pK3#122 protein #2 . . | +1 | 776 | 0.0 |
| patp:B36283 Human protein fragment PN765—Homo Sapiens . . | +1 | 209 | 2.7e−108 |

For example, a BLAST against W90878, a 790 amino acid regulatory polypeptide from Homo sapiens, produced 776/783 (99%) identity, and 777/783 (99%) positives (E=0.0), with long segments of amino acid identity, as shown in Table 10D. See WO 00/17232-A1.

TABLE 10D
Blast Results of NOV10 and W90878 (SEQ ID NO:90)

Score = 4022 (1415.8 bits), Expect = 0.0, P = 0.0
Identities = 776/783 (99%), Positives = 777/783 (99%), Frame = +30 1

| | | | |
|---|---|---|---|
| NOV10: | 1 | MVIMSEFSADPAGQGQGQQKPLRVGFYDIERTLGKGNFAVVKLARHRVTKTQVAIKIIDK | 60 |
| | | \|\|\|\|\|\|\|\|\|\|\|\|\|\| \|\|\|\|\|\|\|\|\|\|\|\|\|\|\|\|\|\|\|\|\|\|\|\|\|\|\|\|\|\|\|\|\|\|\|\|\| \|\|\|\|\|\|\| | |
| W90878: | 8 | MVIMSEFSADPAGQSQGQQKPLRVGFYDIERTLGKGNFAVVKLARHRVTKTQNAIKIIDK | 67 |
| NOV10: | 61 | TRLDSSNLEKIYREVQLMKLLNHPHIIKLYQVMETKDMLYIVTEFAKNGEMY-YLTSNGH | 119 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|+ \|\|\|\|\|\|\| | |
| W90878: | 68 | TRLDSSNLEKIYREVQLMKLLNHPHIIKLYQVMETKDMLYIVTEFAKNGEMFDYLTSNGH | 127 |
| NOV10: | 120 | LSENEARKKFWQILSAVEYCHDHHIVHRDLKTENLLLDGMMDIKLADFGFGNFYKSGEPL | 179 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| W90878: | 128 | LSENEARKKFWQILSAVEYCHDHHIVHRDLKTENLLLDGNMDIKLADFGFGNFYKSGEPL | 187 |
| NOV10: | 180 | STWCGSPPYAAPEVFEGKEYEGPQLDIWVGLGVVLYVLVCGSLPFDGPNLPTLRQRVLEG | 238 |
| | | \|\|\|\|\|\|\|\|\|\|\|\|\|\|\|\|\|\|\|\|\|\|\|\|\|\|\|\| \|\|\|\|\|\|\|\|\|\|\|\|\|\|\|\|\|\|\|\|\|\|\|\|\|\|\|\|\|\| | |
| W90878: | 188 | STWCGSPPYAAPEVFEGKEYEGPQLDIW-SLGVVLYVLVCGSLPFDGPNLPTLRQRVLEG | 246 |
| NOV10: | 239 | RFRIPFFMSQDCESLIRRMLVVDPARRITIAQIRQHRWMRAEPCLPGPACPAFSAHSYTS | 298 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| W90878: | 247 | RFRIPFFMSQDCESLIRRMLVVDPARRITIAQIRQHRWMRAEPCLPGPACPAFSAHSYTS | 306 |
| NOV10: | 299 | NLGDYDEQALGIMQTLGVDRQRTVESLQNSSYNHFAAIYYLLLERLKEYRNAQCARPGPA | 358 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| W90878: | 307 | NLGDYDEQALGIMQTLGVDRQRTVESLQNSSYNHFAAIYYLLLERLKEYRNAQCARPGPA | 366 |
| NOV10: | 359 | RQPRPRSSDLSGLEVPQEGLSTDPFRPALLCPQPQTLVQSVLQAEMDCELQSSLQ-PLFF | 417 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| \|\|\|\| | |
| W90878: | 367 | RQPRPRSSDLSGLEVPQEGLSTDPFRPALLCPQPQTLVQSVLQAEMDCELQSSLQWPLFF | 426 |
| NOV10: | 418 | PVDASCSGVFRPRPVSPSSLLDTAISEEARQGPGLEEEQDTQESLPSSTGRRHTLAEVST | 477 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| W90878: | 427 | PVDASCSGVFRPRPVSPSSLLDTAISEEARQGPGLEEEQDTQESLPSSTGRRHTLAEVST | 486 |
| NOV10: | 478 | RLSPLTAPCIVVSPSTTASPAEGTSSDSCLTFSASKSPAGLSGTPATQGLLGACSPVRLA | 537 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| W90878: | 487 | RLSPLTAPCIVVSPSTTASPAEGTSSDSCLTFSASKSPAGLSGTPATQGLLGACSPVRLA | 546 |
| NOV10: | 538 | SPFLGSQSATPVLQAQGGLGGAVLLPVSFQEGRRASDTSLTQGLKAFRQQLRKTTRTKGF | 597 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| W90878: | 547 | SPFLGSQSATPVLQAQGGLGGAVLLPVSFQEGRRASDTSLTQGLKAFRQQLRKTTRTKGF | 606 |
| NOV10: | 598 | LGLNKIKGLARQVCQAPASRASRGGLSPFHAPAQSPGLHGGAAGSREGWSLLEEVLEQQR | 657 |
| | | \|\|\|\|\|\|\|\|\|\|\|\|\|\|\| \|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| W90878: | 607 | LGLNKIKGLARQVCQVPASRASRGGLSPFHAPAQSPGLHGGAAGSREGWSLLEEVLEQQR | 666 |
| NOV10: | 658 | LLQLQHHPAAAPGCSQAPQPAPAPFVIAPCDGPGAAPLPSTLLTSGLPLLPPPLLQTGAS | 717 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| W90878: | 667 | LLQLQHHPAAAPGCSQAPQPAPAPFVIAPCDGPGAAPLPSTLLTSGLPLLPPPLLQTGAS | 726 |
| NOV10: | 718 | PVASAAQLLDTHLHIGTGPTALPAVPPPRLARLAPGCEPLGLLQGDCEMEDLMPCSLGTF | 777 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| W90878: | 727 | PVASAAQLLDTHLHIGTGPTALPAVPPPRLARLAPGCEPLGLLQGDCEMEDLMPCSLGTF | 786 |
| NOV10: | 778 | VLVQ | 781 |
| | | \|\|\|\| | |
| W90878: | 787 | VLVQ | 790 |

Additionally, NOV10 also showed a large degree of homology with W90879, an 823 amino acid regulatory polypeptide from Homo sapiens. Specifically, a BLAST produced 776/783 (99%) identity, and 777/783 (99%) positives (E=0.0), with long segments of amino acid identity. See WO 00/17232-A2.

A BLAST against B36283, a 213 amino acid human protein fragment from Homo sapiens, produced 209/212 (98%) identity and 209/212 (98%) positives (E=2.7e-108), with long segments of amino acid identity. See WO 00/65340-A1.

The disclosed NOV10 protein (SEQ ID NO: 26) has good identity with a number of kinase proteins. The identity information used for ClustalW analysis is presented in Table 10E.

TABLE 10E

BLAST results for NOV10

| Gene Index/ | | Length | Identity | Positives | | |
|---|---|---|---|---|---|---|
| Identifier | Protein/Organism | (aa) | (%) | (%) | Expect | Gaps |
| Gi\|9978891\| | SN1L_HUMAN | 786 | 670/797 | 671/787 | 0.0 | 6/787 |
| sp\| | PROBABLE SERINE/ | | (85%) | (85%) | | (0%) |
| P57059\| | THREONINE KINASE | | | | | |
| (AP001751) | SNF1LK | | | | | |
| | Homo sapiens | | | | | |
| Gi\|12643489 | SN1L_RAT PROBABLE | 776 | 561/787 | 591/787 | 0.0 | 16/787 |
| \|sp\|Q9R1U5 | SERINE/ | | (71%) | (74%) | | (2%) |
| (AB020480) | THREONINE PROTEIN | | | | | |
| | KINASE SNF1LK | | | | | |
| | (SALT-INDUCIBLE | | | | | |
| | PROTEIN KINASE) | | | | | |
| | (PROTEIN KINASE | | | | | |
| | KID2) | | | | | |
| | Rattus norvegicus | | | | | |
| Gi\|11067425 | Salt-inducible | 776 | 560/787 | 591/787 | 0.0 | 16/787 |
| \|ref\| | protein kinase | | (71%) | (74%) | | (2%) |
| NP_067725.1 | Rattus norvegicus | | | | | |
| (AF106937) | | | | | | |
| Gi\|675474\| | Myocardial SNF1- | 779 | 554/790 | 588/790 | 0.0 | 19/790 |
| ref\| | like kinase | | (70%) | (74%) | | (2%) |
| NP_034961.1 | Mus musculus | | | | | |
| (U11494) | | | | | | |
| Gi\|6760436\| | Gin-induced kinase | 798 | 472/803 | 540/803 | 0.0 | 26/803 |
| gb\| | Gallus gallus | | (58%) | (66%) | | (3%) |
| AAF28351.1\| | | | | | | |
| A2219232_1 | | | | | | |
| (AF219232) | | | | | | |

This information is presented graphically in the multiple sequence alignment given in Table 10F (with NOV10 being shown on line 1) as a ClustalW analysis comparing NOV10 with related protein sequences.

TABLE 10
Information for the ClustalW proteins:

1) NOV10 (SEQ ID NO:26)
2) gi|9978891|sp|P57059|SNIL_HUMAN PROBABLE SERINE/THREONINE PROTEIN KINASE SNF1LK (SEQ ID NO:91)
3) gi|12643489|sp|Q9R1U5|SN1L_RAT PROBABLE SERINE/THREONINE PROTEIN KINASE SNF1LK (SEQ ID NO:92)
4) gi|11067425|ref|NP_067725.1|salt-inducible protein kinase (Rattus noriegicus) (SEQ ID NO:93)
5) gi|6754746|ref|NP_034961.1|myocardial SNF1-like kinase (Mus musculus) (SEQ ID NO:94)
6) gi|6760436|gb|AAF28351.1|AF219232_1 (AF219232) (Gallus gallus) (SEQ ID NO:95)

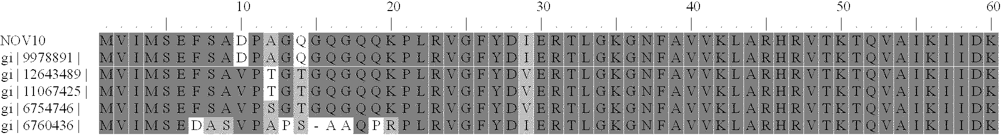

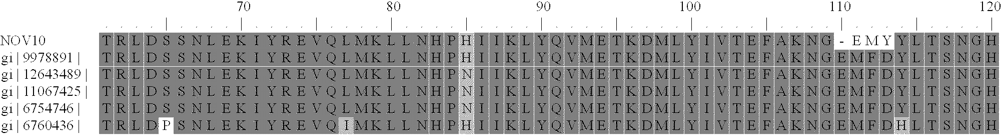

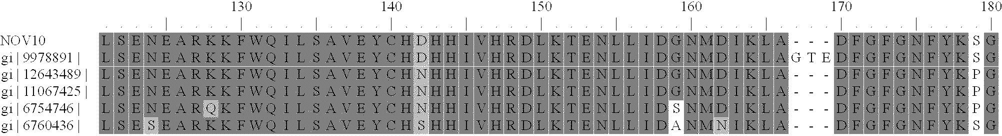

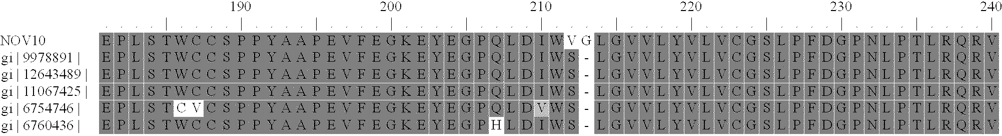

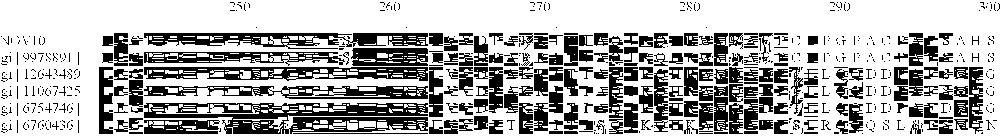

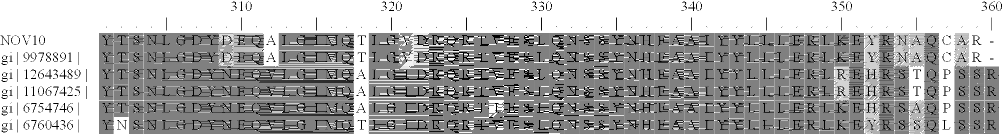

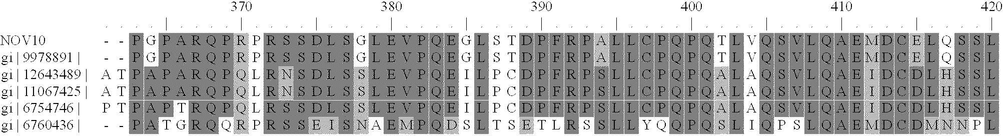

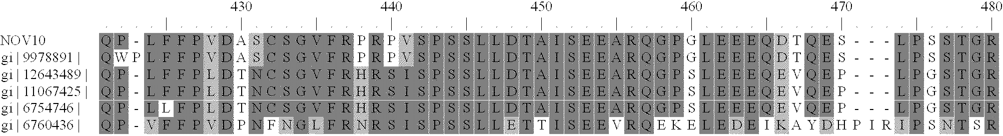

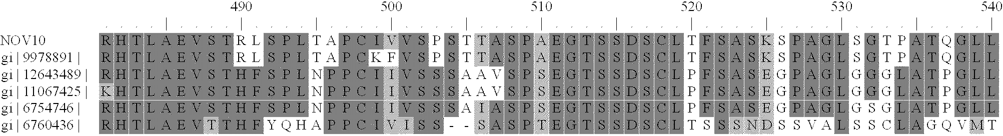

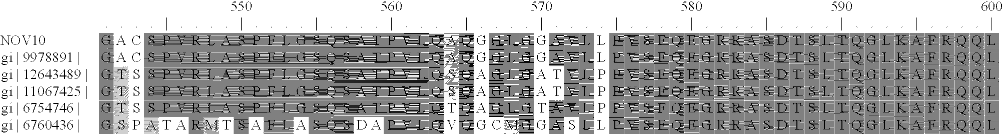

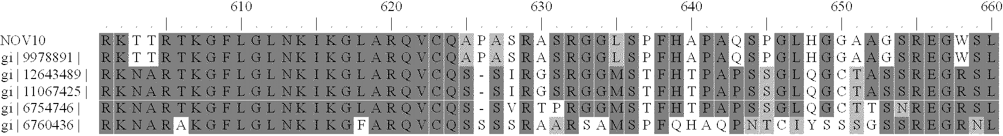

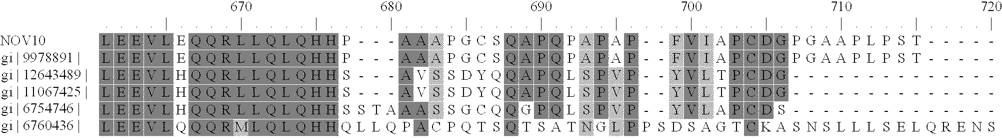

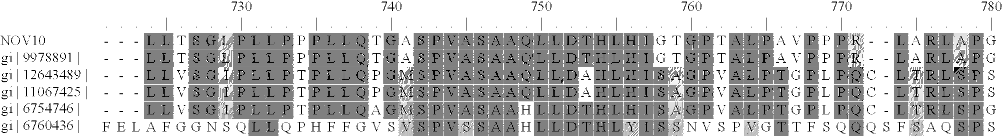

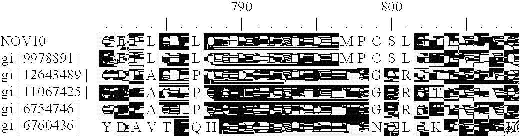

The presence of identifiable domains in NOV10 was determined by searches using algorithms such as PROSITE, Blocks, Pfam, ProDomain, Prints and then determining the Interpro number by crossing the domain match (or numbers) using the Interpro website (http:www.ebi.ac.uk/interpro/).

DOMAIN results for NOV10 were collected from the Conserved Domain Database (CDD) with Reverse Position Specific BLAST. This BLAST samples domains found in the Smart and Pfam collections. The results are listed in Table 10G with the statistics and domain description. The results indicate that this protein contains the following protein domains (as defined by Interpro) at the indicated positions: serine/threonine protein kinases, catalytic domain (at amino acid positions 27-278); pkinase, eukaryotic protein kinase domain (at amino acid positions 27-278); tyrosine kinase, catalytic domain (at amino acid positions 29-274); RIO-like kinase (at amino acid positions 32-167). This indicates that the sequence of NOV10 has properties similar to those of other proteins known to contain this domain and similar to the properties of this domain.

TABLE 10G
DOMAIN results for NOV10

| | | Score | |
|---|---|---|---|
| Domain | Name | (bits) | E Value |
| Gn1\|smart\|S_Tkc | Serine/Threonine pro- | 279 | 3e-76 |
| | tein kinases, cataly- | | |
| | tic domain; Phospho- | | |
| | transferases. Serine | | |
| | or threonine-specific | | |
| | kinase | | |
| Gn1\|Pfam\| | Pkinase, Eukaryotic | 248 | 6e-67 |
| pfam00069 | protein kinase domain | | |
| Gn1\|Smart\|TyrKc | Tyrosine kinase, cata- | 144 | 2e-35 |
| | lytic domain; | | |
| | Phosphotransferases. | | |
| | Tyrosine-specific ki- | | |
| | nase subfamily. | | |
| Gn1\|Smart\|RIO | RIO-like kinase | 36.6 | 0.005 |

For example, the results of a BLAST of NOV10 against gn1|Smart|S_TKc (SEQ ID NO: 96) are shown in Table 10H.

TABLE 10H
BLAST of NOV10 against gn1|Smart|S_TKc

CD-Length = 256 residues, 100.0% aligned
Score = 279 bits (714), Expect = 3e-76

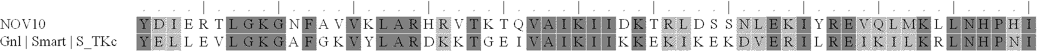

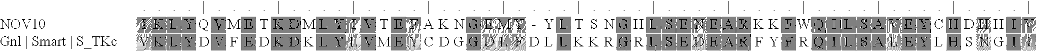

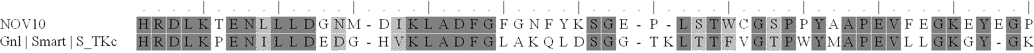

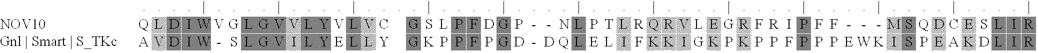

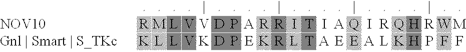

The similarity information for the NOV10 protein and nucleic acid disclosed herein suggest that NOV10 may have important structural and/or physiological functions characteristic of the protein kinase family and the NOV10 family. The expression pattern, map location, and protein similarity information for the invention suggest that the human salt-inducible protein kinase-like protein described in this invention may function as a protein kinase.

NOV10 has been analyzed for tissue expression profiles using the methods described for in the Examples. Various collections of samples are assembled on the plates, and referred to as Panel 1 (containing cells and cell lines from normal and cancer sources), Panel 2 (containing samples derived from tissues, in particular from surgical samples, from normal and cancer sources), Panel 3 (containing samples derived from a wide variety of cancer sources) and Panel 4 (containing cells and cell lines from normal cells and cells related to inflammatory conditions). TaqMan oligo set Ag1542 for the NOV10 gene include the forward probe and reverse oligomers shown in Table 10I.

TABLE 10I
TaqMan oligo set Ag1542

| | | SEQ |
|---|---|---|
| | | ID |
| Primers | Sequences | NO: |
| Forward | 5′-CTATCGTGAGGTTCAGCTGATG-3′ | 97 |
| Probe | FAM-5′-AAGCTTCTGAACCATCCACACATCAT-3′- | 98 |
| | TAMRA | |
| Reverse | 5′-CCTTTGTTTCCATAACCTGGTA-3′ | 99 |

TaqMan oligo set Ag2369 for the NOV10 gene include the forward probe and reverse oligomers shown in Table 10J.

TABLE 10J
TaqMan oligo set Ag2369

| | | SEQ |
|---|---|---|
| Pri- | | ID |
| mers | Sequences | NO: |
| For- | 5′-TCAGCTGATGAAGCTTCTGAAC-3′ | 100 |
| ward | | |
| Probe | FAM-5′-CATCCACACATCATAAAGCTTTACCAGG-3′-TAMRA | 101 |
| verse | 5′-CGATGTAAAGCATGTCCTTTGT-3′ | 102 |
| Re- | | |

The results of the TaqMan expression profile of transcript with these probes are shown below in Tables 10K-10N. Specifically, for Panel 1.3, the expression of Ag1542 is in normal adipose, ovary, lung, and trachea. It is also highly expressed in one renal tumor. For Panel 2, Most normal tissue and tumor margins do not express appreciable levels of this transcript. The highest levels are in the TCC 3. For Panel 4D, small airway epithelium expresses very low levels of this transcript unless it is activated with TNF alpha/IL-1, which increases expression greater than four-fold. Lymphokine activated killer cells (LAK cells) also upregulate this transcript greater than twelve-fold when treated with PMA and ionomycin.

This transcript is up-regulated in small airway epithelium stimulated with proinflammatory cytokines and in activated LAK cells suggesting that it may be involved in the inflammatory process in these two tissues. Blocking the action of this molecule with antibody or small molecule therapeutics may reduce or eliminate inflammation in diseases which target the small airway epithelium such as allergy/asthma and viral infections. Reducing the activity of this molecule in LAK cells during transplantation may prevent organ rejection.

TABLE 10K
TaqMan Results, Probe Ag1542 (Panel 1.3)

| | % Relative |
|---|---|
| Tissue Name | Expression |
| Liver adenocarcinoma | 27.9 |
| Heart (fetal) | 38.7 |
| Pancreas | 2.6 |
| Pancreatic caCAPAN 2 | 2.6 |
| Adrenal gland | 16.5 |
| Thyroid | 4.6 |
| Salivary gland | 1.9 |
| Pituitary gland | 9.7 |
| Brain (fetal) | 2.9 |
| Brain (whole) | 2.1 |
| Brain (amygdala) | 3.6 |
| Brain (cerebellum) | 1.0 |
| Brain (hippocampus) | 20.2 |
| Brain (thalamus) | 3.3 |
| Cerebral Cortex | 10.7 |
| Spinal cord | 6.3 |
| CNS ca. (glio/astro) U87-MG | 6.2 |
| CNS ca. (glio/astro) U-118-MG | 7.1 |
| CNS ca. (astro) 5W1783 | 10.6 |
| CNS ca.* (neuro; met)SK-N-AS | 6.0 |
| CNS ca. (astro) SF-539 | 3.9 |
| CNS ca. (astro) SNB-75 | 11.1 |
| CNS Ca. (glio) SNB-19 | 0.4 |
| CNS Ca. (gilo) U251 | 2.4 |
| CNS ca. (gilo) SF-295 | 2.9 |
| Heart | 6.1 |
| Skeletal muscle | 3.4 |
| Bone marrow | 3.7 |
| Thymus | 2.1 |
| Spleen | 16.8 |
| Lymph node | 6.3 |
| Colorectal | 13.8 |
| Stomach | 4.8 |
| Small intestine | 2.4 |
| Colon ca. 5W480 | 4.8 |
| Colon ca.* (5W480 met)SW620 | 5.9 |
| Colon ca. HT29 | 3.2 |
| Colon ca. HCT-116 | 3.9 |
| Colon ca. CaCo-2 | 8.8 |
| 83219 CC Well to Mod Diff(0D03866) | 20.0 |
| Colon ca. HCC-2998 | 42.3 |
| Gastric ca.* (liver met) NCI-N87 | 37.6 |
| Bladder | 3.2 |
| Trachea | 40.6 |
| Kidney | 1.4 |
| Kidney (fetal) | 14.4 |
| Renal ca. 786-0 | 5.4 |
| Renal ca. A498 | 100.0 |
| Renal ca. RXF 393 | 13.9 |
| Renal ca. ACHN | 13.8 |
| Renal ca. UO-31 | 8.4 |
| Renal ca. TK-10 | 8.0 |
| Liver | 2.0 |
| Liver (fetal) | 17.0 |
| Liver ca. (hepatoblast) HepG2 | 17.8 |
| Lung | 43.5 |
| Lung (fetal) | 16.2 |
| Lung ca. (small cell) LX-1 | 2.8 |
| Lung ca. (small cell) NCI-H69 | 10.3 |
| Lung ca. (scell var.) SHP-77 | 20.6 |
| Lung ca. (large cell) NCI-H460 | 25.5 |
| Lung ca. (non-sm. cell) A549 | 63.3 |
| Lung ca. (non-s.cell) NCI-H23 | 25.5 |
| Lung ca (non-s.cell) HOP-62 | 2.0 |
| Lung ca. (non-s.cl) NCI-H522 | 0.5 |
| Lung ca. (squam.)SW 900 | 8.5 |
| Lung ca. (squam.) NCL-H596 | 3.9 |
| Mammary gland | 25.0 |
| Breast ca.* (pl. effusion)MCF-7 | 13.2 |
| Breast ca.* (pl.ef) MDA-MB-231 | 48.6 |
| Breast ca.* (pl. effusion)T47D | 0.9 |
| Breast ca. BT-549 | 15.4 |
| Breast ca. MDA-N | 0.8 |
| Ovary | 57.0 |
| Ovarian ca. OVCAR-3 | 8.4 |
| Ovarian ca. OVCAR-4 | 1.9 |
| Ovarian ca. OVCAR-5 | 5.3 |
| Ovarian ca. OVCAR-8 | 7.9 |
| Ovarian ca. IGROV-1 | 1.4 |
| Ovarian ca.* (ascites) SK-OV-3 | 10.8 |
| Uterus | 3.5 |
| Placenta | 15.8 |
| Prostate | 4.9 |
| Prostate ca. * (bone met)PC-3 | 6.1 |
| Testis | 10.7 |
| Melanoma Hs688(A).T | 0.4 |
| Melanoma* (met) Hs688(B).T | 1.0 |
| Melanoma UACC-62 | 0.4 |
| Melanoma M14 | 0.6 |
| Melanoma LOX IMVI | 2.9 |
| Melanoma* (met) SK-MEL-5 | 2.7 |
| Adipose | 55.1 |

TABLE 10L
TaqMan Results, Probe Ag1542 (Panel 2D)

| | % Relative |
|---|---|
| Tissue Name | Expression |
| Normal Colon GENPAK 061003 | 17.8 |
| 83219 CC Well to Mod Diff(ODO3866) | 8.0 |
| 83220 CC NAT (ODO3866) | 21.5 |
| 83221 CC Gr.2 rectosigmoid (ODO3868) | 2.2 |
| 83222 CC NAT (ODO3868) | 0.4 |
| 83235 CC Mod Diff (ODO3920) | 3.3 |
| 83236 CC NAT (ODO3920) | 1.7 |
| 83237 CC Gr.2 ascend colon (ODO3921) | 40.1 |
| 83238 CC NAT (ODO3921) | 13.9 |
| 83241 CC from Partial Hepatectomy (ODO4309) | 16.4 |
| 83242 Liver NAT (ODO4309) | 31.2 |
| 87472 Colon mets to lung (ODO4451-01) | 6.7 |
| 87473 Lung NAT (ODO4451-02) | 10.8 |
| Normal Prostate Clontech A+ 6546-1 | 4.1 |
| 84140 Prostate Cancer (OD04410) | 7.6 |
| 84141 Prostate NAT (OD04410) | 6.4 |
| 87073 Prostate Cancer (OD04720-01) | 23.5 |
| 87074 Prostate NAT (OD04720-02) | 50.4 |
| Normal Lung GENPAK 061010 | 34.2 |
| 83239 Lung Met to Muscle (0DO4286) | 16.8 |
| 83240 Muscle NAT (ODO4286) | 16.6 |
| 84136 Lung Malignant Cancer (OD03126) | 25.5 |
| 84137 Lung NAT (OD03126) | 58.2 |
| 84871 Lung Cancer (OD04404) | 27.4 |
| 84872 Lung NAT (OD04404) | 16.6 |
| 84875 Lung Cancer (OD04565) | 18.4 |
| 85950 Lung Cancer (OD04237-01) | 18.4 |
| 85970 Lung NAT (OD04237-02) | 31.9 |
| 83255 Ocular Mel Met to Liver (ODO4310) | 8.5 |
| 83256 Liver NAT (ODO4310) | 37.1 |
| 84139 Melanoma Mets to Lung (OD04321) | 5.5 |
| 84138 Lung NAT (OD04321) | 33.5 |
| Normal Kidney GENPAK 061008 | 4.3 |
| 83786 Kidney Ca, Nuclear grade 2 (OD04338) | 9.6 |
| 83787 Kidney NAT (OD04338) | 21.8 |
| 83788 Kidney Ca Nuclear grade 1/2 (OD04339) | 6.0 |
| 83789 Kidney NAT (OD04339) | 17.9 |
| 83790 Kidney Ca, Clear cell type (OD04340) | 18.2 |
| 83791 Kidney NAT (OD04340) | 29.1 |
| 83792 Kidney Ca, Nuclear grade 3 (OD04348) | 11.0 |
| 83793 Kidney NAT (OD04348) | 8.4 |
| 87474 Kidney Cancer (OD04622-01) | 19.1 |
| 87475 Kidney NAT (OD04622-03) | 7.8 |
| 85973 Kidney Cancer (OD04450-0 1) | 4.5 |
| 85974 Kidney NAT (OD04450-03) | 11.5 |
| Kidney Cancer Clontech 8120607 | 2.5 |
| Kidney NAT Clontech 8120608 | 5.9 |
| Kidney Cancer Clontech 8120613 | 4.8 |
| Kidney NAT Clontech 8120614 | 5.9 |
| Kidney Cancer Clontech 9010320 | 18.8 |
| Kidney NAT Clontech 9010321 | 11.3 |
| Normal Uterus GENPAK 061018 | 2.4 |
| Uterus Cancer GENPAK 064011 | 27.2 |
| Normal Thyroid Clontech A+ 6570-1 | 4.1 |
| Thyroid Cancer GENPAK 064010 | 9.6 |
| Thyroid Cancer INVITROGEN A302152 | 7.3 |
| Thyroid NAT INVITROGEN A302153 | 4.6 |
| Normal Breast GENPAK 061019 | 22.4 |
| 84877 Breast Cancer (OD04566) | 17.3 |
| 85975 Breast Cancer (OD04590-01) | 13.4 |
| 85976 Breast Cancer Mets (OD04590-03) | 13.4 |
| 87070 Breast Cancer Metastasis (OD04655-05) | 4.2 |
| GENPAK Breast Cancer 064006 | 4.6 |
| Breast Cancer Clontech 9100266 | 7.4 |
| Breast NAT Clontech 9100265 | 7.3 |
| Breast Cancer INVITROGEN A209073 | 4.0 |
| Breast NAT INVITROGEN A2090734 | 3.0 |
| Normal Liver GENPAK 061009 | 0.3 |
| Liver Cancer GENPAK 064003 | 5.6 |
| Liver Cancer Research Genetics RNA 1025 | 36.6 |
| Liver Cancer Research Genetics RNA 1026 | 10.3 |
| Paired Liver Cancer Tissue Research Genetics RNA 6004-T | 52.1 |
| Paired Liver Tissue Research Genetics RNA 6004-N | 19.2 |
| Paired Liver Cancer Tissue Research Genetics RNA 6005-T | 8.4 |
| Paired Liver Tissue Research Genetics RNA 6005-N | 12.9 |
| Normal Bladder GENPAK 061001 | 13.5 |
| Bladder Cancer Research Genetics RNA 1023 | 5.0 |
| Bladder Cancer INVITROGEN A302 173 | 5.6 |
| 87071 Bladder Cancer (OD04718-01) | 100.0 |
| 87072 Bladder Normal Adjacent (OD04718-03) | 23.2 |
| Normal Ovary Res. Gen. | 21.9 |
| Ovarian Cancer GENPAK 064008 | 17.9 |
| 87492 Ovary Cancer (OD04768-07) | 5.3 |
| 87493 Ovary NAT (OD04768-08) | 18.2 |
| Normal Stomach GENPAK 061017 | 31.2 |
| NAT Stomach Clontech 9060359 | 21.6 |
| Gastric Cancer Clontech 9060395 | 14.5 |
| NAT Stomach Clontech 9060394 | 41.2 |
| Gastric Cancer Clontech 9060397 | 11.3 |
| NAT Stomach Clontech 9060396 | 6.4 |
| Gastric Cancer GENPAK 064005 | 20.3 |

TABLE 10M
TaqMan Results, Probe Ag1542 (Panel 4D)

| | % Relative |
|---|---|
| Tissue Name | Expression |
| 93768_Secondary Th1_anti-CD28/anti-CD3 | 0.6 |
| 93769_Secondary Th2_anti-CD28/anti-CD3 | 1.0 |
| 93770_Secondary Tr1_anti-CD28/anti-CD3 | 0.8 |
| 93573_Secondary Th1_resting day 4-6 in IL-2 | 0.0 |
| 93572_Secondary Th2_resting day 4-6 in IL-2 | 0.1 |
| 93571_Secondary Tr1_resting day 4-6 in IL-2 | 0.1 |
| 93568_primary Tr1_anti-CD28/anti-CD3 | 2.2 |
| 93569_primary Th2_anti-CD28/anti-CD3 | 1.5 |
| 93570_primary Tr1_anti-CD28/anti-CD3 | 3.0 |
| 93565_primary Th1_resting dy 4-6 in IL-2 | 1.3 |
| 93566_primary Th2_resting dy 4-6 in IL-2 | 0.5 |
| 93567_primary Tn resting dy 4-6 in IL-2 | 1.4 |
| 93351_CD45RA CD4 lymphocyte_anti-CD28/anti-CD3 | 1.3 |
| 93352_CD45RO CD4 lymphocyte_anti-CD28/anti-CD3 | 0.9 |
| 93251_CD8 Lymphocytes_anti-CD28/anti-CD3 | 0.7 |
| 93353_chronic CD8 Lymphocytes 2ry_resting | 0.7 |
| dy 4-6 in IL-2 | |
| 93574_chronic CD8 Lymphocytes 2ry_activated | 0.4 |
| CD3/CD28 | |
| 93354_CD4_none | 1.5 |
| 93252_Secondary Th1/Th2/Trl_anti-CD95 CH11 | 0.2 |
| 93103_LAK cells_resting | 1.3 |
| 93788_LAK cells_IL-2 | 0.4 |
| 93787_LAK cells_IL-2+IL-12 | 1.5 |
| 93789_LAK cells_IL-2+IFN gamma | 2.2 |
| 93790_LAK cells_IL-2+IL-18 | 1.9 |
| 93104_LAK cells_PMA/ionomycin and IL-18 | 12.8 |
| 93578_NK Cells IL-2_resting | 0.4 |
| 93109_Mixed Lymphocyte Reaction_Two Way MLR | 1.3 |
| 93110_Mixed Lymphocyte Reaction_Two Way MLR | 0.8 |
| 93111_Mixed Lymphocyte Reaction_Two Way MLR | 0.2 |
| 93112_Mononuclear Cells (PBMCs)_resting | 3.0 |
| 93113_Mononuclear Cells (PBMCs)_PWM | 4.4 |
| 93114_Mononuclear Cells (PBMCs)_PHA-L | 1.1 |
| 93249_Ramos (B cell)_none | 1.0 |
| 93250_Ramos (B cell)_ionomycin | 2.0 |
| 93349_B lymphocytes_PWM | 5.9 |
| 93350_B lymphoytes_CD40L and IL-4 | 3.0 |
| 92665_EOL-1 (Eosinophil)_dbcAMP differentiated | 1.0 |
| 93248_EOL-1 (Eosinophil)_dbcAMP/PMAionomycin | 1.9 |
| 93356_Dendritic Cells_none | 0.3 |
| 93355_Dendnitic Cells_LPS 100 ng/ml | 0.2 |
| 93775_Dendritic Cells_anti-CD40 | 0.1 |
| 93774_Monocytes_resting | 0.6 |
| 93776_Monocytes_LPS 50 nglml | 0.5 |
| 93581_Macrophages_resting | 1.1 |
| 93582_Macrophages_LPS 100 ng/ml | 0.6 |
| 93098_HUVEC (Endothelial)_none | 0.8 |
| 93099_HUVEC (Endothelial)_starved | 1.0 |
| 93100_HUVEC (Endothelial)_IL-1b | 0.7 |
| 93779_HUVEC (Endothelial)_IFN gamma | 0.3 |
| 93102_HUVEC (Endothelial)_TNF alpha +IFN gamma | 1.3 |
| 93101_HUVEC (Endothelial)_TNF alpha +1L4 | 0.9 |
| 93781_HUVEC (Endothelial)_IL-11 | 0.3 |
| 93583_Lung Microvascular Endothelial Cells_none | 1.1 |
| 93584_Lung Microvascular Endothelial Cells_TNFa | 3.2 |
| (4 ng/ml) and IL1b (1 ng/ml) | |
| 92662_Microvascular Dermal endothelium_none | 2.1 |
| 92663_Microsvasular Dermal endothelium_TNFa | 2.6 |
| (4 ng/ml) and IL1b (1 ng/ml) | |
| 93773_Bronchial epithelium_TNFa | 18.4 |
| (4 ng/ml) and IL1b (1 ng/ml)** | |
| 93347_Small Airway Epithelium_none | 5.1 |
| 93348_Small Airway Epithelium_TNFa | 21.9 |
| (4 ng/ml) and IL1b (1 ng/ml) | |
| 92668_Coronery Artery SMC_resting | 1.1 |
| 92669_Coronery Artery SMC_TNFa | 0.5 |
| (4 ng/ml) and IL1b (1 ng/ml) | |
| 93107_astrocytes_resting | 1.5 |
| 93108_astrocytes_TNFa (4 ng/ml) and IL1b (1 ng/ml) | 1.1 |
| 92666_KU-812 (Basophil)_resting | 0.7 |
| 92667_KU-812 (Basophil)_PMA/ionoycin | 0.9 |
| 93579_CCD1106 (Keratinocytes)_none | 10.6 |
| 93580_CCD1106 (Keratinocytes)_TNFa and IFNg ** | 5.2 |
| 93791_Liver Cirrhosis | 4.2 |
| 93792_Lupus Kidney | 1.1 |
| 93577_NCI-H292 | 100.0 |
| 93358_NCI-H292_IL-4 | 90.1 |
| 93360_NCI-H292_IL-9 | 100.0 |
| 93359_NCI-H292_IL-13 | 52.5 |
| 93357_NCI-H292_IFN gamma | 67.4 |
| 93777_HPAEC_- | 0.7 |
| 93778_HPAEC_IL-1 beta/TNA alpha | 2.8 |
| 93254_Normal Human Lung Fibroblast_none | 0.1 |
| 93253_Normal Human Lung Fibroblast_TNFa | 0.4 |
| (4 ng/ml) and IL-1b (1 ng/ml) | |
| 93257_Normal Human Lung Fibroblast_IL-4 | 0.4 |
| 93256_Normal Human Lung Fibroblast_IL-9 | 0.2 |
| 93255_Normal Human Lung Fibroblast_IL- 13 | 0.3 |
| 93258_Normal Human Lung Fibroblast_IFN gamma | 0.7 |
| 93106_Dermal Fibroblasts CCD1070_resting | 0.5 |
| 93361_Dermal Fibroblasts CCD1070_TNF alpha 4 ng/ml | 0.7 |
| 93105_Dermal Fibroblasts CCD1070_IL-1 beta 1 ng/ml | 0.4 |
| 93772_dermal fibroblast_IFN gamma | 0.1 |
| 93771_dermal fibroblast_IL-4 | 0.1 |
| 93259_IBD Colitis 1** | 1.5 |
| 93260_IBD Colitis 2 | 0.7 |
| 93261_IBD Crohns | 1.4 |
| 735010_Colon_normal | 2.9 |
| 735019_Lung_none | 6.8 |
| 64028-1_Thymus_none | 2.7 |
| 64030-1_Kidney_none | 9.6 |

TABLE 10N
TaqMan Results, Probe Ag2369 (Panel 4D)

| | % Relative |
|---|---|
| Tissue Name | Expression |
| 93768_Secondary Th1_anti-CD28/anti-CD3 | 0.3 |
| 93769_Secondary Th2_anti-CD28/anti-CD3 | 0.6 |
| 93770_Secondary Tr1_anti-CD28/anti-CD3 | 0.4 |
| 93573_Secondary Th1_resting day 4-6 in IL-2 | 0.0 |
| 93572_Secondary Th2_resting day 4-6 in IL-2 | 0.1 |
| 93571_Secondary Tr1_resting day 4-6 in IL-2 | 0.0 |
| 93568_primary Th1_anti-CD28/anti-CD3 | 1.3 |
| 93569_primary Th2_anti-CD28/anti-CD3 | 1.0 |
| 93570_primary Tr1_anti-CD28/anti-CD3 | 1.4 |
| 93565_primary Th1_resting dy 4-6 in IL-2 | 0.7 |
| 93566_primary Th2_resting dy 4-6 in IL-2 | 0.3 |
| 93567_primary Tr1_resting dy 4-6 in IL-2 | 1.0 |
| 93351_CD45RA CD4 lymphocyte_anti-CD28/anti-CD3 | 0.6 |
| 93352_CD45RO CD4 lymphocyte_anti-CD28/anti-CD3 | 0.5 |
| 93251_CD8 Lymphocytes_anti-CD28/anti-CD3 | 0.7 |
| 93353_chronic CD8 Lymphocytes 2ry_resting dy 4-6 in IL-2 | 0.8 |
| 93574_chronic CD8 Lymphocytes 2ry_activated CD3/CD28 | 0.3 |
| 93354_CD4_none | 0.5 |
| 93252_Secondary Th1/Th2/Tr1_anti-CD95 CH11 | 0.1 |
| 93103_LAK cells_resting | 0.9 |
| 93788_LAK cells_IL-2 | 0.3 |
| 93787_LAK cells_IL-2 + IL-12 | 1.3 |
| 93789_LAK cells_IL-2 + IFN gamma | 1.3 |
| 93790_LAK cells_IL-2 + IL-18 | 1.0 |
| 93104_LAK cells_PMA/ionomycin and IL-18 | 9.3 |
| 93578_NK Cells IL-2_resting | 0.2 |
| 93109_Mixed Lymphocyte Reaction_Two Way MLR | 0.7 |
| 93110_Mixed Lymphocyte Reaction_Two Way MLR | 0.5 |
| 93111_Mixed Lymphocyte Reaction_Two Way MLR | 0.3 |
| 93112_Mononuclear Cells (PBMCs)_resting | 1.9 |
| 93113_Mononuclear Cells (PBMCs)_PWM | 3.0 |
| 93114_Mononuclear Cells (PBMCs)_PHA-L | 0.7 |
| 93249_Ramos (B cell)_none | 0.6 |
| 93250_Ramos (B cell)_ionomycin | 1.7 |
| 93349_B lymphocytes_PWM | 5.7 |
| 93350_B lymphoytes_CD40L and IL-4 | 2.7 |
| 92665_EOL-1 (Eosinophil)_dbcAMP differentiated | 0.8 |
| 93248_EOL-1 (Eosinophil)_dbcAMP/PMAionomycin | 1.5 |
| 93356_Dendritic Cells_none | 0.2 |
| 93355_Dendritic Cells_LPS 100 ng/ml | 0.1 |
| 93775_Dendritic Cells_anti-CD40 | 0.2 |
| 93774_Monocytes_resting | 0.6 |
| 93776_Monocytes_LPS 50 ng/ml | 0.4 |
| 93581_Macrophages_resting | 0.6 |
| 93582_Macrophages_LPS 100 ng/ml | 0.4 |
| 93098_HUVEC (Endothelial)_none | 0.6 |
| 93099_HUVEC (Endothelial)_starved | 0.6 |
| 93100_HUVEC (Endothelial)_IL-1b | 0.4 |
| 93779_HUVEC (Endothelial)_IFN gamma | 0.3 |
| 93102_HUVEC (Endothelial)_TNF alpha + IFN gamma | 1.1 |
| 93101_HUVEC (Endothelial)_TNF alpha + IL4 | 1.1 |
| 93781_HUVEC (Endothelial) IL-11 | 0.2 |
| 93583_Lung Microvascular Endothelial Cells_none | 2.4 |
| 93584_Lung Microvascular Endothelial Cells_TNFa (4 ng/ml) and IL1b (1 ng/ml) | 3.4 |
| 92662_Microvascular Dermal endothelium_none | 2.1 |
| 92663_Microsvasular Dermal endothelium_TNFa (4 ng/ml) and IL1b (1 ng/ml) | 1.8 |
| 93773_Bronchial epithelium_TNFa (4 ng/ml) and IL1b (1 ng/ml)** | 3.2 |
| 93347_Small Airway Epithelium_none | 3.1 |
| 93348_Small Airway Epithelium_TNFa (4 ng/ml) and IL1b (1 ng/ml) | 20.8 |
| 92668_Coronery Artery SMC_resting | 0.8 |
| 92669_Coronery Artery SMC_TNFa (4 ng/ml) and IL1b (1 ng/ml) | 0.4 |
| 93107_astrocytes_resting | 1.4 |
| 93108_astrocytes_TNFa (4 ng/ml) and IL1b (1 ng/ml) | 1.3 |
| 92666_KU-812 (Basophil)_resting | 0.6 |
| 92667_KU-812 (Basophil)_PMA/ionoycin | 1.2 |
| 93579_CCD1106 (Keratinocytes)_none | 11.7 |
| 93580_CCD1106 (Keratinocytes)_TNFa and IFNg** | 1.4 |
| 93791_Liver Cirrhosis | 3.0 |
| 93792_Lupus Kidney | 0.7 |
| 93577_NCI-H292 | 96.6 |
| 93358_NCI-H292_IL-4 | 92.2 |
| 93360_NCI-H292_IL-9 | 100.0 |
| 93359_NCI-H292_IL-13 | 56.7 |
| 93357_NCI-H292_IFN gamma | 75.0 |
| 93777_HPAEC_- | 0.4 |
| 93778_HPAEC_IL-1 beta/TNA alpha | 1.4 |
| 93254_Normal Human Lung Fibroblast_none | 0.2 |
| 93253_Normal Human Lung Fibroblast_TNFa (4 ng/ml) and IL-1b (1 ng/ml) | 0.2 |
| 93257_Normal Human Lung Fibroblast_IL-4 | 0.5 |
| 93256_Normal Human Lung Fibroblast_IL-9 | 0.3 |
| 93255_Normal Human Lung Fibroblast_IL-13 | 0.2 |
| 93258_Normal Human Lung Fibroblast_IFN gamma | 0.7 |
| 93106_Dermal Fibroblasts CCD1070_resting | 0.3 |
| 93361_Dermal Fibroblasts CCD1070_TNF alpha 4 ng/ml | 0.5 |
| 93105_Dermal Fibroblasts CCD1070_IL-1 beta 1 ng/ml | 0.2 |
| 93772_dermal fibroblast_IFN gamma | 0.2 |
| 93771_dermal fibroblast_IL-4 | 0.0 |
| 93259_IBD Colitis 1** | 0.3 |
| 93260_IBD Colitis 2 | 0.5 |
| 93261_IBD Crohns | 1.1 |
| 735010_Colon_normal | 2.6 |
| 735019_Lung_none | 6.2 |
| 64028-1_Thymus_none | 1.8 |
| 64030-1_Kidney_none | 8.4 |

The nucleic acid and protein of the invention are useful in potential therapeutic applications implicated, for example but not limited to, in Adrenoleukodystrophy, Congenital Adrenal Hyperplacia, Polycystic Kidney Disease, Stenosis, Interstitial Nephritis, Glomerulonephritis, Atherosclerosis, Hypertension, Congenital Heart Defects, Aortic Stenosis, Atrial Septal Defect, Alzheimer's Disease, Stroke, Tuberous Sclerosis, Hypercalceimia, Parkinson's Disease, and other diseases and disorders. Potential therapeutic uses for the invention(s) are, for example but not limited to, the following: (i) Protein therapeutic, (ii) small molecule drug target, (iii) antibody target (therapeutic, diagnostic, drug targeting/cytotoxic antibody), (iv) diagnostic and/or prognostic marker, (v) gene therapy (gene delivery/gene ablation), (vi) research tools, and (vii) tissue regeneration in vitro and in vivo (regeneration for all these tissues and cell types composing these tissues and cell types derived from these tissues e.g., adrenal gland, kidney, brain, and heart.

The nucleic acids and proteins of the invention are useful in potential therapeutic applications implicated in various diseases and disorders described below and/or other pathologies and disorders. For example, but not limited to, a cDNA encoding the human salt-inducible protein kinase-like protein may be useful in gene therapy, and the Human salt-inducible protein kinase-like protein may be useful when administered to a subject in need thereof. By way of non-limiting example, the compositions of the present invention will have efficacy for treatment of patients suffering from, for example, but not limited to, Adrenoleukodystrophy, Congenital Adrenal Hyperplacia, Polycystic Kidney Disease, Stenosis, Interstitial Nephritis, Glomerulonephritis, Atherosclerosis, Hypertension, Congenital Heart Defects, Aortic Stenosis, Atrial Septal Defect, Alzheimer's Disease, Stroke, Tuberous Sclerosis, Hypercalceimia, Parkinson's Disease, and other diseases and disorders. The novel nucleic acid encoding the Human salt-inducible protein kinase-like protein, and the human salt-inducible protein kinase-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

These materials are further useful in the generation of antibodies that bind immuno-specifically to the novel NOV10 substances for use in therapeutic or diagnostic methods. These antibodies may be generated according to methods known in the art, using prediction from hydrophobicity charts, as described in the “Anti-NOVX Antibodies” section below. For example the disclosed NOV10 protein has multiple hydrophilic regions, each of which can be used as an immunogen. In one embodiment, a contemplated NOV10 epitope is from about amino acids 5 to about amino acid 40. In another embodiment, a NOV10 epitope is from about amino acids 225 to 240. In additional embodiments, NOV10 epitopes are from about amino acids 50 to 90; from about amino acids 105 to 175; from about amino acids 180 to 210; from about amino acids 280 to 400; from about amino acids 450 to 490; and from about amino acids 580 to 680. These novel proteins can also be used to develop assay systems for functional analysis.

### EXAMPLE 1

### Quantitative Expression Analysis of Clones in Various Cells and Tissues

The quantitative expression of various clones was assessed using microtiter plates containing RNA samples from a variety of normal and pathology-derived cells, cell lines and tissues using real time quantitative PCR (RTQ PCR; TAQMAN®). RTQ PCR was performed on a Perkin-Elmer Biosystems ABI PRISM® 7700 Sequence Detection System. Various collections of samples are assembled on the plates, and referred to as Panel 1 (containing cells and cell lines from normal and cancer sources), Panel 2 (containing samples derived from tissues, in particular from surgical samples, from normal and cancer sources), Panel 3 (containing samples derived from a wide variety of cancer sources) and Panel 4 (containing cells and cell lines from normal cells and cells related to inflammatory conditions).

First, the RNA samples were normalized to constitutively expressed genes such as actin and GAPDH. RNA (˜50 ng total or ˜1 ng polyA+) was converted to cDNA using the TAQMAN® Reverse Transcription Reagents Kit (PE Biosystems, Foster City, Calif.; Catalog No. N808-0234) and random hexamers according to the manufacturer's protocol. Reactions were performed in 20 ul and incubated for 30 min. at 48° C. cDNA (5 ul) was then transferred to a separate plate for the TAQMAN® reaction using β-actin and GAPDH TAQMAN® Assay Reagents (PE Biosystems; Catalog Nos. 4310881E and 4310884E, respectively) and TAQMAN® universal PCR Master Mix (PE Biosystems; Catalog No. 4304447) according to the manufacturer's protocol. Reactions were performed in 25 ul using the following parameters: 2 min. at 50° C.; 10 min. at 95° C.; 15 sec. at 95° C./1 min. at 60° C. (40 cycles). Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100. The average CT values obtained for β-actin and GAPDH were used to normalize RNA samples. The RNA sample generating the highest CT value required no further diluting, while all other samples were diluted relative to this sample according to their β-actin /GAPDH average CT values.

Normalized RNA (5 ul) was converted to cDNA and analyzed via TAQMAN® using One Step RT-PCR Master Mix Reagents (PE Biosystems; Catalog No. 4309169) and gene-specific primers according to the manufacturer's instructions. Probes and primers were designed for each assay according to Perkin Elmer Biosystem's Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tₘ) range=58°-60° C., primer optimal Tm=59° C., maximum primer difference=2° C., probe does not have 5′ G, probe Tₘ must be 10° C. greater than primer Tₘ, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: Normalized RNA from each tissue and each cell line was spotted in each well of a 96 well PCR plate (Perkin Elmer Biosystems). PCR cocktails including two probes (a probe specific for the target clone and another gene-specific probe multiplexed with the target probe) were set up using 1×TaqMan™ PCR Master Mix for the PE Biosystems 7700, with 5 mM MgC12, dNTPs (dA, G, C, U at 1:1:1:2 ratios), 0.25 U/ml AmpliTaq Gold™ (PE Biosystems), and 0.4 U/μl RNase inhibitor, and 0.25 U/μl reverse transcriptase. Reverse transcription was performed at 48° C. for 30 minutes followed by amplification/PCR cycles as follows: 95° C. 10 min, then 40 cycles of 95° C. for 15 seconds, 60° C. for 1 minute.

The following abbreviations are used in the panels: ca. =carcinoma, *=established from metastasis, met=metastasis, s cell var=small cell variant, non-s =non-sm=non-small, squam=squamous, pl. eff=pl effusion=pleural effusion, glio=glioma, astro=astrocytoma, and neuro=neuroblastoma.

Panel 2

The plates for Panel 2 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologists at NDRI or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissue were ascertained to be free of disease and were purchased from various commercial sources such as Clontech (Palo Alto, Calif.), Research Genetics, and Invitrogen.

RNA integrity from all samples is controlled for quality by visual assessment of agarose gel electropherograms using 28S and 18S ribosomal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

Panel 4

Panel 4 includes samples on a 96 well plate (2 control wells, 94 test samples) composed of RNA (Panel 4r) or cDNA (Panel 4d) isolated from various human cell lines or tissues related to inflammatory conditions. Total RNA from control normal tissues such as colon and lung (Stratagene ,La Jolla, Calif.) and thymus and kidney (Clontech) were employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), 1 mM sodium pyruvate (Gibco), mercaptoethanol 5.5×10⁻⁵ M (Gibco), and 10 mM Hepes (Gibco) and Interleukin 2 for 4-6 days. Cells were then either activated with 10-20 ng/ml PMA and 1-2 μg/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶ cells/ml in DMEM 5% FCS (Hyclone), 100 μM non essential amino acids (Gibco), 1 mM sodium pyruvate (Gibco), mercaptoethanol (5.5×10⁻⁵ M) (Gibco), and 10 mM Hepes (Gibco). The MLR was cultured and samples taken at various time points ranging from 1- 7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100 μM non essential amino acids (Gibco), 1 mM sodium pyruvate (Gibco), mercaptoethanol 5.5×10⁻⁵ M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and +ve selection. Then CD45RO beads were used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and plated at 10⁶ cells/ml onto Falcon 6 well tissue culture plates that had been coated overnight with 0.5 μg/ml anti-CD28 (Pharmingen) and 3 ug/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶ cells/ml in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24,48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶ cells/ml in DMEM 5% FCS (Hyclone), 100 μM non essential amino acids (Gibco),1 mM sodium pyruvate (Gibco), mercaptoethanol 5.5×10⁻⁵ M (Gibco), 10 mM Hepes (Gibco) and IL-2 (4 ng/ml). IL-12 (5 ng/ml) and anti-IL4 (1 μg/ml) were used to direct to Th1, while IL-4 (5 ng/ml) and anti-IFN gamma (1 μg/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 μg/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁵ cells/ml for 8 days, changing the media every 3 days and adjusting the cell concentration to 5×10⁵ cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD106 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 10⁷ cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20 degrees C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, 7 μl RNAsin and 8 μl DNAse were added. The tube was incubated at 37 degrees C. for 30 minutes to remove contaminating genomic DNA, extracted once with phenol chloroform and re-precipitated with 1/10 volume of 3 M sodium acetate and 2 volumes of 100% ethanol. The RNA was spun down and placed in RNAse free water. RNA was stored at −80 degrees C.

NOVX Nucleic Acids and Polypeptides

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e.g., NOVX mRNAs) and fragments for use as PCR primers for the amplification and/or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

An NOVX nucleic acid can encode a mature NOVX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product, encoded by the corresponding gene. Alternatively, it may be defined as the polypeptide, precursor or proprotein encoded by an ORF described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

The term “probes”, as utilized herein, refers to nucleic acid sequences of variable length, preferably between at least about 10 nucleotides (nt), 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies.

The term “isolated” nucleic acid molecule, as utilized herein, is one, which is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOVX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence SEQ ID NOS: 1, 3, 5, 7, 9, 11, 13, 15, 17, 19, 21, 23, 25, or a complement of this aforementioned nucleotide sequence, can be isolated using standard molecular biology techniques and the sequence information provided herein. Using all or a portion ofthe nucleic acid sequence of SEQ ID NOS: 1, 3, 5, 7, 9, 11,13,15,17, 19, 21, 23, 25 as a hybridization probe, NOVX molecules can be isolated using standard hybridization and cloning techniques (e.g., as described in Sambrook, et al., (eds.), MOLECULAR CLONING: A LABORATORY MANUAL ₂^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides SEQ ID NOS: 1, 3, 5, 7, 9, 11, 13, 15, 17, 19, 21, 23, and 25, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NOS: 1, 3, 5, 7, 9, 11, 13, 15, 17, 19, 21, 23, and 25, or a portion of this nucleotide sequence (e.g., a fragment that can be used as a probe or primer or a fragment encoding a biologically-active portion of an NOVX polypeptide). A nucleic acid molecule that is complementary to the nucleotide sequence shown SEQ ID NOS: 1, 3, 5, 7, 9, 11, 13, 15, 17, 19, 21, 23, or 25 is one that is sufficiently complementary to the nucleotide sequence shown SEQ ID NOS: 1, 3, 5, 7, 9, 11, 13, 15, 17, 19, 21, 23, or 25 that it can hydrogen bond with little or no mismatches to the nucleotide sequence shown SEQ ID NOS: 1, 3, 5, 7, 9, 11, 13, 15, 17, 19, 21, 23, and 25, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for an NOVX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human NOVX protein. Homologous nucleic acid sequences include those nucleic acid sequences that encode conservative amino acid substitutions (see below) in SEQ ID NOS: 1, 3, 5, 7, 9, 11, 13, 15, 17, 19, 21, 23, and 25, as well as a polypeptide possessing NOVX biological activity. Various biological activities of the NOVX proteins are described below.

An NOVX polypeptide is encoded by the open reading frame (“ORF”) of an NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bonafide cellular protein, a minimum size requirement is often set, e.g., a stretch of DNA that would encode a protein of 50 amino acids or more.

The nucleotide sequences determined from the cloning of the human NOVX genes allows for the generation of probes and primers designed for use in identifying and/or cloning NOVX homologues in other cell types, e.g. from other tissues, as well as NOVX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence SEQ ID NOS: 1, 3, 5, 7, 9, 11, 13, 15, 17, 19, 21, 23, or 25; or an anti-sense strand nucleotide sequence of SEQ ID NOS: 1, 3, 5, 7, 9, 11, 13, 15, 17, 19, 21, 23, or 25; or of a naturally occurring mutant of SEQ ID NOS: 1, 3, 5, 7, 9, 11, 13, 15, 17, 19, 21, 23, and 25.

Probes based on the human NOVX nucleotide sequences can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissues which mis-express an NOVX protein, such as by measuring a level of an NOVX-encoding nucleic acid in a sample of cells from a subject e.g., detecting NOVX mRNA levels or determining whether a genomic NOVX gene has been mutated or deleted. “A polypeptide having a biologically-active portion of an NOVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically- active portion of NOVX” can be prepared by isolating a portion SEQ ID NOS: 1, 3, 5, 7, 9, 11, 13, 15, 17, 19,21,23, or 25, that encodes apolypeptide having an NOVX biological activity (the biological activities of the NOVX proteins are described below), expressing the encoded portion of NOVX protein (e.g., by recombinant expression in vitro) and assessing the activity of the encoded portion of NOVX.

NOVX Nucleic Acid and Polypeptide Variants

The invention further encompasses nucleic acid molecules that differ from the nucleotide sequences shown in SEQ ID NOS: 1, 3, 5, 7, 9, 11, 13, 15, 17, 19, 21, 23, and 25 due to degeneracy of the genetic code and thus encode the same NOVX proteins as that encoded by the nucleotide sequences shown in SEQ ID NOS: 1, 3, 5, 7, 9, 11, 13, 15, 17, 19, 21, 23, and 25. In another embodiment, an isolated nucleic acid molecule of the invention has a nucleotide sequence encoding a protein having an amino acid sequence shown in SEQ ID NOS: 2, 4, 6, 8, 10, 12, 14, 16, 18, 20, 22, 24, and 26.

In addition to the human NOVX nucleotide sequences shown in SEQ ID NOS: 1, 3, 5, 7, 9, 11, 13, 15, 17, 19, 21, 23, and 25, it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “gene” and “recombinant gene” refer to nucleic acid molecules comprising an open reading frame (ORF) encoding an NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from the human SEQ ID NOS: 1, 3, 5, 7, 9, 11, 13, 15, 17, 19, 21, 23, and 25 are intended to be within the scope of the invention. Nucleic acid molecules corresponding to natural allelic variants and homologues of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NOS: 1, 3, 5, 7, 9, 11, 13, 15, 17, 19, 21, 23, and 25. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5 ° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to the sequences SEQ ID NOS: 1, 3, 5, 7, 9, 11, 13, 15, 17, 19, 21, 23, and 25, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NOS: 1, 3, 5, 7, 9, 11, 13, 15, 17, 19, 21, 23, and 25, or fragments, analogs or derivatives thereof, under conditions of moderate stringency is provided. A non-limiting example of moderate stringency hybridization conditions are hybridization in 6×SSC, 5×Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, NY, and Kriegler, 1990; GENE TRANSFER AND EXPRESSION, A LABORATORY MANUAL, Stockton Press, NY.

In a third embodiment, a nucleic acid that is hybridizable to the nucleic acid molecule comprising the nucleotide sequences SEQ ID NOS: 1, 3, 5, 7, 9, 11, 13, 15, 17, 19, 21, 23, and 25, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981. Proc Natl Acad Sci USA 78: 6789-6792.

Conservative Mutations

In addition to naturally-occurring allelic variants of NOVX sequences that may exist in the population, the skilled artisan will further appreciate that changes can be introduced by mutation into the nucleotide sequences SEQ ID NOS: 1, 3, 5, 7, 9, 11, 13, 15, 17, 19, 21, 23, and 25, thereby leading to changes in the amino acid sequences of the encoded NOVX proteins, without altering the functional ability of said NOVX proteins. For example, nucleotide substitutions leading to amino acid substitutions at “non-essential” amino acid residues can be made in the sequence SEQ ID NOS: 2, 4, 6, 8, 10, 12, 14, 16, 18, 20, 22, 24, and 26. A “non-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are predicted to be particularly non-amenable to alteration. Amino acids for which conservative substitutions can be made are well-known within the art.

Another aspect of the invention pertains to nucleic acid molecules encoding NOVX proteins that contain changes in amino acid residues that are not essential for activity. Such NOVX proteins differ in amino acid sequence from SEQ ID NOS: 2, 4, 6, 8, 10, 12, 14, 16, 18, 20, 22, 24, or 26 yet retain biological activity. In one embodiment, the isolated nucleic acid molecule comprises a nucleotide sequence encoding a protein, wherein the protein comprises an amino acid sequence at least about 45% homologous to the amino acid sequences SEQ ID NOS: 2, 4, 6, 8, 10, 12, 14, 16, 18, 20, 22, 24, or 26. Preferably, the protein encoded by the nucleic acid molecule is at least about 60% homologous to SEQ ID NOS: 2, 4, 6, 8, 10, 12, 14, 16, 18, 20, 22, 24, or 26; more preferably at least about 70% homologous SEQ ID NOS: 2, 4, 6, 8, 10, 12, 14, 16, 18, 20, 22, 24, and 26; still more preferably at least about 80% homologous to SEQ ID NOS: 2, 4, 6, 8, 10, 12, 14, 16, 18, 20, 22, 24, and 26; even more preferably at least about 90% homologous to SEQ ID NOS: 2, 4, 6, 8, 10, 12, 14, 16, 18, 20, 22, 24, and 26; and most preferably at least about 95% homologous to SEQ ID NOS: 2, 4, 6, 8, 10, 12, 14, 16, 18, 20, 22, 24, and 26.

An isolated nucleic acid molecule encoding an NOVX protein homologous to the protein of SEQ ID NOS: 2, 4, 6, 8, 10, 12, 14, 16, 18, 20, 22, 24, and 26 can be created by introducing one or more nucleotide substitutions, additions or deletions into the nucleotide sequence of SEQ ID NOS: 1, 3, 5, 7, 9, 11, 13, 15, 17, 19, 21, 23, and 25, such that one or more amino acid substitutions, additions or deletions are introduced into the encoded protein.

Mutations can be introduced into SEQ ID NOS: 2, 4, 6, 8, 10, 12, 14, 16, 18, 20, 22, 24, and 26 by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the NOVX protein is replaced with another amino acid residue from the same side chain family. Alternatively, in another embodiment, mutations can be introduced randomly along all or part of an NOVX coding sequence, such as by saturation mutagenesis, and the resultant mutants can be screened for NOVX biological activity to identify mutants that retain activity. Following mutagenesis SEQ ID NOS: 1, 3, 5, 7, 9, 11, 13, 15, 17, 19, 21, 23, and 25, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, VLIM, HFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and an NOVX ligand; or (iii) the ability of a mutant NOVX protein to bind to an intracellular target protein or biologically-active portion thereof, (e.g. avidin proteins).

In yet another embodiment, a mutant NOVX protein can be assayed for the ability to regulate a specific biological function (e.g., regulation of insulin release).

Antisense Nucleic Acids

Another aspect of the invention pertains to isolated antisense nucleic acid molecules that are hybridizable to or complementary to the nucleic acid molecule comprising the nucleotide sequence of SEQ ID NOS: 1, 3, 5, 7, 9, 11, 13, 15, 17, 19, 21, 23, and25, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of an NOVX protein of SEQ ID NOS: 2, 4, 6, 8, 10, 12, 14, 16, 18, 20, 22, 24, and 26, or antisense nucleic acids complementary to an NOVX nucleic acid sequence of SEQ ID NOS: 1, 3, 5, 7, 9, 11, 13, 15, 17, 19, 21, 23, and 25, are additionally provided.

In one embodiment, an antisense nucleic acid molecule is antisense to a “coding region” of the coding strand of a nucleotide sequence encoding an NOVX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOVX protein. The term “noncoding region” refers to 5′and 3′ sequences which flank the coding region that are not translated into amino acids (i.e., also referred to as 5′ and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an NOVX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (see, e.g., Inoue, et al. 1987. Nucl. Acids Res. 15: 6131-6148) or a chimeric RNA-DNA analogue (see, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOVX mRNA transcripts to thereby inhibit translation of NOVX mRNA. A ribozyme having specificity for an NOVX-encoding nucleic acid can be designed based upon the nucleotide sequence of an NOVX cDNA disclosed herein (i.e., SEQ ID NOS: 1, 3, 5, 7, 9, 11, 13, 15, 17, 19, 21, 23, and 25). For example, a derivative of a Tetrahymena L-19 IVS RNA can be constructed in which the nucleotide sequence of the active site is complementary to the nucleotide sequence to be cleaved in an NOVX-encoding mRNA. See, e.g., U.S. Pat. No. 4,987,071 to Cech, et al. and U.S. Pat. No. 5,116,742 to Cech, et al. NOVX mRNA can also be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX nucleic acid (e.g., the NOVX promoter and/or enhancers) to form triple helical structures that prevent transcription of the NOVX gene in target cells. See, e.g., Helene, 1991. Anticancer Drug Des. 6: 569-84; Helene, et al. 1992. Ann. N.Y Acad. Sci. 660: 27-36; Maher, 1992. Bioassays 14: 807-15.

In various embodiments, the NOVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S₁ nucleases (see, Hyrup, et al., 1996.supra); or as probes or primers for DNA sequence and hybridization (see, Hyrup, et al., 1996, supra; Perry-O'Keefe, et al., 1996. supra).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOVX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

NOVX Polypeptides

A polypeptide according to the invention includes a polypeptide including the amino acid sequence of NOVX polypeptides whose sequences are provided in SEQ ID NOS: 2, 4, 6, 8, 10, 12, 14, 16, 18, 20, 22, 24, and 26. The invention also includes a mutant or variant protein any of whose residues may be changed from the corresponding residues shown in SEQ ID NOS: 2, 4, 6, 8, 10, 12, 14, 16, 18, 20, 22, 24, and 26 while still encoding a protein that maintains its NOVX activities and physiological functions, or a functional fragment thereof.

In general, an NOVX variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically-active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, an NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated from cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein”), more preferably less than about 20% of non-NOVX proteins, still more preferably less than about 10% of non-NOVX proteins, and most preferably less than about 5% of non-NOVX proteins. When the NOVX protein or biologically-active portion thereof is recombinantly-produced, it is also preferably substantially free of culture medium, i.e., culture medium represents less than about 20%, more preferably less than about 10%, and most preferably less than about 5% of the volume of the NOVX protein preparation.

The language “substantially free of chemical precursors or other chemicals” includes preparations of NOVX proteins in which the protein is separated from chemical precursors or other chemicals that are involved in the synthesis of the protein. In one embodiment, the language “substantially free of chemical precursors or other chemicals” includes preparations of NOVX proteins having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically-active portions of NOVX proteins include peptides comprising amino acid sequences sufficiently homologous to or derived from the amino acid sequences of the NOVX proteins (e.g., the amino acid sequence shown in SEQ ID NOS: 2, 4, 6, 8, 10, 12, 14, 16, 18, 20, 22, 24, and 26) that include fewer amino acids than the full-length NOVX proteins, and exhibit at least one activity of an NOVX protein. Typically, biologically-active portions comprise a domain or motif with at least one activity of the NOVX protein. A biologically-active portion of an NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence shown SEQ ID NOS: 2, 4, 6, 8, 10, 12, 14, 16, 18, 20, 22, 24, and 26. In other embodiments, the NOVX protein is substantially homologous to SEQ ID NOS: 2, 4, 6, 8, 10, 12, 14, 16, 18, 20, 22, 24, and 26, and retains the functional activity of the protein of SEQ ID NOS: 2, 4, 6, 8, 10, 12, 14, 16, 18, 20, 22, 24, and 26, yet differs in amino acid sequence due to natural allelic variation or mutagenesis, as described in detail, below. Accordingly, in another embodiment, the NOVX protein is a protein that comprises an amino acid sequence at least about 45% homologous to the amino acid sequence SEQ ID NOS: 2, 4, 6, 8, 10, 12, 14, 16, 18, 20, 22, 24, and 26, and retains the functional activity of the NOVX proteins of SEQ ID NOS: 2, 4, 6, 8, 10, 12, 14, 16, 18,20,22,24, and 26.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NOS: 1, 3, 5, 7, 9, 11, 13, 15, 17, 19, 21, 23,and 25.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides NOVX chimeric or fusion proteins. As used herein, an NOVX “chimeric protein” or “fusion protein” comprises an NOVX polypeptide operatively-linked to a non-NOVX polypeptide. An “NOVX polypeptide” refers to a polypeptide having an amino acid sequence corresponding to an NOVX protein SEQ ID NOS: 2, 4, 6, 8, 10, 12, 14, 16, 18, 20, 22, 24, and 26), whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within an NOVX fusion protein the NOVX polypeptide can correspond to all or a portion of an NOVX protein. In one embodiment, an NOVX fusion protein comprises at least one biologically-active portion of an NOVX protein. In another embodiment, an NOVX fusion protein comprises at least two biologically-active portions of an NOVX protein. In yet another embodiment, an NOVX fusion protein comprises at least three biologically-active portions of an NOVX protein. Within the fusion protein, the term “operatively-linked” is intended to indicate that the NOVX polypeptide and the non-NOVX polypeptide are fused in-frame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides.

In another embodiment, the fusion protein is an NOVX protein containing a heterologous signal sequence at its N-terminus. In certain host cells (e.g., mammalian host cells), expression and/or secretion of NOVX can be increased through use of a heterologous signal sequence.

In yet another embodiment, the fusion protein is an NOVX-immunoglobulin fusion protein in which the NOVX sequences are fused to sequences derived from a member of the immunoglobulin protein family. The NOVX-immunoglobulin fusion proteins of the invention can be incorporated into pharmaceutical compositions and administered to a subject to inhibit an interaction between an NOVX ligand and an NOVX protein on the surface of a cell, to thereby suppress NOVX-mediated signal transduction in vivo. The NOVX-immunoglobulin fusion proteins can be used to affect the bioavailability of an NOVX cognate ligand. Inhibition of the NOVX ligand/NOVX interaction may be useful therapeutically for both the treatment of proliferative and differentiative disorders, as well as modulating (e.g. promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with an NOVX ligand.

An NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). An NOVX-encoding nucleic acid can be cloned into such an expression vector such that the fusion moiety is linked in-frame to the NOVX protein.

NOVX Agonists and Antagonists

The invention also pertains to variants of the NOVX proteins that function as either NOVX agonists (i.e., mimetics) or as NOVX antagonists. Variants of the NOVX protein can be generated by mutagenesis (e.g., discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX proteins that function as either NOVX agonists (i.e., mimetics) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the NOVX protein coding sequences can be used to generate a variegated population of NOVX fragments for screening and subsequent selection of variants of an NOVX protein. In one embodiment, a library of coding sequence fragments can be generated by treating a double stranded PCR fragment of an NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S₁ nuclease, and ligating the resulting fragment library into an expression vector. By this method, expression libraries can be derived which encodes N-terminal and internal fragments of various sizes of the NOVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX variants. See, e.g., Arkin and Yourvan, 1992. Proc. Natl. Acad. Sci. USA 89: 7811-7815; Delgrave, et al., 1993. Protein Engineering 6:327-331.

Anti-NOVX Antibodies

The invention encompasses antibodies and antibody fragments, such as F_{ab} or (F_{ab})₂, that bind immunospecifically to any of the NOVX polypeptides of said invention.

An isolated NOVX protein, or a portion or fragment thereof, can be used as an immunogen to generate antibodies that bind to NOVX polypeptides using standard techniques for polyclonal and monoclonal antibody preparation. The full-length NOVX proteins can be used or, alternatively, the invention provides antigenic peptide fragments of NOVX proteins for use as immunogens. The antigenic NOVX peptides comprises at least 4 amino acid residues of the amino acid sequence shown SEQ ID NOS: 2, 4, 6, 8, 10, 12, 14, 16, 18, 20, 22, 24, and 26 and encompasses an epitope of NOVX such that an antibody raised against the peptide forms a specific immune complex with NOVX. Preferably, the antigenic peptide comprises at least 6, 8, 10, 15, 20, or 30 amino acid residues. Longer antigenic peptides are sometimes preferable over shorter antigenic peptides, depending on use and according to methods well known to someone skilled in the art.

In certain embodiments of the invention, at least one epitope encompassed by the antigenic peptide is a region of NOVX that is located on the surface of the protein (e.g., a hydrophilic region). As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation (see, e.g., Hopp and Woods, 1981. Proc. Nat. Acad. Sci. USA 78: 3824-3828; Kyte and Doolittle, 1982. J. Mol. Biol. 157: 105-142, each incorporated herein by reference in their entirety).

As disclosed herein, NOVX protein sequences of SEQ ID NOS: 2, 4, 6, 8, 10, 12, 14, 16, 18, 20, 22, 24, 26, or derivatives, fragments, analogs or homologs thereof, may be utilized as immunogens in the generation of antibodies that immunospecifically-bind these protein components. The term “antibody” as used herein refers to immunoglobulin molecules and immunologically-active portions of immunoglobulin molecules, i.e., molecules that contain an antigen binding site that specifically-binds (immunoreacts with) an antigen, such as NOVX. Such antibodies include, but are not limited to, polyclonal, monoclonal, chimeric, single chain, F_{ab} and F(_{ab′})₂ fragments, and an F_{ab} expression library. In a specific embodiment, antibodies to human NOVX proteins are disclosed. Various procedures known within the art may be used for the production of polyclonal or monoclonal antibodies to an NOVX protein sequence of SEQ ID NOS: 2, 4, 6, 8, 10, 12, 14, 16, 18, 20, 22, 24, 26, or a derivative, fragment, analog or homolog thereof. Some of these proteins are discussed below.

For the production of polyclonal antibodies, various suitable host animals (e.g., rabbit, goat, mouse or other mammal) may be immunized by injection with the native protein, or a synthetic variant thereof, or a derivative of the foregoing. An appropriate immunogenic preparation can contain, for example, recombinantly-expressed NOVX protein or a chemically-synthesized NOVX polypeptide. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), human adjuvants such as Bacille Calmette-Guerin and Corynebacterium parvum, or similar immunostimulatory agents. If desired, the antibody molecules directed against NOVX can be isolated from the mammal (e.g., from the blood) and further purified by well known techniques, such as protein A chromatography to obtain the IgG fraction.

The term “monoclonal antibody” or “monoclonal antibody composition”, as used herein, refers to a population of antibody molecules that contain only one species of an antigen binding site capable of immunoreacting with a particular epitope of NOVX. A monoclonal antibody composition thus typically displays a single binding affinity for a particular NOVX protein with which it immunoreacts. For preparation of monoclonal antibodies directed towards a particular NOVX protein, or derivatives, fragments, analogs or homologs thereof, any technique that provides for the production of antibody molecules by continuous cell line culture may be utilized. Such techniques include, but are not limited to, the hybridoma technique (see, e.g., Kohler & Milstein, 1975. Nature 256: 495-497); the trioma technique; the human B-cell hybridoma technique (see, e.g., Kozbor, et al., 1983. Immunol. Today 4: 72) and the EBV hybridoma technique to produce human monoclonal antibodies (see, e.g., Cole, et al., 1985. In: MONOCLONAL ANTIBODIES AND CANCER THERAPY, Alan R. Liss, Inc., pp. 77-96). Human monoclonal antibodies may be utilized in the practice of the invention and may be produced by using human hybridomas (see, e.g., Cote, et al., 1983. Proc Natl,4cad Sci USA 80: 2026-2030) or by transforming human B-cells with Epstein Barr Virus in vitro (see, e.g., Cole, et aL, 1985. In: MONOCLONAL ANTIBODIES AND CANCER THERAPY, Alan R. Liss, Inc., pp. 77-96). Each of the above citations is incorporated herein by reference in their entirety.

According to the invention, techniques can be adapted for the production of single-chain antibodies specific to an NOVX protein (see, e.g., U.S. Pat. No. 4,946,778). In addition, methods can be adapted for the construction of F_{ab} expression libraries (see, e.g., Huse, et al., 1989. Science 246: 1275-1281) to allow rapid and effective identification of monoclonal F_{ab} fragments with the desired specificity for an NOVX protein or derivatives, fragments, analogs or homologs thereof. Non-human antibodies can be “humanized” by techniques well known in the art. See, e.g., U.S. Pat. No. 5,225,539. Antibody fragments that contain the idiotypes to an NOVX protein may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent; and (iv) Fᵥ fragments.

Additionally, recombinant anti-NOVX antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in International Application No. PCT/US86/02269; European Patent Application No. 184,187; European Patent Application No. 171,496; European Patent Application No. 173,494; PCT International Publication No. WO 86/01533; U.S. Pat. No. 4,816,567; U.S. Pat. No. 5,225,539; European Patent Application No. 125,023; Better, et al., 1988. Science 240: 1041-1043; Liu, et al., 1987. Proc. Natl. Acad. Sci. USA 84: 3439-3443; Liu, et al., 1987. J. Immunol. 139: 3521-3526; Sun, et al., 1987. Proc. Natl. Acad. Sci. USA 84: 214-218; Nishimura, et al., 1987. Cancer Res. 47: 999-1005; Wood, et al., 1985. Nature 314 :446-449; Shaw, etal., 1988. J. Natl. Cancer Inst. 80: 1553-1559); Morrison(1985) Science 229:1202-1207; Oi, et al. (1986) BioTechniques 4:214; Jones, et al., 1986. Nature 321: 552-525; Verhoeyan, et al., 1988. Science 239: 1534; and Beidler, et al., 1988. J. Immunol. 141: 4053-4060. Each of the above citations are incorporated herein by reference in their entirety.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of an NOVX protein is facilitated by generation of hybridomas that bind to the fragment of an NOVX protein possessing such a domain. Thus, antibodies that are specific for a desired domain within an NOVX protein, or derivatives, fragments, analogs or homologs thereof, are also provided herein.

Anti-NOVX antibodies may be used in methods known within the art relating to the localization and/or quantitation of an NOVX protein (e.g., for use in measuring levels of the NOVX protein within appropriate physiological samples, for use in diagnostic methods, for use in imaging the protein, and the like). In a given embodiment, antibodies for NOVX proteins, or derivatives, fragments, analogs or homologs thereof, that contain the antibody derived binding domain, are utilized as pbarmacologically-active compounds (hereinafter “Therapeutics”).

An anti-NOVX antibody (e.g., monoclonal antibody) can be used to isolate an NOVX polypeptide by standard techniques, such as affinity chromatography or immunoprecipitation. An anti-NOVX antibody can facilitate the purification of natural NOVX polypeptide from cells and of recombinantly-produced NOVX polypeptide expressed in host cells. Moreover, an anti-NOVX antibody can be used to detect NOVX protein (e.g., in a cellular lysate or cell supernatant) in order to evaluate the abundance and pattern of expression of the NOVX protein. Anti-NOVX antibodies can be used diagnostically to monitor protein levels in tissue as part of a clinical testing procedure, e.g., to, for example, determine the efficacy of a given treatment regimen. Detection can be facilitated by coupling (i. e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

NOVX Recombinant Expression Vectors and Host Cells

Another aspect of the invention pertains to vectors, preferably expression vectors, containing a nucleic acid encoding an NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRITS (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOVX expression vector is a yeast expression vector. Examples of vectors for expression in yeast Saccharomyces cerivisae include pYepSec1 (Baldari, et al., 1987. EMBO J. 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, NOVX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOVX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

Transgenic NOVX Animals

The host cells of the invention can also be used to produce non-human transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOVX-encoding nucleic acid into the male pronuclei of a fertilized oocyte (e.g., by microinjection, retroviral infection) and allowing the oocyte to develop in a pseudopregnant female foster animal. The human NOVX cDNA sequences SEQ ID NOS: 1, 3, 5, 7, 9, 11, 13, 15, 17, 19, 21, 23, and 25 can be introduced as a transgene into the genome of a non-human animal. Alternatively, a non-human homologue of the human NOVX gene, such as a mouse NOVX gene, can be isolated based on hybridization to the human NOVX cDNA (described further supra) and used as a transgene. Intronic sequences and polyadenylation signals can also be included in the transgene to increase the efficiency of expression of the transgene. A tissue-specific regulatory sequence(s) can be operably-linked to the NOVX transgene to direct expression of NOVX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the NOVX transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of an NOVX gene into which a deletion, addition or substitution has been introduced to thereby alter, e.g., functionally disrupt, the NOVX gene. The NOVX gene can be a human gene (e.g., the cDNA of SEQ ID NOS: 1, 3, 5, 7, 9, 11, 13, 15, 17, 19, 21, 23, and 25), but more preferably, is a non-human homologue of a human NOVX gene. For example, amouse homologue of human NOVX gene of SEQ ID NOS: 1, 3, 5, 7, 9, 11, 13, 15, 17, 19, 21, 23, and 25 can be used to construct a homologous recombination vector suitable for altering an endogenous NOVX gene in the mouse genome. In one embodiment, the vector is designed such that, upon homologous recombination, the endogenous NOVX gene is functionally disrupted (i.e., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′- and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113- 152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g. through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g. U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in an NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease(possesses anti-microbial activity) and the various dyslipidemias. In addition, the anti-NOVX antibodies of the invention can be used to detect and isolate NOVX proteins and modulate NOVX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein.

In one embodiment, the invention provides assays for screening candidate or test compounds which bind to or modulate the activity of the membrane-bound form of an NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37:1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOVX protein, or a biologically-active portion thereof, on the cell surface is contacted with a test compound and the ability of the test compound to bind to an NOVX protein determined. The cell, for example, can of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity of the NOVX protein or biologically-active portion thereof. Determining the ability of the test compound to modulate the activity of NOVX or a biologically-active portion thereof can be accomplished, for example, by determining the ability of the NOVX protein to bind to or interact with an NOVX target molecule. As used herein, a “target molecule” is a molecule with which an NOVX protein binds or interacts in nature, for example, a molecule on the surface of a cell which expresses an NOVX interacting protein, a molecule on the surface of a second cell, a molecule in the extracellular milieu, a molecule associated with the internal surface of a cell membrane or a cytoplasmic molecule. An NOVX target molecule can be a non-NOVX molecule or an NOVX protein or polypeptide of the invention. In one embodiment, an NOVX target molecule is a component of a signal transduction pathway that facilitates transduction of an extracellular signal (e.g. a signal generated by binding of a compound to a membrane-bound NOVX molecule) through the cell membrane and into the cell. The target, for example, can be a second intercellular protein that has catalytic activity or a protein that facilitates the association of downstream signaling molecules with NOVX.

Determining the ability of the NOVX protein to bind to or interact with an NOVX target molecule can be accomplished by one of the methods described above for determining direct binding. In one embodiment, determining the ability of the NOVX protein to bind to or interact with an NOVX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising an NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting an NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining the ability of the test compound to modulate (e.g. stimulate or inhibit) the activity of the NOVX protein or biologically-active portion thereof. Determining the ability of the test compound to modulate the activity of NOVX can be accomplished, for example, by determining the ability of the NOVX protein to bind to an NOVX target molecule by one of the methods described above for determining direct binding. In an alternative embodiment, determining the ability of the test compound to modulate the activity of NOVX protein can be accomplished by determining the ability of the NOVX protein further modulate an NOVX target molecule. For example, the catalytic/enzymatic activity of the target molecule on an appropriate substrate can be determined as described, supra.

In yet another embodiment, the cell-free assay comprises contacting the NOVX protein or biologically-active portion thereof with a known compound which binds NOVX protein to form an assay mixture, contacting the assay mixture with a test compound, and determining the ability of the test compound to interact with an NOVX protein, wherein determining the ability of the test compound to interact with an NOVX protein comprises determining the ability of the NOVX protein to preferentially bind to or modulate the activity of an NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl—N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptavidin. Biotinylated NOVX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (i.e., statistically significantly greater) in the presence of the candidate compound than in its absence, the candidate compound is identified as a stimulator of NOVX mRNA or protein expression. Alternatively, when expression of NOVX mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also likely to be involved in the propagation of signals by the NOVX proteins as, for example, upstream or downstream elements of the NOVX pathway.

The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an NOVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the NOVX sequences, SEQ ID NOS: 1, 3, 5, 7, 9, 11, 13, 15, 17, 19, 21, 23, and 25, or fragments or derivatives thereof, can be used to map the location of the NOVX genes, respectively, on a chromosome. The mapping of the NOVX sequences to chromosomes is an important first step in correlating these sequences with genes associated with disease.

Briefly, NOVX genes can be mapped to chromosomes by preparing PCR primers (preferably 15-25 bp in length) from the NOVX sequences. Computer analysis of the NOVX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOVX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the NOVX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The NOVX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the NOVX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If predicted coding sequences, such as those in SEQ ID NOS: 1, 3, 5, 7, 9, 11, 13, 15, 17, 19, 21, 23, and 25 are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with NOVX protein, nucleic acid expression or activity. For example, mutations in an NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of NOVX in clinical trials.

These and other agents are described in further detail in the following sections.

Diagnostic Assays

An exemplary method for detecting the presence or absence of NOVX in a biological sample involves obtaining a biological sample from a test subject and contacting the biological sample with a compound or an agent capable of detecting NOVX protein or nucleic acid (e.g., mRNA, genomic DNA) that encodes NOVX protein such that the presence of NOVX is detected in the biological sample. An agent for detecting NOVX mRNA or genomic DNA is a labeled nucleic acid probe capable of hybridizing to NOVX mRNA or genomic DNA. The nucleic acid probe can be, for example, a full-length NOVX nucleic acid, such as the nucleic acid of SEQ ID NOS: 1, 3, 5, 7, 9, 11, 13, 15, 17, 19, 21, 23, and 25, or a portion thereof, such as an oligonucleotide of at least 15, 30, 50, 100, 250 or 500 nucleotides in length and sufficient to specifically hybridize under stringent conditions to NOVX mRNA or genomic DNA. Other suitable probes for use in the diagnostic assays of the invention are described herein.

An agent for detecting NOVX protein is an antibody capable of binding to NOVX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vitro techniques for detection of NOVX mRNA include Northern hybridizations and in situ hybridizations. In vitro techniques for detection of NOVX protein include enzyme linked immunosorbent assays (ELISAs), Western blots, immunoprecipitations, and immunofluorescence. In vitro techniques for detection of NOVX genomic DNA include Southern hybridizations. Furthermore, in vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting NOVX protein, mRNA, or genomic DNA, such that the presence of NOVX protein, mRNA or genomic DNA is detected in the biological sample, and comparing the presence of NOVX protein, mRNA or genomic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and means for comparing the amount of NOVX in the sample with a standard. The compound or agent can be packaged in a suitable container. The kit can further comprise instructions for using the kit to detect NOVX protein or nucleic acid.

Prognostic Assays

The diagnostic methods described herein can furthermore be utilized to identify subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Alternatively, the prognostic assays can be utilized to identify a subject having or at risk for developing a disease or disorder. Thus, the invention provides a method for identifying a disease or disorder associated with aberrant NOVX expression or activity in which a test sample is obtained from a subject and NOVX protein or nucleic acid (e.g., mRNA, genomic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated with an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in an NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding an NOVX-protein, or the misexpression of the NOVX gene. For example, such genetic lesions can be detected by ascertaining the existence of at least one of: (i) a deletion of one or more nucleotides from an NOVX gene; (ii) an addition of one or more nucleotides to an NOVX gene; (iii) a substitution of one or more nucleotides of an NOVX gene, (iv) a chromosomal rearrangement of an NOVX gene; (v) an alteration in the level of a messenger RNA transcript of an NOVX gene, (vi) aberrant modification of an NOVX gene, such as of the methylation pattern of the genomic DNA, (vii) the presence of a non-wild-type splicing pattern of a messenger RNA transcript of an NOVX gene, (viii) a non-wild-type level of an NOVX protein, (ix) allelic loss of an NOVX gene, and (x) inappropriate post-translational modification of an NOVX protein. As described herein, there are a large number of assay techniques known in the art which can be used for detecting lesions in an NOVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOVX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to an NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, etal., 1990. Proc. NatL. Acad. Sci. USA 87:1874-1878), transcriptional amplification system (see, Kwoh, et al., 1989. Proc. Nat. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in NOVX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the NOVX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on an NOVX sequence, e.g., a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOVX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control NOVX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOVX activity (e.g., NOVX gene expression), as identified by a screening assay described herein can be administered to individuals to treat (prophylactically or therapeutically) disorders (The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers.) In conjunction with such treatment, the pharrnacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C 19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with an NOVX modulator, such as a modulator identified by one of the exemplary screening assays described herein.

Monitoring of Effects During Clinical Trials

Monitoring the influence of agents (e.g., drugs, compounds) on the expression or activity of NOVX (e.g., the ability to modulate aberrant cell proliferation and/or differentiation) can be applied not only in basic drug screening, but also in clinical trials. For example, the effectiveness of an agent determined by a screening assay as described herein to increase NOVX gene expression, protein levels, or upregulate NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markers of the immune responsiveness of a particular cell.

By way of example, and not of limitation, genes, including NOVX, that are modulated in cells by treatment with an agent (e.g., compound, drug or small molecule) that modulates NOVX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of an NOVX protein, mRNA, or genomic DNA in the preadministration sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of NOVX to higher levels than detected, i.e., to increase the effectiveness of the agent. Alternatively, decreased administration of the agent may be desirable to decrease expression or activity of NOVX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOVX expression or activity. The disorders include cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, prostate cancer, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, and other diseases, disorders and conditions of the like.

These methods of treatment will be discussed more fully, below.

Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators ( i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i. e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, an NOVX agonist or NOVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of an NOVX protein, a peptide, an NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of an NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering an NOVX protein or nucleic acid molecule as therapy to compensate for reduced or aberrant NOVX expression or activity.

Stimulation of NOVX activity is desirable in situations in which NOVX is abnormally downregulated and/or in which increased NOVX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The NOVX nucleic acids and proteins of the invention are useful in potential prophylactic and therapeutic applications implicated in a variety of disorders including, but not limited to: metabolic disorders, diabetes, obesity, infectious disease, anorexia, cancer-associated cancer, neurodegenerative disorders, Alzheimer's Disease, Parkinson's Disorder, immune disorders, hematopoietic disorders, and the various dyslipidemias, metabolic disturbances associated with obesity, the metabolic syndrome X and wasting disorders associated with chronic diseases and various cancers.

As an example, a cDNA encoding the NOVX protein of the invention may be useful in gene therapy, and the protein may be useful when administered to a subject in need thereof. By way of non-limiting example, the compositions of the invention will have efficacy for treatment of patients suffering from: metabolic disorders, diabetes, obesity, infectious disease, anorexia, cancer-associated cachexia, cancer, neurodegenerative disorders, Alzheimer's Disease, Parkinson's Disorder, immune disorders, hematopoietic disorders, and the various dyslipidemias.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies which immunospecifically-bind to the novel substances of the invention for use in therapeutic or diagnostic methods.

### EQUIVALENTS

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims.

What is claimed is:

1. An isolated polypeptide comprising an amino acid sequence selected from the group consisting of:
(a) a mature form of an amino acid sequence selected from the group consisting of SEQ ID NOS: 2, 4, 6, 8, 10, 12, 14, 16, 18, 20, 22, 24, and 26;
(b) a variant of a mature form of an amino acid sequence selected from the group consisting of SEQ ID NOS: 2, 4, 6, 8, 10, 12, 14, 16, 18, 20, 22, 24, and 26, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting SEQ ID NOS: 2, 4, 6, 8, 10, 12, 14, 16, 18, 20, 22, 24, and 26; and
(d) a variant of an amino acid sequence selected from the group consisting of SEQ ID NOS: 2, 4, 6, 8, 10, 12, 14, 16, 18, 20, 22, 24, and 26, wherein one or more amino acid residues in said variant differs from the amino acid sequence of said mature form, provided that said variant differs in no more than 15% of amino acid residues from said amino acid sequence.

2 The polypeptide of claim 1, wherein said polypeptide comprises the amino acid sequence of a naturally-occurring allelic variant of an amino acid sequence selected from the group consisting SEQ ID NOS: 2, 4, 6, 8, 10, 12, 14, 16, 18, 20, 22, 24, and 26.

3. The polypeptide of claim 2, wherein said allelic variant comprises an amino acid sequence that is the translation of a nucleic acid sequence differing by a single nucleotide from a nucleic acid sequence selected from the group consisting of SEQ ID NOS: 1, 3, 5, 7, 9, 11, 13, 15, 17, 19, 21, 23, and 25.

4. The polypeptide of claim 1, wherein the amino acid sequence of said variant comprises a conservative amino acid substitution.

5. An isolated nucleic acid molecule comprising a nucleic acid sequence encoding a polypeptide comprising an amino acid sequence selected from the group consisting of:
(a) a mature form of an amino acid sequence selected from the group consisting of SEQ ID NOS: 2, 4, 6, 8, 10, 12, 14, 16, 18, 20, 22, 24, and 26;
(b) a variant of a mature form of an amino acid sequence selected from the group consisting of SEQ ID NOS: 2, 4, 6, 8, 10, 12, 17, 19, 21, 23, 25, 29, 31, 33, 35, 37, 83, and 85, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS: 2, 4, 6, 8, 10, 12, 14, 16, 18, 20, 22, 24, and 26;
(d) a variant of an amino acid sequence selected from the group consisting SEQ ID NOS: 2, 4, 6, 8, 10, 12, 14, 16, 18, 20, 22, 24, and 26, wherein one or more amino acid residues in said variant differs from the amino acid sequence of said mature form, provided that said variant differs in no more than 15% of amino acid residues from said amino acid sequence;
(e) a nucleic acid fragment encoding at least a portion of a polypeptide comprising an amino acid sequence chosen from the group consisting of SEQ ID NOS: 2, 4, 6, 8, 10, 12, 14, 16, 18, 20, 22, 24, and 26, or a variant of said polypeptide, wherein one or more amino acid residues in said variant differs from the amino acid sequence of said mature form, provided that said variant differs in no more than 15% of amino acid residues from said amino acid sequence; and
(f) a nucleic acid molecule comprising the complement of (a), (b), (c), (d) or (e).

6. The nucleic acid molecule of claim 5, wherein the nucleic acid molecule comprises the nucleotide sequence of a naturally-occurring allelic nucleic acid variant.

7. The nucleic acid molecule of claim 5, wherein the nucleic acid molecule encodes a polypeptide comprising the amino acid sequence of a naturally-occurring polypeptide variant.

8. The nucleic acid molecule of claim 5, wherein the nucleic acid molecule differs by a single nucleotide from a nucleic acid sequence selected from the group consisting of SEQ ID NOS: 1, 3, 5, 7, 9, 11, 13, 15, 17, 19, 21, 23, and 25.

9. The nucleic acid molecule of claim 5, wherein said nucleic acid molecule comprises a nucleotide sequence selected from the group consisting of:
(a) a nucleotide sequence selected from the group consisting of SEQ ID NOS: 1, 3, 5, 7, 9, 11, 13, 15, 17, 19, 21, 23, and 25;
(b) a nucleotide sequence differing by one or more nucleotides from a nucleotide sequence selected from the group consisting of SEQ ID NOS: 1, 3, 5, 7, 9, 11, 13, 15, 17, 19, 21, 23, and 25, provided that no more than 20% of the nucleotides differ from said nucleotide sequence;
(c) a nucleic acid fragment of (a); and
(d) a nucleic acid fragment of (b).

10. The nucleic acid molecule of claim 5, wherein said nucleic acid molecule hybridizes under stringent conditions to a nucleotide sequence chosen from the group consisting SEQ ID NOS: 1, 3, 5, 7, 9, 11, 13, 15, 17, 19, 21, 23, and25,oracomplementofsaid nucleotide sequence.

11. The nucleic acid molecule of claim 5, wherein the nucleic acid molecule comprises a nucleotide sequence selected from the group consisting of:
(a) a first nucleotide sequence comprising a coding sequence differing by one or more nucleotide sequences from a coding sequence encoding said amino acid sequence, provided that no more than 20% of the nucleotides in the coding sequence in said first nucleotide sequence differ from said coding sequence;
(b) an isolated second polynucleotide that is a complement of the first polynucleotide; and
(c) a nucleic acid fragment of (a) or (b).

12. A vector comprising the nucleic acid molecule of claim 11.

13. The vector of claim 12, further comprising a promoter operably-linked to said nucleic acid molecule.

14. A cell comprising the vector of claim 12.

15. An antibody that binds immunospecifically to the polypeptide of claim 1.

16. The antibody of claim 15, wherein said antibody is a monoclonal antibody.

17. The antibody of claim 15, wherein the antibody is a humanized antibody.

18. A method for determining the presence or amount of the polypeptide of claim 1 in a sample, the method comprising:
(a) providing the sample;
(b) contacting the sample with an antibody that binds immunospecifically to the polypeptide; and
(c) determining the presence or amount of antibody bound to said polypeptide, thereby determining the presence or amount of polypeptide in said sample.

19. A method for determining the presence or amount of the nucleic acid molecule of claim 5 in a sample, the method comprising:
(a) providing the sample;
(b) contacting the sample with a probe that binds to said nucleic acid molecule;
and
(c) determining the presence or amount of the probe bound to said nucleic acid molecule,
thereby determining the presence or amount of the nucleic acid molecule in said sample.

20. The method of claim 19 wherein presence or amount of the nucleic acid molecule is used as a marker for cell or tissue type.

21. The method of claim 20 wherein the cell or tissue type is cancerous.

22. A method of identifying an agent that binds to a polypeptide of claim 1, the method comprisng:
(a) contacting said polypeptide with said agent; and
(b) determining whether said agent binds to said polypeptide.

23. The method of claim 22 wherein the agent is a cellular receptor or a downstream effector.

24. A method for identifying an agent that modulates the expression or activity of the polypeptide of claim 1, the method comprising:
(a) providing a cell expressing said polypeptide;
(b) contacting the cell with said agent, and
(c) determining whether the agent modulates expression or activity of said polypeptide,
whereby an alteration in expression or activity of said peptide indicates said agent modulates expression or activity of said polypeptide.

25. A method for modulating the activity of the polypeptide of claim 1, the method comprising contacting a cell sample expressing the polypeptide of said claim with a compound that binds to said polypeptide in an amount sufficient to modulate the activity of the polypeptide.

26. A method of treating or preventing a NOVX-associated disorder, said method comprising administering to a subject in which such treatment or prevention is desired the polypeptide of claim 1 in an amount sufficient to treat or prevent said NOVX-associated disorder in said subject.

27. The method of claim 26 wherein the disorder is selected from the group consisting of cardiomyopathy and atherosclerosis.

28. The method of claim 26 wherein the disorder is related to cell signal processing and metabolic pathway modulation.

29. The method of claim 26, wherein said subject is a human.

30. A method of treating or preventing a NOVX-associated disorder, said method comprising administering to a subject in which such treatment or prevention is desired the nucleic acid of claim 5 in an amount sufficient to treat or prevent said NOVX-associated disorder in said subject.

31. The method of claim 30 wherein the disorder is selected from the group consisting of cardiomyopathy and atherosclerosis.

32. The method of claim 30 wherein the disorder is related to cell signal processing and metabolic pathway modulation.

33. The method of claim 30, wherein said subject is a human.

34. A method of treating or preventing a NOVX-associated disorder, said method comprising administering to a subject in which such treatment or prevention is desired the antibody of claim 15 in an amount sufficient to treat or prevent said NOVX-associated disorder in said subject.

35. The method of claim 34 wherein the disorder is diabetes.

36. The method of claim 34 wherein the disorder is related to cell signal processing and metabolic pathway modulation.

37. The method of claim 34, wherein the subject is a human.

38. A pharmaceutical composition comprising the polypeptide of claim 1 and a pharmaceutically-acceptable carrier.

39. A pharmaceutical composition comprising the nucleic acid molecule of claim 5 and a pharmaceutically-acceptable carrier.

40. A pharmaceutical composition comprising the antibody of claim 15 and a pharmaceutically-acceptable carrier.

41. A kit comprising in one or more containers, the pharmaceutical composition of claim 38.

42. A kit comprising in one or more containers, the pharmaceutical composition of claim 39.

43. A kit comprising in one or more containers, the pharmaceutical composition of claim 40.

44. A method for determining the presence of or predisposition to a disease associated with altered levels of the polypeptide of claim 1 in a first mammalian subject, the method comprising:
(a) measuring the level of expression of the polypeptide in a sample from the first mammalian subject; and
(b) comparing the amount of said polypeptide in the sample of step (a) to the amount of the polypeptide present in a control sample from a second mammalian subject known not to have, or not to be predisposed to, said disease;
wherein an alteration in the expression level of the polypeptide in the first subject as compared to the control sample indicates the presence of or predisposition to said disease.

45. The method of claim 44 wherein the predisposition is to cancers.

46. A method for determining the presence of or predisposition to a disease associated with altered levels of the nucleic acid molecule of claim 5 in a first mammalian subject, the method comprising:
(a) measuring the amount of the nucleic acid in a sample from the first mammalian subject; and
(b) comparing the amount of said nucleic acid in the sample of step (a) to the amount of the nucleic acid present in a control sample from a second mammalian subject known not to have or not be predisposed to, the disease;
wherein an alteration in the level of the nucleic acid in the first subject as compared to the control sample indicates the presence of or predisposition to the disease.

47. The method of claim 46 wherein the predisposition is to a cancer.

48. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising an amino acid sequence of at least one of SEQ ID NOS: 2, 4, 6, 8, 10, 12, 14, 16, 18, 20, 22, 24, and 26, or a biologically active fragment thereof.

49. A method of treating a pathological state in a mammal, the method comprising administering to the mammal the antibody of claim 15 in an amount sufficient to alleviate the pathological state.
